(19) United States
(12) Patent Application Publication
(10) Pub. No.: US 2024/0342185 A1
(43) Pub. Date: Oct. 17, 2024

(54) SMALL MOLECULES INHIBITORS OF CYCLIC GMP-AMP SYNTHASE (cGAS)

(71) Applicants: THE SCRIPPS RESEARCH INSTITUTE, La Jolla, CA (US); SHANGPHARMA INNOVATION INC., South San Francisco, CA (US)

(72) Inventors: Luke L. LAIRSON, San Diego, CA (US); Emily CHIN, San Diego, CA (US); Brett BOOKSER, Kensington, CA (US); David LAPOINTE, South San Francisco, CA (US)

(21) Appl. No.: 18/578,875

(22) PCT Filed: Jul. 13, 2022
(86) PCT No.: PCT/US22/73696

§ 371 Date: Jan. 12, 2024

Related U.S. Application Data
(60) Provisional application No. 63/203,219, filed on Jul. 13, 2021.

(51) Int. Cl. A61K 31/5377; A61K 31/422; A61K 31/454; A61K 31/4545; C07D 413/04; C07D 413/06; C07D 413/14

(52) U.S. Cl. CPC A61K 31/5377; A61K 31/422; A61K 31/454; A61K 31/4545; C07D 413/04; C07D 413/06; C07D 413/14

(57) ABSTRACT

Provided herein are compounds of Formula (I), their pharmaceutically acceptable salts, and their pharmaceutical compositions: (I) wherein R¹, R², R³, L, X, and Ar are defined in the present disclosure. The compounds are inhibitors of cyclic gmp-amp synthase (cGAS) or CGAS-related cGAMP production, and they are useful in treating or preventing inflammatory diseases or conditions in a subject.

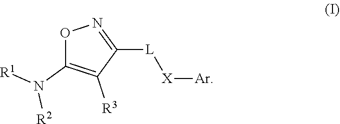

This application claims the benefit of priority to U.S. Provisional Patent Application No. 63/203,219 filed on Jul. 13, 2021, which application is incorporated as if fully set forth herein.

### BACKGROUND

Upon binding to cytosolic dsDNA, the nucleotide cyclase cGAS uses ATP and GTP to synthesize the cyclic dinucleotide secondary messenger, 2′3′-cyclic GMP-AMP (cGAMP).

This results in activation of type I interferon signaling via the endoplasmic reticulum (ER) localized membrane associated stimulator of interferon genes (STING) receptor protein, which facilitates TBK1-dependent phosphorylation of transcription factor, IRF-3, and subsequent activation of type I interferon transcription. In addition to its clear role in inducing an innate immune response to pathogenic infection, the cGAS-STING pathway also serves as a direct link between inflammation and diverse physiological processes including: micronuclei surveillance in the context of DNA damage, age-associated inflammation, mitochondrial DNA-related inflammatory phenotypes, and microbiome-dependent intestinal homeostasis.

Aberrant activation of this pathway has been implicated in a large number of inflammatory disease settings.¹ These include type I interferonopathies and related diseases,² as well as inflammatory diseases associated with pathological presentation of nucleic acids derived from the mitochondria or nucleus.³ Excess activation of the cGAS-STING pathway is operative in autoimmune diseases, such as systemic lupus erythematosus⁴⁻⁶ and rheumatoid arthritis⁷. Inhibiting cGAS or the pathway is a viable target in treating various neurological disorders, including ischaemic brain injury,⁸ Parkinson's disease,⁹ general neurodegeneration,¹⁰ Huntington's disease,¹¹ amyotrophic lateral sclerosis and frontotemporal dementia,^{12,13} and traumatic brain injury.¹⁴ Loss of cGAS expression or inhibition of cGAS function also plays an essential role in senescence-associated inflammatory diseases or disorders,¹⁵⁻¹⁷ including age-dependent macular degeneration,¹⁸ atherosclerosis,¹⁹ and osteoarthritis²⁰.

### SUMMARY

There is a need for non-toxic small molecule inhibitors of cGAS possessing broad utilities in diverse inflammatory disease settings. The present disclosure addresses these needs and others by providing direct inhibitors of cGAS-dependent cGAMP production and downstream pathway activation in human cells. In various embodiments, the inhibitors are useful in a method of treating an inflammatory disease or condition in a subject suffering therefrom. The method comprises administering to the subject a compound of Formula (I) or a pharmaceutically acceptable salt thereof:

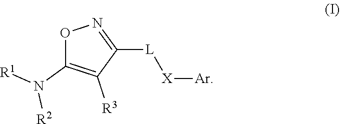

Substituents R¹ and R² are independently selected from the group consisting of H, C₁-C₆-alkyl (optionally substituted by one to five substituents independently selected from halo, CN, and OH), —C(O)C₁-C₆-alkyl, —C(O)H, —C₁-C₆-alkyl-(C₆-C₁₀-aryl), —C₁-C₆-alkyl-(C₁-C₆-alkoxy), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S), and —(C₁-C₆-alkyl)-SO₂—(C₁-C₆-alkyl), —(CH₂CH₂O)ₙ—R (wherein n is an integer selected from 2, 3, 4, 5, 6, 7, 8, 9, and 10, and R is H or C₁-C₆-alkyl).

Substituent R³ is selected from the group consisting of H, halo, C₁-C₆-alkyl, C₁-C₆-haloalkyl, and C₁-C₆-alkoxy.

L is a moiety selected from the group consisting of:

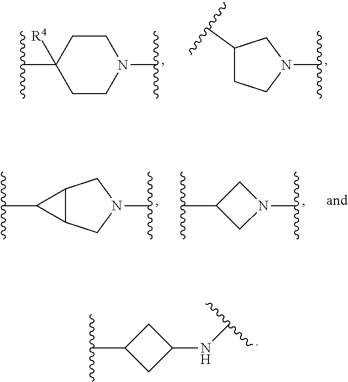

Substituent R⁴ is selected from the group consisting of H, halo, and C₁-C₆-alkyl.

X is —C(O)— or —SO₂—.

Ar is phenyl or indolyl. Ar is optionally substituted with one to five substituents selected from the group consisting of halo, C₁-C₆-alkyl, C₁-C₆-haloalkyl, C₁-C₆-alkoxy, C₁-C₆-haloalkoxy, CN, OH, NO₂, —NRR′ (wherein R and R′ are independently selected from H and C₁-C₆-alkyl), —(C₁-C₆-alkyl)NRR′, —C(O)NRR′, —SO₂R, C₆-C₁₀-aryl, 3- to 6-membered heterocycloalkyl (wherein 1-4 ring members are independently selected from N, O, and S), 5- to 10-membered heteroaryl (wherein 1-4 heteroaryl members are independently selected from N, O, and S), and C₃-C₁₀-cycloalkyl.

In additional embodiments, the present disclosure also provides a compound of Formula (I) or a pharmaceutically acceptable salt thereof:

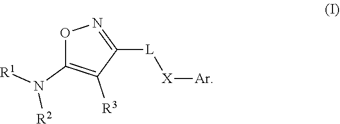

Substituents R¹ and R² are independently selected from the group consisting of H, C₁-C₆-alkyl (optionally substituted by one to five substituents independently selected from halo and OH), —C(O)C₁-C₆-alkyl, —C(O)H, —C₁-C₆-alkyl-(C₆-C₁₀-aryl), —C₁-C₆-alkyl-(C₁-C₆-alkoxy), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S), and —(C₁-C₆-alkyl)-SO₂—(C₁-C₆-alkyl), —(CH₂CH₂O)ₙ—R (wherein n is an integer selected from 2, 3, 4, 5, 6, 7, 8, 9, and 10, and R is H or C₁-C₆-alkyl);

Substituent R³ is selected from the group consisting of H, halo, C₁-C₆-alkyl, C₁-C₆-haloalkyl, and C₁-C₆-alkoxy.

L is a moiety selected from the group consisting of:

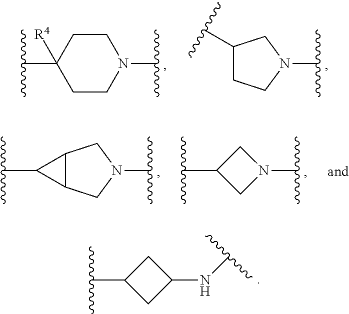

Substituent R⁴ is selected from the group consisting of H, halo, and C₁-C₆-alkyl.

X is —C(O)— or —SO₂—.

Ar is phenyl or indolyl. In some embodiments, Ar is

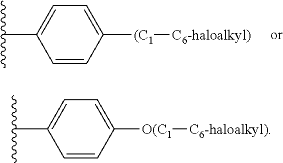

Ar is optionally substituted with one to five, or one to four, substituents selected from the group consisting of halo, C₁-C₆-alkyl, C₁-C₆-alkoxy, CN, OH, NO₂, —NRR′ (wherein R and R′ are independently selected from H and C₁-C₆-alkyl), —(C₁-C₆-alkyl)NRR′, —C(O)NRR′, —SO₂R, C₆-C₁₀-aryl, 3- to 6-membered heterocycloalkyl (wherein 1-4 ring members are independently selected from N, O, and S), 5- to 10-membered heteroaryl (wherein 1-4 heteroaryl members are independently selected from N, O, and S), and C₃-C₁₀-cycloalkyl.

The present disclosure also provides a pharmaceutical composition. The composition comprises a compound as described herein and a pharmaceutically acceptable carrier.

### BRIEF DESCRIPTION OF THE DRAWINGS

FIGS. 1A-1C. Dose response curves of ISRE reporter signal (FIG. 1A) and inhibition of 2′3′-cGAMP formation (FIG. 1B) for illustrative compounds 39, 54, and 63, and dose response curves of ISRE reporter signal for compound 2 (FIG. 1C).

### DETAILED DESCRIPTION

The present disclosure provides potent and non-cytotoxic cGAS inhibitor compounds, in accordance with Formula (I), that are therapeutically useful in diverse inflammatory disease settings.

### Definitions

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂ CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃) CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein.

Each of the terms “halogen,” “halide,” and “halo” refers to —F or fluoro, —Cl or chloro, —Br or bromo, or —I or iodo.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein.

“Alkyne or “alkynyl” refers to a straight or branched chain unsaturated hydrocarbon having the indicated number of carbon atoms and at least one triple bond. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein.

The term “alkoxy” or “alkoxyl” refers to an —O-alkyl group having the indicated number of carbon atoms. For example, a (C₁-C₆)-alkoxy group includes —O-methyl, —O-ethyl, —O-propyl, —O-isopropyl, —O-butyl, —O-sec-butyl, —O-tert-butyl, —O-pentyl, —O-isopentyl, —O— neopentyl, —O-hexyl, —O-isohexyl, and —O-neohexyl.

The term “cycloalkyl” refers to a saturated monocyclic, bicyclic, tricyclic, or polycyclic, 3- to 14-membered ring system, such as a C₃-C₈-cycloalkyl. The cycloalkyl may be attached via any atom. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein.

“Aryl” when used alone or as part of another term means a carbocyclic aromatic group whether or not fused having the number of carbon atoms designated or if no number is designated, up to 14 carbon atoms, such as a C₆-C₁₀-aryl or C₆-C₁₄-aryl. Examples of aryl groups include phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). “Aryl” also contemplates an aryl ring that is part of a fused polycyclic system, such as aryl fused to cycloalkyl as defined herein. An exemplary aryl is phenyl. An aryl group can be unsubstituted or optionally substituted with one or more substituents as described herein.

The term “heteroatom” refers to N, O, and S. Compounds of the present disclosure that contain N or S atoms can be optionally oxidized to the corresponding N-oxide, sulfoxide, or sulfone compounds.

“Heteroaryl,” alone or in combination with any other moiety described herein, is a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein.

“Heterocycloalkyl” is a saturated or partially unsaturated non-aromatic monocyclic, bicyclic, tricyclic or polycyclic ring system that has from 3 to 14, such as 3 to 6, atoms in which 1 to 3 carbon atoms in the ring are replaced by heteroatoms of O, S or N. A heterocycloalkyl is optionally fused with aryl or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A heterocycloalkyl group can be unsubstituted or optionally substituted with one or more substituents as described herein.

The term “nitrile” or “cyano” can be used interchangeably and refers to a —CN group.

The term “oxo” refers to a ═O atom bound to an atom that is part of a saturated or unsaturated moiety. Thus, the ═O atom can be bound to a carbon, sulfur, or nitrogen atom that is part of a cyclic or acyclic moiety.

A “hydroxyl” or “hydroxy” refers to an —OH group.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified to the contrary, the term “compound” is inclusive in that it encompasses a compound or a pharmaceutically acceptable salt, stereoisomer, and/or tautomer thereof. Thus, for instance, a compound of Formula (I) includes a pharmaceutically acceptable salt of a tautomer of the compound.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In various embodiments, the terms refer to minimizing or slowing the spread, progression, or worsening of the disease resulting from the administration of one or more prophylactic or therapeutic compounds described herein to a patient with such a disease.

The terms “prevent,” “preventing,” and “prevention” refer to the prevention of the onset, recurrence, or spread of the disease in a patient resulting from the administration of a compound described herein.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### Compounds

As described in summary above, the present disclosure provides compounds according to Formula (I), their pharmaceutically acceptable salts, and/or tautomers thereof:

The compound of Formula (I), in some embodiments, is one wherein Ar is:

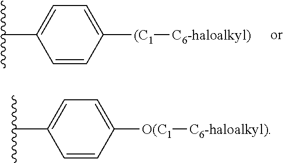

Ar is optionally substituted with one to four substituents selected from the group consisting of halo, C₁-C₆-alkyl, C₁-C₆-alkoxy, CN, OH, NO₂, —NRR′ (wherein R and R′ are independently selected from H and C₁-C₆-alkyl), —(C₁-C₆-alkyl)NRR′, —C(O)NRR′, —SO₂R, C₆-C₁₀-aryl, 3- to 6-membered heterocycloalkyl (wherein 1-4 ring members are independently selected from N, O, and S), 5- to 10-membered heteroaryl (wherein 1-4 heteroaryl members are independently selected from N, O, and S), and C₃-C₁₀-cycloalkyl.

In some embodiments, Ar is optionally substituted

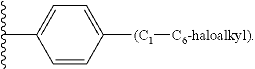

In other embodiments, Ar is optionally substituted

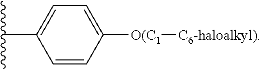

In combination with these embodiments, examples of C₁-C₆-haloalkyl include C₁-C₆-fluoroalkyl, such as mono-, di-, tri-, and perfluorinated alkyl. Illustrative examples of C₁-C₆-haloalkyl include —CFH₂, —CF₂H, and —CF₃. In additional embodiments, Ar is substituted with one or two substituents selected from halo, such as Cl and F, and C₁-C₆-alkyl. All these combinations are contemplated. Examples of Ar include the following:

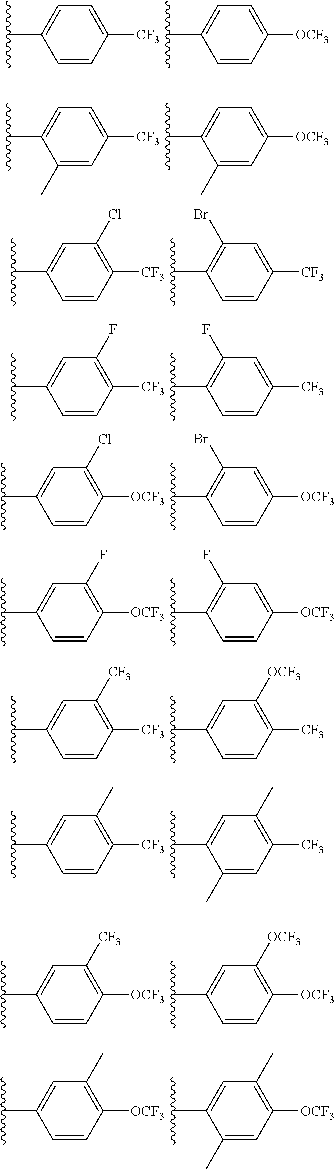

In additional embodiments, L is a moiety selected from

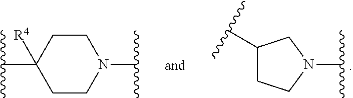

Illustrative compounds, according to some embodiments, are those wherein L is

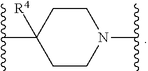

Further, in various embodiments, R⁴ is H.

In some embodiments, X is —C(O)—. In other embodiments, X is —SO₂—.

In still further embodiments, R¹ and R² are independently selected from the group consisting of H and C₁-C₆-alkyl that is optionally substituted as described herein. In various exemplary embodiments, each of R¹ and R² is H.

In additional embodiments, R³ is H.

Exemplary compounds of the present disclosure, according to Formula (I), include those wherein each of R¹, R², R³, and R⁴ is H; L is

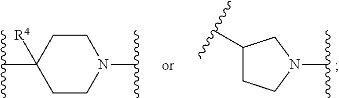

### and X is —C(O)—.

Specific examples of Formula (I) compounds include those throughout the examples and shown in Table 1 below.

TABLE 1
Examples of Formula (I) Compounds.

| | |
|---|---|
| 1 | 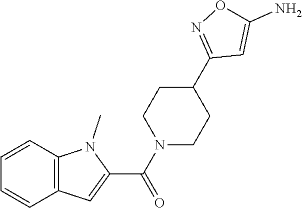 [SMILES: Cn1c(C(=O)N2CCC(c3cc(N)on3)CC2)cc2ccccc21] |
| 2 | 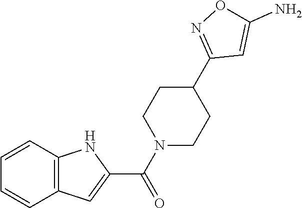 [SMILES: Nc1cc(C2CCN(C(=O)c3cc4ccccc4[nH]3)CC2)no1] |
| 3 | 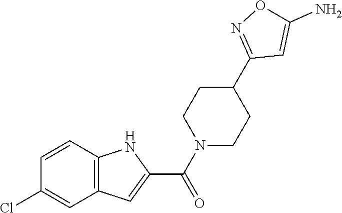 [SMILES: Nc1cc(C2CCN(C(=O)c3cc4cc(Cl)ccc4[nH]3)CC2)no1] |
| 4 | 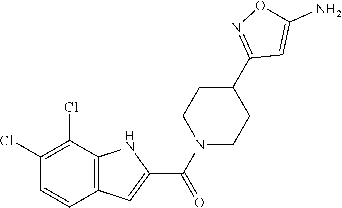 [SMILES: Nc1cc(C2CCN(C(=O)c3cc4ccc(Cl)c(Cl)c4[nH]3)CC2)no1] |
| 5 | 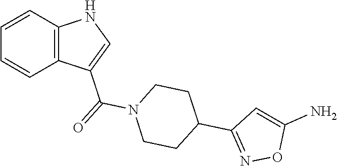 [SMILES: Nc1cc(C2CCN(C(=O)c3c[nH]c4ccccc34)CC2)no1] |
| 6 | 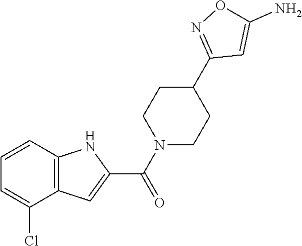 [SMILES: Nc1cc(C2CCN(C(=O)c3cc4c(Cl)cccc4[nH]3)CC2)no1] |
| 7 | 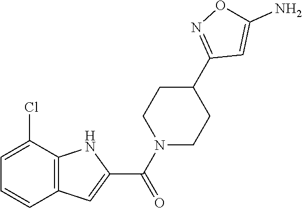 [SMILES: Nc1cc(C2CCN(C(=O)c3cc4cccc(Cl)c4[nH]3)CC2)no1] |
| 8 | 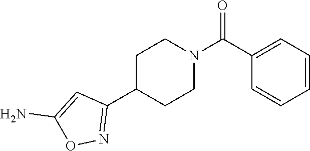 [SMILES: Nc1cc(C2CCN(C(=O)c3ccccc3)CC2)no1] |
| 9 | 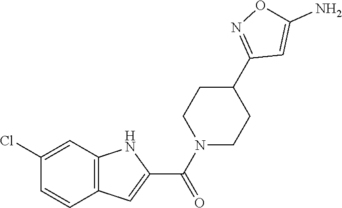 [SMILES: Nc1cc(C2CCN(C(=O)c3cc4ccc(Cl)cc4[nH]3)CC2)no1] |
| 10 | 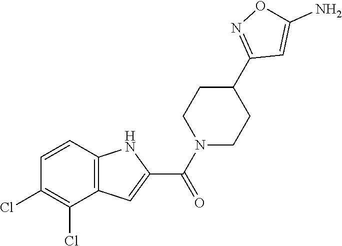 [SMILES: Nc1cc(C2CCN(C(=O)c3cc4c(Cl)c(Cl)ccc4[nH]3)CC2)no1] |
| 11 | 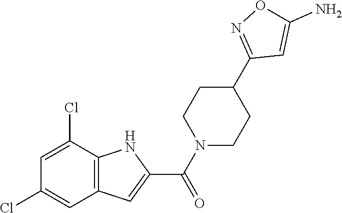 [SMILES: Nc1cc(C2CCN(C(=O)c3cc4cc(Cl)cc(Cl)c4[nH]3)CC2)no1] |
| 12 | 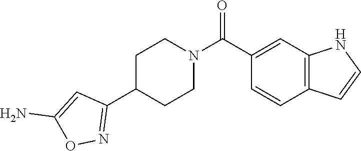 [SMILES: Nc1cc(C2CCN(C(=O)c3ccc4cc[nH]c4c3)CC2)no1] |
| 13 | 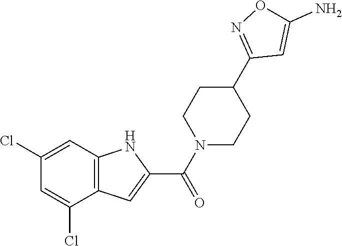 [SMILES: Nc1cc(C2CCN(C(=O)c3cc4c(Cl)cc(Cl)cc4[nH]3)CC2)no1] |
| 14 | 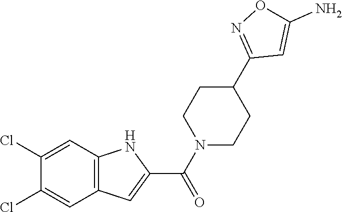 [SMILES: Nc1cc(C2CCN(C(=O)c3cc4cc(Cl)c(Cl)cc4[nH]3)CC2)no1] |
| 15 | 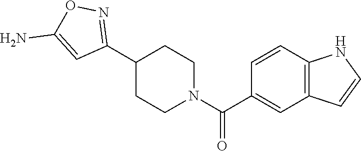 [SMILES: Nc1cc(C2CCN(C(=O)c3ccc4[nH]ccc4c3)CC2)no1] |
| 16 | 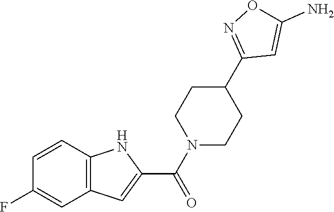 [SMILES: Nc1cc(C2CCN(C(=O)c3cc4cc(F)ccc4[nH]3)CC2)no1] |
| 17 | 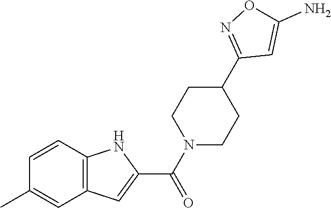 [SMILES: Cc1ccc2[nH]c(C(=O)N3CCC(c4cc(N)on4)CC3)cc2c1] |
| 18 | 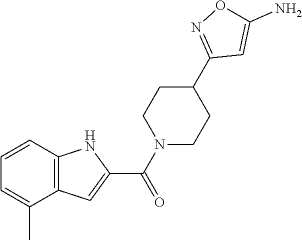 [SMILES: Cc1cccc2[nH]c(C(=O)N3CCC(c4cc(N)on4)CC3)cc12] |
| 19 | 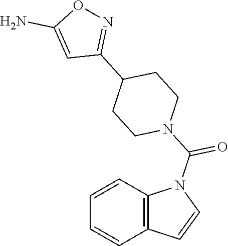 [SMILES: Nc1cc(C2CCN(C(=O)n3ccc4ccccc43)CC2)no1] |
| 20 | 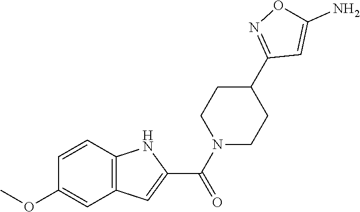 [SMILES: COc1ccc2[nH]c(C(=O)N3CCC(c4cc(N)on4)CC3)cc2c1] |
| 21 | 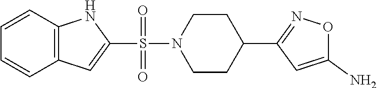 [SMILES: Nc1cc(C2CCN(S(=O)(=O)c3cc4ccccc4[nH]3)CC2)no1] |
| 22 | 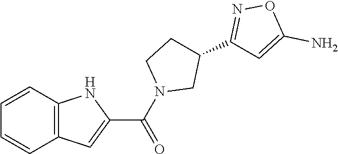 [SMILES: Nc1cc([C@H]2CCN(C(=O)c3cc4ccccc4[nH]3)C2)no1] |
| 23 | 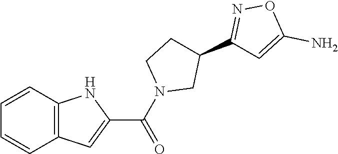 [SMILES: Nc1cc([C@@H]2CCN(C(=O)c3cc4ccccc4[nH]3)C2)no1] |
| 24 | 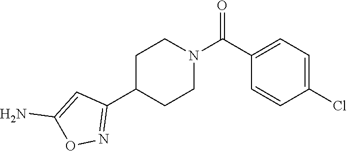 [SMILES: Nc1cc(C2CCN(C(=O)c3ccc(Cl)cc3)CC2)no1] |
| 25 | 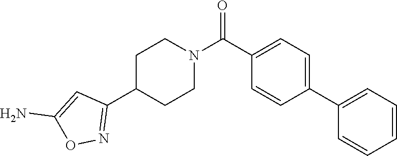 [SMILES: Nc1cc(C2CCN(C(=O)c3ccc(-c4ccccc4)cc3)CC2)no1] |
| 26 | 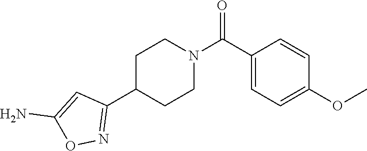 [SMILES: COc1ccc(C(=O)N2CCC(c3cc(N)on3)CC2)cc1] |
| 27 | 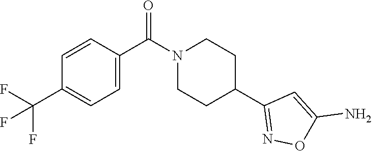 [SMILES: Nc1cc(C2CCN(C(=O)c3ccc(C(F)(F)F)cc3)CC2)no1] |
| 28 | 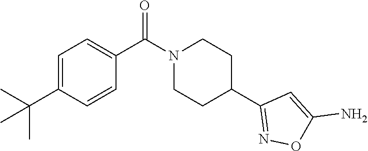 [SMILES: CC(C)(C)c1ccc(C(=O)N2CCC(c3cc(N)on3)CC2)cc1] |
| 29 | 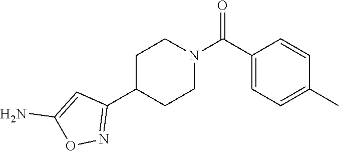 [SMILES: Cc1ccc(C(=O)N2CCC(c3cc(N)on3)CC2)cc1] |
| 30 | 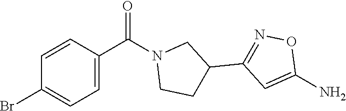 [SMILES: Nc1cc(C2CCN(C(=O)c3ccc(Br)cc3)C2)no1] |
| 31 | 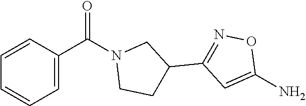 [SMILES: Nc1cc(C2CCN(C(=O)c3ccccc3)C2)no1] |
| 32 | 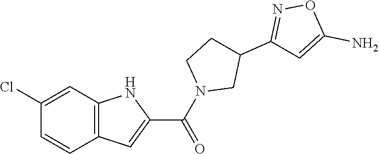 [SMILES: Nc1cc(C2CCN(C(=O)c3cc4ccc(Cl)cc4[nH]3)C2)no1] |
| 33 | 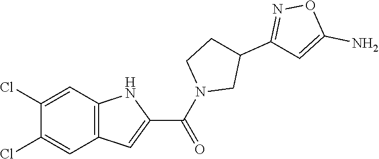 [SMILES: Nc1cc(C2CCN(C(=O)c3cc4cc(Cl)c(Cl)cc4[nH]3)C2)no1] |
| 34 | 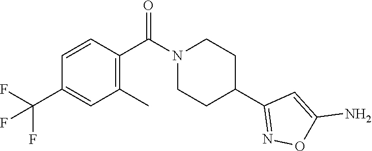 [SMILES: Cc1cc(C(F)(F)F)ccc1C(=O)N1CCC(c2cc(N)on2)CC1] |
| 35 | 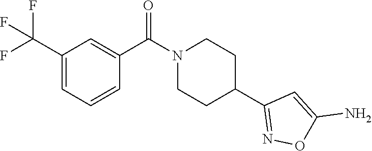 [SMILES: Nc1cc(C2CCN(C(=O)c3cccc(C(F)(F)F)c3)CC2)no1] |
| 36 | 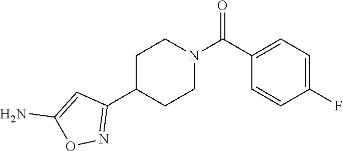 [SMILES: Nc1cc(C2CCN(C(=O)c3ccc(F)cc3)CC2)no1] |
| 37 | 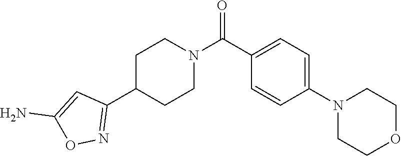 [SMILES: Nc1cc(C2CCN(C(=O)c3ccc(N4CCOCC4)cc3)CC2)no1] |
| 38 | 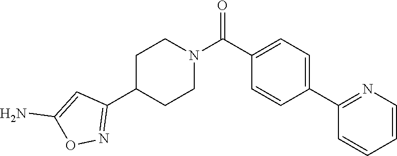 [SMILES: Nc1cc(C2CCN(C(=O)c3ccc(-c4ccccn4)cc3)CC2)no1] |
| 39 | 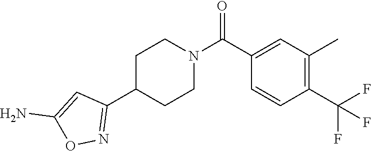 [SMILES: Cc1cc(C(=O)N2CCC(c3cc(N)on3)CC2)ccc1C(F)(F)F] |
| 40 | 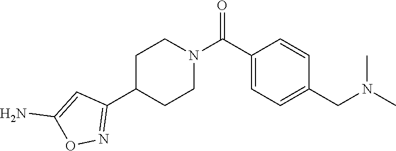 [SMILES: CN(C)Cc1ccc(C(=O)N2CCC(c3cc(N)on3)CC2)cc1] |
| 41 | 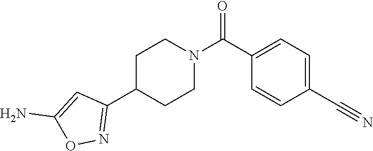 [SMILES: N#Cc1ccc(C(=O)N2CCC(c3cc(N)on3)CC2)cc1] |
| 42 | 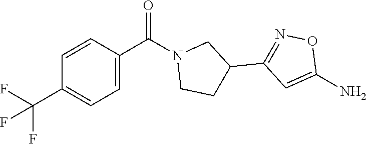 [SMILES: Nc1cc(C2CCN(C(=O)c3ccc(C(F)(F)F)cc3)C2)no1] |
| 43 | 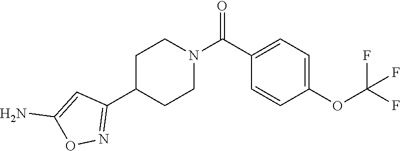 [SMILES: Nc1cc(C2CCN(C(=O)c3ccc(OC(F)(F)F)cc3)CC2)no1] |
| 44 | 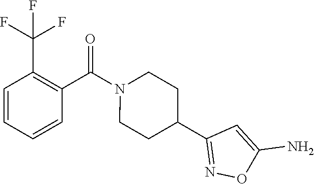 [SMILES: Nc1cc(C2CCN(C(=O)c3ccccc3C(F)(F)F)CC2)no1] |
| 45 | 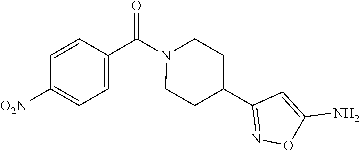 [SMILES: Nc1cc(C2CCN(C(=O)c3ccc([N+](=O)[O-])cc3)CC2)no1] |
| 46 | 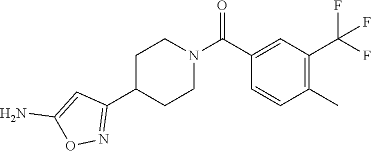 [SMILES: Cc1ccc(C(=O)N2CCC(c3cc(N)on3)CC2)cc1C(F)(F)F] |
| 47 | 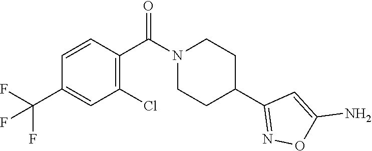 [SMILES: Nc1cc(C2CCN(C(=O)c3ccc(C(F)(F)F)cc3Cl)CC2)no1] |
| 48 | 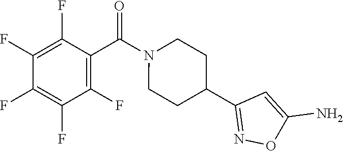 [SMILES: Nc1cc(C2CCN(C(=O)c3c(F)c(F)c(F)c(F)c3F)CC2)no1] |
| 49 | 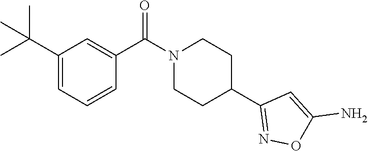 [SMILES: CC(C)(C)c1cccc(C(=O)N2CCC(c3cc(N)on3)CC2)c1] |
| 50 | 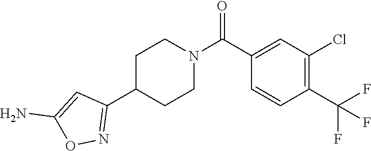 [SMILES: Nc1cc(C2CCN(C(=O)c3ccc(C(F)(F)F)c(Cl)c3)CC2)no1] |
| 51 | 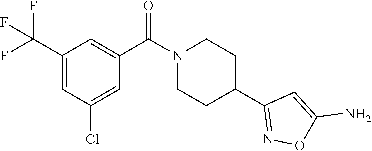 [SMILES: Nc1cc(C2CCN(C(=O)c3cc(Cl)cc(C(F)(F)F)c3)CC2)no1] |
| 52 | 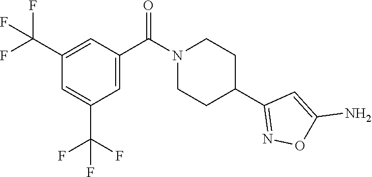 [SMILES: Nc1cc(C2CCN(C(=O)c3cc(C(F)(F)F)cc(C(F)(F)F)c3)CC2)no1] |
| 53 | 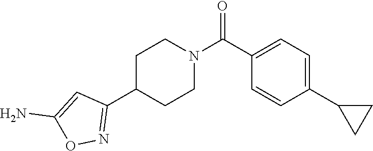 [SMILES: Nc1cc(C2CCN(C(=O)c3ccc(C4CC4)cc3)CC2)no1] |
| 54 | 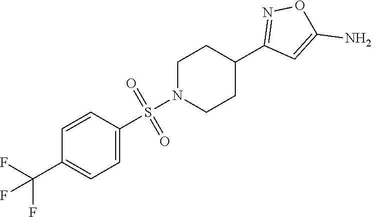 [SMILES: Nc1cc(C2CCN(S(=O)(=O)c3ccc(C(F)(F)F)cc3)CC2)no1] |
| 55 | 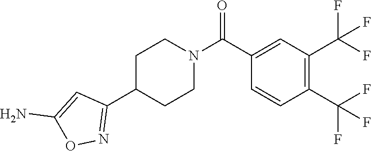 [SMILES: Nc1cc(C2CCN(C(=O)c3ccc(C(F)(F)F)c(C(F)(F)F)c3)CC2)no1] |
| 56 | 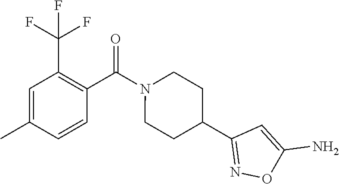 [SMILES: Cc1ccc(C(=O)N2CCC(c3cc(N)on3)CC2)c(C(F)(F)F)c1] |
| 57 | 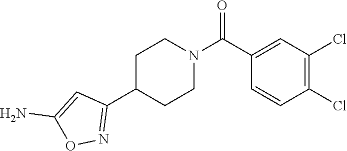 [SMILES: Nc1cc(C2CCN(C(=O)c3ccc(Cl)c(Cl)c3)CC2)no1] |
| 58 | 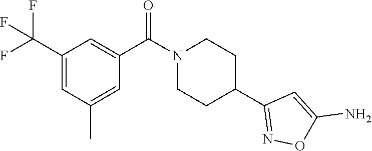 [SMILES: Cc1cc(C(=O)N2CCC(c3cc(N)on3)CC2)cc(C(F)(F)F)c1] |
| 59 | 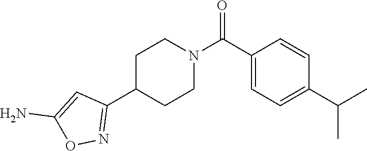 [SMILES: CC(C)c1ccc(C(=O)N2CCC(c3cc(N)on3)CC2)cc1] |
| 60 | 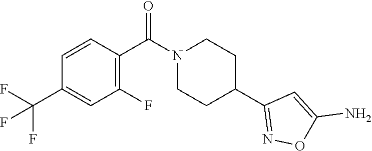 [SMILES: Nc1cc(C2CCN(C(=O)c3ccc(C(F)(F)F)cc3F)CC2)no1] |
| 61 | 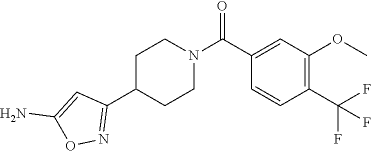 [SMILES: COc1cc(C(=O)N2CCC(c3cc(N)on3)CC2)ccc1C(F)(F)F] |
| 62 | 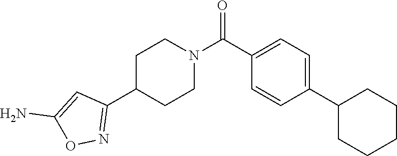 [SMILES: Nc1cc(C2CCN(C(=O)c3ccc(C4CCCCC4)cc3)CC2)no1] |
| 63 | 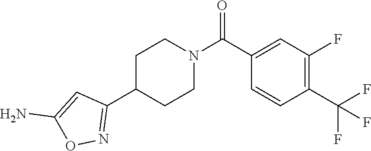 [SMILES: Nc1cc(C2CCN(C(=O)c3ccc(C(F)(F)F)c(F)c3)CC2)no1] |
| 64 | 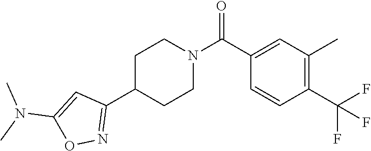 [SMILES: Cc1cc(C(=O)N2CCC(c3cc(N(C)C)on3)CC2)ccc1C(F)(F)F] |
| 65 | 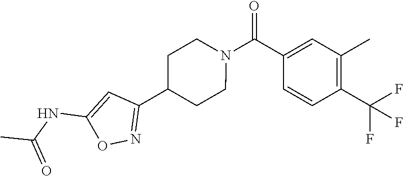 [SMILES: CC(=O)Nc1cc(C2CCN(C(=O)c3ccc(C(F)(F)F)c(C)c3)CC2)no1] |
| 66 | 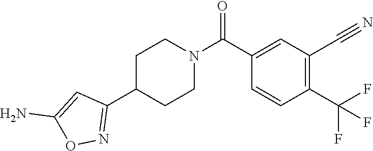 [SMILES: N#Cc1cc(C(=O)N2CCC(c3cc(N)on3)CC2)ccc1C(F)(F)F] |
| 67 | 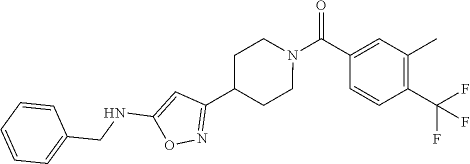 [SMILES: Cc1cc(C(=O)N2CCC(c3cc(NCc4ccccc4)on3)CC2)ccc1C(F)(F)F] |
| 68 | 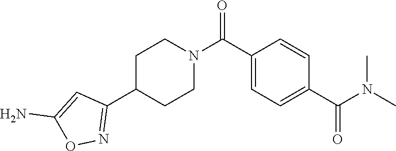 [SMILES: CN(C)C(=O)c1ccc(C(=O)N2CCC(c3cc(N)on3)CC2)cc1] |
| 69 | 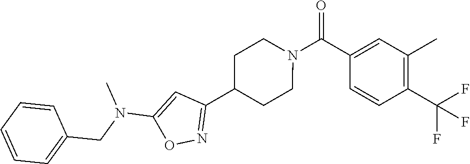 [SMILES: Cc1cc(C(=O)N2CCC(c3cc(N(C)Cc4ccccc4)on3)CC2)ccc1C(F)(F)F] |
| 70 | 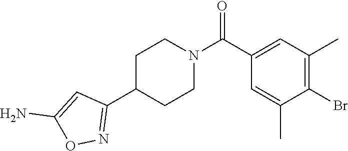 [SMILES: Cc1cc(C(=O)N2CCC(c3cc(N)on3)CC2)cc(C)c1Br] |
| 71 | 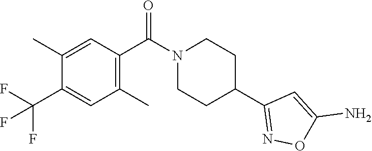 [SMILES: Cc1cc(C(F)(F)F)c(C)cc1C(=O)N1CCC(c2cc(N)on2)CC1] |
| 72 | 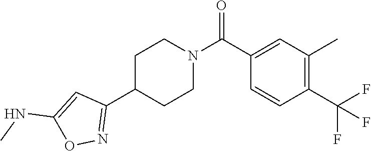 [SMILES: CNc1cc(C2CCN(C(=O)c3ccc(C(F)(F)F)c(C)c3)CC2)no1] |
| 73 | 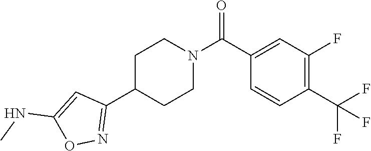 [SMILES: CNc1cc(C2CCN(C(=O)c3ccc(C(F)(F)F)c(F)c3)CC2)no1] |
| 74 | 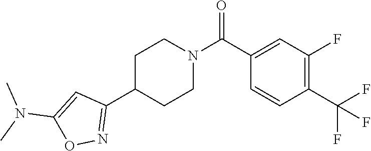 [SMILES: CN(C)c1cc(C2CCN(C(=O)c3ccc(C(F)(F)F)c(F)c3)CC2)no1] |
| 75 | 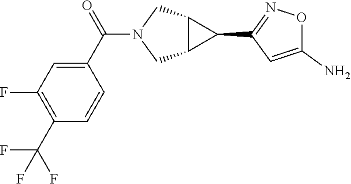 [SMILES: Nc1cc([C@H]2[C@@H]3CN(C(=O)c4ccc(C(F)(F)F)c(F)c4)C[C@@H]32)no1] |
| 76 | 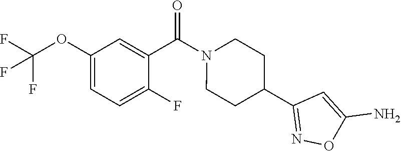 [SMILES: Nc1cc(C2CCN(C(=O)c3cc(OC(F)(F)F)ccc3F)CC2)no1] |
| 77 | 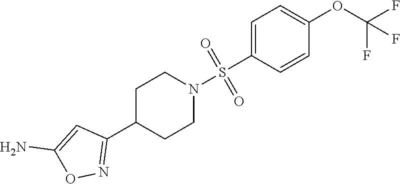 [SMILES: Nc1cc(C2CCN(S(=O)(=O)c3ccc(OC(F)(F)F)cc3)CC2)no1] |
| 78 | 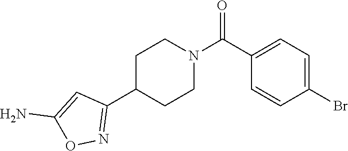 [SMILES: Nc1cc(C2CCN(C(=O)c3ccc(Br)cc3)CC2)no1] |
| 79 | 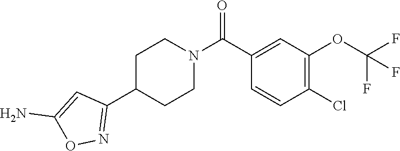 [SMILES: Nc1cc(C2CCN(C(=O)c3ccc(Cl)c(OC(F)(F)F)c3)CC2)no1] |
| 80 | 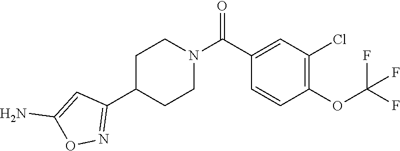 [SMILES: Nc1cc(C2CCN(C(=O)c3ccc(OC(F)(F)F)c(Cl)c3)CC2)no1] |
| 81 | 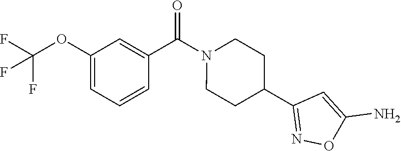 [SMILES: Nc1cc(C2CCN(C(=O)c3cccc(OC(F)(F)F)c3)CC2)no1] |
| 82 | 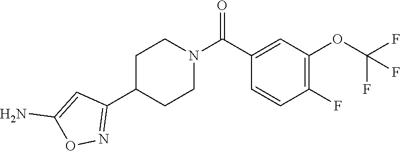 [SMILES: Nc1cc(C2CCN(C(=O)c3ccc(F)c(OC(F)(F)F)c3)CC2)no1] |
| 83 | 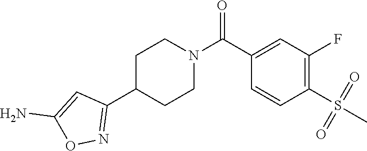 [SMILES: CS(=O)(=O)c1ccc(C(=O)N2CCC(c3cc(N)on3)CC2)cc1F] |
| 84 | 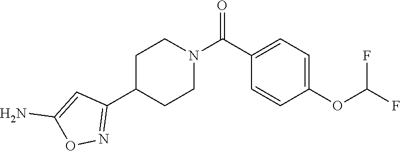 [SMILES: Nc1cc(C2CCN(C(=O)c3ccc(OC(F)F)cc3)CC2)no1] |
| 85 | 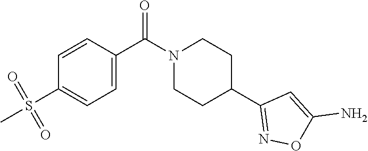 [SMILES: CS(=O)(=O)c1ccc(C(=O)N2CCC(c3cc(N)on3)CC2)cc1] |
| 86 | 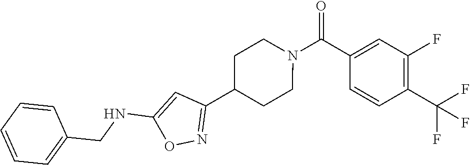 [SMILES: O=C(c1ccc(C(F)(F)F)c(F)c1)N1CCC(c2cc(NCc3ccccc3)on2)CC1] |
| 87 | 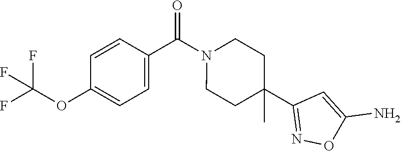 [SMILES: CC1(c2cc(N)on2)CCN(C(=O)c2ccc(OC(F)(F)F)cc2)CC1] |
| 88 | 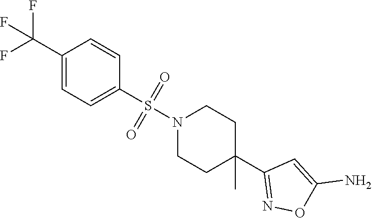 [SMILES: CC1(c2cc(N)on2)CCN(S(=O)(=O)c2ccc(C(F)(F)F)cc2)CC1] |
| 89 | 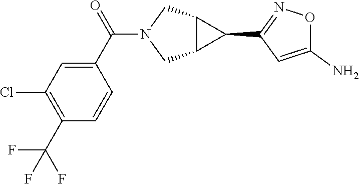 [SMILES: Nc1cc([C@H]2[C@@H]3CN(C(=O)c4ccc(C(F)(F)F)c(Cl)c4)C[C@@H]32)no1] |
| 90 | 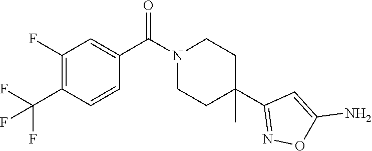 [SMILES: CC1(c2cc(N)on2)CCN(C(=O)c2ccc(C(F)(F)F)c(F)c2)CC1] |
| 91 | 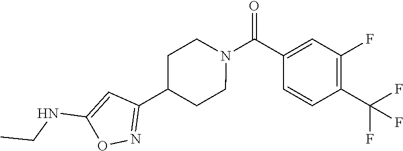 [SMILES: CCNc1cc(C2CCN(C(=O)c3ccc(C(F)(F)F)c(F)c3)CC2)no1] |
| 92 | 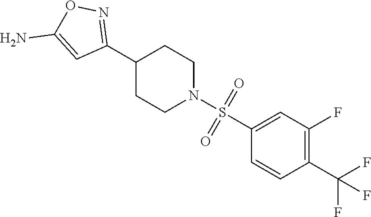 [SMILES: Nc1cc(C2CCN(S(=O)(=O)c3ccc(C(F)(F)F)c(F)c3)CC2)no1] |
| 93 | 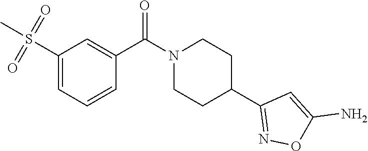 [SMILES: CS(=O)(=O)c1cccc(C(=O)N2CCC(c3cc(N)on3)CC2)c1] |
| 94 | 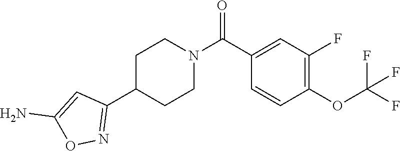 [SMILES: Nc1cc(C2CCN(C(=O)c3ccc(OC(F)(F)F)c(F)c3)CC2)no1] |
| 95 | 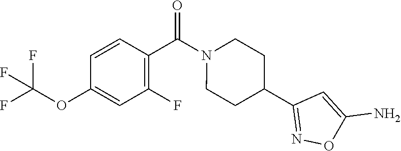 [SMILES: Nc1cc(C2CCN(C(=O)c3ccc(OC(F)(F)F)cc3F)CC2)no1] |
| 96 | 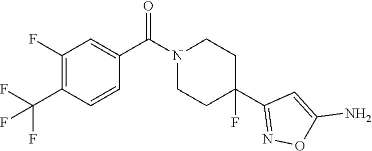 [SMILES: Nc1cc(C2(F)CCN(C(=O)c3ccc(C(F)(F)F)c(F)c3)CC2)no1] |
| 97 | 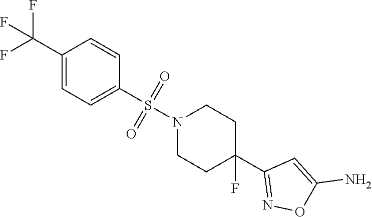 [SMILES: Nc1cc(C2(F)CCN(S(=O)(=O)c3ccc(C(F)(F)F)cc3)CC2)no1] |
| 98 | 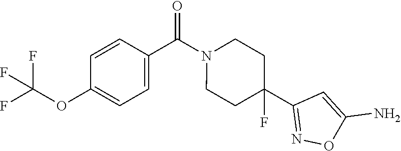 [SMILES: Nc1cc(C2(F)CCN(C(=O)c3ccc(OC(F)(F)F)cc3)CC2)no1] |
| 99 | 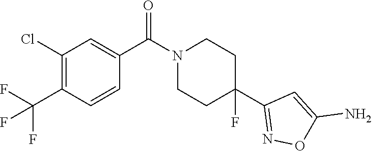 [SMILES: Nc1cc(C2(F)CCN(C(=O)c3ccc(C(F)(F)F)c(Cl)c3)CC2)no1] |
| 100 | 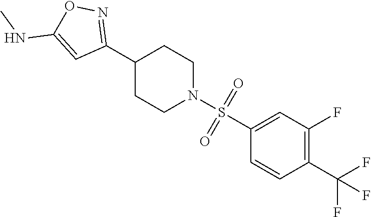 [SMILES: CNc1cc(C2CCN(S(=O)(=O)c3ccc(C(F)(F)F)c(F)c3)CC2)no1] |
| 101 | 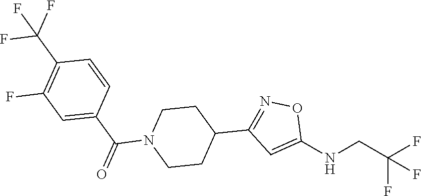 [SMILES: O=C(c1ccc(C(F)(F)F)c(F)c1)N1CCC(c2cc(NCC(F)(F)F)on2)CC1] |
| 102 | 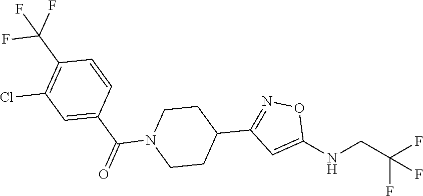 [SMILES: O=C(c1ccc(C(F)(F)F)c(Cl)c1)N1CCC(c2cc(NCC(F)(F)F)on2)CC1] |
| 103 | 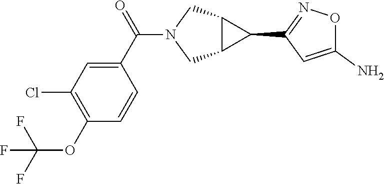 [SMILES: Nc1cc([C@H]2[C@@H]3CN(C(=O)c4ccc(OC(F)(F)F)c(Cl)c4)C[C@@H]32)no1] |
| 104 | 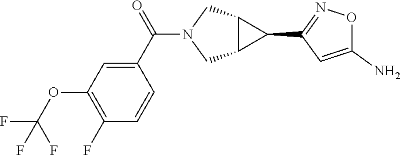 [SMILES: Nc1cc([C@H]2[C@@H]3CN(C(=O)c4ccc(F)c(OC(F)(F)F)c4)C[C@@H]32)no1] |
| 105 | 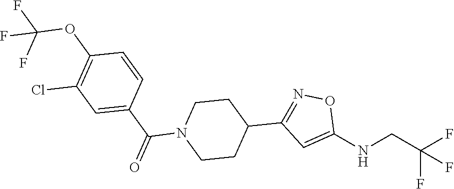 [SMILES: O=C(c1ccc(OC(F)(F)F)c(Cl)c1)N1CCC(c2cc(NCC(F)(F)F)on2)CC1] |
| 106 | 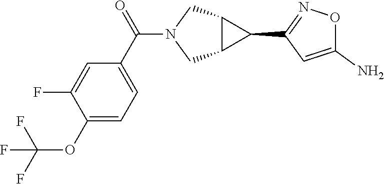 [SMILES: Nc1cc([C@H]2[C@@H]3CN(C(=O)c4ccc(OC(F)(F)F)c(F)c4)C[C@@H]32)no1] |
| 107 | 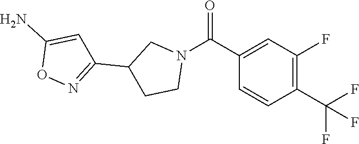 [SMILES: Nc1cc(C2CCN(C(=O)c3ccc(C(F)(F)F)c(F)c3)C2)no1] |
| 108 | 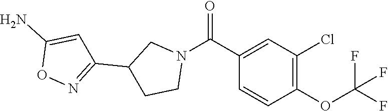 [SMILES: Nc1cc(C2CCN(C(=O)c3ccc(OC(F)(F)F)c(Cl)c3)C2)no1] |
| 109 | 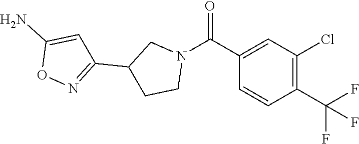 [SMILES: Nc1cc(C2CCN(C(=O)c3ccc(C(F)(F)F)c(Cl)c3)C2)no1] |
| 110 | 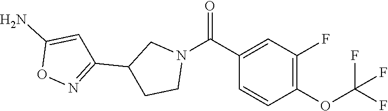 [SMILES: Nc1cc(C2CCN(C(=O)c3ccc(OC(F)(F)F)c(F)c3)C2)no1] |
| 111 | 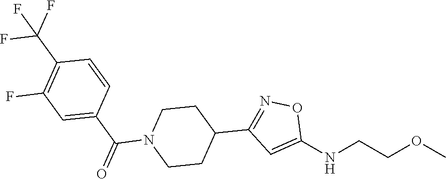 [SMILES: COCCNc1cc(C2CCN(C(=O)c3ccc(C(F)(F)F)c(F)c3)CC2)no1] |
| 112 | 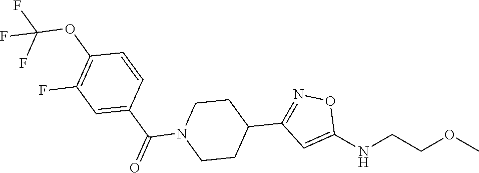 [SMILES: COCCNc1cc(C2CCN(C(=O)c3ccc(OC(F)(F)F)c(F)c3)CC2)no1] |
| 113 | 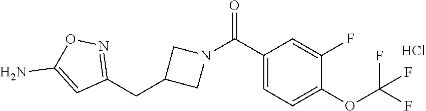 [SMILES: Nc1cc(CC2CN(C(=O)c3ccc(OC(F)(F)F)c(F)c3)C2)no1 ; Cl] |
| 114 | 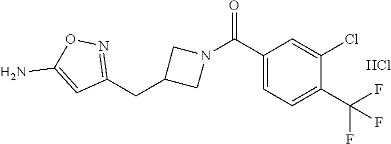 [SMILES: Nc1cc(CC2CN(C(=O)c3ccc(C(F)(F)F)c(Cl)c3)C2)no1 ; Cl] |
| 115 | 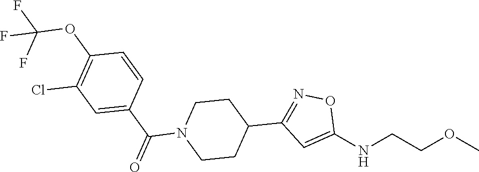 [SMILES: COCCNc1cc(C2CCN(C(=O)c3ccc(OC(F)(F)F)c(Cl)c3)CC2)no1] |
| 116 | 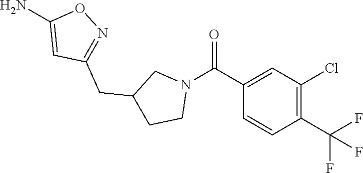 [SMILES: Nc1cc(CC2CCN(C(=O)c3ccc(C(F)(F)F)c(Cl)c3)C2)no1] |
| 117 | 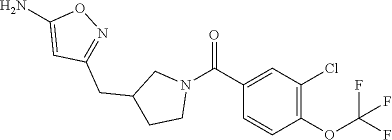 [SMILES: Nc1cc(CC2CCN(C(=O)c3ccc(OC(F)(F)F)c(Cl)c3)C2)no1] |
| 118 | 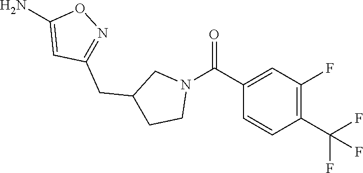 [SMILES: Nc1cc(CC2CCN(C(=O)c3ccc(C(F)(F)F)c(F)c3)C2)no1] |
| 119 | 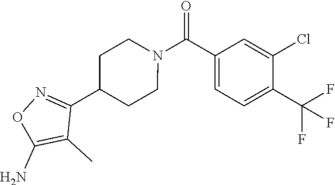 [SMILES: Cc1c(C2CCN(C(=O)c3ccc(C(F)(F)F)c(Cl)c3)CC2)noc1N] |
| 120 | 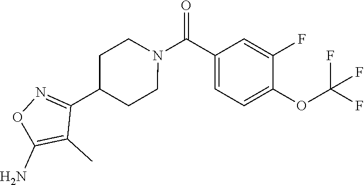 [SMILES: Cc1c(C2CCN(C(=O)c3ccc(OC(F)(F)F)c(F)c3)CC2)noc1N] |
| 121 | 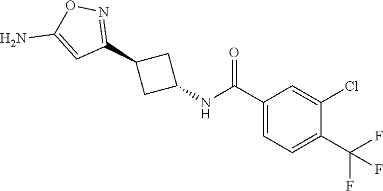 [SMILES: Nc1cc([C@H]2C[C@H](NC(=O)c3ccc(C(F)(F)F)c(Cl)c3)C2)no1] |
| 122 | 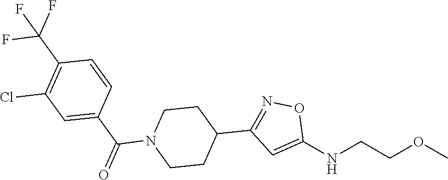 [SMILES: COCCNc1cc(C2CCN(C(=O)c3ccc(C(F)(F)F)c(Cl)c3)CC2)no1] |
| 123 | 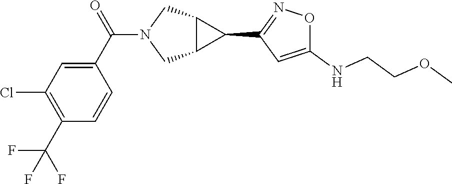 [SMILES: COCCNc1cc([C@H]2[C@@H]3CN(C(=O)c4ccc(C(F)(F)F)c(Cl)c4)C[C@@H]32)no1] |
| 124 | 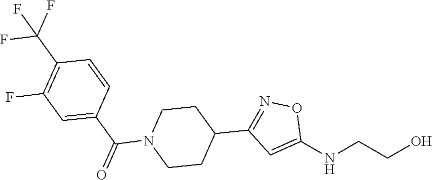 [SMILES: O=C(c1ccc(C(F)(F)F)c(F)c1)N1CCC(c2cc(NCCO)on2)CC1] |
| 125 | 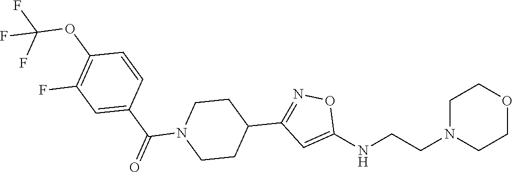 [SMILES: O=C(c1ccc(OC(F)(F)F)c(F)c1)N1CCC(c2cc(NCCN3CCOCC3)on2)CC1] |
| 126 | 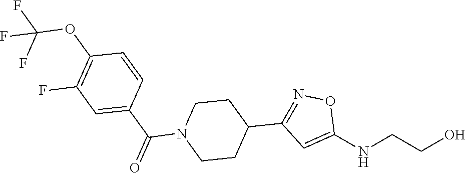 [SMILES: O=C(c1ccc(OC(F)(F)F)c(F)c1)N1CCC(c2cc(NCCO)on2)CC1] |
| 127 | 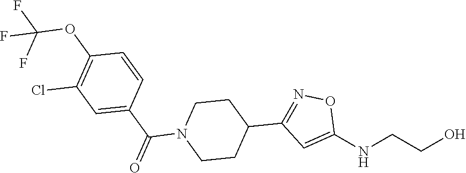 [SMILES: O=C(c1ccc(OC(F)(F)F)c(Cl)c1)N1CCC(c2cc(NCCO)on2)CC1] |
| 128 | 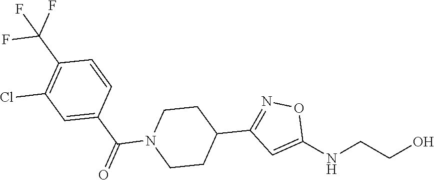 [SMILES: O=C(c1ccc(C(F)(F)F)c(Cl)c1)N1CCC(c2cc(NCCO)on2)CC1] |
| 129 | 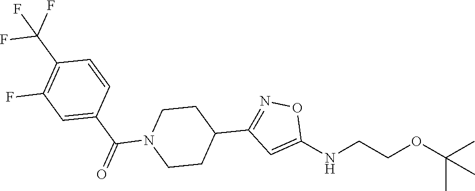 [SMILES: CC(C)(C)OCCNc1cc(C2CCN(C(=O)c3ccc(C(F)(F)F)c(F)c3)CC2)no1] |
| 130 | 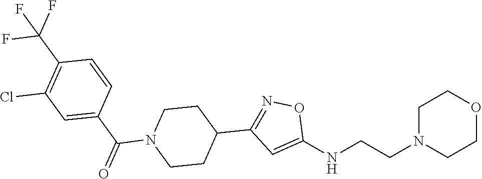 [SMILES: O=C(c1ccc(C(F)(F)F)c(Cl)c1)N1CCC(c2cc(NCCN3CCOCC3)on2)CC1] |
| 131 | 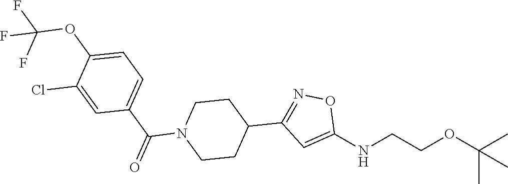 [SMILES: CC(C)(C)OCCNc1cc(C2CCN(C(=O)c3ccc(OC(F)(F)F)c(Cl)c3)CC2)no1] |
| 132 | 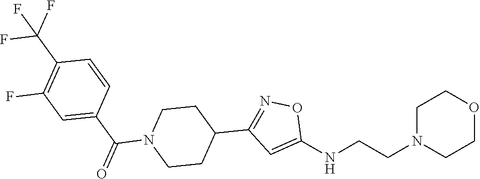 [SMILES: O=C(c1ccc(C(F)(F)F)c(F)c1)N1CCC(c2cc(NCCN3CCOCC3)on2)CC1] |
| 133 | 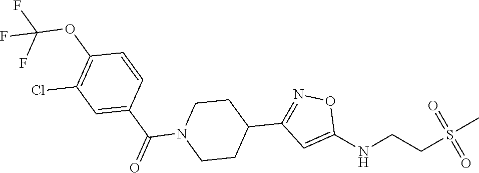 [SMILES: CS(=O)(=O)CCNc1cc(C2CCN(C(=O)c3ccc(OC(F)(F)F)c(Cl)c3)CC2)no1] |
| 134 | 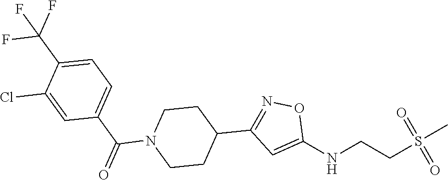 [SMILES: CS(=O)(=O)CCNc1cc(C2CCN(C(=O)c3ccc(C(F)(F)F)c(Cl)c3)CC2)no1] |
| 135 | 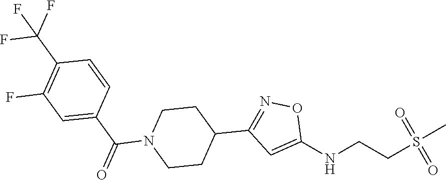 [SMILES: CS(=O)(=O)CCNc1cc(C2CCN(C(=O)c3ccc(C(F)(F)F)c(F)c3)CC2)no1] |
| 136 | 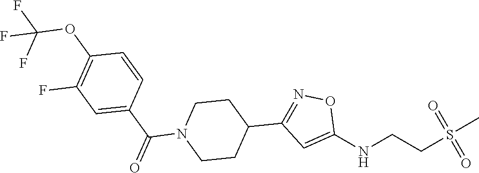 [SMILES: CS(=O)(=O)CCNc1cc(C2CCN(C(=O)c3ccc(OC(F)(F)F)c(F)c3)CC2)no1] |
| 137 | 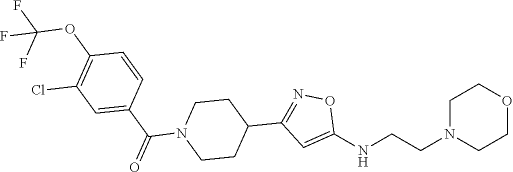 [SMILES: O=C(c1ccc(OC(F)(F)F)c(Cl)c1)N1CCC(c2cc(NCCN3CCOCC3)on2)CC1] |
| 138 | 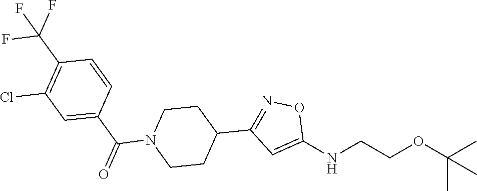 [SMILES: CC(C)(C)OCCNc1cc(C2CCN(C(=O)c3ccc(C(F)(F)F)c(Cl)c3)CC2)no1] |
| 139 | 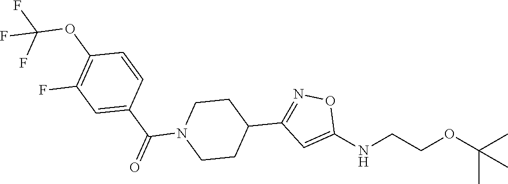 [SMILES: CC(C)(C)OCCNc1cc(C2CCN(C(=O)c3ccc(OC(F)(F)F)c(F)c3)CC2)no1] |
| 140 | 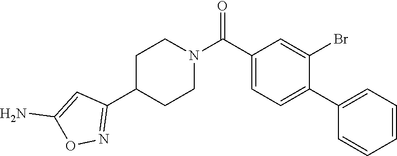 [SMILES: Nc1cc(C2CCN(C(=O)c3ccc(-c4ccccc4)c(Br)c3)CC2)no1] |
| 141 | 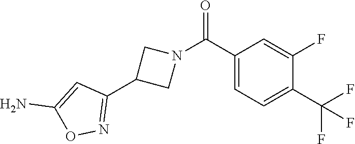 [SMILES: Nc1cc(C2CN(C(=O)c3ccc(C(F)(F)F)c(F)c3)C2)no1] |
| 142 | 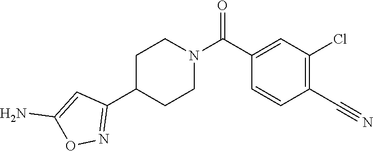 [SMILES: N#Cc1ccc(C(=O)N2CCC(c3cc(N)on3)CC2)cc1Cl] |
| 143 | 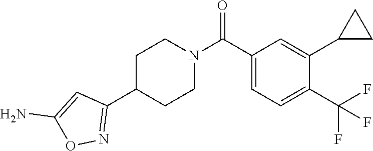 [SMILES: Nc1cc(C2CCN(C(=O)c3ccc(C(F)(F)F)c(C4CC4)c3)CC2)no1] |
| 144 | 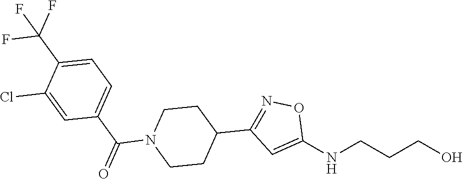 [SMILES: O=C(c1ccc(C(F)(F)F)c(Cl)c1)N1CCC(c2cc(NCCCO)on2)CC1] |
| 145 | 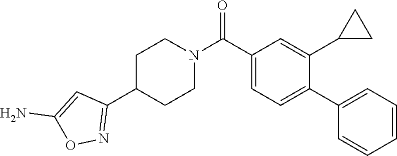 [SMILES: Nc1cc(C2CCN(C(=O)c3ccc(-c4ccccc4)c(C4CC4)c3)CC2)no1] |
| 146 | 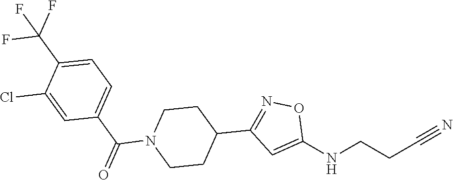 [SMILES: N#CCCNc1cc(C2CCN(C(=O)c3ccc(C(F)(F)F)c(Cl)c3)CC2)no1] |
| 147 | 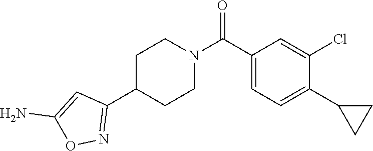 [SMILES: Nc1cc(C2CCN(C(=O)c3ccc(C4CC4)c(Cl)c3)CC2)no1] |
| 149 | 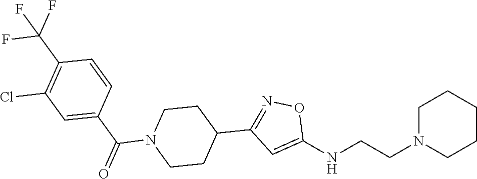 [SMILES: O=C(c1ccc(C(F)(F)F)c(Cl)c1)N1CCC(c2cc(NCCN3CCCCC3)on2)CC1] |
| 150 | 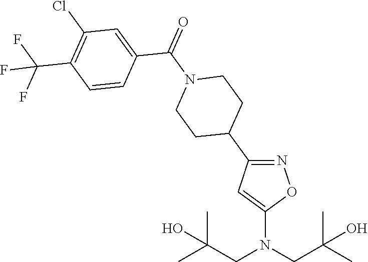 [SMILES: CC(C)(O)CN(CC(C)(C)O)c1cc(C2CCN(C(=O)c3ccc(C(F)(F)F)c(Cl)c3)CC2)no1] |
| 151 | 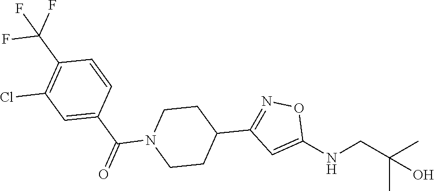 [SMILES: CC(C)(O)CNc1cc(C2CCN(C(=O)c3ccc(C(F)(F)F)c(Cl)c3)CC2)no1] |
| 152 | 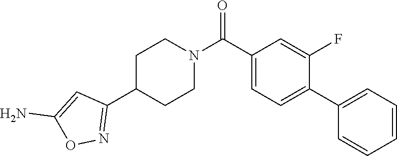 [SMILES: Nc1cc(C2CCN(C(=O)c3ccc(-c4ccccc4)c(F)c3)CC2)no1] |
| 153 | 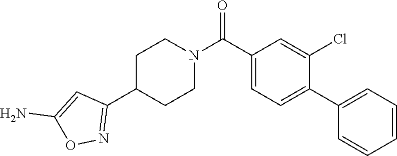 [SMILES: Nc1cc(C2CCN(C(=O)c3ccc(-c4ccccc4)c(Cl)c3)CC2)no1] |
| 154 | 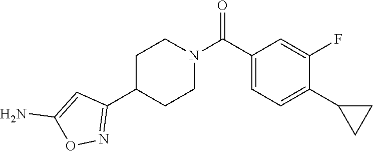 [SMILES: Nc1cc(C2CCN(C(=O)c3ccc(C4CC4)c(F)c3)CC2)no1] |
| 155 | 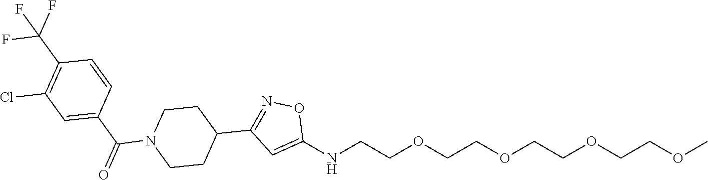 [SMILES: COCCOCCOCCOCCNc1cc(C2CCN(C(=O)c3ccc(C(F)(F)F)c(Cl)c3)CC2)no1] |
| 156 | 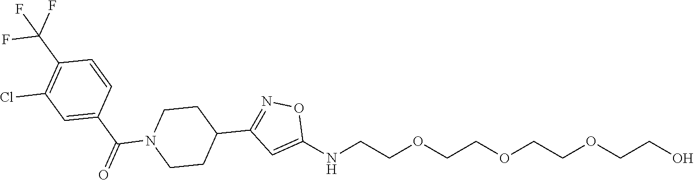 [SMILES: O=C(c1ccc(C(F)(F)F)c(Cl)c1)N1CCC(c2cc(NCCOCCOCCOCCO)on2)CC1] |
| 157 | 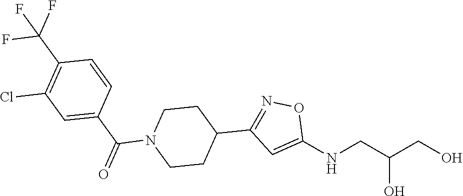 [SMILES: O=C(c1ccc(C(F)(F)F)c(Cl)c1)N1CCC(c2cc(NCC(O)CO)on2)CC1] |
| 158 | 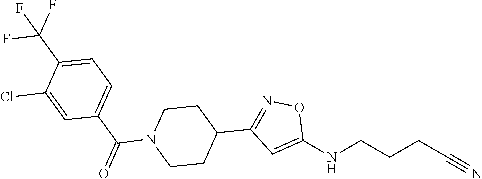 [SMILES: N#CCCCNc1cc(C2CCN(C(=O)c3ccc(C(F)(F)F)c(Cl)c3)CC2)no1] |
| 159 | 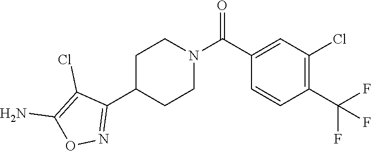 [SMILES: Nc1onc(C2CCN(C(=O)c3ccc(C(F)(F)F)c(Cl)c3)CC2)c1Cl] |
| 160 | 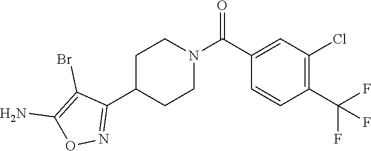 [SMILES: Nc1onc(C2CCN(C(=O)c3ccc(C(F)(F)F)c(Cl)c3)CC2)c1Br] |
| 161 | 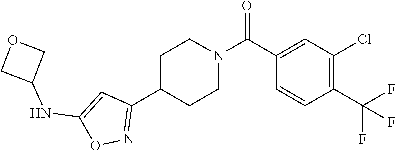 [SMILES: O=C(c1ccc(C(F)(F)F)c(Cl)c1)N1CCC(c2cc(NC3COC3)on2)CC1] |
| 162 | 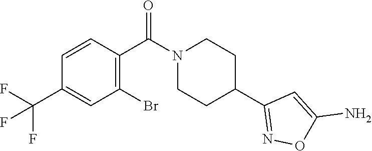 [SMILES: Nc1cc(C2CCN(C(=O)c3ccc(C(F)(F)F)cc3Br)CC2)no1] |
| 163 | 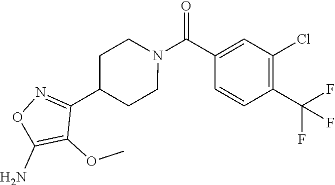 [SMILES: COc1c(C2CCN(C(=O)c3ccc(C(F)(F)F)c(Cl)c3)CC2)noc1N] |
| 164 | 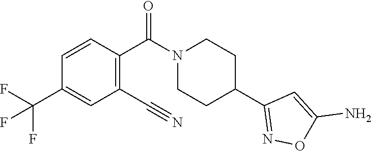 [SMILES: N#Cc1cc(C(F)(F)F)ccc1C(=O)N1CCC(c2cc(N)on2)CC1] |
| 165 | 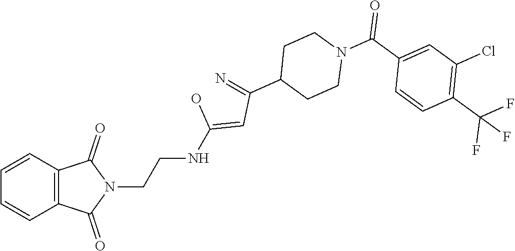 [SMILES: N=C(/C=C(\O)NCCN1C(=O)c2ccccc2C1=O)C1CCN(C(=O)c2ccc(C(F)(F)F)c(Cl)c2)CC1] |

### Pharmaceutical Composition

The present disclosure also provides a pharmaceutical composition comprising a therapeutically effective amount of one or more compounds according to Formula I or a pharmaceutically acceptable salt, stereoisomer, and/or tautomer thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Table 1 or a pharmaceutically acceptable salt, stereoisomer, and/or tautomer thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound or a pharmaceutically acceptable salt, stereoisomer, and/or tautomer thereof that is administered is governed by such considerations, and is the minimum amount necessary to inhibit cGAS, inhibit cGAS enzyme activity, inhibit synthesis of cGAMP dinucleotide, or any combination thereof. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment, such dosage forms contain from about 5 mg to about 50 mg of a compound (or a pharmaceutically acceptable salt, stereoisomer, or tautomer thereof) of the present disclosure. In any of the foregoing embodiments the dosage form can be administered once a day or twice per day.

The compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In another aspect, also encompassed are pharmaceutical compositions suitable for single unit dosages that comprise a compound of the disclosure or its pharmaceutically acceptable stereoisomer, salt, or tautomer and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of a compound of the present disclosure.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydroxpropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### Methods of Use

Compounds of Formula (I) according to the present disclosure are direct inhibitors of cGAS, as illustrated in the appended examples, and are therefore useful in inhibiting aberrant activation of the cGAS-STING pathway. Accordingly, the compounds are useful in the treatment of pathologies that are predicated upon the inhibition of the cGAS-STING pathway. Thus, in various embodiments, the present disclosure provides a method for treating an inflammatory disease or condition in a subject suffering therefrom. The method comprises administering to the subject a compound or pharmaceutically acceptable salt thereof as described herein. In some embodiments, the inflammatory disease is one associated with cytosolic double stranded nucleic acid. The compound or pharmaceutically acceptable salt thereof is administered optionally in a pharmaceutical composition in accordance with the present disclosure, and by any of the routes of administration as described herein.

Additional pathologies that are susceptible to attenuation of the cGAS-STING pathway, per the methods described herein as additional embodiments, are inflammatory diseases or conditions that include Type I interferonopathies, autoimmune diseases, neurological disorders, silica-induced fibrosis, and senescence-associated inflammatory diseases or disorders.

In some embodiments, the inflammatory disease is a Type I interferonopathy. Examples of Type I interferonopathies include Aicardi-Goutieres syndrome, spondyloenchondro-dysplasia with immune dysregulation, stimulator of interferon genes-associated vasculopathy with onset in infancy, X-linked reticulate pigmentary disorder, ubiquitin-specific peptidase 18 deficiency, chronic atypical neutrophilic dermatitis with lipodystrophy, Singleton-Merten syndrome, interferon-stimulated gene 15 deficiency, and DNAse II deficiency.

In other embodiments, the inflammatory disease is an autoimmune disease. Examples of autoimmune diseases include systemic lupus erythematosus and rheumatoid arthritis.

The inflammatory disease, per additional embodiments, is a neurological disorder or neuroinflammatory disease. Susceptible to the methods described herein are diseases and disorders that include ischaemic brain injury, Parkinson's disease, general neurodegeneration, Huntington's disease, amyotrophic lateral sclerosis, frontotemporal dementia, Alzheimer's disease, and traumatic brain injury.

In additional embodiments, the inflammatory disease or disorder is a senescence-associated inflammatory disease or disorder. Various senescence-associated inflammatory diseases or disorder are contemplated, including age-dependent macular degeneration, atherosclerosis, and osteoarthritis.

### EXAMPLES

The following non-limiting examples are additional embodiments that further illustrate the present disclosure.

### Synthesis of Compounds

General information: All evaporations were carried out in vacuo with a rotary evaporator. Analytical samples were dried in vacuo (1-5 mmHg) at room temperature. Thin layer chromatography (TLC) was performed on silica gel plates, spots were visualized by UV light (214 and 254 nm). Purification by column and flash chromatography were carried out using silica gel (200-300 mesh). Solvent systems are reported as mixtures by volume. All NMR spectra were recorded on a Bruker 400 (400 MHz) spectrometer. ¹H chemical shifts are reported in 6 values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration.

### Example 1: Synthesis of (4-(5-aminoisoxazol-3-yl)piperidin-1-yl)(1H-indol-3-yl)methanone (1)

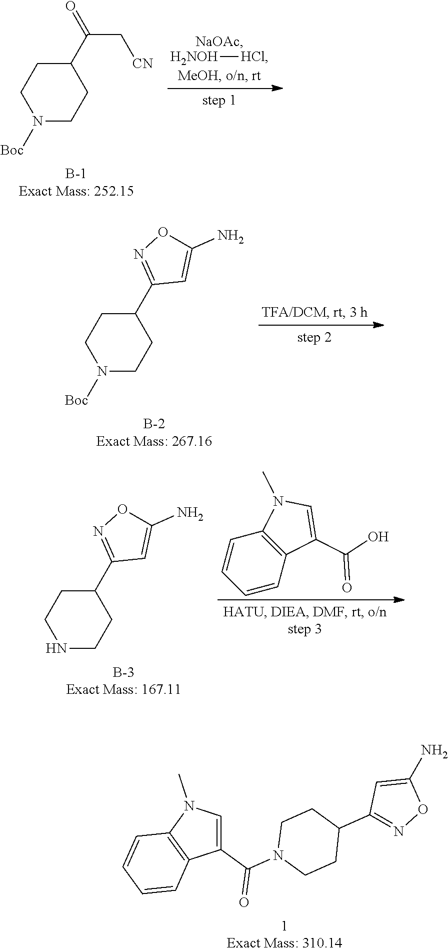

Synthesis of B-2 (step 1): To a solution of tert-butyl 4-(2-cyanoacetyl) piperidine-1-carboxylate (3.00 g, 11.9 mmol) in MeOH (50 mL), was added Sodium acetate (2.93 g, 35.7 mmol) and Hydroxyl ammonium hydrochloride (2.50 g, 35.7 mmol). The mixture was stirred at room temperature overnight. The reaction mixture was poured into water (100 mL) and extracted with EtOAc (60 mL×3). The organic layer was washed with brine and evaporated, the residue crude product was purified by column chromatography (PE/EtOAc=5/1) to give tert-butyl 4-(5-aminoisoxazol-3-yl) piperidine-1-carboxylate (B-2) (1.3 g, 41%) as a yellow solid.

Synthesis of B-3 (step 2): To a solution of tert-butyl 4-(5-aminoisoxazol-3-yl) piperidine-1-carboxylate (100 mg, 0.37 mmol) in DCM (5 mL), was added TFA (411 mg, 3.70 mmol). The mixture was stirred at room temperature for 3 h, The solvent was removed to give compound 3-(piperidin-4-yl)isoxazol-5-amine (B-3), it was used to next step directly without further purification.

Synthesis of 1 (step 3): The mixture of 1-methyl-1H-indole-3-carboxylic acid (59.6 mg, 0.37 mmol), 3-(piperidin-4-yl) isoxazol-5-amine (0.37 mmol), HATU (212.8 mg, 0.56 mmol) and DIEA (143.2 mg, 1.11 mmol) in DMF (3.00 mL) was stirred at room temperature overnight. The reaction mixture was poured into water (100 mL) and extracted with EtOAc (60 mL×3). The organic layer was washed with brine and evaporated, the residue crude product was purified by Prep-HPLC provided 1 (13.52 mg, yield: 12%, 2 steps) as a white solid. ¹HNMR (400 MHz, DMSO-d₆) δ 7.59 (d, J=7.6 Hz, 1H), 7.49 (d, J=8.4 Hz, 1H), 7.23 (t, J=7.6 Hz, 1H), 7.08 (t, J=7.6 Hz, 1H), 6.64 (s, 1H), 6.53 (s, 2H), 4.88 (s, 1H), 3.90-4.60 (m, 2H), 3.74 (s, 3H), 3.01-3.20 (m, 2H), 2.78-2.84 (m, 1H), 1.81-1.92 (m, 2H), 1.52-1.62 (m, 2H). ESI-MS: m/z=325.2 (M+1).

### Example 2: Synthesis of (4-(5-aminoisoxazol-3-yl)piperidin-1-yl)(1H-indol-2-yl)methanone (2)

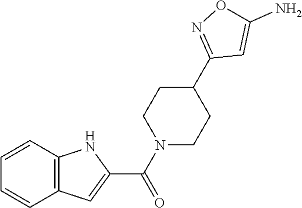 [SMILES: Nc1cc(C2CCN(C(=O)c3cc4ccccc4[nH]3)CC2)no1]

Similar to the preparation of 1, purification by Prep-HPLC provided the target compound 2 (21 mg, yield: 45%, 2 steps) as a pale yellow solid. ¹HNMR (400 MHz, MeOD-d₄) δ 7.60 (d, J=8.0 Hz, 1H), 7.42 (d, J=8.0 Hz, 1H), 7.20 (t, J=8.0 Hz, 1H), 7.05 (t, J=8.0 Hz, 1H), 6.81 (s, 1H), 4.99 (s, 1H), 4.80-4.90 (m, 2H), 4.58-4.61 (m, 2H), 2.90-3.00 (m, 1H), 2.00-2.03 (m, 2H), 1.71-1.75 (m, 2H). ESI-MS: m/z=311.3 (M+1).

### Example 3: Synthesis of (4-(5-aminoisoxazol-3-yl)piperidin-1-yl)(5-chloro-1H-indol-2-yl) methanone (3)

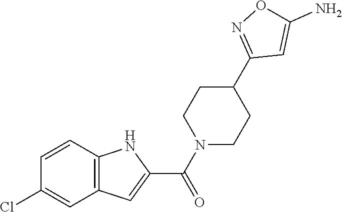 [SMILES: Nc1cc(C2CCN(C(=O)c3cc4cc(Cl)ccc4[nH]3)CC2)no1]

Similar to the preparation of 1, purification by Prep-HPLC provided the target compound 3 (10.23 mg, yield: 25%) as a white solid.

¹HNMR (400 MHz, DMSO-d₆) δ 11.75 (brs, 1H), 7.63 (d, J=2.0 Hz, 1H), 7.40 (d, J=8.8 Hz, 1H), 7.15 (dd, J=8.8, 2.0 Hz, 1H), 6.75 (s, 1H), 6.53 (s, 2H), 4.86 (s, 1H), 4.38-4.41 (m, 2H), 3.14-3.20 (m, 2H), 2.80-2.85 (m, 1H), 1.87-1.90 (m, 2H), 1.52-1.62 (m, 2H). ESI-MS: m/z=345.2 (M+1).

### Example 4: Synthesis of (4-(5-aminoisoxazol-3-yl)piperidin-1-yl)(6,7-dichloro-1H-indol-2-yl) methanone (4)

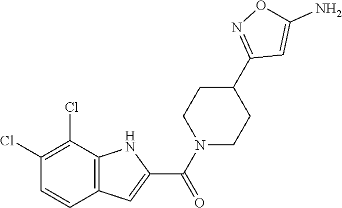 [SMILES: Nc1cc(C2CCN(C(=O)c3cc4ccc(Cl)c(Cl)c4[nH]3)CC2)no1]

Similar to the preparation of 1, purification by Prep-HPLC provided the target compound 4 (4.91 mg, yield: 11%) as a white solid. ¹HNMR (400 MHz, DMSO-d₆) δ 11.90 (brs, 1H), 7.53-7.55 (m, 1H), 7.05-7.18 (m, 1H), 6.75-6.80 (m, 1H), 6.53 (s, 2H), 4.86 (s, 1H), 4.32-4.56 (m, 2H), 2.93-3.22 (m, 2H), 2.79-2.93 (m, 1H), 1.80-1.92 (m, 2H), 1.51-1.61 (m, 2H). ESI-MS: m/z=379.2 (M+1).

### Example 5: Synthesis of (4-(5-aminoisoxazol-3-yl)piperidin-1-yl)(1H-indol-3-yl)methanone (5)

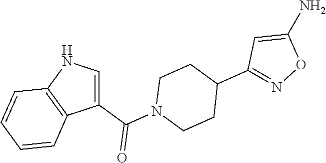 [SMILES: Nc1cc(C2CCN(C(=O)c3c[nH]c4ccccc34)CC2)no1]

Similar to the preparation of 1, purification by prep-HPLC provided the target compound 5 (10.0 mg, yield: 5.4%) as a white solid. ¹H NMR (400 MHz, DMSO-d₆) δ 11.56 (s, 1H), 7.61-7.67 (m, 2H), 7.41-7.44 (m, 1H), 7.05-7.14 (m, 2H), 6.51 (s, 2H), 4.86 (s, 1H), 4.26 (d, J=13.2 Hz, 2H), 3.01-3.07 (m, 2H), 2.73-2.79 (m, 1H), 1.83 (d, J=14.0 Hz, 2H), 1.48-1.59 (m, 2H). ESI-MS: m/z=311.4 (M+1).

### Example 6: Synthesis of (4-(5-aminoisoxazol-3-yl)piperidin-1-yl)(4-chloro-1H-indol-2-yl)methanone (6)

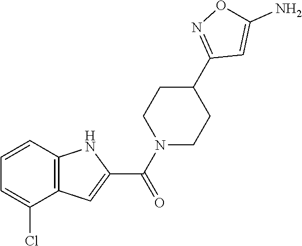 [SMILES: Nc1cc(C2CCN(C(=O)c3cc4c(Cl)cccc4[nH]3)CC2)no1]

Similar to the preparation of 1, purification by prep-HPLC provided the target compound 6 (15.0 mg, yield: 7.3%) as a white solid. ¹H NMR (400 MHz, DMSO-d₆) δ 11.97 (s, 1H), 7.37 (d, J=8.0 Hz, 1H), 7.07-7.13 (m, 2H), 6.72 (s, 1H), 6.52 (s, 2H), 4.87 (s, 1H), 4.44 (d, J=10.8 Hz, 2H), 2.78-2.87 (m, 1H), 2.39-2.42 (m, 2H), 1.88 (d, J=13.2 Hz, 2H), 1.52-1.62 (m, 2H). ESI-MS: m/z=345.3 (M+1).

### Example 7: Synthesis of (4-(5-aminoisoxazol-3-yl)piperidin-1-yl)(7-chloro-1H-indol-2-yl)methanone (7)

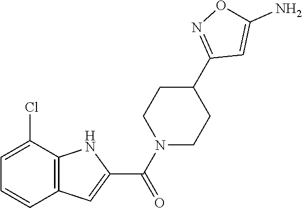 [SMILES: Nc1cc(C2CCN(C(=O)c3cc4cccc(Cl)c4[nH]3)CC2)no1]

Similar to the preparation of 1, purification by prep-HPLC provided the target compound 7 (11.0 mg, yield: 5.4%) as a white solid. ¹H NMR (400 MHz, DMSO-d₆) δ 11.79 (s, 1H), 7.55 (d, J=7.6 Hz, 1H), 7.23 (d, J=7.6 Hz, 1H), 7.03 (t, J=8.0 Hz, 1H), 6.77 (s, 1H), 6.53 (s, 2H), 4.85 (s, 1H), 4.26 (bs, 2H), 3.17-3.23 (m, 1H), 2.78-2.86 (m, 2H), 1.82-1.90 (m, 2H), 1.52-1.64 (m, 2H). ESI-MS: m/z=345.4 (M+1).

### Example 8: Synthesis of (4-(5-aminoisoxazol-3-yl)piperidin-1-yl)(phenyl)methanone (8)

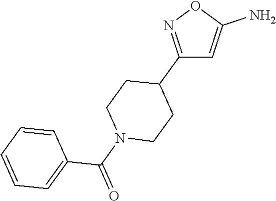 [SMILES: Nc1cc(C2CCN(C(=O)c3ccccc3)CC2)no1]

Similar to the preparation of 1, purification by Prep-HPLC provided the target compound 8 (26.13 mg, yield: 16%) as a yellow solid. ¹HNMR (400 MHz, DMSO-d₆) δ 7.41-7.44 (m, 3H), 7.36-7.39 (m, 2H), 6.51 (s, 2H), 4.86 (s, 1H), 4.38-4.50 (m, 1H), 3.49-3.55 (m, 1H), 3.03-3.18 (m, 1H), 2.84-2.96 (m, 1H), 2.72-2.79 (m, 1H), 1.69-1.92 (m, 2H), 1.46-1.59 (m, 2H). ESI-MS: m/z=272.2 (M+1).

### Example 9: Synthesis of (4-(5-aminoisoxazol-3-yl)piperidin-1-yl)(6-chloro-1H-indol-2-yl) methanone (9)

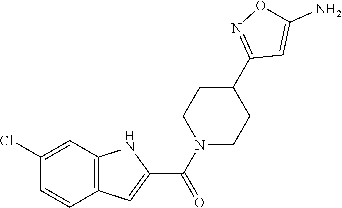 [SMILES: Nc1cc(C2CCN(C(=O)c3cc4ccc(Cl)cc4[nH]3)CC2)no1]

Similar to the preparation of 1, purification by Prep-HPLC provided the target compound 9 (13.39 mg, yield: 6%) as a pale yellow solid. ¹HNMR (400 MHz, DMSO-d₆) δ 11.71 (brs, 1H), 7.62 (d, J=8.4 Hz, 1H), 7.42 (s, 1H), 7.05 (dd, J=8.4, 2.0 Hz, 1H), 6.82 (s, 1H), 6.54 (s, 2H), 4.88 (s, 1H), 4.42-4.45 (m, 2H), 3.00-3.20 (m, 2H), 2.82-2.87 (m, 1H), 1.89-1.92 (m, 2H), 1.58-1.63 (m, 2H). ESI-MS: m/z=345.2 (M+1).

### Example 10: Synthesis of (4-(5-aminoisoxazol-3-yl)piperidin-1-yl)(4,5-dichloro-1H-indol-2-yl)methanone (10)

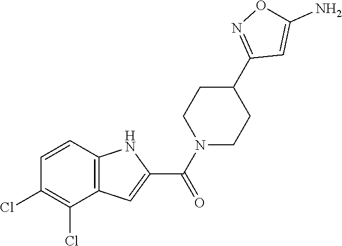 [SMILES: Nc1cc(C2CCN(C(=O)c3cc4c(Cl)c(Cl)ccc4[nH]3)CC2)no1]

Similar to the preparation of 1, purification by prep-HPLC provided the target compound 10 (10.0 mg, yield: 4.4%) as a white solid. ¹H NMR (400 MHz, DMSO-d₆) δ 12.09 (s, 1H), 7.32 (d, J=8.4 Hz, 1H), 7.14 (s, 1H), 6.69 (s, 1H), 6.51 (s, 2H), 4.85 (s, 1H), 4.49 (brs, 2H), 3.24 (brs, 2H), 2.76-2.80 (m, 1H), 1.86 (d, J=12.0 Hz, 2H), 1.53-1.56 (m, 2H). ESI-MS: m/z=379.2 (M+1).

### Example 11: Synthesis of (4-(5-aminoisoxazol-3-yl)piperidin-1-yl)(5,7-dichloro-1H-indol-2-yl)methanone (11)

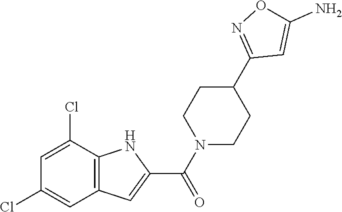 [SMILES: Nc1cc(C2CCN(C(=O)c3cc4cc(Cl)cc(Cl)c4[nH]3)CC2)no1]

Similar to the preparation of 1, purification by prep-HPLC provided the target compound 11 (20.0 mg, yield: 8.8%) as a white solid. ¹H NMR (400 MHz, DMSO-d₆) δ 12.04 (s, 1H), 7.59 (s, 1H), 7.26 (s, 1H), 6.73 (s, 1H), 6.52 (s, 2H), 4.85 (s, 1H), 4.45 (brs, 2H), 3.21 (s, 2H), 2.78-2.83 (m, 1H), 1.84 (s, 2H), 1.52-1.59 (m, 2H). ESI-MS: m/z=379.2 (M+1).

### Example 12: Synthesis of (4-(5-aminoisoxazol-3-yl)piperidin-1-yl)(1H-indol-6-yl)methanone (12)

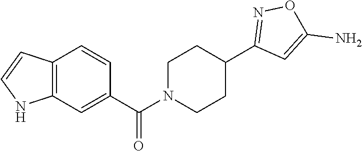 [SMILES: Nc1cc(C2CCN(C(=O)c3ccc4cc[nH]c4c3)CC2)no1]

Similar to the preparation of 1, purification by prep-HPLC provided the target compound 12 (15.0 mg, yield: 8.1%) as a white solid. ¹H NMR (400 MHz, DMSO-d₆) δ 11.25 (s, 1H), 7.55 (d, J=8.0 Hz, 1H), 7.43 (s, 2H), 6.99-7.02 (m, 1H), 6.52 (s, 2H), 6.45 (d, J=3.2 Hz, 1H), 4.87 (s, 1H), 4.15 (brs, 2H), 2.99 (s, 2H), 2.70-2.79 (m, 1H), 1.80 (s, 2H), 1.51-1.55 (m, 2H). ESI-MS: m/z=311.4 (M+1).

### Example 13: Synthesis of (4-(5-aminoisoxazol-3-yl)piperidin-1-yl)(4,6-dichloro-1H-indol-2-yl) methanone (13)

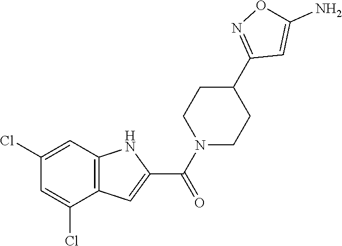 [SMILES: Nc1cc(C2CCN(C(=O)c3cc4c(Cl)cc(Cl)cc4[nH]3)CC2)no1]

Similar to the preparation of 1, purification by Prep-HPLC provided the target compound 13 (37.47 mg, yield: 17%) as a pale yellow solid. ¹HNMR (400 MHz, DMSO-d₆) δ 12.10 (brs, 1H), 7.39 (s, 1H), 7.15 (s, 1H), 6.76 (s, 1H), 6.54 (s, 2H), 4.88 (s, 1H), 4.47-4.51 (m, 2H), 2.90-3.20 (m, 2H), 2.79-2.86 (m, 1H), 1.88-1.91 (m, 2H), 1.54-1.62 (m, 2H). ESI-MS: m/z=379.1 (M+1).

### Example 14: Synthesis of (4-(5-aminoisoxazol-3-yl)piperidin-1-yl)(5,6-dichloro-1H-indol-2-yl)methanone (14)

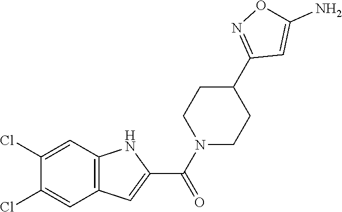 [SMILES: Nc1cc(C2CCN(C(=O)c3cc4cc(Cl)c(Cl)cc4[nH]3)CC2)no1]

Similar to the preparation of 1, purification by prep-HPLC provided the target compound 14 (21 mg, yield: 9.3%) as a white solid. ¹H NMR (400 MHz, DMSO-d₆) δ 11.87 (s, 1H), 7.83 (s, 1H), 7.58 (s, 1H), 6.77 (s, 1H), 6.52 (s, 2H), 4.86 (s, 1H), 4.42 (d, J=12.8 Hz, 2H), 3.23 (s, 2H), 2.78-2.86 (m, 1H), 1.89 (d, J=13.2 Hz, 2H), 1.50-1.61 (m, 2H). ESI-MS: m/z=379.3 (M+1).

### Example 15: Synthesis of (4-(5-aminoisoxazol-3-yl)piperidin-1-yl)(1H-indol-5-yl)methanone (15)

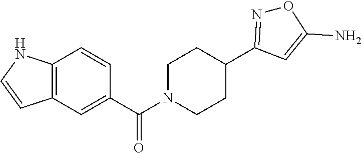 [SMILES: Nc1cc(C2CCN(C(=O)c3ccc4[nH]ccc4c3)CC2)no1]

Similar to the preparation of 1, purification by Prep-HPLC provided the target compound 15 (19.44 mg, yield: 10%) as a white solid. ¹HNMR (400 MHz, DMSO-d₆) δ 11.28 (s, 1H), 7.59 (s, 1H), 7.39-7.41 (m, 2H), 7.11 (dd, J=8.4, 1.6 Hz, 1H), 6.51 (s, 2H), 6.47 (d, J=3.2 Hz, 1H), 4.87 (s, 1H), 3.90-4.30 (brs, 2H), 2.90-3.10 (m, 2H), 2.74-2.76 (m, 1H), 1.78-1.81 (m, 2H), 1.51-1.55 (m, 2H). ESI-MS: m/z=311.3 (M+1).

### Example 16: Synthesis of (4-(5-aminoisoxazol-3-yl)piperidin-1-yl)(5-fluoro-1H-indol-2-yl)methanonenone (16)

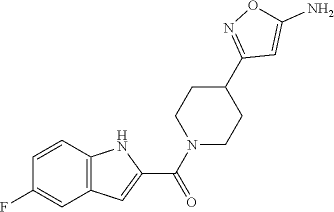 [SMILES: Nc1cc(C2CCN(C(=O)c3cc4cc(F)ccc4[nH]3)CC2)no1]

Similar to the preparation of 1, purification by prep-HPLC provided the target compound 16 (18.0 mg, yield: 9.2%) as a white solid. ¹H NMR (400 MHz, DMSO-d₆) δ 11.65 (s, 1H), 7.32-7.40 (m, 2H), 6.98-7.04 (m, 1H), 6.74 (s, 1H), 6.53 (s, 2H), 4.86 (s, 1H), 4.40 (d, J=12.4 Hz, 2H), 3.22 (s, 2H), 2.79-2.86 (m, 1H), 1.89 (d, J=16.0 Hz, 2H), 1.51-1.62 (m, 2H). ESI-MS: m/z=329.4 (M+1).

### Example 17: Synthesis of (4-(5-aminoisoxazol-3-yl)piperidin-1-yl)(5-methyl-1H-indol-2-yl) methanone (17)

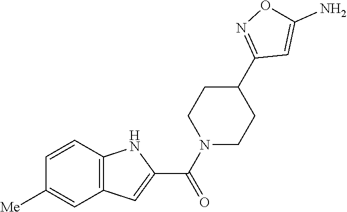 [SMILES: Cc1ccc2[nH]c(C(=O)N3CCC(c4cc(N)on4)CC3)cc2c1]

Similar to the preparation of 1, purification by Prep-HPLC provided the target compound 17 (15.20 mg, yield: 8%) as a white solid. ¹HNMR (400 MHz, DMSO-d₆) δ 11.40 (s, 1H), 7.35 (s, 1H), 7.28 (d, J=8.4 Hz, 1H), 6.98 (dd, J=8.0, 1.6 Hz, 1H), 6.66 (s, 1H), 6.52 (s, 2H), 4.86 (s, 1H), 4.40-4.43 (m, 2H), 3.10-3.20 (m, 2H), 2.81-2.83 (m, 1H), 2.34 (s, 3H), 1.87-1.90 (m, 2H), 1.55-1.58 (m, 2H). ESI-MS: m/z=325.3 (M+1).

### Example 18: Synthesis of (4-(5-aminoisoxazol-3-yl)piperidin-1-yl)(4-methyl-1H-indol-2-yl) methanone (18)

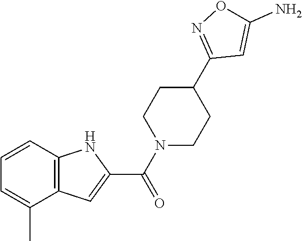 [SMILES: Cc1cccc2[nH]c(C(=O)N3CCC(c4cc(N)on4)CC3)cc12]

Similar to the preparation of 1, purification by Prep-HPLC provided the target compound 18 (22.53 mg, yield: 12%) as a pale pink solid. ¹HNMR (400 MHz, DMSO-d₆) δ 11.50 (s, 1H), 7.21 (d, J=8.4 Hz, 1H), 7.05 (t, J=6.8 Hz, 1H), 6.81 (d, J=6.8 Hz, 1H), 6.77 (s, 1H), 6.52 (s, 2H), 4.87 (s, 1H), 4.43-4.46 (m, 2H), 3.10-3.20 (m, 2H), 2.82-2.83 (m, 1H), 2.46 (s, 3H), 1.88-1.91 (m, 2H), 1.56-1.59 (m, 2H). ESI-MS: m/z=325.3 (M+1).

### Example 19: Synthesis of (4-(5-aminoisoxazol-3-yl)piperidin-1-yl)(1H-indol-1-yl)methanone (19)

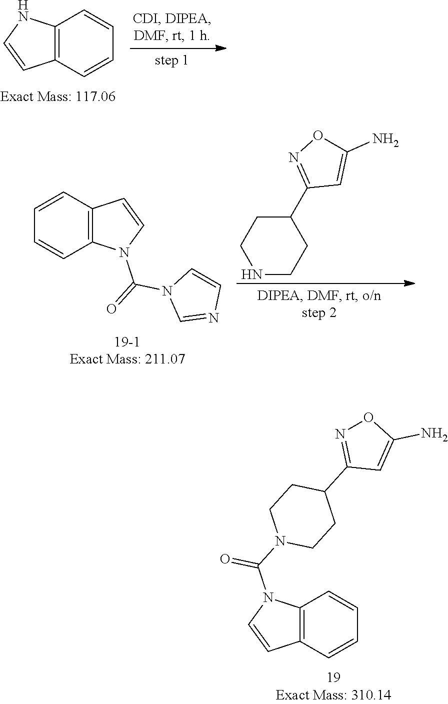

Synthesis of 19-1 (step 1): To a solution of 1H-indole (100 mg, 0.85 mmol) in DCM (5 mL), was added CDI (151.5 mg, 0.94 mmol) and DIPEA (219.3 mg, 1.70 mmol). The mixture was stirred at room temperature for 1 h, the mixture was used to next step directly without further purification.

Synthesis of 19 (step 2): To the solution of 19-1 was added 3-(piperidin-4-yl)isoxazol-5-amine (142.0 mg, 0.85 mmol). The mixture was stirred at room temperature overnight. The reaction mixture was poured into water (10 mL) and extracted with EtOAc (20 mL×3). The organic layer was washed with brine and evaporated, the residue crude product was purified by Prep-HPLC provided 19 (20.18 mg, yield: 8%) as a white solid. ¹HNMR (400 MHz, DMSO-d₆) δ 7.60 (t, J=7.6 Hz, 2H), 7.53 (d, J=3.6 Hz, 1H), 7.24 (t, J=8.0 Hz, 1H), 7.15 (t, J=8.0 Hz, 1H), 6.64 (d, J=3.2 Hz, 1H), 6.53 (s, 2H), 4.88 (s, 1H), 3.87-3.90 (m, 2H), 3.15-3.22 (m, 2H), 2.77-2.79 (m, 1H), 1.84-1.88 (m, 2H), 1.63-1.69 (m, 2H). ESI-MS: m/z=311.3 (M+1).

### Example 20: Synthesis of (4-(5-aminoisoxazol-3-yl)piperidin-1-yl)(5-methoxy-1H-indol-2-yl)methanone (20)

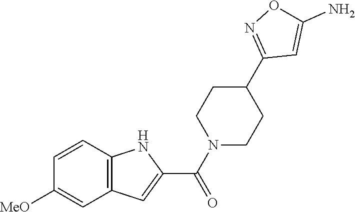 [SMILES: COc1ccc2[nH]c(C(=O)N3CCC(c4cc(N)on4)CC3)cc2c1]

Similar to the preparation of 1, purification by prep-HPLC provided the target compound 20 (18.6 mg, yield: 10.1%) as a white solid. ¹H NMR (400 MHz, DMSO-d₆) δ 11.38 (s, 1H), 7.28 (d, J=8.8 Hz, 1H), 7.05 (d, J=2.4 Hz, 1H), 6.81 (dd, J=9.2 Hz, J=2.8 Hz, 1H), 6.66 (s, 1H), 6.52 (s, 2H), 4.86 (s, 1H), 4.41 (d, J=13.2 Hz, 2H), 3.72 (s, 3H), 3.01-3.25 (m, 2H), 2.75-2.90 (m, 1H), 1.86-1.89 (m, 2H), 1.55-1.57 (m, 2H). ESI-MS: m/z=341.4 (M+1).

### Example 21: Synthesis of 3-(1-(1H-indol-2-ylsulfonyl)piperidin-4-yl)isoxazol-5-amine (21)

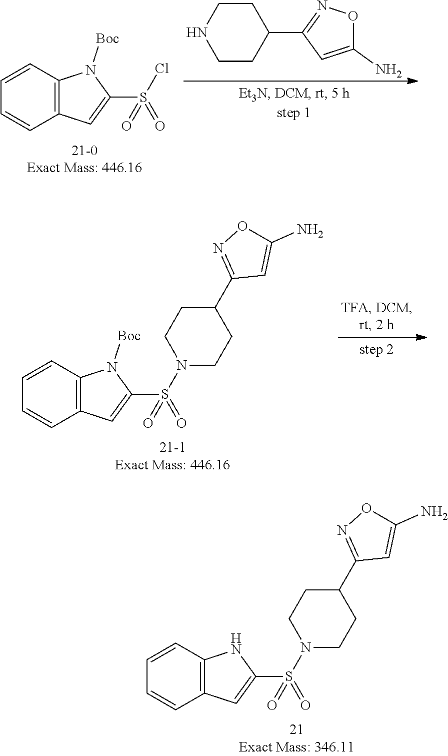 [SMILES: CC(C)(C)OC(=O)n1c(S(=O)(=O)Cl)cc2ccccc21 ; Nc1cc(C2CCNCC2)no1 ; CC(C)(C)OC(=O)n1c(S(=O)(=O)N2CCC(c3cc(N)on3)CC2)cc2ccccc21 ; Nc1cc(C2CCN(S(=O)(=O)c3cc4ccccc4[nH]3)CC2)no1]

Synthesis of 21-1 (step 1): The mixture of 21-0 (50 mg, 0.36 mmol), 3-(piperidin-4-yl)isoxazol-5-amine (90.2 mg, 0.54 mmol) and Et₃N (109.1 mg, 1.08 mmol) in DCM (3.00 mL) was stirred at room temperature for 5 h. The reaction mixture was poured into water (10 mL) and extracted with EtOAc (20 mL×3). The organic layer was washed with brine and evaporated, the residue crude product was purified by CC to give 21-1 (35 mg, yield: 22%) as a yellow solid.

Synthesis of 21 (step 2): The mixture of 21-1 (35 mg, 0.08 mmol) and TFA (45.6 mg, 0.40 mmol) in DCM (3.00 mL) was stirred at room temperature overnight. The reaction mixture was poured into water (10 mL) and extracted with EtOAc (20 mL×3). The organic layer was washed with brine and evaporated, the residue crude product was purified by Prep-HPLC provided 21 (13.09 yield: 47%) as a pink solid. ¹HNMR (400 MHz, DMSO-d₆) δ 12.15 (s, 1H), 7.70 (d, J=8.0 Hz, 1H), 7.48 (dd, J=8.4, 0.8 Hz, 1H), 7.28-7.33 (m, 1H), 7.13 (dt, J=8.0, 0.8 Hz, 1H), 7.01 (d, J=1.2 Hz, 1H), 6.51 (s, 2H), 4.78 (s, 1H), 3.67-3.70 (m, 2H), 3.30-3.31 (m, 2H), 2.51-2.52 (m, 1H), 1.86-1.90 (m, 2H), 1.57-1.61 (m, 2H). ESI-MS: m/z=347.2 (M+1).

### Example 22: Synthesis of (S)-(3-(5-aminoisoxazol-3-yl)pyrrolidin-1-yl)(1H-indol-2-yl)methanone (22)

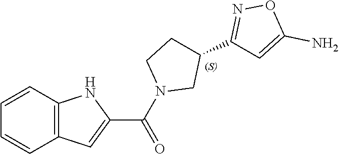

Similar to the preparation of 1, purification by Prep-HPLC gave 22 (12.8 mg, yield: 24%) as a white solid. ¹H NMR (400 MHz, DMSO-d₆) δ 11.56 (s, 1H), 7.63 (d, J=8.0 Hz, 1H), 7.44 (d, J=8.0 Hz, 1H), 7.19 (t, J=7.6 Hz, 1H), 7.04 (t, J=7.6 Hz, 1H), 6.98 (d, J=5.6 Hz, 1H), 6.62 (s, 2H), 4.95 (d, J=4.4 Hz, 1H), 4.15-4.17 (m, 1H), 3.87-3.98 (m, 2H), 3.41-3.71 (m, 2H), 2.00-2.24 (m, 2H). ESI-MS: m/z=297.4 (M+1).

### Example 23: Synthesis of (R)-(3-(5-aminoisoxazol-3-yl)pyrrolidin-1-yl)(1H-indol-2-yl)methanone (23)

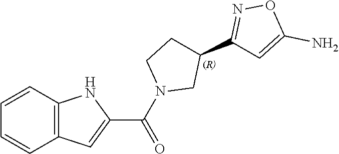

Similar to the preparation of 1, purification by Prep-HPLC gave 23 (17.8 mg, yield: 33%) as a white solid. ¹H NMR (400 MHz, DMSO-d₆) δ 11.56 (s, 1H), 7.63 (d, J=8.0 Hz, 1H), 7.44 (d, J=8.0 Hz, 1H), 7.19 (t, J=7.6 Hz, 1H), 7.04 (t, J=7.6 Hz, 1H), 6.98 (d, J=5.6 Hz, 1H), 6.62 (s, 2H), 4.95 (d, J=4.4 Hz, 1H), 4.15-4.17 (m, 1H), 3.87-3.98 (m, 2H), 3.41-3.71 (m, 2H), 2.00-2.24 (m, 2H). ESI-MS: m/z=297.4 (M+1).

### Example 24: Synthesis of (4-(5-aminoisoxazol-3-yl)piperidin-1-yl)(4-chlorophenyl)methanone (24)

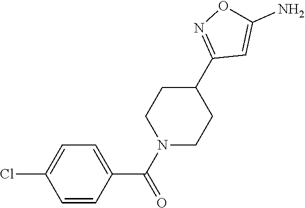 [SMILES: Nc1cc(C2CCN(C(=O)c3ccc(Cl)cc3)CC2)no1]

Similar to the preparation of 1, purification by Prep-HPLC provided the target compound 24 (23.91 mg, yield: 20.6%) as a white solid. ¹HNMR (400 MHz, DMSO-d₆) δ 7.49 (d, J=8.4 Hz, 2H), 7.41 (d, J=8.4 Hz, 2H), 6.52 (s, 2H), 4.86 (s, 1H), 4.38-4.46 (m, 1H), 3.51-3.59 (m, 1H), 3.08-3.16 (m, 1H), 2.88-2.94 (m, 1H), 2.72-2.79 (m, 1H), 1.70-1.89 (m, 2H), 1.48-1.56 (m, 2H). ESI-MS: m/z=306.0 (M+1).

### Example 25: Synthesis of (4-(5-aminoisoxazol-3-yl)piperidin-1-yl)(biphenyl-4-yl)methanone (25)

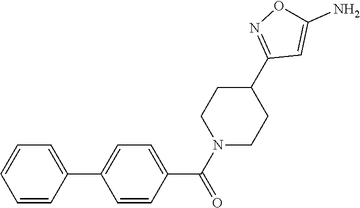 [SMILES: Nc1cc(C2CCN(C(=O)c3ccc(-c4ccccc4)cc3)CC2)no1]

Similar to the preparation of 1, purification by Prep-HPLC provided the target compound 25 (24.59 mg, yield: 17.4%) as a white solid. ¹HNMR (400 MHz, DMSO-d₆): δ 7.68-7.72 (m, 4H), 7.45-7.48 (m, 4H), 7.36-7.40 (m, 1H), 6.52 (s, 2H), 4.87 (s, 1H), 4.42-4.52 (m, 1H), 3.60-3.72 (m, 1H), 2.90-3.21 (m, 2H), 2.72-2.81 (m, 1H), 1.73-1.91 (m, 2H), 1.49-1.61 (m, 2H). ESI-MS: m/z=348.3 (M+1).

### Example 26: Synthesis of (4-(5-aminoisoxazol-3-yl)piperidin-1-yl)(4-methoxyphenyl)methanone (26)

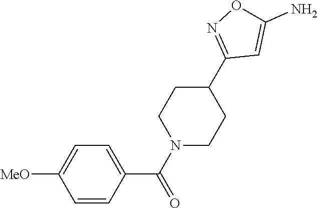 [SMILES: COc1ccc(C(=O)N2CCC(c3cc(N)on3)CC2)cc1]

Similar to the preparation of 1, purification by Prep-HPLC provided the target compound 26 (23.02 mg, yield: 20%) as a yellow solid. ¹HNMR (400 MHz, DMSO-d₆): δ 7.32-7.36 (m, 2H), 6.94-6.97 (m, 2H), 6.51 (s, 2H), 4.86 (s, 1H), 4.19-4.51 (m, 1H), 3.77 (s, 3H), 3.72-3.78 (m, 1H), 2.91-3.11 (m, 2H), 2.72-2.77 (m, 1H), 1.74-1.85 (m, 2H), 1.46-1.55 (m, 2H). ESI-MS: m/z=302.2 (M+1).

### Example 27: Synthesis of (4-(5-aminoisoxazol-3-yl)piperidin-1-yl)(4-(trifluoromethyl)phenyl)methanone (27)

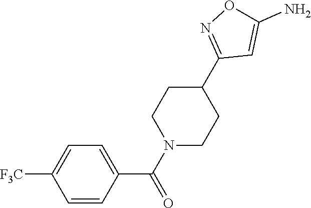 [SMILES: Nc1cc(C2CCN(C(=O)c3ccc(C(F)(F)F)cc3)CC2)no1]

Similar to the preparation of 1, purification by prep-HPLC provided the target compound 27 (15.0 mg, yield: 7.4%) as a yellow solid. ¹H NMR (400 MHz, DMSO-d₆) δ 7.80 (d, J=8.0 Hz, 2H), 7.63 (d, J=8.4 Hz, 2H), 6.54 (s, 2H), 4.88 (s, 1H), 4.46 (d, J=11.2 Hz, 1H), 3.48 (d, J=12.0 Hz, 1H), 3.14 (t, J=15.6 Hz, 1H), 2.87-3.00 (m, 1H), 2.75-2.83 (m, 1H), 1.90 (d, J=12.8 Hz, 1H), 1.74 (d, J=16.0 Hz, 1H), 1.48-1.62 (m, 2H). ESI-MS: m/z=340.3 (M+1).

### Example 28: Synthesis of (4-(5-aminoisoxazol-3-yl)piperidin-1-yl)(4-tert-butylphenyl)methanone (28)

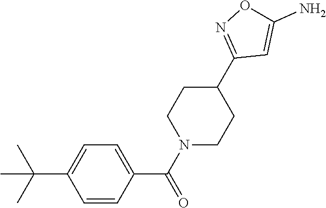 [SMILES: CC(C)(C)c1ccc(C(=O)N2CCC(c3cc(N)on3)CC2)cc1]

Similar to the preparation of 1, purification by Prep-HPLC provided the target compound 28 (10 mg, yield: 10%) as a pale white solid. ¹HNMR (400 MHz, DMSO-d₆) δ 7.43 (d, J=8.4 Hz, 2H), 7.3 (d, J=8.4 Hz, 2H), 6.50 (s, 2H), 4.85 (s, 1H), 4.48-4.52 (m, 1H), 3.53-3.75 (m, 1H), 2.85-3.12 (m, 2H), 2.71-2.78 (m, 1H), 1.86-1.73 (m, 2H), 1.50 (d, J=12.8 Hz, 2H), 1.27 (s, 9H). ESI-MS: m/z=328.4 (M+1).

### Example 29: Synthesis of (4-(5-aminoisoxazol-3-yl)piperidin-1-yl)(p-tolyl)methanone (29)

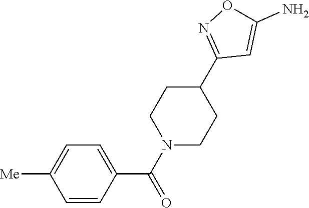 [SMILES: Cc1ccc(C(=O)N2CCC(c3cc(N)on3)CC2)cc1]

Similar to the preparation of 1, purification by Prep-HPLC provided the target compound 29 (6.51 mg, yield: 6%) as a pale white solid. ¹HNMR (400 MHz, DMSO) δ 7.28 (d, J=8 Hz, 2H), 7.24 (d, J=7.6 Hz, 2H), 6.54 (s, 2H), 4.87 (s, 1H), 4.73 (brs, 1H), 3.64-3.59 (m, 2H), 3.05-2.88 (m, 2H), 2.80˜2.75 (m, 1H), 1.87-1.73 (m, 4H), 1.52 (s, 2H). ESI-MS: m/z=286.2 (M+1).

### Example 30: Synthesis of (3-(5-aminoisoxazol-3-yl)pyrrolidin-1-yl)(4-bromophenyl)methanone (30)

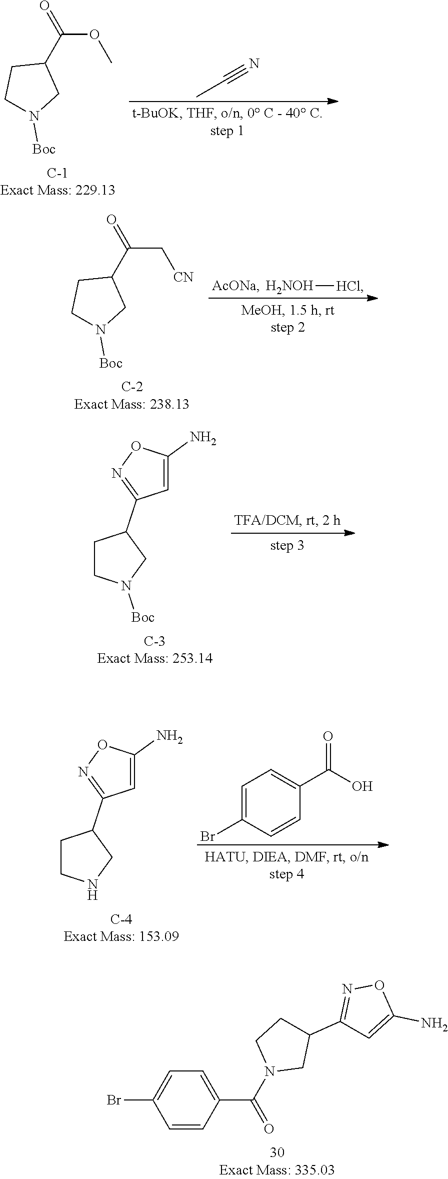

Synthesis of C-2 (step 1): To a solution of t-BuOK (14.6 g, 131 mmol) in THF (200 mL), was added MeCN (17.8 g, 43.6 mmol). The mixture was stirred at 0° C. for 0.5 h. Then added compound C-1 (10 g, 43.6 mmol) and the reaction mixture was stirred overnight at 40° C. Then added water, the aqueous phase was extracted with EtOAc, the combined organic phases were dried over anhydrous sodium sulfate, filtered, and concentrated, the crude was purified by c.c. to give compound C-2 (980 mg, 9.5%) as a yellow oil.

Synthesis of C-3 (step 2): To a solution of C-2 (980 mg, 4.12 mmol) in MeOH (20 mL), was added Sodium acetate (1.01 g, 12.4 mmol) and Hydroxyl ammonium hydrochloride (572.6 mg, 8.24 mmol). The mixture was stirred at room temperature overnight. The reaction mixture was poured into water (50 mL) and extracted with EtOAc (40 mL×3). The organic layer was washed with brine and evaporated, the residue crude product was purified by column chromatography (PE/EtOAc=5/1) to give C-3 (730 mg, 70.1%) as a yellow solid.

Synthesis of C-4 (step 3): To a solution of C-3 (730 mg, 2.88 mmol) in DCM (10 mL), was added TFA (10 mL). The mixture was stirred at room temperature for 3 h, The solvent was removed in vacuo to give compound C-4 (400 mg, 90.9%) as a yellow solid.

Synthesis of 30 (step 4): To a stirred solution of compound C-4 (100 mg, 0.65 mmol) in DMF (5 ml) was added 4-bromobenzoic acid (130 mg, 0.65 mmol), HATU (370 mg, 0.98 mmol) and DIEA (252 mg, 1.95 mmol). The resulting reaction mixture was stirred at rt for 16 h. Then added water, the aqueous phase was extracted with dichloromethane, the combined organic phases were dried over anhydrous sodium sulfate, filtered, and concentrated in vacuo, purified by prep-HPLC provided 30 (35 mg, yield: 16.0%) as a white solid. ¹H NMR (400 MHz, DMSO-d₆) δ 7.64 (t, J=8.4 Hz, 2H), 7.49 (d, J=8.4 Hz, 2H), 6.63-6.60 (m, 2H), 4.85-4.94 (m, 1H), 3.45-3.83 (m, 4H), 3.24-3.31 (m, 1H), 2.14-2.27 (m, 1H), 1.89-2.04 (m, 1H). ESI-MS: m/z=338.3 (M+1).

### Example 31: Synthesis of (3-(5-aminoisoxazol-3-yl)pyrrolidin-1-yl)(phenyl)methanone (31)

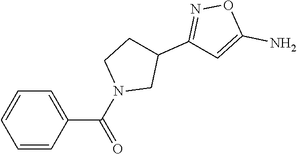 [SMILES: Nc1cc(C2CCN(C(=O)c3ccccc3)C2)no1]

Similar to the preparation of 30, purification by prep-HPLC provided the target compound 31 (25.0 mg, yield: 7.9%) as a yellow solid. ¹H NMR (400 MHz, DMSO-d₆) δ 7.44-7.53 (m, 5H), 6.62 (d, J=10.8 Hz, 2H), 4.84-4.94 (m, 1H), 3.49-3.83 (m, 4H), 3.21-3.28 (m, 1H), 2.12-2.26 (m, 1H), 1.87-2.03 (m, 1H). ESI-MS: m/z=258.4 (M+1).

### Example 32: Synthesis of (3-(5-aminoisoxazol-3-yl)pyrrolidin-1-yl)(6-chloro-1H-indol-2-yl)methanone (32)

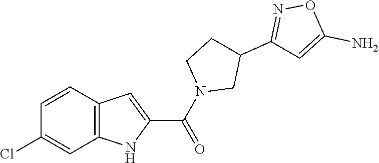

Similar to the preparation of 30, purification by prep-HPLC provided the target compound 32 (35.0 mg, yield: 20.7%) as a white solid. ¹H NMR (400 MHz, DMSO-d₆) δ 11.71 (s, 1H), 7.66 (d, J=8.4 Hz, 1H), 7.45 (s, 1H), 7.02-7.07 (m, 2H), 6.63 (s, 2H), 4.95 (d, J=6.8 Hz, 1H), 3.41-4.19 (m, 5H), 1.97-2.24 (m, 2H). ESI-MS: m/z=331.1 (M+1)

### Example 33: Synthesis of (3-(5-aminoisoxazol-3-yl)pyrrolidin-1-yl)(5,6-dichloro-1H-indol-2-yl)methanone (33)

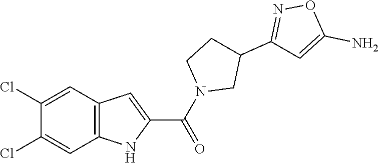

Similar to the preparation of 30, purification by prep-HPLC provided the target compound 33 (15.0 mg, yield: 9.4%) as a white solid. ¹H NMR (400 MHz, DMSO-d₆) δ 11.88 (s, 1H), 7.92 (s, 1H), 7.63 (s, 1H), 7.01 (s, 1H), 6.64 (d, J=3.6 Hz, 2H), 4.95 (d, J=6.4 Hz, 1H), 3.62-4.20 (m, 5H), 1.98-2.24 (m, 2H). ESI-MS: m/z=365.2 (M+1)

### Example 34: Synthesis of (4-(5-aminoisoxazol-3-yl)piperidin-1-yl)(2-methyl-4-(trifluoromethyl)phenyl)methanone (34)

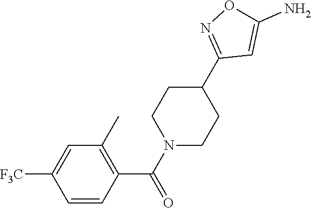 [SMILES: Cc1cc(C(F)(F)F)ccc1C(=O)N1CCC(c2cc(N)on2)CC1]

Similar to the preparation of 1, purification by Prep-HPLC provided the target compound 34 (20 mg, yield: 15%) as a white solid. ¹HNMR (400 MHz, DMSO-d₆): δ 7.68 (s, 1H), 7.60-7.62 (m, 1H), 7.38-7.49 (m, 1H), 6.55 (s, 2H), 4.85-4.89 (m, 1H), 4.50-4.53 (m, 1H), 3.21-3.28 (m, 1H), 3.07-3.13 (m, 1H), 2.91-3.02 (m, 1H), 2.68-2.81 (m, 1H), 2.28-2.33 (m, 3H), 1.79-1.96 (m, 1H), 1.37-1.77 (m, 3H). ESI-MS: m/z=354.3 (M+1)

### Example 35: Synthesis of (4-(5-aminoisoxazol-3-yl)piperidin-1-yl)(3-(trifluoromethyl)phenyl)methanone (35)

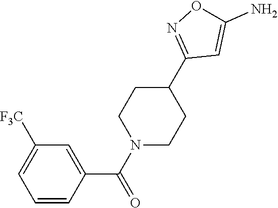 [SMILES: Nc1cc(C2CCN(C(=O)c3cccc(C(F)(F)F)c3)CC2)no1]

Similar to the preparation of 1, purification by prep-HPLC provided the target compound 35 (35.0 mg, yield: 13.1%) as a white solid. ¹H NMR (400 MHz, DMSO-d₆) δ 7.83 (d, J=7.2 Hz, 1H), 7.67-7.76 (m, 3H), 6.55 (s, 2H), 4.89 (s, 1H), 4.46 (d, J=9.2 Hz, 1H), 3.49 (t, J=5.6 Hz, 1H), 3.19 (s, 1H), 2.94 (s, 1H), 2.75-2.82 (m, 1H), 1.83-1.91 (m, 1H), 1.73 (t, J=11.2 Hz, 1H), 1.58 (s, 2H). ESI-MS: m/z=340.1 (M+1).

### Example 36: Synthesis of (4-(5-aminoisoxazol-3-yl)piperidin-1-yl)(4-fluorophenyl)methanone (36)

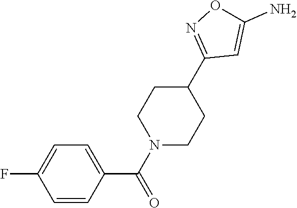 [SMILES: Nc1cc(C2CCN(C(=O)c3ccc(F)cc3)CC2)no1]

Similar to the preparation of 1, purification by prep-HPLC provided the target compound 36 (43.0 mg, yield: 13.9%) as a white solid. ¹H NMR (400 MHz, DMSO-d₆) δ 7.46-7.49 (m, 2H), 7.23-7.30 (m, 2H), 6.55 (s, 2H), 4.88 (s, 1H), 4.43 (brs, 1H), 3.58 (brs, 1H), 3.13 (brs, 1H), 2.75-2.81 (m, 1H) 2.81-2.92 (m, 1H), 1.76-1.86 (m, 2H), 1.50-1.58 (m, 2H). ESI-MS: m/z=290.2 (M+1).

### Example 37: Synthesis of (4-(5-aminoisoxazol-3-yl)piperidin-1-yl)(4-morpholinophenyl) methanone (37)

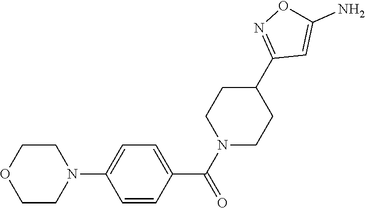 [SMILES: Nc1cc(C2CCN(C(=O)c3ccc(N4CCOCC4)cc3)CC2)no1]

Similar to the preparation of 1, purification by Prep-HPLC provided the target compound 37 (24.11 mg, yield: 11%) as a white solid. ¹HNMR (400 MHz, DMSO-d₆) δ 7.29 (d, J=8.8 Hz, 2H), 6.96 (d, J=8.8 Hz, 2H), 6.54 (s, 2H), 4.88 (s, 1H), 3.93-4.38 (m, 2H), 3.74 (t, J=5.2 Hz, 4H), 3.17 (t, J=5.2 Hz, 4H), 2.94-3.08 (m, 2H), 2.72-2.80 (m, 1H), 1.77-1.86 (m, 2H), 1.46-1.57 (m, 2H). ESI-MS: m/z=357.3 (M+1).

### Example 38: Synthesis of (4-(5-aminoisoxazol-3-yl)piperidin-1-yl)(4-(pyridin-2-yl)phenyl)methanone (38)

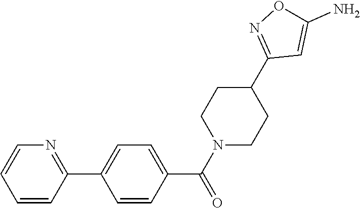 [SMILES: Nc1cc(C2CCN(C(=O)c3ccc(-c4ccccn4)cc3)CC2)no1]

Similar to the preparation of 1, purification by Prep-HPLC provided the target compound 38 (40 mg, yield: 40%) as a pale white solid. ¹HNMR (400 MHz, DMSO-d₆) δ 8.69 (d, J=4 Hz, 1H), 8.15 (d, J=8 Hz, 2H), 8.02 (d, J=8 Hz, 1H), 7.93-7.89 (m, 1H), 7.52 (d, J=8.4 Hz, 2H), 7.40-7.37 (m, 1H), 6.55 (s, 2H), 4.90 (s, 1H), 4.52-4.45 (m, 1H), 3.69-3.62 (m, 1H), 3.21-3.12 (m, 1H), 2.80-2.96 (m, 1H), 2.75-2.80 (m, 1H), 1.90-1.78 (m, 2H), 1.57 (d, J=10.8 Hz, 2H). ESI-MS: m/z=349.1 (M+1).

### Example 39: Synthesis of (4-(5-aminoisoxazol-3-yl)piperidin-1-yl)(³-methyl-4-(trifluoromethyl)phenyl)methanone (39)

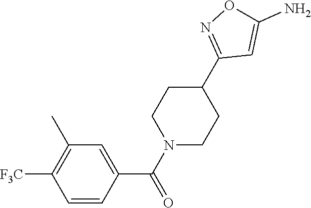 [SMILES: Cc1cc(C(=O)N2CCC(c3cc(N)on3)CC2)ccc1C(F)(F)F]

Similar to the preparation of 1, purification by Prep-HPLC provided the target compound 39 (27 mg, yield: 27%) as a pale white solid. ¹HNMR (400 MHz, DMSO-d₆) δ 7.73 (d, J=8 Hz, 1H), 7.47 (s, 1H), 7.40 (d, J=8 Hz, 1H), 6.54 (s, 2H), 4.88 (s, 1H), 4.45 (d, J=12 Hz, 1H), 3.49 (d, J=12.8 Hz, 1H), 3.14 (t, J=12.6 Hz, 1H), 2.99-2.87 (m, 1H), 2.81-2.75 (m, 1H), 2.47 (s, 3H), 1.90 (d, J=12.4 Hz, 1H), 1.74 (d, J=12 Hz, 1H), 1.55 (t, J=11.6 Hz, 2H). ESI-MS: m/z=354.1 (M+1).

### Example 40: Synthesis of (4-(5-aminoisoxazol-3-yl)piperidin-1-yl)(4-((dimethylamino)methyl) phenyl)methanone (40)

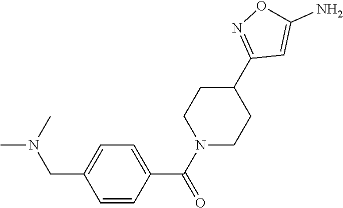 [SMILES: CN(C)Cc1ccc(C(=O)N2CCC(c3cc(N)on3)CC2)cc1]

Similar to the preparation of 1, purification by Prep-HPLC provided the target compound 40 (37.74 mg, yield: 19%) as a pale yellow solid. ¹HNMR (400 MHz, DMSO-d₆) δ 7.35 (s, 4H), 6.55 (s, 2H), 4.88 (s, 1H), 4.38-4.55 (m, 1H), 3.53-3.65 (m, 1H), 3.43 (s, 2H), 3.06-3.21 (m, 1H), 2.85-2.96 (m, 1H), 2.73-2.80 (m, 1H), 2.16 (s, 6H), 1.70-1.73 (m, 2H), 1.46-1.60 (m, 2H). ESI-MS: m/z=329.4 (M+1).

### Example 41: Synthesis of 4-(4-(5-aminoisoxazol-3-yl)piperidine-1-carbonyl)benzonitrile (41)

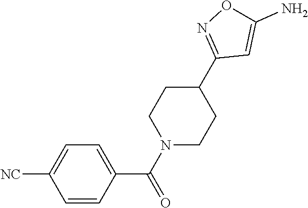 [SMILES: N#Cc1ccc(C(=O)N2CCC(c3cc(N)on3)CC2)cc1]

Similar to the preparation of 1, purification by prep-HPLC provided the target compound 41 (23.0 mg, yield: 7.6%) as a white solid. ¹H NMR (400 MHz, DMSO-d₆) δ 8.33 (d, J=8.4 Hz, 2H), 8.10 (d, J=8.4 Hz, 2H), 6.40 (s, 2H), 5.47 (s, 1H), 5.03 (bs, 1H), 4.11 (bs, 1H), 3.68 (bs, 1H), 3.45 (bs, 1H), 3.27-3.38 (m, 1H), 2.12-2.42 (m, 4H). ESI-MS: m/z=297.3 (M+1).

### Example 42: Synthesis of (3-(5-aminoisoxazol-3-yl)pyrrolidin-1-yl)(4-(trifluoromethyl)phenyl)methanone (42)

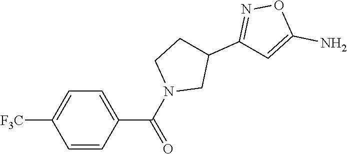 [SMILES: Nc1cc(C2CCN(C(=O)c3ccc(C(F)(F)F)cc3)C2)no1]

Similar to the preparation of 30, purification by pre-HPLC to provide the target compound 42 (10.0 mg, yield: 5.8%) as a yellow solid. ¹H NMR (400 MHz, DMSO-d₆) δ 8.21-8.27 (m, 4H), 6.48 (d, J=13.2 Hz, 2H), 5.45-5.54 (m, 1H), 3.79-4.40 (m, 5H), 2.69-2.83 (m, 1H), 1.76 (s, 1H). ESI-MS: m/z=326.3 (M+1).

### Example 43: Synthesis of (4-(5-aminoisoxazol-3-yl)piperidin-1-yl)(4-(trifluoromethoxy)phenyl)methanoneone (43)

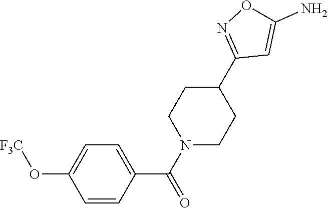 [SMILES: Nc1cc(C2CCN(C(=O)c3ccc(OC(F)(F)F)cc3)CC2)no1]

Similar to the preparation of 1, purification by prep-HPLC provided the target compound 43 (15.0 mg, yield: 7.3%) as a white solid. ¹H NMR (400 MHz, DMSO-d₆) δ 7.54-7.56 (m, 2H), 7.44 (d, J=7.6 Hz, 2H), 6.55 (s, 2H), 4.88 (s, 1H), 4.62 (bs, 1H), 3.55 (bs, 1H), 3.15 (bs, 1H), 2.92 (bs, 1H), 2.74-2.82 (m, 1H), 1.88 (bs, 1H), 1.76 (bs, 1H), 1.55 (d, J=15.6 Hz, 2H). ESI-MS: m/z=356.1 (M+1).

### Example 44: Synthesis of (4-(5-aminoisoxazol-3-yl)piperidin-1-yl)(2-(trifluoromethyl)phenyl)methanone (44)

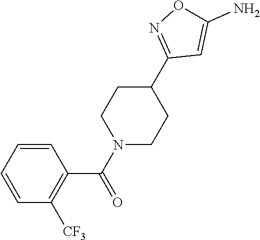 [SMILES: Nc1cc(C2CCN(C(=O)c3ccccc3C(F)(F)F)CC2)no1]

Similar to the preparation of 1, purification by prep-HPLC provided the target compound 44 (16.8 mg, yield: 7.9%) as a white solid. ¹H NMR (400 MHz, DMSO-d₆) δ 7.80-7.83 (m, 1H), 7.55 (t, J=7.4 Hz, 1H), 7.65 (t, J=7.6 Hz, 1H), 7.44-7.56 (m, 1H), 6.55 (s, 2H), 4.-79-4.89 (m, 1H), 4.48 (d, J=13.2 Hz, 1H), 2.86-3.22 (m, 3H), 2.76-2.82 (m, 1H), 1.91 (t, J=12.4 Hz, 1H), 1.71 (t, J=14.8 Hz, 1H), 1.31-1.62 (m, 2H). ESI-MS: m/z=340.2 (M+1).

### Example 45: Synthesis of (4-(5-aminoisoxazol-3-yl)piperidin-1-yl)(4-nitrophenyl)methanone (45)

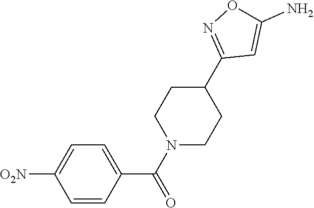 [SMILES: Nc1cc(C2CCN(C(=O)c3ccc([N+](=O)[O-])cc3)CC2)no1]

Similar to the preparation of 1, purification by prep-HPLC provided the target compound 45 (20.0 mg, yield: 8.8%) as a yellow solid. ¹H NMR (400 MHz, DMSO-d₆) δ 8.29 (d, J=8.8 Hz, 2H), 7.69 (d, J=8.8 Hz, 2H), 6.55 (s, 2H), 4.89 (s, 1H), 4.46 (d, J=13.2 Hz, 1H), 3.45 (d, J=13.2 Hz, 1H), 3.16 (t, J=13.4 Hz, 1H), 2.96 (t, J=12.2 Hz, 1H), 2.77-2.84 (m, 1H), 1.92 (d, J=16.0 Hz, 1H), 1.74 (d, J=13.6 Hz, 1H), 1.51-1.64 (m, 2H). ESI-MS: m/z=317.3 (M+1).

### Example 46: Synthesis of (4-(5-aminoisoxazol-3-yl)piperidin-1-yl)(4-methyl-3-(trifluoromethyl)phenyl)methanone (46)

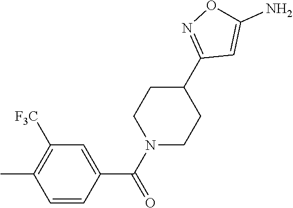 [SMILES: Cc1ccc(C(=O)N2CCC(c3cc(N)on3)CC2)cc1C(F)(F)F]

Similar to the preparation of 1, purification by prep-HPLC provided the target compound 46 (42 mg, yield: 20.3%) as a white solid. ¹H NMR (400 MHz, DMSO-d₆) δ 7.67 (s, 1H), 7.61 (d, J=7.6 Hz, 1H), 7.52 (d, J=7.6 Hz, 1H), 6.54 (s, 2H), 4.87 (s, 1H), 4.43 (s, 1H), 3.54 (s, 1H), 3.16 (s, 1H), 2.94 (s, 1H), 2.75-2.81 (m, 1H), 2.48-2.51 (m, 3H), 1.88 (s, 1H), 1.75 (s, 1H), 1.56 (s, 2H). ESI-MS: m/z=354.3 (M+1).

### Example 47: Synthesis of (4-(5-aminoisoxazol-3-yl)piperidin-1-yl)(2-chloro-4-(trifluoromethyl)phenyl)methanone (47)

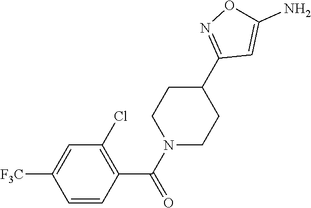 [SMILES: Nc1cc(C2CCN(C(=O)c3ccc(C(F)(F)F)cc3Cl)CC2)no1]

Similar to the preparation of 1, purification by prep-HPLC provided the target compound 47 (45.0 mg, yield: 22.5%) as a white solid. ¹H NMR (400 MHz, DMSO-d₆) δ 7.99 (s, 1H), 7.81 (d, J=7.6 Hz, 1H), 7.60-7.73 (m, 1H), 6.55 (s, 2H), 4.86 (d, J=18 Hz, 1H), 4.48 (d, J=13.2 Hz, 1H), 2.23-3.27 (m, 1H), 3.08-3.18 (m, 1H), 2.93-3.02 (m, 1H), 2.77-2.84 (m, 1H), 1.93 (t, J=14.8 Hz, 1H), 1.70-1.82 (m, 1H), 1.47-1.62 (m, 2H). ESI-MS: m/z=374.2 (M+1).

### Example 48: Synthesis of (4-(5-aminoisoxazol-3-yl)piperidin-1-yl)(perfluorophenyl)methanone (48)

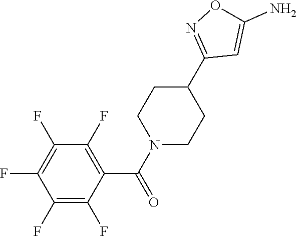 [SMILES: Nc1cc(C2CCN(C(=O)c3c(F)c(F)c(F)c(F)c3F)CC2)no1]

Similar to the preparation of 1, purification by prep-HPLC provided the target compound 48 (33.6 mg, yield: 16.5%) as a white solid. ¹H NMR (400 MHz, DMSO-d₆) δ 6.56 (s, 2H), 4.86 (s, 1H), 4.45 (d, J=13.2 Hz, 1H), 3.59 (d, J=13.6 Hz, 1H), 3.19-3.26 (m, 1H), 3.00-3.07 (m, 1H), 2.79-2.87 (m, 1H), 1.96 (d, J=13.2 Hz, 1H), 1.82 (d, J=13.2 Hz, 1H), 1.38-1.55 (m, 2H). ESI-MS: m/z=362.1 (M+1).

### Example 49: Synthesis of (4-(5-aminoisoxazol-3-yl)piperidin-1-yl)(3-tert-butylphenyl)methanone (49)

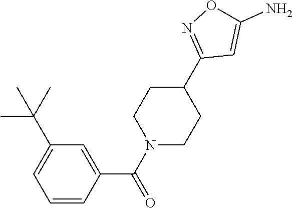 [SMILES: CC(C)(C)c1cccc(C(=O)N2CCC(c3cc(N)on3)CC2)c1]

Similar to the preparation of 1, purification by prep-HPLC provided the target compound 49 (19.8 mg, yield: 9.0%) as a white solid. ¹H NMR (400 MHz, DMSO-d₆) δ 7.46-7.49 (m, 1H), 7.35-7.38 (m, 2H), 7.18-7.21 (m, 1H), 6.54 (s, 2H), 4.89 (s, 1H), 4.46 (s, 1H), 3.58 (s, 1H), 3.11 (s, 1H), 2.94 (s, 1H), 2.74-2.80 (m, 1H), 1.88 (s, 2H), 1.75 (s, 2H), 1.29 (s, 9H). ESI-MS: m/z=328.4 (M+1).

### Example 50: Synthesis of (4-(5-aminoisoxazol-3-yl)piperidin-1-yl)(3-chloro-4-(trifluoromethyl)phenyl)methanone (50)

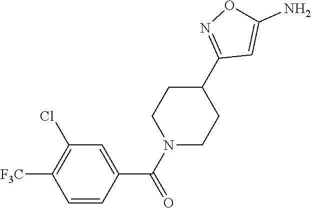 [SMILES: Nc1cc(C2CCN(C(=O)c3ccc(C(F)(F)F)c(Cl)c3)CC2)no1]

Similar to the preparation of 1, purification by Prep-HPLC provided the target compound 50 (13.07 mg, yield: 13%) as a pale white solid. ¹HNMR (400 MHz, DMSO) δ 7.93 (d, J=8.4 Hz, 1H), 7.80 (s, 1H), 7.57 (d, J=8 Hz, 1H), 6.54 (s, 2H), 4.88 (s, 1H), 4.44 (d, J=13.6 Hz, 1H), 3.48-3.44 (m, 1H), 3.18-3.11 (m, 1H), 2.97-2.90 (m, 1H), 2.81-2.75 (m, 1H), 1.91-1.88 (m, 1H), 1.76-1.72 (m, 1H), 1.60-1.55 (m, 2H). ESI-MS: m/z=374.1 (M+1).

### Example 51: Synthesis of (4-(5-aminoisoxazol-3-yl)piperidin-1-yl)(3-chloro-5-(trifluoromethyl)phenyl)methanone (51)

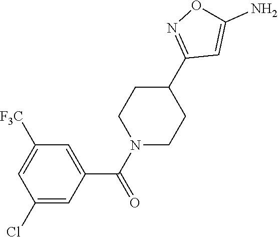 [SMILES: Nc1cc(C2CCN(C(=O)c3cc(Cl)cc(C(F)(F)F)c3)CC2)no1]

Similar to the preparation of 1, purification by Prep-HPLC provided the target compound 51 (15.95 mg, yield: 15%) as a pale white solid. ¹HNMR (400 MHz, DMSO-d₆) δ 8.20 (s, 1H), 8.15 (s, 2H), 6.54 (s, 2H), 4.89 (s, 1H), 4.47-4.44 (m, 1H), 3.45-3.39 (m, 1H), 3.22-3.15 (m, 1H), 2.99-2.93 (m, 1H), 2.82-2.76 (m, 1H), 1.95-1.85 (m, 1H), 1.77-1.57 (m, 3H). ESI-MS: m/z=374.1 (M+1).

### Example 52: Synthesis of (4-(5-aminoisoxazol-3-yl)piperidin-1-yl)(3,5-bis(trifluoromethyl)phenyl)methanone (52)

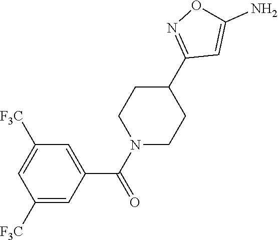 [SMILES: Nc1cc(C2CCN(C(=O)c3cc(C(F)(F)F)cc(C(F)(F)F)c3)CC2)no1]

Similar to the preparation of 1, purification by Prep-HPLC provided the target compound 52 (14.88 mg, yield: 15%) as a pale white solid. ¹HNMR (400 MHz, DMSO) δ 7.96 (s, 1H), 7.85 (s, 1H), 7.77 (s, 1H), 6.54 (s, 2H), 4.89 (s, 1H), 4.45-4.42 (m, 1H), 3.47-3.42 (m, 1H), 3.20-3.14 (m, 1H), 2.96-2.91 (m, 1H), 2.81-2.74 (m, 1H), 1.90-1.87 (m, 1H), 1.74-1.71 (m, 1H), 1.62-1.57 (m, 2H). ESI-MS: m/z=408.1 (M+1).

### Example 53: Synthesis of (4-(5-aminoisoxazol-3-yl)piperidin-1-yl)(4-cyclopropylphenyl)methanone (53)

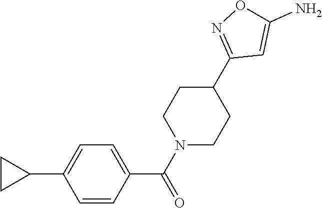 [SMILES: Nc1cc(C2CCN(C(=O)c3ccc(C4CC4)cc3)CC2)no1]

Similar to the preparation of 1, purification by Prep-HPLC provided the target compound 53 (24.87 mg, yield: 21%) as a white solid. ¹HNMR (400 MHz, DMSO-d₆): δ 7.27 (d, J 8.4 Hz, 2H), 7.12 (d, J 8.4 Hz, 2H), 6.54 (s, 2H), 4.86 (s, 1H), 4.37-4.48 (m, 1H), 3.60-3.68 (m, 1H), 2.89-3.15 (m, 2H), 2.73-2.79 (m, 1H), 1.92-1.97 (m, 1H), 1.75-1.84 (m, 2H), 1.50-1.56 (m, 2H), 0.96-1.00 (m, 2H), 0.69-0.73 (m, 2H). ESI-MS: m/z=312.3 (M+1).

### Example 54: Synthesis of 3-(1-(4-(trifluoromethyl)phenylsulfonyl)piperidin-4-yl)isoxazol-5-amine (54)

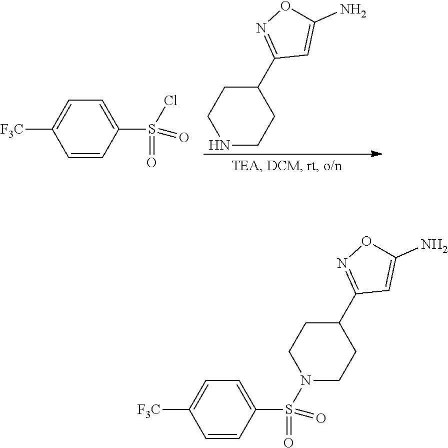

Synthesis 54: The mixture of 3-(piperidin-4-yl)isoxazol-5-amine (123 mg, 0.73 mmol), 4-(trifluoromethyl)benzene-1-sulfonyl chloride (150 mg, 0.61 mmol), TEA (185 mg, 1.83 mmol) in DCM (5.00 mL) was stirred at room temperature overnight. The reaction mixture was evaporated, the residue crude product was purified by Prep-HPLC provided 54 (23.40 mg, yield: 10%) as a white solid. ¹HNMR (400 MHz, DMSO-d₆) δ 7.97-8.06 (m, 4H), 6.53 (s, 2H), 4.81 (s, 1H), 3.65-3.68 (m, 2H), 2.42-2.55 (m, 3H), 1.85-1.89 (m, 2H), 1.54-1.64 (m, 2H). ESI-MS: m/z=376.2 (M+1).

### Example 55: Synthesis of (4-(5-aminoisoxazol-3-yl)piperidin-1-yl)(3,4-bis(trifluoromethyl)phenyl)methanone (55)

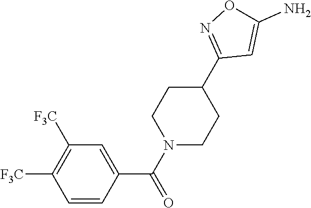 [SMILES: Nc1cc(C2CCN(C(=O)c3ccc(C(F)(F)F)c(C(F)(F)F)c3)CC2)no1]

Similar to the preparation of 1, purification by prep-HPLC provided the target compound 55 (9.4 mg, yield: 7.5%) as a pale yellow solid. ¹H NMR (400 MHz, DMSO-d₆) δ 8.13 (d, J=8.4 Hz, 1H), 8.07 (s, 1H), 7.96 (d, J=8.0 Hz, 1H), 6.55 (s, 2H), 4.89 (s, 1H), 4.46 (d, J=12.0 Hz, 1H), 3.46 (d, J=13.6 Hz, 1H), 3.18 (t, J=12.2 Hz, 1H), 2.97 (t, J=11.6 Hz, 1H), 2.76-2.83 (m, 1H), 1.91 (d, J=12.8 Hz, 1H), 1.74 (d, J=12.0 Hz, 1H), 1.57-1.64 (m, 2H). ESI-MS: m/z=407.8 (M+1).

### Example 56: Synthesis of (4-(5-aminoisoxazol-3-yl)piperidin-1-yl)(4-methyl-2-(trifluoromethyl) phenyl)methanone (56)

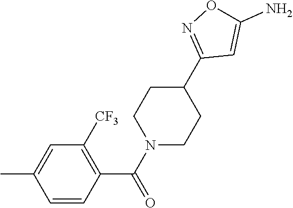 [SMILES: Cc1ccc(C(=O)N2CCC(c3cc(N)on3)CC2)c(C(F)(F)F)c1]

Similar to the preparation of 1, purification by Prep-HPLC provided the target compound 56 (29.48 mg, yield: 14%) as a white solid. ¹HNMR (400 MHz, DMSO-d₆) δ 7.62-7.63 (m, 1H), 7.54 (d, J=7.6 Hz, 1H), 7.32-7.42 (m, 1H), 6.55 (s, 2H), 4.78-4.89 (m, 1H), 4.47 (d, J=13.2 Hz, 1H), 2.88-3.27 (m, 3H), 2.75-2.81 (m, 1H), 2.41 (s, 3H), 1.88-1.93 (m, 1H), 1.66-1.73 (m, 1H), 1.30-1.60 (m, 2H). ESI-MS: m/z=354.2 (M+1).

### Example 57: Synthesis of (4-(5-aminoisoxazol-3-yl)piperidin-1-yl)(3,4-dichlorophenyl)methanone (57)

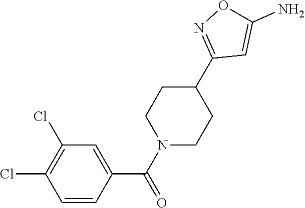 [SMILES: Nc1cc(C2CCN(C(=O)c3ccc(Cl)c(Cl)c3)CC2)no1]

Similar to the preparation of 1, purification by Prep-HPLC provided the target compound 57 (29.39 mg, yield: 29%) as a pale white solid. ¹HNMR (400 MHz, DMSO-d₆) δ 7.72-7.70 (m, 2H), 7.41-7.39 (m, 1H), 6.54 (s, 2H), 4.88 (s, 1H), 4.45-4.37 (m, 1H), 3.53-3.49 (m, 1H), 3.23-2.59 (m, 3H), 1.86 (s, 1H), 1.73 (s, 1H), 1.60-1.50 (m, 2H). ESI-MS: m/z=340.2 (M+1).

### Example 58: Synthesis of (4-(5-aminoisoxazol-3-yl)piperidin-1-yl)(3-methyl-5-(trifluoromethyl)phenyl)methanone (58)

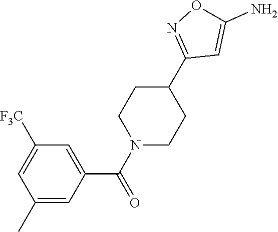 [SMILES: Cc1cc(C(=O)N2CCC(c3cc(N)on3)CC2)cc(C(F)(F)F)c1]

Similar to the preparation of 1, purification by Prep-HPLC provided the target compound 58 (18.57 mg, yield: 18%) as a pale white solid. ¹HNMR (400 MHz, DMSO-d₆) δ 7.64 (s, 1H), 7.53 (s, 2H), 6.54 (s, 2H), 4.89 (s, 1H), 4.50-4.40 (m, 1H), 3.54-3.43 (m, 1H), 3.25-3.09 (m, 1H), 3.01-2.63 (m, 2H), 2.43 (s, 3H), 1.93-1.83 (m, 1H), 1.78-1.71 (m, 1H), 1.61-1.49 (m, 2H). ESI-MS: m/z=354.1 (M+1).

### Example 59: Synthesis of (4-(5-aminoisoxazol-3-yl)piperidin-1-yl)(4-isopropylphenyl)methanone (59)

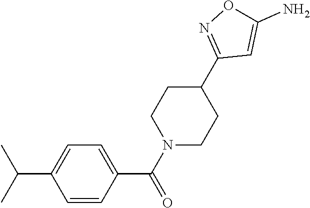 [SMILES: CC(C)c1ccc(C(=O)N2CCC(c3cc(N)on3)CC2)cc1]

Similar to the preparation of 1, purification by Prep-HPLC provided the target compound 59 (10.57 mg, yield: 10%) as a pale white solid. ¹HNMR (400 MHz, DMSO) δ 7.31 (s, 4H), 6.54 (s, 2H), 4.87 (s, 1H), 4.53-4.34 (m, 1H), 3.73-3.53 (m, 1H), 3.21-3.02 (m, 1H), 2.96-2.63 (m, 3H), 1.95-1.73 (m, 2H), 1.58-1.48 (m, 2H), 1.21 (d, J=6.8 Hz, 6H). ESI-MS: m/z=314.3 (M+1).

### Example 60: Synthesis of (4-(5-aminoisoxazol-3-yl)piperidin-1-yl)(2-fluoro-4-(trifluoromethyl)phenyl)methanone (60)

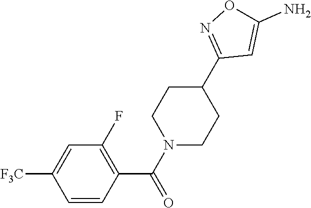 [SMILES: Nc1cc(C2CCN(C(=O)c3ccc(C(F)(F)F)cc3F)CC2)no1]

Similar to the preparation of 1, purification by Prep-HPLC provided the target compound 60 (23.59 mg, yield: 23%) as a pale white solid. T HNMR (400 MHz, DMSO-d₆) δ 7.83 (d, J=9.6 Hz, 1H), 7.69 (s, 2H), 6.55 (s, 2H), 4.86 (s, 1H), 4.48 (d, J=12.4 Hz, 1H), 3.41-3.35 (m, 1H), 3.20-3.13 (m, 1H), 3.00-2.94 (m, 1H), 2.83-2.76 (m, 1H), 1.93-1.88 (m, 1H), 1.77-1.73 (m, 1H), 1.58-1.43 (m, 2H). ESI-MS: m/z=358.1 (M+1).

### Example 61: Synthesis of (4-(5-aminoisoxazol-3-yl)piperidin-1-yl)(3-methoxy-4-(trifluoromethyl)phenyl)methanone (61)

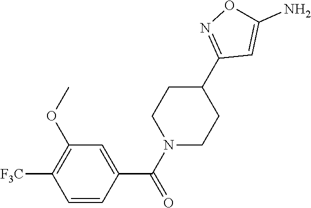 [SMILES: COc1cc(C(=O)N2CCC(c3cc(N)on3)CC2)ccc1C(F)(F)F]

Similar to the preparation of 1, purification by Prep-HPLC provided the target compound 61 (16.11 mg, yield: 16%) as a pale white solid. ¹HNMR (400 MHz, DMSO-d₆) δ 7.67 (d, J=8 Hz, 1H), 7.26 (s, 1H), 7.09 (d, J=8.4 Hz, 1H), 6.54 (s, 2H), 4.88 (s, 1H), 4.47-4.44 (m, 1H), 3.91 (s, 3H), 3.51-3.48 (m, 1H), 3.17-3.10 (m, 1H), 2.97-2.90 (m, 1H), 2.83-2.75 (m, 1H), 1.94-1.87 (m, 1H), 1.77-1.71 (m, 1H), 1.64-1.51 (m, 2H). ESI-MS: m/z=370.1

### Example 62: Synthesis of (4-(5-aminoisoxazol-3-yl)piperidin-1-yl)(4-cyclohexylphenyl)methanone (62)

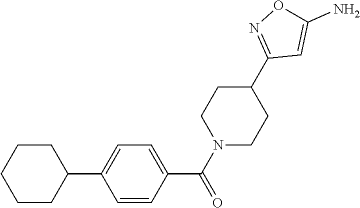 [SMILES: Nc1cc(C2CCN(C(=O)c3ccc(C4CCCCC4)cc3)CC2)no1]

Similar to the preparation of 1, purification by prep-HPLC provided the target compound 62 (36.4 mg, yield: 14.1%) as a white solid. ¹H NMR (400 MHz, DMSO-d₆) δ 7.27-7.32 (m, 4H), 6.54 (s, 2H), 4.88 (s, 1H), 4.44 (s, 1H), 3.62 (s, 1H), 2.73-2.90 (m, 3H), 2.50-2.56 (m, 1H), 1.69-1.80 (m, 7H), 1.22-1.57 (m, 7H). ESI-MS: m/z=354.3 (M+1).

### Example 63: Synthesis of (4-(5-aminoisoxazol-3-yl)piperidin-1-yl)(3-fluoro-4-(trifluoromethyl)phenyl)methanone (63)

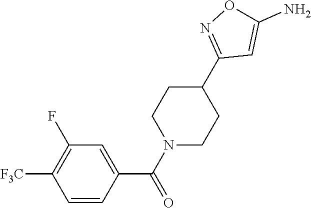 [SMILES: Nc1cc(C2CCN(C(=O)c3ccc(C(F)(F)F)c(F)c3)CC2)no1]

Similar to the preparation of 1, purification by prep-HPLC provided the target compound 63 (30.2 mg, yield: 11.8%) as a white solid. ¹H NMR (400 MHz, DMSO-d₆) δ 7.87 (t, J=7.8 Hz, 1H), 7.63 (d, J=11.2 Hz, 1H), 7.44 (d, J=8.4 Hz, 1H), 6.55 (s, 2H), 4.89 (s, 1H), 4.44 (d, J=13.6 Hz, 1H), 3.47 (d, J=12.4 Hz, 1H), 3.15 (t, J=12.0 Hz, 1H), 2.94 (t, J=11.6 Hz, 1H), 2.75-2.82 (m, 1H), 1.90 (d, J=13.6 Hz, 1H), 1.73 (d, J=11.2 Hz, 1H), 1.55-1.63 (m, 2H). ESI-MS: m/z=358.3 (M+1).

### Example 64: Synthesis of (4-(5-(dimethylamino)isoxazol-3-yl)piperidin-1-yl)(3-methyl-4-(trifluoromethyl)phenyl)methanone (64)

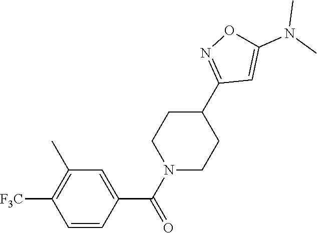 [SMILES: Cc1cc(C(=O)N2CCC(c3cc(N(C)C)on3)CC2)ccc1C(F)(F)F]

Similar to the preparation of 69, purification by prep-HPLC provided the target compound 64 (9.7 mg, yield: 3.3%) as a yellow solid. ¹H NMR (400 MHz, DMSO-d₆) δ 7.73 (d, J=8.0 Hz, 1H), 7.47 (s, 1H), 7.40 (d, J=8.0 Hz, 1H), 5.14 (s, 1H), 4.47 (d, J=12.0 Hz, 1H), 3.51 (d, J=15.6 Hz, 1H), 3.16 (t, J=12.0 Hz, 1H), 2.91-2.88 (m, 1H), 2.88 (s, 6H), 2.81-2.86 (m, 1H), 2.48 (s, 3H), 1.92 (d, J=12.0 Hz, 1H), 1.75 (d, J=16.0 Hz, 1H), 1.57 (t, J=13.4 Hz, 2H). ESI-MS: m/z=382.1 (M+1).

### Example 65: Synthesis of N-(3-(1-(3-methyl-4-(trifluoromethyl)benzoyl)piperidin-4-yl) isoxazol-5-yl)acetamide (65)

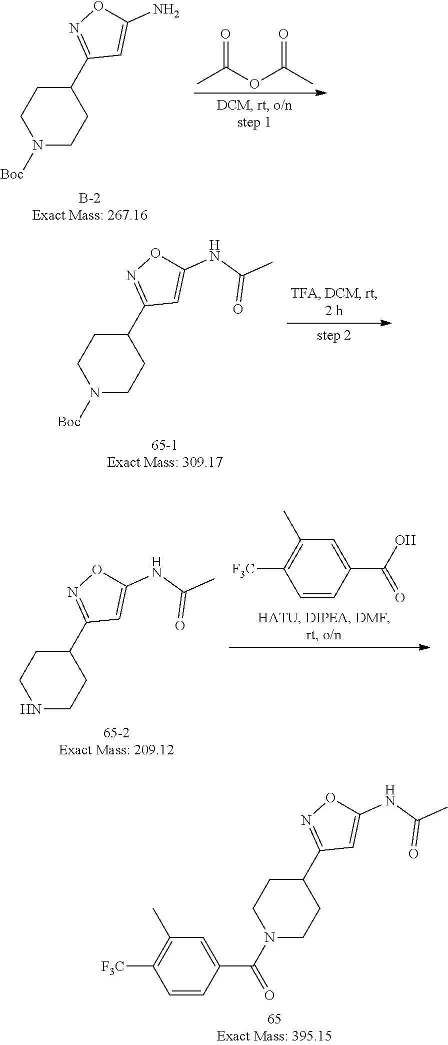

Synthesis of 65-1 (step 1): To a solution of B-2 (150 mg, 0.56 mmol) in DCM (5 mL), was added acetic anhydride (68.5 mg, 0.67 mmol). The mixture was stirred at room temperature overnight. The reaction mixture was poured into water (20 mL) and extracted with EtOAc (30 mL×3). The organic layer was washed with brine and evaporated, the residue crude product was purified by reverse CC to give 65-1 (100 mg, 58%) as a white solid.

Synthesis of 65-2 (step 2): To a solution of 65-1 (100 mg, 0.32 mmol) in DCM (5 mL), was added TFA (411 mg, 3.70 mmol). The mixture was stirred at room temperature for 3 h. The solvent was removed to give compound 65-2. It was used in next step directly without further purification.

Synthesis of 65 (step 3): The mixture of 3-methyl-4-(trifluoromethyl)benzoic acid (62.3 mg, 0.32 mmol), 65-2 (0.32 mmol), HATU (212.8 mg, 0.56 mmol) and DIEA (143.2 mg, 1.11 mmol) in DMF (3.0 mL) was stirred at room temperature overnight. The reaction mixture was poured into water (100 mL) and extracted with EtOAc (60 mL×3). The organic layer was washed with brine and evaporated, the residue crude product was purified by Prep-HPLC provided 65 (45.01 mg, yield: 36%, 2 steps) as a white solid. ¹HNMR (400 MHz, DMSO-d₆) δ 11.55 (s, 1H), 7.73 (d, J=8.0 Hz, 1H), 7.50 (s, 1H), 7.43 (d, J=8.0 Hz, 1H), 6.23 (s, 1H), 4.49 (d, J=10.8 Hz, 1H), 3.51 (d, J=11.6 Hz, 1H), 3.16 (t, J=11.2 Hz, 1H), 2.92-3.00 (m, 2H), 2.48 (s, 3H), 2.08 (s, 3H), 1.96-1.97 (m, 1H), 1.78-1.81 (m, 1H), 1.59-1.68 (m, 2H). ESI-MS: m/z=396.2 (M+1).

### Example 66: Synthesis of 5-(4-(5-aminoisoxazol-3-yl)piperidine-1-carbonyl)-2-(trifluoromethyl)benzonitrile (66)

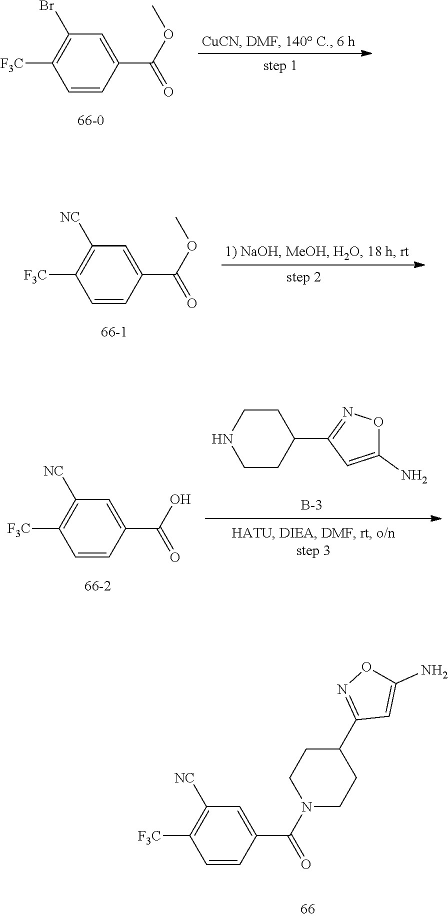

Synthesis of 66-1 (step 1): To a solution of methyl 3-bromo-4-(trifluoromethyl)benzoate (2 g, 7.1 mmol) in DMF (30 mL) was added CuCN (957 mg, 10.64 mmol). The resulting reaction mixture was heated to 140° C. and stirred for 6 h and concentrated in vacuo to remove the solvent, then added water, the aqueous phase was extracted with ethyl acetate, the combined organic phases were dried over anhydrous sodium sulfate, filtered, and concentrated, the crude was purified by c.c. to give 66-1 (470 mg, yield: 28.9%) as a white solid.

Synthesis of 66-2 (step 2): The mixture of 66-1 (250 mg, 1.09 mmol) and NaOH (218 mg, 5.46 mmol) in MeOH (10 mL) and H2O (2 mL) was stirred at room temperature overnight. Then the reaction mixture was neutralized with 1N aqueous HCl solution to pH 6.0. After the reaction mixture was collected by filtration and washed with water. The solid was dried in vacuo to give compound 423-2 (200 mg, 85.5%) as a white solid.

Synthesis of 66 (step 3): The mixture of 66-2 (150 mg, 0.69 mmol), B-3 (115 mg, 0.69 mmol), HATU (393 mg, 1.04 mmol) and DIEA (267 mg, 2.07 mmol) in DMF (5 mL) was stirred at room temperature overnight. The reaction mixture was poured into water (100 mL) and extracted with EtOAc (60 mL×3). The organic layer was washed with brine and evaporated, the residue crude product was purified by Prep-HPLC provided 66 (28.7 mg, yield: 11.4%) as a white solid. ¹H NMR (400 MHz, DMSO-d₆) δ 8.27 (s, 1H), 8.08 (d, J=8.4 Hz, 1H), 7.96 (d, J=8.4 Hz, 1H), 6.55 (s, 2H), 4.89 (s, 1H), 4.45 (d, J=11.2 Hz, 1H), 3.45 (d, J=14.8 Hz, 1H), 3.16 (t, J=12.0 Hz, 1H), 2.96 (t, J=12.4 Hz, 1H), 2.76-2.83 (m, 1H), 1.91 (d, J=12.4 Hz, 1H), 1.73 (d, J=16.0 Hz, 1H), 1.55-1.65 (m, 2H). ESI-MS: m/z=365.2 (M+1).

### Example 67: Synthesis of (4-(5-(benzylamino)isoxazol-3-yl)piperidin-1-yl)(3-methyl-4-(trifluoromethyl)phenyl)methanone (67)

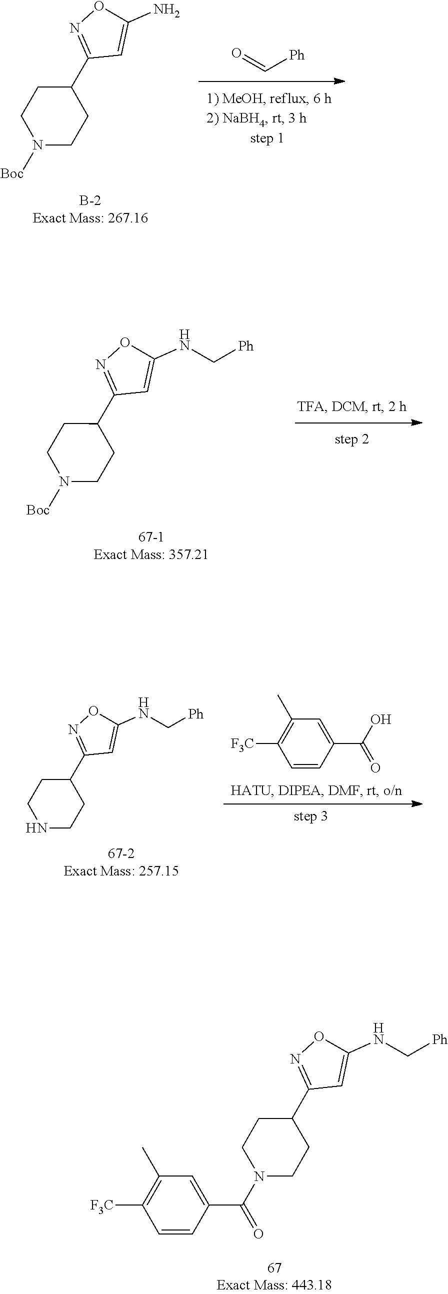 [SMILES: CC(C)(C)OC(=O)N1CCC(c2cc(N)on2)CC1 ; O=Cc1ccccc1 ; CC(C)(C)OC(=O)N1CCC(c2cc(NCc3ccccc3)on2)CC1 ; c1ccc(CNc2cc(C3CCNCC3)no2)cc1 ; Cc1cc(C(=O)O)ccc1C(F)(F)F ; Cc1cc(C(=O)N2CCC(c3cc(NCc4ccccc4)on3)CC2)ccc1C(F)(F)F]

Synthesis of 67-1 (step 1): A mixture of B-2 (150 mg, 0.56 mmol) and benzaldehyde (71.0 mg, 0.67 mmol) in MeOH (5 mL) was stirred at 80° C. for 6 h. To the mixture was added NaBH₄ (25.5 mg, 0.67 mmol). The mixture was stirred at room temperature for 3 h. The reaction mixture was poured into water (20 mL) and extracted with EtOAc (30 mL×3). The organic layer was washed with brine and evaporated, the residue crude product was purified by reverse CC to give 67-1 (100 mg, 50%) as a white solid.

Synthesis of 67-2 (step 2): To a solution of 67-1 (100 mg, 0.28 mmol) in DCM (5 mL), was added TFA (411 mg, 3.70 mmol). The mixture was stirred at room temperature for 3 h, The solvent was removed to give compound 67-2, It was used to next step directly without further purification.

Synthesis of 67 (step 3): The mixture of 3-methyl-4-(trifluoromethyl)benzoic acid (57.1 mg, 0.28 mmol), 67-2 (0.28 mmol), HATU (212.8 mg, 0.56 mmol) and DIEA (143.2 mg, 1.11 mmol) in DMF (3.00 mL) was stirred at room temperature overnight. The reaction mixture was poured into water (100 mL) and extracted with EtOAc (60 mL×3). The organic layer was washed with brine and evaporated, the residue crude product was purified by Prep-HPLC provided 67 (7.16 mg, yield: 6%, 2 steps) as a white solid. ¹HNMR (400 MHz, DMSO-d₆) δ 7.68-7.74 (m, 2H), 7.47 (s, 1H), 7.39 (d, J=8.0 Hz, 1H), 7.33-7.34 (m, 4H), 7.23-7.28 (m, 1H), 5.02 (s, 1H), 4.43-4.47 (m, 1H), 4.25 (d, J=6.0 Hz, 2H), 3.47-3.50 (m, 1H), 3.10-3.16 (m, 1H), 2.91-2.94 (m, 1H), 2.77-2.82 (m, 1H), 2.47 (s, 3H), 1.88-1.91 (m, 1H), 1.72-1.75 (m, 1H), 1.48-1.56 (m, 2H). ESI-MS: m/z=444.3 (M+1).

### Example 68: Synthesis of 4-(4-(5-aminoisoxazol-3-yl)piperidine-1-carbonyl)-N,N-dimethylbenzamide (68)

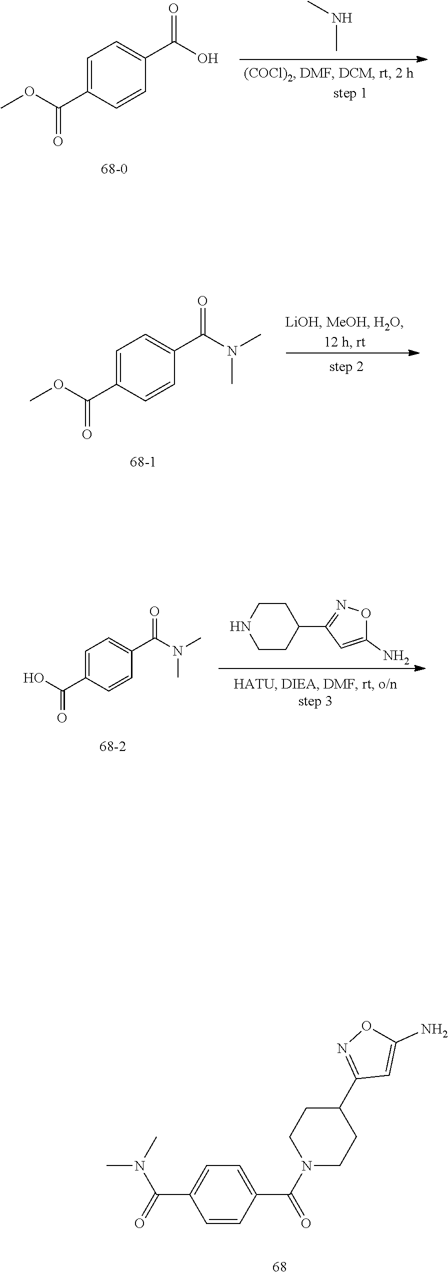

Synthesis of 68-1 (step 1): To a solution of 4-(methoxycarbonyl)benzoic acid (2 g, 11.11 mmol) in DCM (30 mL) and DMF (0.5 mL), was added (COCl)₂ (1.41 g, 11.11 mmol). The resulting reaction mixture was stirred at rt for 2 h and then added dimethylamine (4.5 g, 55.6 mmol). The reaction mixture was stirred overnight at rt. After concentrated in vacuo to remove the solvent, the crude was purified by c.c. to give 68-1 (200 mg, yield: 8.7%) as a white solid.

Synthesis of 68-2 (step 2): The mixture of 68-1 (200 mg, 0.97 mmol) and LiOH (203 mg, 4.83 mmol) in MeOH (10 mL) and H2O (2 mL) was stirred at room temperature for 12 h. Then the reaction mixture was neutralized with 1N aqueous HCl solution to pH 6.0. After the reaction mixture was collected by filtration and washed with water. The solid was dried in vacuo to give compound 68-2 (140 mg, 74.9%) as a white solid.

Synthesis of 68 (step 3): The mixture of 68-2 (140 mg, 0.73 mmol), B-3 (121 mg, 0.73 mmol), HATU (413 mg, 1.09 mmol) and DIEA (280 mg, 2.18 mmol) in DMF (5 mL) was stirred at room temperature overnight. The reaction mixture was poured into water (100 mL) and extracted with EtOAc (60 mL×3). The organic layer was washed with brine and evaporated, the residue crude product was purified by Prep-HPLC provided 68 (16.0 mg, yield: 6.4%) as a yellow solid. ¹H NMR (400 MHz, DMSO-d₆) δ 7.45 (s, 4H), 6.54 (s, 2H), 4.89 (s, 1H), 4.47 (s, 1H), 3.56 (s, 1H), 3.31 (s, 1H), 3.13 (s, 1H), 2.99 (s, 3H), 2.91 (s, 3H), 2.76-2.81 (m, 1H), 1.91 (s, 1H), 1.72 (s, 1H), 1.55 (s, 2H). ESI-MS: m/z=343.2 (M+1).

### Example 69: Synthesis of (4-(5-(benzyl(methyl)amino)isoxazol-3-yl)piperidin-1-yl)(3-methyl-4-(trifluoromethyl)phenyl)methanone (69)

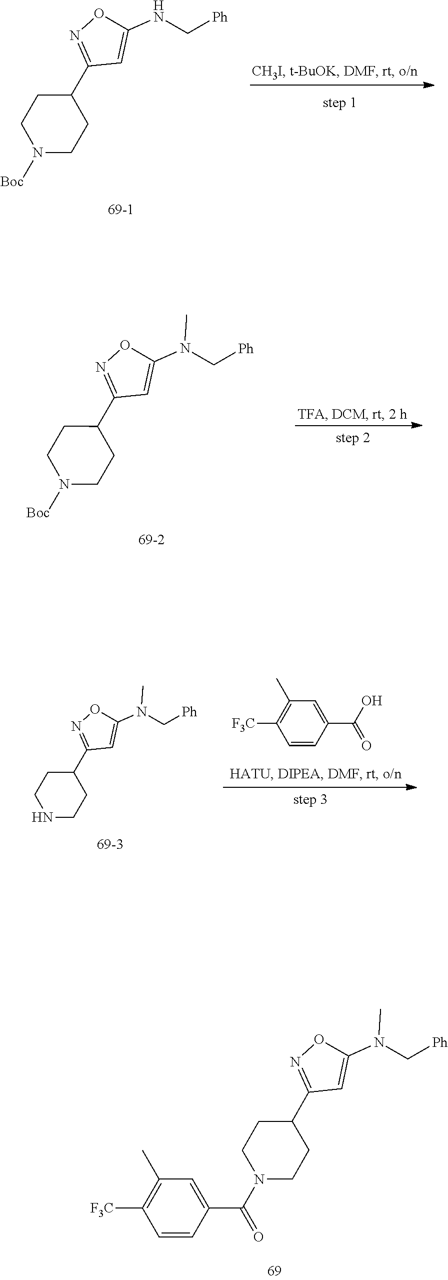 [SMILES: CC(C)(C)OC(=O)N1CCC(c2cc(NCc3ccccc3)on2)CC1 ; CN(Cc1ccccc1)c1cc(C2CCN(C(=O)OC(C)(C)C)CC2)no1 ; CN(Cc1ccccc1)c1cc(C2CCNCC2)no1 ; Cc1cc(C(=O)O)ccc1C(F)(F)F ; Cc1cc(C(=O)N2CCC(c3cc(N(C)Cc4ccccc4)on3)CC2)ccc1C(F)(F)F]

Synthesis of 69-2 (step 1): To a solution of tert-butyl 4-(5-(benzylamino)isoxazol-3-yl)piperidine-1-carboxylate (500 mg, 1.4 mmol) in DMF (10 mL), was added CH₃I (298 mg, 2.1 mmol) and t-BuOK (392 mg, 3.5 mmol). The resulting reaction mixture was stirred overnight at rt. Then added water, the aqueous phase was extracted with ethyl acetate, the combined organic phases were dried over anhydrous sodium sulfate, filtered, and concentrated. The organic layer was concentrated, purified by C.C. to afford the product 434-2 (400 mg, 77.1% yield) as a yellow solid.

Synthesis of 69-3 (step 2): To a stirred solution of compound 69-2 (400 mg, 1.08 mmol) in DCM (8 mL) was added TFA (4 mL) at rt. The resulting reaction mixture was further stirred for 2 h at rt, then concentrated in vacuo to give the desired product 69-3 (240 mg, yield: 82.2%) as a yellow oil.

Synthesis of 69 (step 3): The mixture of 69-3 (150 mg, 0.55 mmol), 3-methyl-4-(trifluoromethyl)benzoic acid (113 mg, 0.55 mmol), HATU (314 mg, 0.83 mmol) and DIEA (213 mg, 1.65 mmol) in DMF (5 mL) was stirred at room temperature overnight. The reaction mixture was poured into water (100 mL) and extracted with EtOAc (60 mL×3). The organic layer was washed with brine and evaporated, the residue crude product was purified by Prep-HPLC provided 69 (15.0 mg, yield: 6.0%) as a yellow solid. ¹H NMR (400 MHz, DMSO-d₆) δ 7.31 (d, J=8.0 Hz, 1H), 7.47 (s, 1H), 7.34-7.41 (m, 3H), 7.25-7.31 (m, 3H), 5.22 (s, 1H), 4.46 (s, 3H), 3.51 (d, J=12.0 Hz, 1H), 3.16 (t, J=11.2 Hz, 1H), 2.95 (d, J=12.4 Hz, 1H), 2.89 (s, 3H), 2.81-2.87 (m, 1H), 2.48 (s, 3H), 1.93 (d, J=14.0 Hz, 1H), 1.77 (d, J=11.2 Hz, 1H), 1.58 (t, J=11.8 Hz, 2H). ESI-MS: m/z=458.2 (M+1).

### Example 70: Synthesis of (4-(5-aminoisoxazol-3-yl)piperidin-1-yl)(4-bromo-3,5-dimethylphenyl)methanone (70)

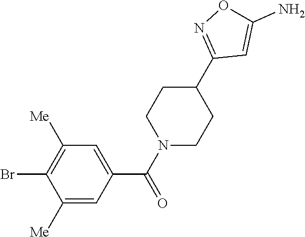 [SMILES: Cc1cc(C(=O)N2CCC(c3cc(N)on3)CC2)cc(C)c1Br]

Similar to the preparation of 1, purification by prep-HPLC provided the target compound 70 (27.1 mg, yield: 10.9%) as a white solid. ¹H NMR (400 MHz, DMSO-d₆) δ 7.66 (s, 2H), 6.39 (s, 2H), 5.46 (s, 1H), 4.99 (bs, 1H), 4.23 (bs, 1H), 3.30-3.45 (m, 3H), 2.89 (s, 6H), 2.35-2.37 (m, 2H), 2.05-2.14 (m, 2H). ESI-MS: m/z=380.3 (M+1).

### Example 71: Synthesis of (4-(5-aminoisoxazol-3-yl)piperidin-1-yl)(2,5-dimethyl-4-(trifluoromethyl)phenyl)methanone (71)

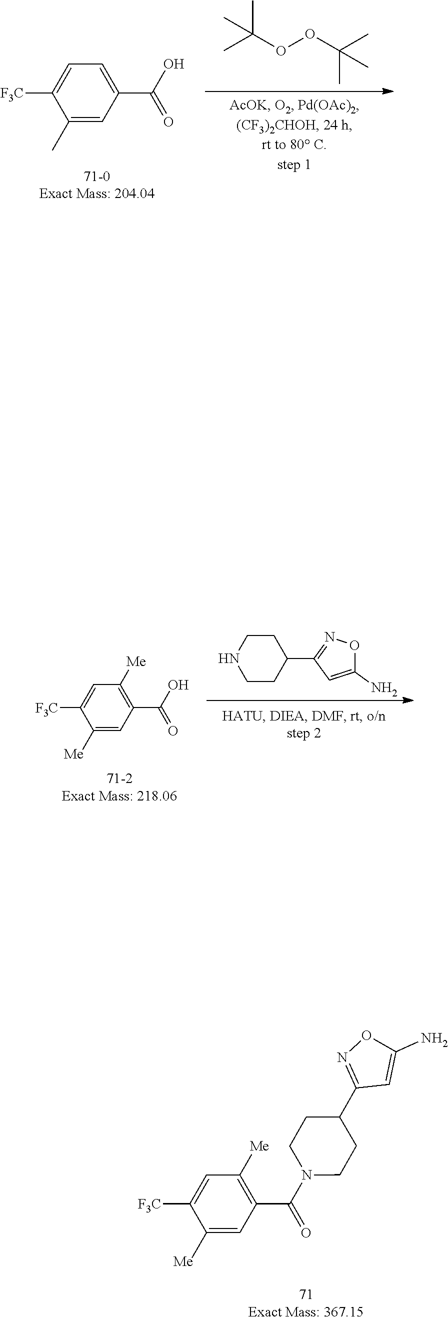

Synthesis of 71-1 (step 1): To a solution of 71-0 (200 mg, 0.98 mmol) in HFIP (5 mL), was added 2-(tert-butylperoxy)-2-methylpropane (286.2 mg, 1.96 mmol), Pd(OAc)₂ (22.4 mg, 0.10 mmol) and AcOK (192.1 mg, 1.96 mmol). The mixture was stirred at 80° C. overnight. The reaction mixture was poured into water (100 mL) and extracted with EtOAc (60 mL×3). The organic layer was washed with brine and evaporated, the residue crude product was purified by reverse CC to give 71-1 (70 mg, 33%) as a yellow solid.

Synthesis of 71 (step 2): The mixture of 71-1 (70 mg, 0.32 mmol), 3-(piperidin-4-yl) isoxazol-5-amine (61.8 mg, 0.37 mmol), HATU (182.4 mg, 0.48 mmol) and DIEA (123.8 mg, 0.96 mmol) in DMF (3.00 mL) was stirred at room temperature overnight. The reaction mixture was poured into water (100 mL) and extracted with EtOAc (60 mL×3). The organic layer was washed with brine and evaporated, the residue crude product was purified by Prep-HPLC provided 71 (21.37 mg, yield: 18%) as a white solid. ¹HNMR (400 MHz, DMSO-d₆) δ 7.58 (s, 1H), 7.24-7.33 (m, 1H), 6.54 (s, 2H), 4.84-4.89 (m, 1H), 4.49-4.52 (m, 1H), 3.23-3.27 (m, 1H), 3.07-3.13 (m, 1H), 2.92-2.95 (m, 1H), 2.75-2.81 (m, 1H), 2.41 (s, 3H), 2.21-2.27 (m, 3H), 1.89-1.95 (m, 1H), 1.69-1.77 (m, 1H), 1.34-1.58 (m, 2H). ESI-MS: m/z=368.2 (M+1).

### Example 72: Synthesis of (3-methyl-4-(trifluoromethyl)phenyl) (4-(5-(methylamino)isoxazol-3-yl)piperidin-1-yl)methanone (72)

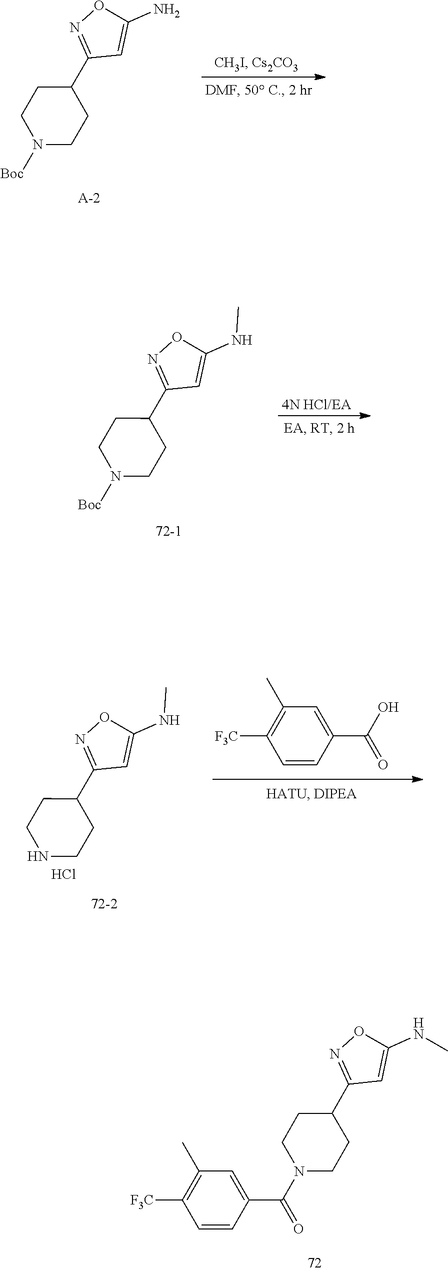

Synthesis of 72-1 (Step 1): To a solution of tert-butyl 4-(5-aminoisoxazol-3-yl)piperidine-1-carboxylate (500 mg, 1.87 mmol) and dicesium; carbonate (914.12 mg, 2.81 mmol) in DMF (5 mL), the reaction was stirred at 50° C. for 30 min. Then iodomethane (398.22 mg, 2.81 mmol, 174.66 uL) in DMF (5 mL) was injected. Then the reaction was stirred at 50° C. for 1.5 hr. The solvent was concentrated, the residue extracted with DCM (20 mL*2), dried over Na2SO4 and concentrated. The residue was purified by flash chromatography (PE:EA=8:1) to give tert-butyl 4-[5-(methylamino)isoxazol-3-yl]piperidine-1-carboxylate (140 mg, 497.60 umol, 26.60% yield) as a yellow oil.

Synthesis of 72-2 (Step 2): To a solution of tert-butyl 4-[5-(methylamino)isoxazol-3-yl]piperidine-1-carboxylate (140 mg, 497.60 umol) in 4N HCl/Ethyl acetate (995.20 umol, 10 mL), the reaction was stirred at RT for 2 hr. The solvent was concentrated to give N-methyl-3-(4-piperidyl)isoxazol-5-amine (67 mg, 307.77 umol, HCl) as a brown oil.

Synthesis of 72 (Step 3): To a solution of 3-methyl-4-(trifluoromethyl)benzoic acid (63 mg, 308.60 umol) in DMF (3 mL) were added [dimethylamino(triazolo[4,5-b]pyridin-3-yloxy)methylene]-dimethyl-ammonium hexafluorophosphate (176.01 mg, 462.90 umol), N-ethyl-N-isopropyl-propan-2-amine (119.65 mg, 925.81 umol, 161.26 uL), the reaction was stirred for 15 min, and N-methyl-3-(4-piperidyl)isoxazol-5-amine (67.18 mg, 308.60 umol, HCl) was added. The reaction was stirred at RT for 2 hr. Extracted with EA (20 ml*2) and H₂O (20 ml*2). The combined organic layers were dried over Na2SO4 and was purified by prep-HPLC to afford 72 (20 mg, 54.44 umol) as a solid. ¹H NMR (400 MHz, CDCl₃) δ 7.59 (t, J=7.4 Hz, 1H), 7.21 (d, J=11.9 Hz, 2H), 4.78 (s, 1H), 4.61 (s, 1H), 4.43 (d, J=4.4 Hz, 1H), 3.67 (d, J=8.3 Hz, 1H), 2.90-2.74 (m, 4H), 2.45 (s, 3H), 1.91 (d, J=58.2 Hz, 2H), 1.64 (d, J=37.7 Hz, 3H), 1.19 (s, 1H). M+H calc'd 367.2. M+H found 368.2.

### Example 73: Synthesis of [3-fluoro-4-(trifluoromethyl)phenyl]-[4-[5-(methylamino)isoxazol-3-yl]-1-piperidyl]methanone (73)

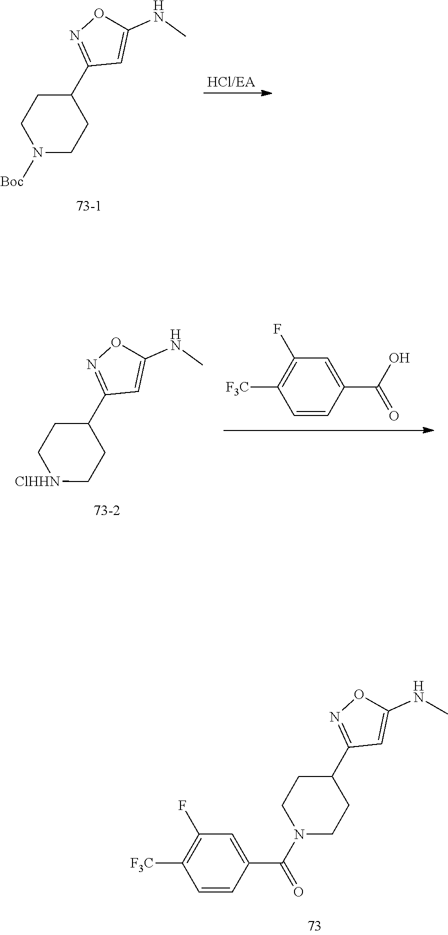

Synthesis of 73-1 (Step 1): Tert-butyl 4-[5-(methylamino)isoxazol-3-yl]piperidine-1-carboxylate (380 mg, 1.35 mmol) was dissolved in 4 M HCl/dioxane (10 mL) and stirred at room temperature for 2 hours. The solvent was evaporated to provide the product residue.

Synthesis of 73 (Step 2): Dissolve the 3-methyl-4-(trifluoromethyl)benzoic acid (135.17 mg, 662.13 umol), N-ethyl-N-isopropyl-propan-2-amine (342.30 mg, 2.65 mmol, 461.32 uL) and N,N,N′,N′-tetramethyl-1-(3-oxido-2,3-dihydrotriazolo[4,5-b]pyridin-3-ium-1-yl)methanediamine hexafluorophosphate (377.64 mg, 993.19 umol) in the DCM (5 mL). Then N-methyl-3-(4-piperidyl)isoxazol-5-amine (120 mg, 662.13 umol) was added. It was stirred at room temperature for 16 hours. The reaction was concentrated, the residue purified by prep-HPLC to 73 as a white solid. ¹H NMR (400 MHz, CDCl₃) δ 7.67 (t, J=7.4 Hz, 1H), 7.29 (s, 1H), 7.24 (s, 1H), 4.84 (s, 1H), 4.73-4.50 (m, 2H), 3.70 (s, 1H), 3.17 (s, 1H), 3.10-2.98 (m, 1H), 2.97-2.90 (m, 1H), 2.89 (d, J=5.2 Hz, 3H), 2.00 (dd, J=29.1, 23.3 Hz, 2H), 1.76 (s, 2H). ESI-MS: m/z=371.9 (M+1).

### Example 74: Synthesis of [4-[5-(dimethylamino)isoxazol-3-yl]-1-piperidyl]-[3-fluoro-4-(trifluoromethyl)phenyl]methanone (74)

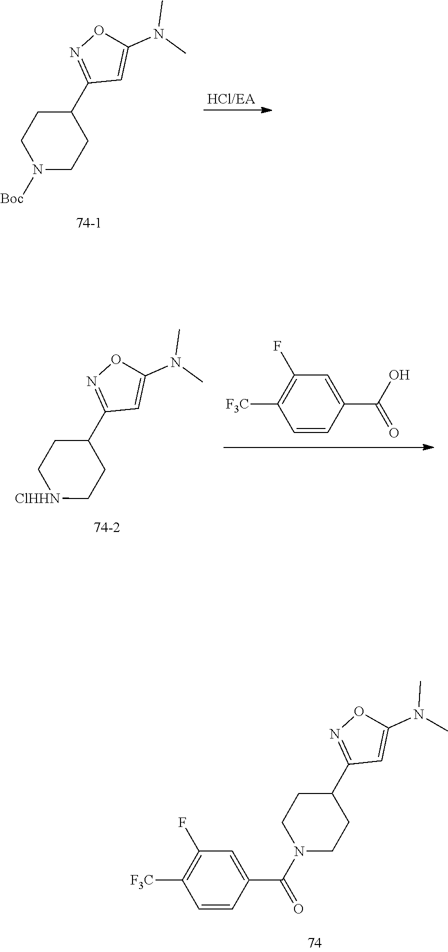

Similar to the preparation of 73, purification by prep-HPLC provided the target compound 74 (25.8 mg, yield: 1.9%) as a light yellow oil. ¹H NMR (400 MHz, CDCl₃) δ 7.67 (t, J=7.4 Hz, 1H), 7.28 (d, J=6.6 Hz, 1H), 7.24 (s, 1H), 4.76 (s, 1H), 4.63 (s, 1H), 3.70 (dd, J=10.2, 3.9 Hz, 1H), 3.26-3.00 (m, 2H), 2.97 (s, 6H), 2.91 (tt, J=11.1, 3.9 Hz, 1H), 2.13-1.91 (m, 2H), 1.78 (s, 2H). ESI-MS: m/z=385.9 (M+1).

### Example 75: Synthesis of (4-(5-aminoisoxazol-3-yl)piperidin-1-yl) (3-chloro-4-(trifluoromethyl)phenyl)methanone (75)

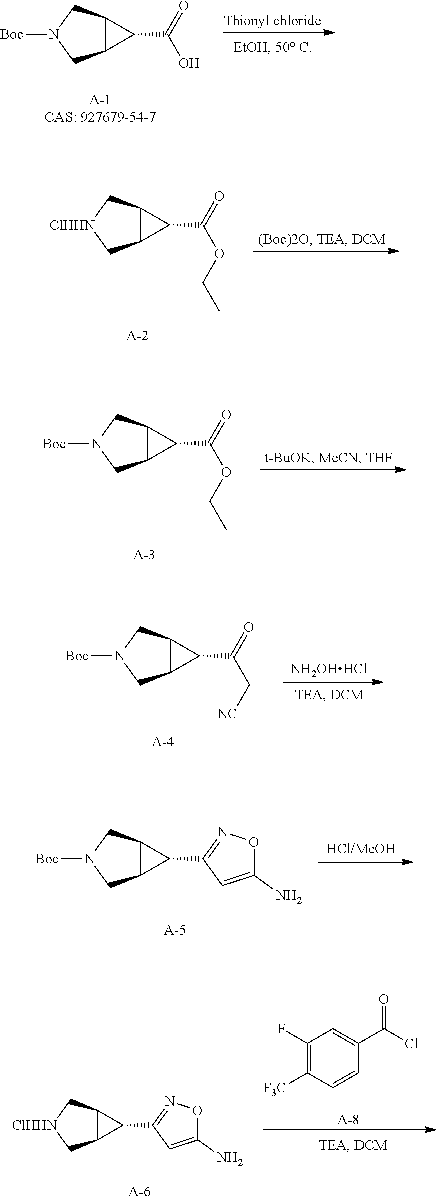

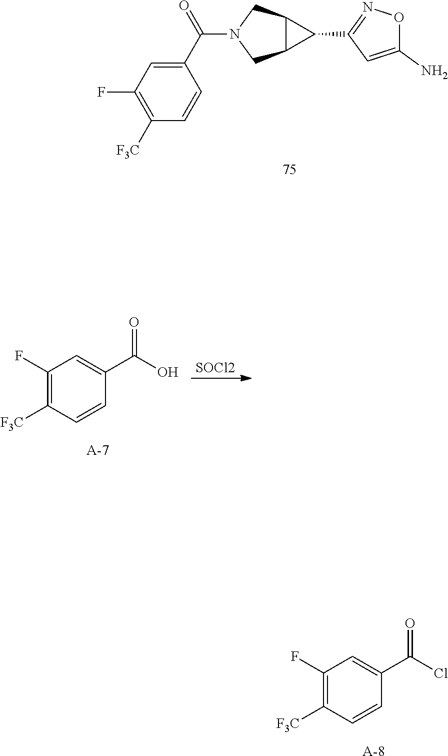

Synthesis of A-2 (step 1): To a stirred solution of (1R,5S)-3-tert-butoxycarbonyl-3-azabicyclo[3.1.0]hexane-6-carboxylic acid (900 mg, 3.96 mmol) in EtOH (20 mL) was added thionyl chloride (471.16 mg, 3.96 mmol, 1.5 mL) at RT. The mixture was stirred at 80° C. for 2 h. The reaction was concentrated. The residue obtained was used in next step directly.

Synthesis of A-3 (step 2): To a stirred solution of A-2 (326 mg, 1.84 mmol) in DCM (8 mL) was added N,N-diethylethanamine (557.14 mg, 5.51 mmol, 767.41 uL) and tert-butoxycarbonyl tert-butyl carbonate (480.66 mg, 2.20 mmol, 505.42 uL) at 25° C. The mixture was stirred at 25° C. for 2 hr. The mixture was concentrated. The residue obtained was purified by column chromatography of silica gel eluting with petroleum ether/EA from 50/1 to 5/1 to give 03-tert-butyl 06-methyl (1R,5S)-3-azabicyclo[3.1.0]hexane-3,6-dicarboxylate (120 mg, 497.34 umol, 27.10% yield) as a colorless oil.

Synthesis of A-4 (step 3): To a solution of A-3 (500 mg, 1.96 mmol) in THE (10 mL) added acetonitrile (401.98 mg, 9.79 mmol, 511.43 uL). Then potassium 2-methylpropan-2-olate (659.27 mg, 5.88 mmol) was added at 0° C. After addition, the reaction was stirred at 25° C. for 1 h. The reaction was quenched with saturated NH4Cl (20 mL) and extracted with EA (30 mL*2). The organic layer was combined, washed with brine, dried over Na2SO4 and concentrated to give crude product. The crude product was purified with column chromatography of silica gel eluting with petroleum ether/EA from 0 to 60% and concentrated to give A-4 (223 mg, 890.96 umol, 45.49% yield)

Synthesis of A-5 (step 4): To a solution of A-4 (280 mg, 1.12 mmol) in DCM (0.5 mL) added hydroxylamine hydrochloride (108.83 mg, 1.57 mmol, 65.17 uL) and N,N-diethylethanamine (226.40 mg, 2.24 mmol, 311.85 uL). Then the reaction was stirred at 55° C. for 12 h. The reaction was diluted with water and extracted with DCM (30 mL*2). The organic layer was combined, washed with brine, dried over Na2SO4 and concentrated to give crude product. The crude product was purified by SiO2 chromatography (PE:EA=0 to 60%) and concentrated to give A-5 (141 mg, 531.46 umol, 47.51% yield).

Synthesis of A-6 (step 5): To a solution of A-5 (143 mg, 539.00 umol) in MeOH (1 mL) added 4M HCl in dioxane (2 mL). Then the reaction was stirred at 25° C. for 2 h. The reaction was directly concentrated to give A-6 (113 mg, 684.05 umol, >99% yield).

Synthesis of A-8 (step 6): To a solution of A-7 (80 mg, 384.41 umol) in SOCl2 (1 mL). Then the reaction was stirred at 60° C. for 1 h. The reaction as directly concentrated to remove solvent and give A-8 (90 mg, 397.25 umol, >99% yield).

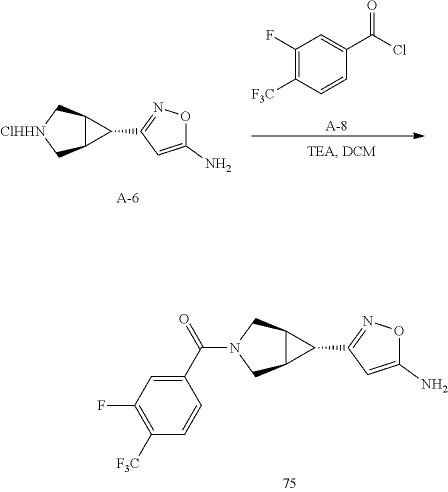

Synthesis of 75 (step 7): To a solution of A-6 (103 mg, 623.52 umol) in DMF (1.5 mL) added N, N-diethylethanamine (160.79 mg, 1.59 mmol, 221.48 uL). Then A-8 (120 mg, 529.67 umol) in DCM (1.5 mL) was added to the mixture. Then the reaction was stirred at 25° C. for 1.5 h. The reaction mixture was directly purified with HPLC to give 75 (30 mg, 84.44 umol, 15.94% yield). H NMR (400 MHz, CDCl₃) δ 7.67 (t, J=7.4 Hz, 1H), 7.31 (t, J=9.9 Hz, 2H), 4.88 (s, 1H), 4.40 (s, 2H), 4.30 (d, J=12.5 Hz, 1H), 3.72 (dd, J=10.8, 3.7 Hz, 1H), 3.67-3.51 (m, 2H), 2.10-1.98 (m, 2H), 1.65 (t, J=3.4 Hz, 1H). ESI-MS: m/z=356.09 (M+1).

### Example 76: Synthesis of [4-(5-aminoisoxazol-3-yl)-1-piperidyl]-[2-fluoro-5-(trifluoromethoxy)phenyl]methanone (76)

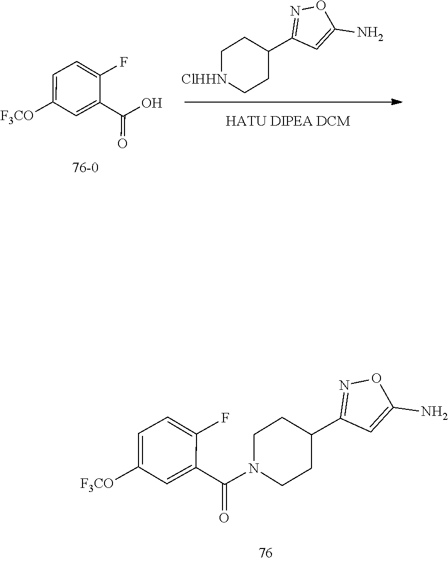

Similar to the preparation of 73, purification by prep-HPLC provided the target compound 76 (25.8 mg, yield: 11.5%) as a white solid. ¹H NMR (400 MHz, CDCl₃) δ 7.48-7.32 (m, 1H), 7.25 (d, J=4.1 Hz, 1H), 7.17-7.10 (m, 1H), 5.00 (s, 1H), 4.69 (d, J=13.4 Hz, 1H), 4.49 (s, 2H), 3.60 (d, J=13.6 Hz, 1H), 3.18 (s, 1H), 3.02 (t, J=11.9 Hz, 1H), 2.90 (tt, J=11.2, 3.8 Hz, 1H), 2.05 (dt, J=13.7, 7.0 Hz, 1H), 1.98-1.88 (m, 1H), 1.84-1.71 (m, 2H). ESI-MS: m/z=373.8 (M+1).

### Example 77: Synthesis of 3-(1-(4-(trifluoromethoxy)phenylsulfonyl)piperidin-4-yl)isoxazol-5-amine (77)

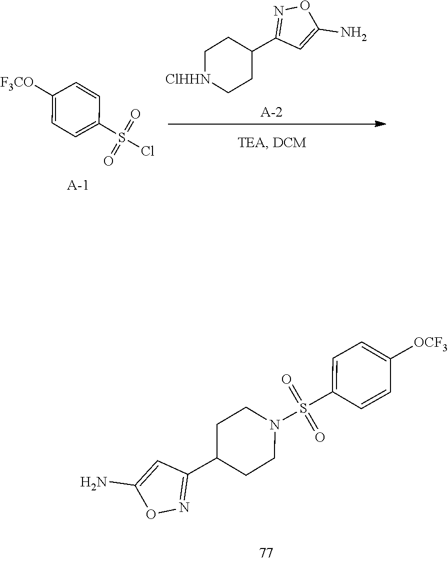

Similar to the preparation of 73, purification by prep-HPLC provided the target compound 77 (24 mg, 61.32 umol, 13.93% yield) as a white solid. ¹H NMR (400 MHz, CDCl₃) δ 7.85-7.79 (m, 2H), 7.43-7.33 (m, 2H), 4.95 (s, 1H), 4.40 (s, 2H), 3.81 (dt, J=11.8, 3.0 Hz, 2H), 2.59 (tt, J=11.3, 3.9 Hz, 1H), 2.49 (td, J=11.7, 2.7 Hz, 2H), 1.99 (dd, J=13.4, 2.9 Hz, 2H), 1.90-1.75 (m, 2H). ESI-MS: m/z=391.8 (M+1).

### Example 78: Synthesis of (4-(5-aminoisoxazol-3-yl)piperidin-1-yl) (4-bromophenyl)methanone (78)

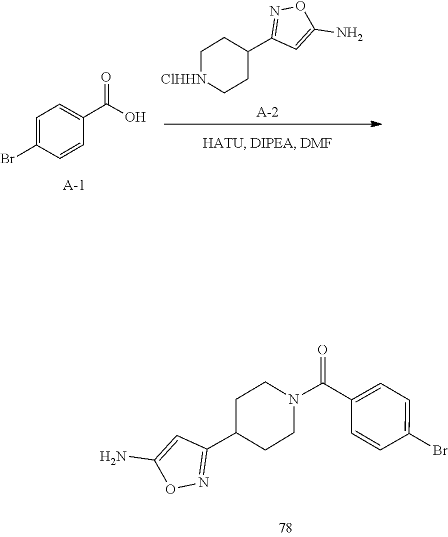

Similar to the preparation of 73, purification by prep-HPLC provided the target compound 78 (33 mg, 94.23 umol, 18.94% yield) as a white solid. ¹H NMR (400 MHz, CDCl₃) δ 7.57-7.53 (m, 2H), 7.32-7.27 (m, 2H), 4.99 (s, 1H), 4.66 (s, 1H), 4.43 (s, 2H), 3.79 (s, 1H), 3.03 (s, 2H), 2.88 (ddd, J=11.2, 7.6, 3.7 Hz, 1H), 2.01 (d, J=5.6 Hz, 2H), 1.69 (s, 2H). ESI-MS: m/z=351.8 (M+1).

### Example 79: Synthesis of [4-(5-aminoisoxazol-3-yl)-1-piperidyl]-[4-chloro-3-(trifluoromethoxy)phenyl]methanone (79)

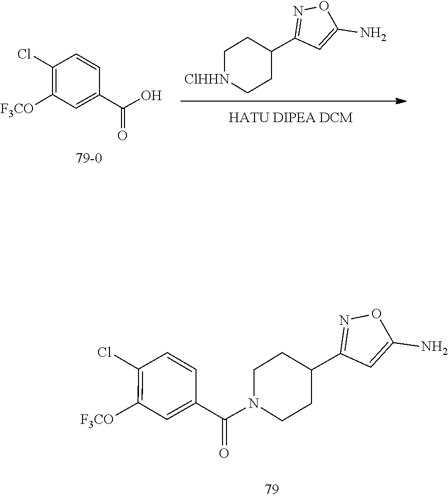

Similar to the preparation of 73, purification by prep-HPLC provided the target compound 79 (24.7 mg, 0.063 mmol, 10.6% yield) as a white solid. ¹H NMR (400 MHz, CDCl₃) δ 7.53 (d, J=8.2 Hz, 1H), 7.39 (s, 1H), 7.31 (d, J=8.2 Hz, 1H), 5.00 (s, 1H), 4.62 (s, 1H), 4.48 (s, 2H), 3.76 (s, 1H), 3.05 (s, 2H), 2.97-2.81 (m, 1H), 2.01 (s, 2H), 1.73 (s, 2H). ESI-MS: m/z=389.8 (M+1).

### Example 80: Synthesis of [4-(5-aminoisoxazol-3-yl)-1-piperidyl]-[3-chloro-4-(trifluoromethoxy)phenyl]methanone (80)

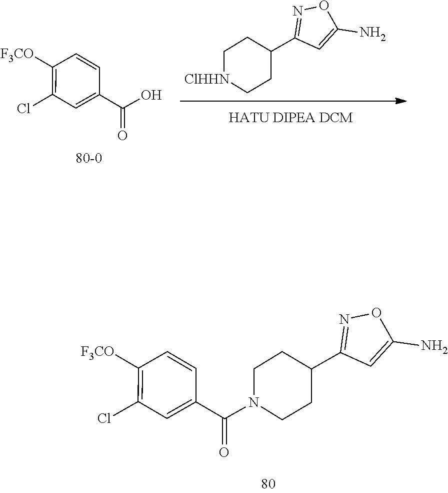

Similar to the preparation of 73, purification by prep-HPLC provided the target compound 80 (24.7 mg, 0.063 mmol, 10.6% yield) as a white solid. ¹H NMR (400 MHz, CDCl₃) δ 7.54 (t, J=4.2 Hz, 1H), 7.40-7.31 (m, 2H), 5.00 (s, 1H), 4.62 (s, 1H), 4.46 (s, 2H), 3.77 (s, 1H), 3.10 (d, J=44.8 Hz, 2H), 2.90 (tt, J=11.2, 3.7 Hz, 1H), 2.01 (d, J=4.9 Hz, 2H), 1.69 (s, 2H). ESI-MS: m/z=389.8 (M+1).

### Example 81: Synthesis of [4-(5-aminoisoxazol-3-yl)-1-piperidyl]-[3-(trifluoromethoxy)phenyl]methanone (81)

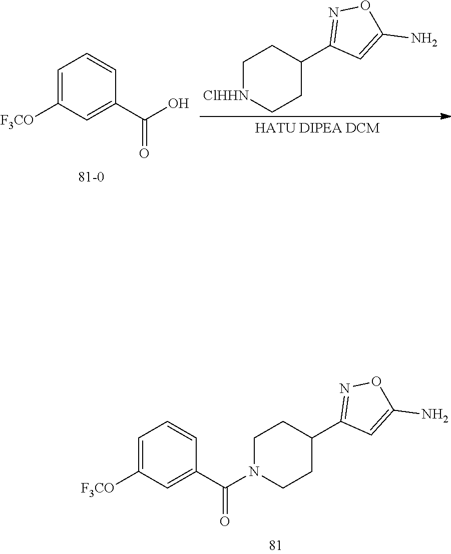

Similar to the preparation of 73, purification by prep-HPLC provided the target compound 81 (38.6 mg, 0.109 mmol, 18.1% yield) as a light yellow solid. ¹H NMR (400 MHz, CDCl₃) δ 7.49-7.28 (m, 2H), 7.27-7.11 (m, 2H), 4.99 (s, 1H), 4.69 (d, J=13.0 Hz, 1H), 4.52 (s, 2H), 3.80-3.56 (m, 1H), 3.15 (s, 1H), 3.09-2.97 (m, 1H), 2.90 (tt, J=11.2, 3.8 Hz, 1H), 2.10-2.01 (m, 1H), 1.93 (d, J=11.4 Hz, 1H), 1.74 (ddd, J=25.0, 11.6, 4.2 Hz, 2H). ESI-MS: m/z=355.9 (M+1).

### Example 82: Synthesis of (4-(5-aminoisoxazol-3-yl)piperidin-1-yl) (4-bromophenyl)methanone (82)

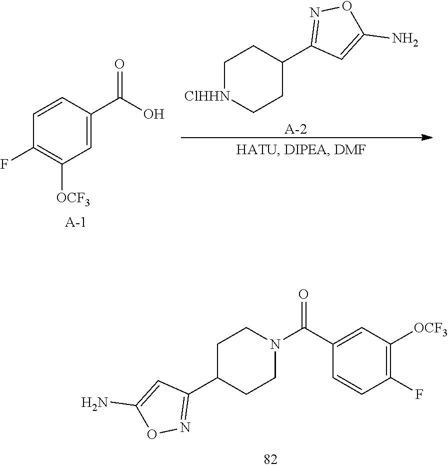

Similar to the preparation of 73, purification by prep-HPLC provided the target compound 82 (23 mg, 61.61 umol, 13.81% yield) as a red solid. ¹H NMR (400 MHz, CDCl₃) δ 7.43-7.34 (m, 2H), 7.28 (s, 1H), 7.26-7.22 (m, 1H), 5.00 (s, 1H), 4.60 (s, 1H), 4.44 (s, 2H), 3.83 (d, J=37.5 Hz, 1H), 3.10 (s, 2H), 2.90 (tt, J=11.2, 3.9 Hz, 1H), 1.99 (s, 2H), 1.76 (m, 1H). ESI-MS: m/z=373.8 (M+1).

### Example 83: Synthesis of [4-(5-aminoisoxazol-3-yl)-1-piperidyl]-(3-fluoro-4-methylsulfonyl-phenyl)methanone (83)

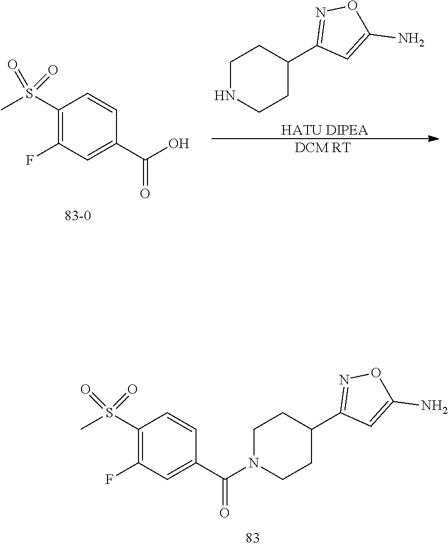

Similar to the preparation of 73, purification by prep-HPLC provided the target compound 83 (14.5 mg, 0.039 mmol, 6.6% yield) as a red solid. ¹H NMR (400 MHz, MeOD) δ 7.92 (t, J=7.5 Hz, 1H), 7.42-7.34 (m, 2H), 4.90 (s, 1H), 4.51 (d, J=12.8 Hz, 1H), 3.55 (d, J=12.8 Hz, 1H), 3.19 (s, 3H), 3.14 (d, J=14.8 Hz, 1H), 2.97 (dd, J=22.9, 10.3 Hz, 1H), 2.79 (tt, J=11.4, 3.8 Hz, 1H), 1.94 (t, J=6.2 Hz, 1H), 1.78 (d, J=10.3 Hz, 1H), 1.70-1.49 (m, 2H). ESI-MS: m/z=367.8 (M+1)

### Example 84: Synthesis of [4-(5-aminoisoxazol-3-yl)-1-piperidyl]-[4-(difluoromethoxy)phenyl]methanone (84)

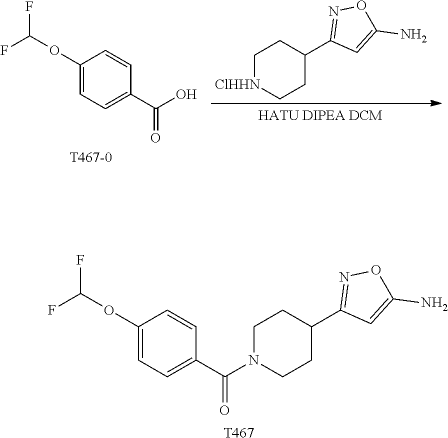

Similar to the preparation of 73, purification by prep-HPLC provided the target compound 84 (21.6 mg, 0.064 mmol, 10.6% yield) as a white solid. ¹H NMR (400 MHz, CDCl₃) δ 7.47-7.40 (m, 2H), 7.16 (d, J=8.6 Hz, 2H), 6.54 (t, J=73.4 Hz, 1H), 4.99 (s, 1H), 4.66 (s, 1H), 4.45 (s, 2H), 3.82 (s, 1H), 3.06 (s, 2H), 2.89 (ddd, J=15.2, 7.6, 3.9 Hz, 1H), 2.08-1.89 (m, 2H), 1.69 (s, 1H). ESI-MS: m/z=337.9 (M+1)

### Example 85: Synthesis of (4-(5-aminoisoxazol-3-yl)piperidin-1-yl) (4-(methylsulfonyl)phenyl)methanone (85)

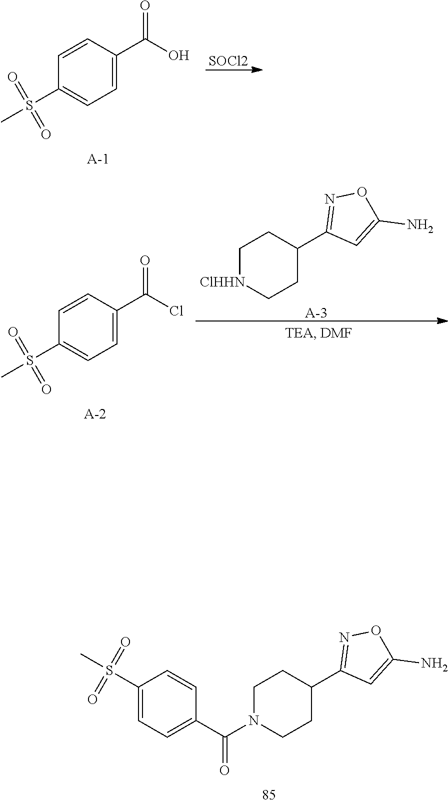

Synthesis of A-2 (step 1): To a mixture of 4-methylsulfonylbenzoic acid (200 mg, 998.94 umol) in SOCl2 (2 mL). The reaction was stirred at 60° C. for 2 h. The reaction was directly concentrated yield crude 4-methylsulfonylbenzoyl chloride (226 mg, 1.03 mmol, >99% yield).

Synthesis of 85 (step 2): To a solution of crude A-2 (160.59 mg, 960.41 umol) in DMF (2 mL) was added N, N-diethylethanamine (485.92 mg, 4.80 mmol, 669.31 uL). The mixture was stirred for 20 min, then A-3 (210 mg, 960.41 umol) was added to the reaction. Then the reaction was stirred at 25° C. for 16 h. When completed, the reaction was directly purified by prep-HPLC to yield 85 (6.51 mg, 18.63 umol, 1.94% yield). ¹H NMR (400 MHz, CDCl₃) δ 8.03-7.98 (m, 2H), 7.62-7.57 (m, 2H), 5.00 (s, 1H), 4.66 (s, 1H), 4.44 (s, 2H), 3.66 (s, 1H), 3.17 (s, 1H), 3.08 (d, J=2.3 Hz, 4H), 2.91 (tt, J=11.0, 3.8 Hz, 1H), 2.06 (s, 1H), 1.91 (s, 1H), 1.76 (s, 1H), 1.67 (s, 1H). ESI-MS: m/z=349.8 (M+1).

### Example 86: Synthesis of [4-[5-(benzylamino)isoxazol-3-yl]-1-piperidyl]-[3-fluoro-4-(trifluoromethyl)phenyl]methanone (86)

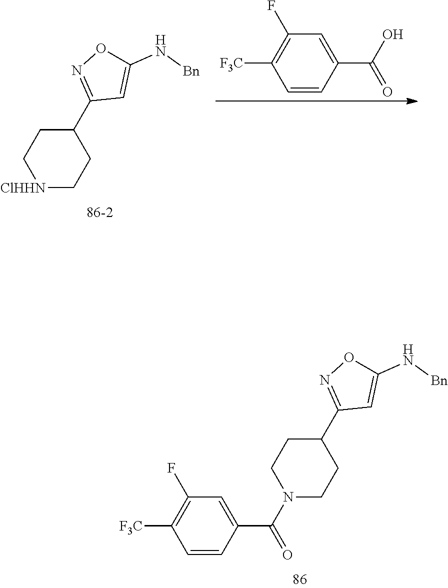

Similar to the preparation of 85, purification by prep-HPLC provided the target compound 86 (20.9 mg, 0.047 mmol, 10.9% yield) as a white solid. ¹H NMR (400 MHz, CDCl₃) δ 7.65 (dd, J=8.5, 2.7 Hz, 1H), 7.37-7.30 (m, 5H), 7.27 (s, 1H), 7.24 (d, J=10.4 Hz, 1H), 4.95 (t, J=5.8 Hz, 1H), 4.84 (s, 1H), 4.63 (t, J=6.0 Hz, 1H), 4.35 (d, J=5.9 Hz, 2H), 3.68 (s, 1H), 3.24-2.96 (m, 2H), 2.89 (tt, J=11.1, 3.8 Hz, 1H), 2.14-1.90 (m, 2H), 1.67 (s, 2H). ESI-MS: m/z=447.9 (M+1).

### Example 87: Synthesis of (4-(5-aminoisoxazol-3-yl)-4-methylpiperidin-1-yl)(4-(trifluoromethoxy)phenyl)methanone (87)

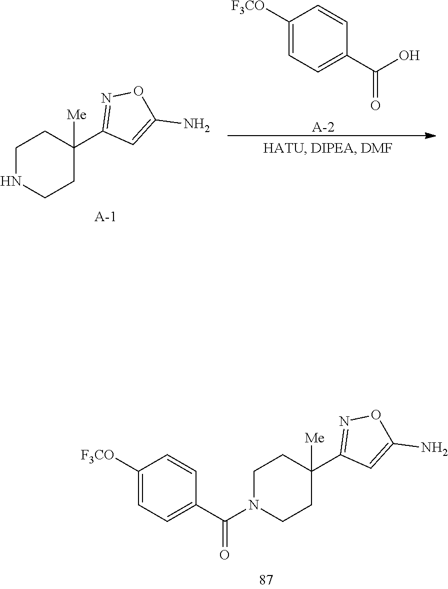

Similar to the preparation of 88, purification by prep-HPLC provided the target compound 87 (29 mg, 78.52 umol, 16.18% yield) as a solid. ¹H NMR (400 MHz, CDCl₃) δ 7.46-7.40 (m, 2H), 7.25-7.21 (m, 2H), 5.00 (s, 1H), 4.42 (s, 2H), 4.25 (s, 1H), 3.49 (s, 1H), 3.39 (s, 1H), 3.28 (s, 1H), 2.09 (d, J=13.0 Hz, 2H), 1.67 (s, 1H), 1.45 (s, 1H), 1.29 (s, 3H). ESI-MS: m/z=369.9 (M+1).

### Example 88: Synthesis of 3-[4-methyl-1-[4-(trifluoromethyl)phenyl]sulfonyl-4-piperidyl]isoxazol-5-amine (88)

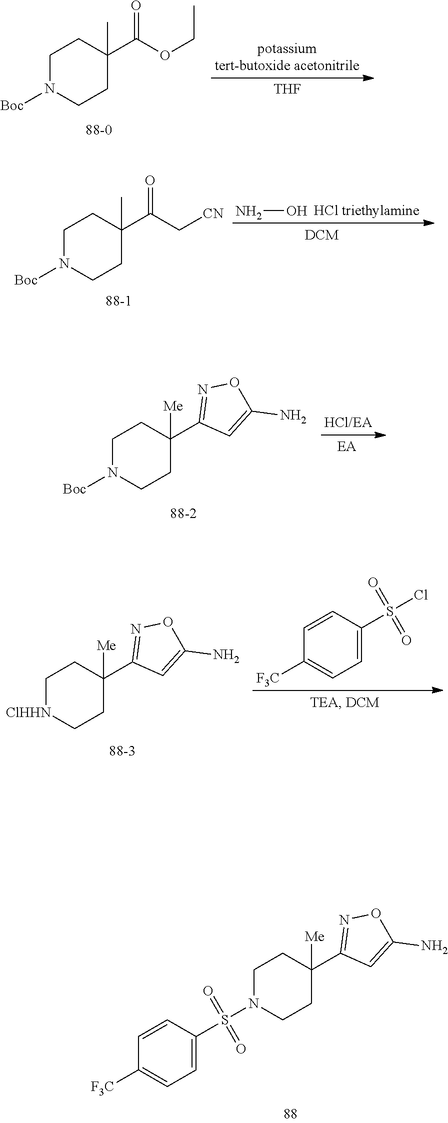

Synthesis of 88-1 (Step 1): To a solution of 01-tert-butyl 04-ethyl 4-methylpiperidine-1,4-dicarboxylate (4 g, 14.74 mmol) and acetonitrile (3.03 g, 73.71 mmol, 3.85 mL) in THE (80 mL) added potassium 2-methylpropan-2-olate (4.96 g, 44.22 mmol) at 0° C. The reaction was stirred at 25° C. for 2 h. TLC (EA:PE=1:4, UV, 254 nm) showed that the started material was consumed and new spots were detected. The reaction was quenched with NH4Cl and extracted with EA (30 mL*3). The organic layer was consumed, washed with brine, dried over Na2SO4 and concentrate to give crude product. The crude product was purified with glass silica gel column (EA:PE=0˜40%) and concentrate to give tert-butyl 4-(2-cyanoacetyl)-4-methyl-piperidine-1-carboxylate (1.8 g, 6.76 mmol, 45.85% yield).

Synthesis of 88-2 (Step 2): To a solution of tert-butyl 4-(2-cyanoacetyl)-4-methyl-piperidine-1-carboxylate (1.8 g, 6.76 mmol) in DCM (18 mL) added hydroxylamine hydrochloride (657.51 mg, 9.46 mmol, 393.72 uL) and N,N-diethylethanamine (1.37 g, 13.52 mmol, 1.88 mL). The reaction was stirred at 55° C. for 16 h. When the reaction was judged completed by LCMS, the reaction was diluted with water and extracted with DCM (30 mL). The organic layers were combined washed with brine, dried over Na2SO4 and concentrated to give crude product. The crude product was purified by silica gel column (EA:EP=0˜100%) and concentrated to give tert-butyl 4-(5-aminoisoxazol-3-yl)-4-methyl-piperidine-1-carboxylate (540 mg, 1.92 mmol, 28.40% yield).

Synthesis of 88-3 (Step 3): To a solution of tert-butyl 4-(5-aminoisoxazol-3-yl)-4-methyl-piperidine-1-carboxylate (540 mg, 1.92 mmol) in EA (2 mL) added 4M HCU/EA (5 mL). The reaction was stirred at 25° C. for 2 h. When the reaction was judged completed by LCMS, the reaction was directly concentrated to move the solvent and to give 3-(4-methyl-4-piperidyl)isoxazol-5-amine (413 mg, 2.28 mmol, 118.73% yield).

Synthesis of 3-[4-methyl-1-[4-(trifluoromethyl)phenyl]sulfonyl-4-piperidyl]isoxazol-5-amine (88, Step 4): 4-(trifluoromethyl)benzenesulfonyl chloride (100 mg, 408.80 umol), N,N-diethylethanamine (165.47 mg, 1.64 mmol, 227.91 uL) were dissolved in DCM (3 mL) and the mixture was stirred for 20 minutes. 3-(4-methyl-4-piperidyl)isoxazol-5-amine (74.09 mg, 408.80 umol) was added to the reaction mixture, then it was stirred at room temperature for 16 hours. When the reaction was judged completed by LCMS, then the reaction was diluted with water (10 mL) and extracted with DCM (20 mL). The organic layers were combined, washed with brine, dried over Na2SO4 and concentrated to give crude product. The crude product was purified with prep-HPLC team to give 3-[4-methyl-1-[4-(trifluoromethyl)phenyl]sulfonyl-4-piperidyl]isoxazol-5-amine 88 (30.53 mg, 78.40 umol, 19.18% yield). ¹H NMR (400 MHz, CDCl₃) δ 7.85 (d, J=8.2 Hz, 2H), 7.77 (d, J=8.3 Hz, 2H), 4.90 (s, 1H), 4.38 (s, 2H), 3.51 (dt, J=12.0, 3.8 Hz, 2H), 2.76 (td, J=11.9, 2.7 Hz, 2H), 2.13 (d, J=13.7 Hz, 2H), 1.80-1.67 (m, 2H), 1.21 (s, 3H). ESI-MS: m/z=389.8 (M+1).

### Example 89: Synthesis of ((1R,5S,6r)-6-(5-aminoisoxazol-3-yl)-3-azabicyclo[3.1.0]hexan-3-yl)(3-chloro-4-(trifluoromethyl)phenyl)methanone (89)

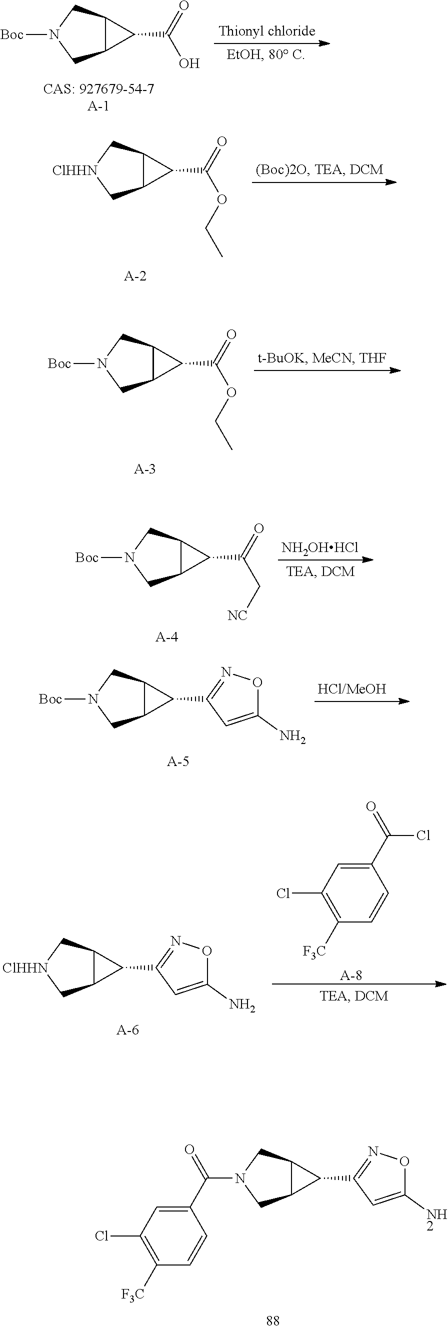

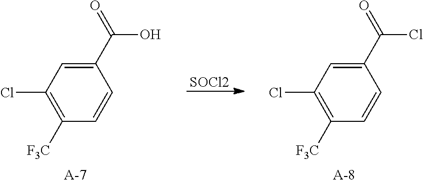

Synthesis of A-2 (step 1): To a solution of A-1 (1 g, 4.40 mmol) in SO2Cl2 (8 mL). The reaction was stirred at 80° C. for 2 h. LCMS showed that starting material was consumed. Then the reaction was directly concentrated to give a crude residue, then EtOH (3 mL) was added to the crude product. The crude solution was then directly concentrated to give A-2 (746 mg, 4.81 mmol). The product was directly used at next step without further purification.

Synthesis of A-3 (step 2): To a solution of A-2 in DCM (10 mL) added N,N-diethylethanamine (1.39 g, 13.76 mmol, 1.92 mL) and di-tert-butyl pyrocarbonate (1.20 g, 5.51 mmol, 1.26 mL). Then the reaction was stirred at 25° C. for 16 h. TLC EA:PE=1:4, UV, 254 nm showed that starting material was consumed and new spot was observed. The reaction was then diluted with water (30 mL) and extracted with DCM (30 mL*2). The organic layers were combined washed with brine, dried over Na2SO4 and concentrated to give crude product. The crude product was purified with silica gel (EA:PE=0-30%) and concentrated to give A-3 (865 mg, 3.39 mmol, 73.85% yield).

Synthesis of A-4 (step 3): To a solution of A-3 (865 mg, 3.39 mmol) in THE (18 mL) added acetonitrile (763.76 mg, 18.60 mmol, 971.71 uL). Then potassium 2-methylpropan-2-olate (1.25 g, 11.16 mmol) was added at 0° C. After addition, the reaction was stirred at 25° C. for 1 h. TLC (EA:PE=1:2, UV, 254 nm showed that started material was consumed and new spot was detected. Then the reaction was diluted with NH4Cl and extracted with EA (30 mL*3) the organic layer was combined, washed with brine, dried over Na2SO4 and concentrated to give crude product. The crude product was purified with silica gel column (EA:PE=0-40) and concentrated to give A-4 (350 mg, 1.4 mmol, 30.06% yield).

Synthesis of A-5 (step 4): To a solution of A-4 (350 mg, 1.40 mmol) in DCM (0.5 mL) added hydroxylamine hydrochloride (108.83 mg, 1.57 mmol, 65.17 uL) and N,N-diethylethanamine (226.40 mg, 2.24 mmol, 311.85 uL). Then the reaction was stirred at 55° C. for 12 h.

When the LCMS showed that starting material was consumed and new peak was detected. The reaction was diluted with water and extracted with DCM (30 mL*2). The organic layer was combined, washed with brine, dried over Na2SO4 and concentrated to give crude product. The crude product was purified with silica gel column (PE:EA=0-60%) and concentrated to give A-5 (243 mg, 915.92 umol).

Synthesis of A-6 (step 5): A-5 (243 mg, 915.92 umol) in bottle was added 4 M HCl/MeOH (3 mL). Then the reaction was stirred at 25° C. for 2 h. TLC (EA:PE=1:1, UV, 254 nm) showed that starting material was consumed. The reaction was directly concentrated to move off the solvent to give A-6 (182 mg, 1.10 mmol, >99% yield).

Synthesis of A-8 (step 6): To a mixture of A-7 (100 mg, 445.31 umol) in SOCl2 (2 mL). The reaction was stirred at 60° C. for 3 h. TLC (EA:PE=1:1, UV, 254 nm) showed that starting material was consumed and new spot was observed. The reaction was directly concentrated to give crude A-8 (112 mg, 460.89 umol, 103.50% yield).

Synthesis of 89 (step 7): To a solution of A-6 (90 mg, 544.82 umol) in DMF (2 mL) added N,N-diethylethanamine (214.45 mg, 2.12 mmol, 295.38 uL). Then A-8 (103 mg, 423.85 umol) was dissolved in DCM (2 mL) and added to the reaction, the reaction was stirred at 25° C. for 2 h. LCMS showed that the starting material was consumed and desired product MS was detected. The reaction was diluted with water (10 mL) and extracted with DCM (20 mL*2). The organic layer was combined, washed with brine, dried over Na2SO4 and concentrated to give crude product. The crude product was purified by prep-HPLC and concentrated to yield 89 (16 mg, 59.41 umol, 14.02% yield). ¹H NMR (400 MHz, CDCl₃) δ 7.74 (d, J=8.1 Hz, 1H), 7.59 (s, 1H), 7.43 (d, J=8.1 Hz, 1H), 4.89 (s, 1H), 4.40 (s, 2H), 4.29 (d, J=12.5 Hz, 1H), 3.72 (dd, J=10.9, 3.7 Hz, 1H), 3.67-3.55 (m, 2H), 2.09-1.96 (m, 2H), 1.66 (t, J=3.4 Hz, 1H). ESI-MS: m/z=371.8 (M+1).

### Example 90: Synthesis of (4-(5-aminoisoxazol-3-yl)-4-methylpiperidin-1-yl)(3-fluoro-4-(trifluoromethyl)phenyl)methanone (90)

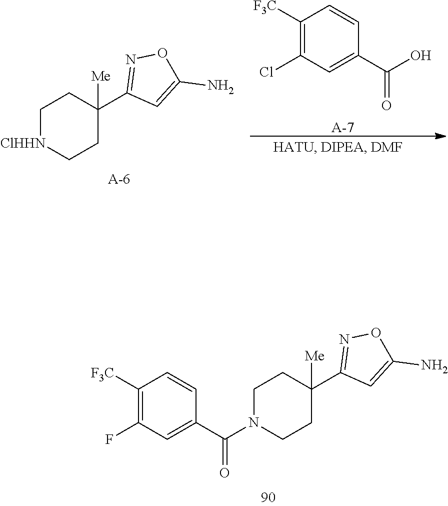

Similar to the preparation of 88, purification by prep-HPLC provided the target compound 90 (9.9 mg, 26.87 umol, 6.2% yield) as a solid. ¹H NMR (400 MHz, CDCl₃) δ 7.65 (t, J=7.6 Hz, 1H), 7.25-7.20 (m, 2H), 5.00 (s, 1H), 4.43 (s, 2H), 4.32-4.24 (m, 1H), 3.50-3.35 (m, 2H), 3.30-3.20 (m, 1H), 2.10 (s, 2H), 1.64 (s, 2H), 1.30 (s, 3H). ESI-MS: m/z=372.13 (M+1).

### Example 91: Synthesis of [4-[5-(ethylamino)isoxazol-3-yl]-1-piperidyl]-[3-fluoro-4-(trifluoromethyl)phenyl]methanone (91)

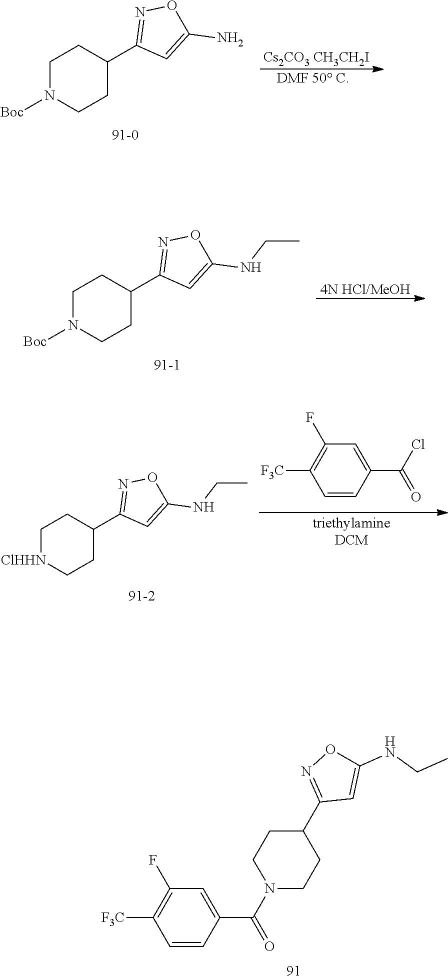

Synthesis of 91-1 (Step 1): Dissolve tert-butyl 4-(5-aminoisoxazol-3-yl)piperidine-1-carboxylate (1.0 g, 3.74 mmol), cesium carbonate (1.83 g, 5.61 mmol) in DMF (8.0 mL) and stir at 50° C. for 1.5 hours. The reaction solution was extracted by EA, concentrated, dried and purified by column chromatography to obtain a pale yellow solid.

Synthesis of 91-2 (Step 2): Dissolve tert-butyl 4-[5-(ethylamino)isoxazol-3-yl]piperidine-1-carboxylate (200 mg, 677.10 umol) in HCl/EA (10 mL) and stir at room temperature for 3 hours. TLC was used to monitor the consumption of the starting material. When judged completed, the reaction liquid was concentrated for the next reaction.

Similar to the preparation of 85, purification by prep-HPLC provided the target compound 91 (12.91 mg, 33.50 umol, 13% yield) as a white solid. ¹H NMR (400 MHz, CDCl₃) δ 7.67 (t, J=7.5 Hz, 1H), 7.29 (s, 1H), 7.24 (s, 1H), 4.84 (s, 1H), 4.64 (s, 1H), 4.48 (s, 1H), 3.70 (s, 1H), 3.28-3.12 (m, 3H), 3.04 (s, 1H), 2.91 (tt, J=11.0, 3.7 Hz, 1H), 2.16-1.88 (m, 2H), 1.76 (s, 2H), 1.25 (d, J=7.2 Hz, 3H). ESI-MS: m/z=385.9 (M+1).

### Example 92: Synthesis of 3-[1-[3-fluoro-4-(trifluoromethyl)phenyl]sulfonyl-4-piperidyl]isoxazol-5-amine (92)

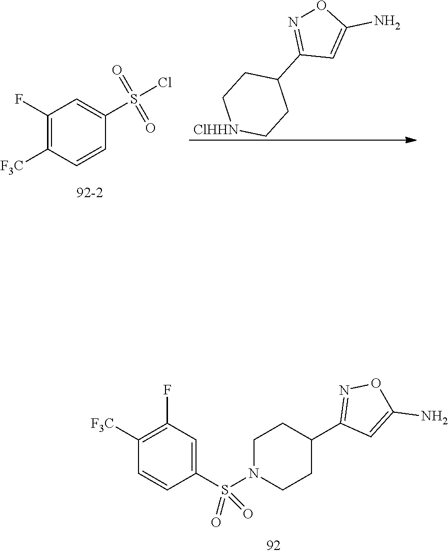

Similar to the preparation of 77, purification by prep-HPLC provided the target compound 92 (12.91 mg, 33.50 umol, 13% yield) as a white solid. ¹H NMR (400 MHz, CDCl₃) δ 7.81 (t, J=7.3 Hz, 1H), 7.69-7.60 (m, 2H), 4.96 (s, 1H), 4.43 (s, 2H), 3.80 (d, J=11.9 Hz, 2H), 2.71-2.53 (m, 3H), 2.05-1.99 (m, 2H), 1.88-1.76 (m, 2H). ESI-MS: m/z=393.8 (M+1)

### Example 93: Synthesis of (4-(5-aminoisoxazol-3-yl)piperidin-1-yl)(3-(methylsulfonyl)phenyl)methanone (93)

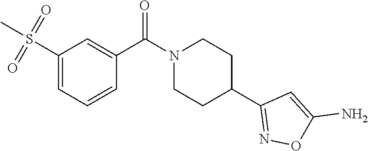 [SMILES: CS(=O)(=O)c1cccc(C(=O)N2CCC(c3cc(N)on3)CC2)c1]

Similar to the preparation of 1, purification by prep-HPLC provided the target compound 93 (33.2 mg, 0.095 mmol, 38% yield) as a white solid. ¹H NMR (400 MHz, CD3SOCD3, 299 K) δ (ppm)=8.53-8.36 (m, 1H), 8.34-8.22 (m, 1H), 8.18 (br d, J=7.5 Hz, 1H), 8.01-7.76 (m, 1H), 6.54 (s, 2H), 4.90 (s, 1H), 4.52-4.36 (m, 1H), 3.59-3.47 (m, 1H), 3.35-3.30 (m, 3H), 3.16-3.12 (m, 1H), 3.00-2.90 (m, 1H), 2.87-2.75 (m, 1H), 2.03-1.85 (m, 1H), 1.82-1.70 (m, 1H), 1.67-1.50 (m, 2H). ESI-MS: m/z=349.9 (M+1).

### Example 94: Synthesis of (4-(5-aminoisoxazol-3-yl)piperidin-1-yl)(3-fluoro-4-(trifluoromethoxy)phenyl)methanone (94)

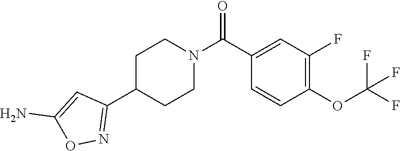 [SMILES: Nc1cc(C2CCN(C(=O)c3ccc(OC(F)(F)F)c(F)c3)CC2)no1]

Similar to the preparation of 1, purification by prep-HPLC provided the target compound 94 (42.7 mg, 0.114 mmol, 46% yield) as a white solid. 1H NMR (400 MHz, CD3SOCD3, 299 K) Shift (ppm)=7.70-7.57 (m, 2H), 7.45-7.30 (m, 1H), 6.54 (s, 2H), 4.90 (s, 1H), 4.52-4.36 (m, 1H), 3.59-3.47 (m, 1H), 3.16-3.12 (m, 1H), 3.00-2.90 (m, 1H), 2.87-2.75 (m, 1H), 2.03-1.85 (m, 1H), 1.82-1.70 (m, 1H), 1.67-1.50 (m, 2H). ESI-MS: m/z=373.9 (M+1).

### Example 95: Synthesis of (4-(5-aminoisoxazol-3-yl)piperidin-1-yl)(2-fluoro-4-(trifluoromethoxy)phenyl)methanone (95)

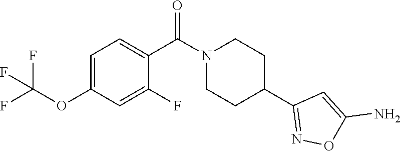 [SMILES: Nc1cc(C2CCN(C(=O)c3ccc(OC(F)(F)F)cc3F)CC2)no1]

Similar to the preparation of 1, purification by prep-HPLC provided the target compound 95 (47.0 mg, 0.126 mmol, 50% yield) as a white solid. 1H NMR (400 MHz, CD3SOCD3, 299 K) Shift (ppm)=7.67-7.56 (m, 1H), 7.56-7.49 (m, 1H), 7.35 (br d, J=9.3 Hz, 1H), 6.55 (s, 2H), 4.87 (s, 1H), 4.56-4.38 (m, 1H), 3.41 (br d, J=13.3 Hz, 1H), 3.14 (br d, J=7.3 Hz, 1H), 2.97 (dt, J=2.5, 12.5 Hz, 1H), 2.81 (tt, J=3.7, 11.4 Hz, 1H), 1.94 (br d, J=12.3 Hz, 1H), 1.80 (br d, J=13.1 Hz, 1H), 1.67-1.39 (m, 2H). ESI-MS: m/z=373.9

### Example 96: Synthesis of (4-(5-aminoisoxazol-3-yl)-4-fluoropiperidin-1-yl)(3-fluoro-4-(trifluoromethyl)phenyl)methanone (96)

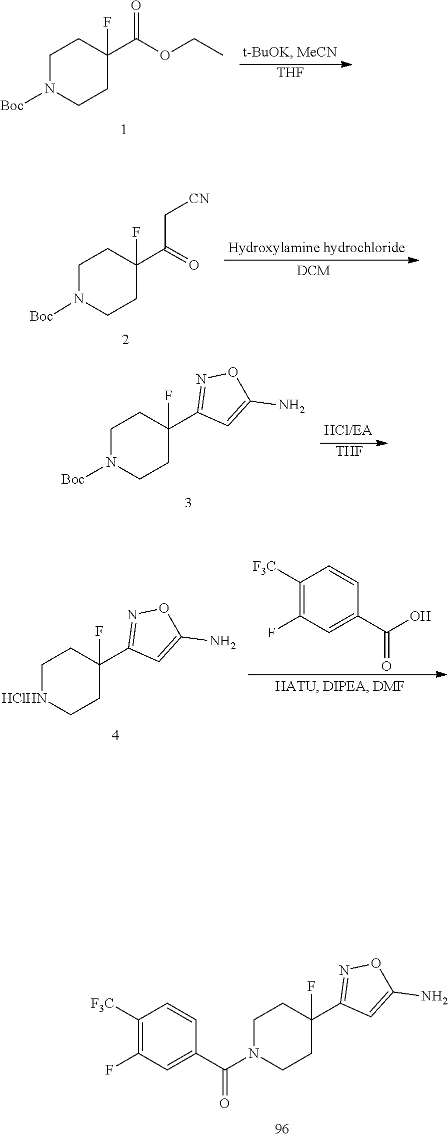

Similar to the preparation of 88, purification by prep-HPLC provided the target compound 96 (22.6 mg, 57.69 umol, 19.43% yield) as a white solid. ¹H NMR (400 MHz, CDCl₃) δ 7.68 (t, J=7.5 Hz, 1H), 7.32-7.26 (m, 2H), 5.22 (s, 1H), 4.55 (s, 3H), 3.60 (s, 1H), 3.50 (s, 1H), 3.37 (s, 1H), 2.37-1.99 (m, 4H). ESI-MS: m/z=375.9 (M+1)

### Example 97: Synthesis of 3-(4-fluoro-1-(4-(trifluoromethyl)phenylsulfonyl)piperidin-4-yl)isoxazol-5-amine (97)

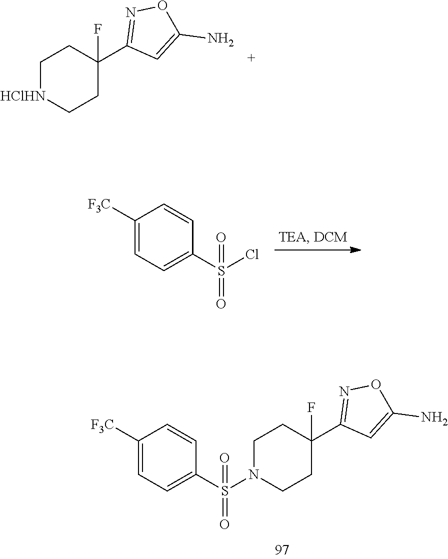

Similar to the preparation of 88, purification by prep-HPLC provided the target compound 97 (3.02 mg, 7.68 umol, 2.59% yield) as a white solid. ¹H NMR (400 MHz, CDCl₃) δ 7.92 (d, J=8.2 Hz, 2H), 7.82 (d, J=8.3 Hz, 2H), 5.15 (s, 1H), 4.50 (s, 2H), 3.77-3.67 (m, 2H), 2.83 (ddd, J=12.0, 7.6, 3.3 Hz, 2H), 2.23-2.16 (m, 3H), 2.06-1.96 (m, 1H). ESI-MS: m/z=393.8 (M+1)

### Example 98: Synthesis of (4-(5-aminoisoxazol-3-yl)-4-fluoropiperidin-1-yl)(4-(trifluoromethoxy)phenyl)methanone (98)

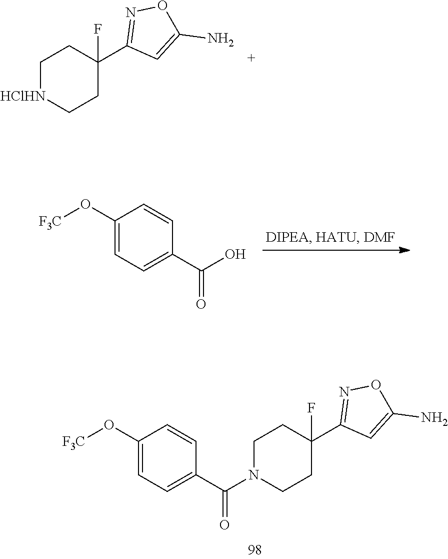

Similar to the preparation of 88, purification by prep-HPLC provided the target compound 98 (18.3 mg, 49.02 umol, 16.51% yield) as a white solid. ¹H NMR (400 MHz, CDCl₃) δ 7.47 (d, J=8.0 Hz, 2H), 7.27 (d, J=8.0 Hz, 2H), 5.22 (s, 1H), 4.53 (s, 3H), 3.69 (s, 1H), 3.43 (s, 2H), 2.22-2.09 (m, 4H). ESI-MS: m/z=373.9 (M+1)

### Example 99: Synthesis of (4-(5-aminoisoxazol-3-yl)-4-fluoropiperidin-1-yl)(3-chloro-4-(trifluoromethyl)phenyl)methanone (99)

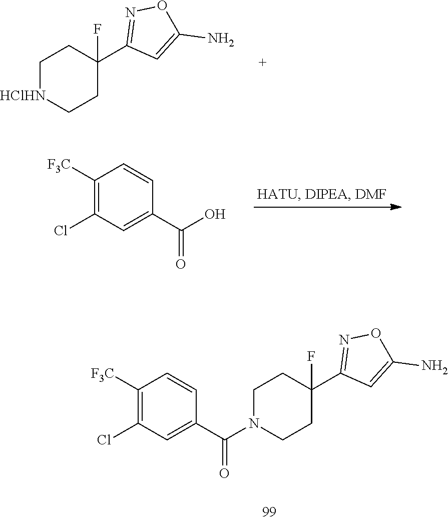

Similar to the preparation of 88, purification by prep-HPLC provided the target compound 99 (22.6 mg, 57.69 umol, 19.43% yield) as a white solid. ¹H NMR (400 MHz, CDCl₃) δ 7.75 (d, J=8.0 Hz, 1H), 7.56 (s, 1H), 7.40 (dd, J=8.0, 0.7 Hz, 1H), 5.23 (s, 1H), 4.54 (s, 3H), 3.66-3.31 (m, 3H), 2.28-2.06 (m, 4H). ESI-MS: m/z=391.8 (M+1).

### Example 100: Synthesis of 3-(1-((3-fluoro-4-(trifluoromethyl)phenyl)sulfonyl)piperidin-4-yl)-N-methylisoxazol-5-amine (100)

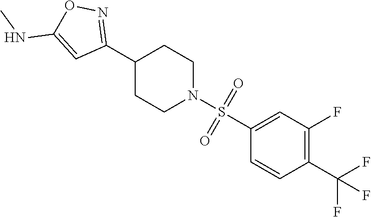 [SMILES: CNc1cc(C2CCN(S(=O)(=O)c3ccc(C(F)(F)F)c(F)c3)CC2)no1]

Similar to the preparation of 77, purification by prep-HPLC provided the target compound 100 (8.0 mg, 0.020 mmol, 2.9% yield) as a white solid. ¹H NMR (400 MHz, CDCl₃) δ 7.83-7.78 (m, 1H), 7.66 (d, J=8.9 Hz, 1H), 7.64 (d, J=8.9 Hz, 1H), 4.80 (s, 1H), 4.41 (d, J=4.7 Hz, 1H), 3.81 (dt, J=12.0, 3.3 Hz, 2H), 2.88 (d, J=5.2 Hz, 2H), 2.68-2.55 (m, 3H), 2.02 (dd, J=13.7, 3.1 Hz, 2H), 1.91-1.80 (m, 2H). ESI-MS: m/z=407.8 (M+1).

### Example 101: Synthesis of (3-fluoro-4-(trifluoromethyl)phenyl)(4-(5-((2,2,2-trifluoroethyl)amino)isoxazol-3-yl)piperidin-1-yl)methanone (101)

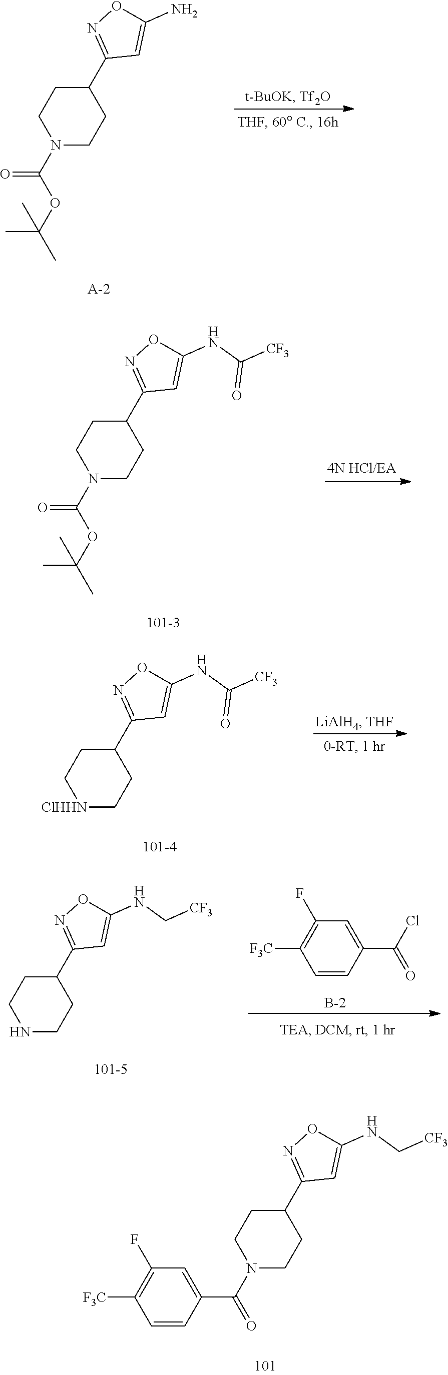

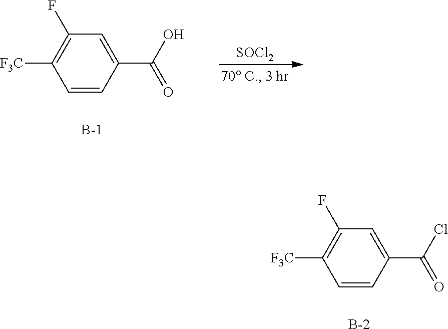

Synthesis of 101-3 (Step 1): To a 100 mL glass vial of (2,2,2-trifluoroacetyl) 2,2,2-trifluoroacetate (942.82 mg, 4.49 mmol, 632.76 uL) and A-2 (600 mg, 2.24 mmol) in THF (12 mL), was added potassium 2-methylpropan-2-olate (755.57 mg, 6.73 mmol). The reaction was stirred at 60° C. for 16 hr. Extracted with EA (20 mL*3), washed with aqueous NaCl, dried over Na₂SO₄, filtered and concentrated to yield crude 101-3 (1 g, 2.75 mmol, 122.63% yield) as a yellow solid.

Synthesis of 101-4 (Step 2): To a 100 mL glass vial was added 101-3 (800 mg, 2.20 mmol) in 4N HCl/EA (20 mL), the reaction was stirred at rt for 3 hr. The solid was filtered and concentrated to get the residue was washed with ether. Removed the solvent to get 101-4 (600 mg, 2.00 mmol, 90.93% yield, HCl) as a light-yellow solid.

Synthesis of 101-5 (Step 3): To a 50 mL glass vial of 2,2,2-trifluoro-N-[3-(4-piperidyl)isoxazol-5-yl]acetamide (400 mg, 1.52 mmol) in THF (5 mL) at 0° C. was added slowly 2,2,2-trifluoro-N-[3-(4-piperidyl)isoxazol-5-yl]acetamide (400 mg, 1.52 mmol). The reaction was stirred at rt for 1 hr under N2. When the reaction is completed, it was quenched by adding saturated potassium sodium tartrate solution, extracted with EA (20 mL*2), dried over Na2SO4 and concentrated to give 3-(4-piperidyl)-N-(2,2,2-trifluoroethyl)isoxazol-5-amine (290 mg, 1.16 mmol, 76.57% yield) as a yellow oil.

Synthesis of 101 (Step 4): Similar to the preparation of 85, purification by prep-HPLC provided the target compound 101 (25 mg, 56.91 umol, 17.8% yield) as a white solid. ¹H NMR (400 MHz, CDCl₃) δ 7.67 (t, J=7.4 Hz, 1H), 7.29 (s, 1H), 7.24 (s, 1H), 5.01 (s, 1H), 4.83 (t, J=6.7 Hz, 1H), 4.64 (s, 1H), 3.80 (tt, J=28.1, 14.1 Hz, 2H), 3.77 (s, 1H), 3.16 (s, 1H), 3.06 (s, 1H), 2.93 (tt, J=11.0, 3.7 Hz, 1H), 2.12-1.90 (m, 2H), 1.66 (s, 2H). ESI-MS: m/z=439.8 (M+1).

### Example 102: Synthesis of (3-chloro-4-(trifluoromethyl)phenyl)(4-(5-((2,2,2-trifluoroethyl)amino)isoxazol-3-yl)piperidin-1-yl)methanone (102)

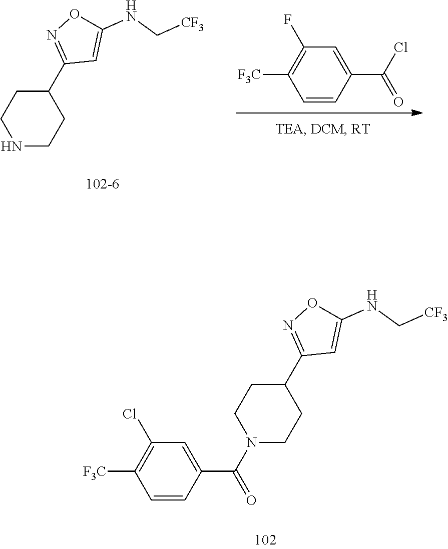 [SMILES: FC(F)(F)CNc1cc(C2CCNCC2)no1 ; O=C(Cl)c1ccc(C(F)(F)F)c(F)c1 ; O=C(c1ccc(C(F)(F)F)c(Cl)c1)N1CCC(c2cc(NCC(F)(F)F)on2)CC1]

Similar to the preparation of 101, purification by prep-HPLC provided the target compound 102 (30 mg, 65.82 umol, 16.4% yield) as a white solid. 1H NMR (400 MHz, CDCl3) δ 7.75 (d, J=8.0 Hz, 1H), 7.55 (s, 1H), 7.39 (d, J=7.9 Hz, 1H), 5.13-4.97 (m, 2H), 4.64 (s, 1H), 3.82-3.70 (m, 3H), 3.27-2.85 (m, 3H), 2.06 (s, 1H), 1.96 (s, 1H), 1.68 (s, 2H). ESI-MS: m/z=455.8 (M+1).

### Example 103: Synthesis of ((1R,5S,6r)-6-(5-aminoisoxazol-3-yl)-3-azabicyclo[3.1.0]hexan-3-yl)(3-chloro-4-(trifluoromethoxy)phenyl)methanone (103)

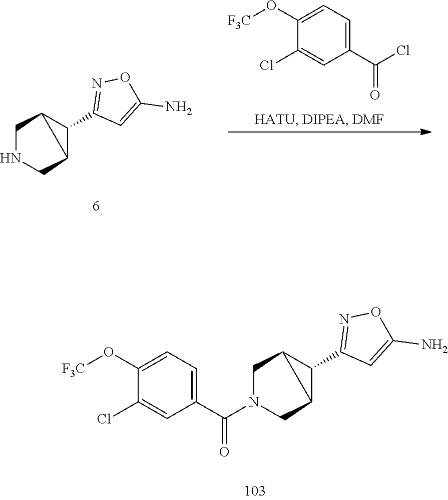 [SMILES: Nc1cc([C@H]2[C@@H]3CNC[C@@H]32)no1 ; O=C(Cl)c1ccc(OC(F)(F)F)c(Cl)c1 ; Nc1cc([C@H]2[C@@H]3CN(C(=O)c4ccc(OC(F)(F)F)c(Cl)c4)C[C@@H]32)no1]

Similar to the preparation of 89, purification by prep-HPLC provided the target compound 103 (43 mg, 111.18 umol, 34.2% yield) as a white solid. ¹H NMR (400 MHz, CDCl3) δ 7.60 (d, J=1.8 Hz, 1H), 7.42-7.33 (m, 2H), 4.89 (s, 1H), 4.70-4.20 (m, 3H), 3.74 (d, J=8.8 Hz, 1H), 3.68-3.55 (m, 2H), 2.06-1.99 (m, 2H), 1.65 (t, J=3.4 Hz, 1H). ESI-MS: m/z=387.8 (M+1).

### Example 104: Synthesis of [3-chloro-4-(trifluoromethyl)phenyl]-[4-[5-(2,2,2-trifluoroethylamino)isoxazol-3-yl]-1-piperidyl]methanone (104)

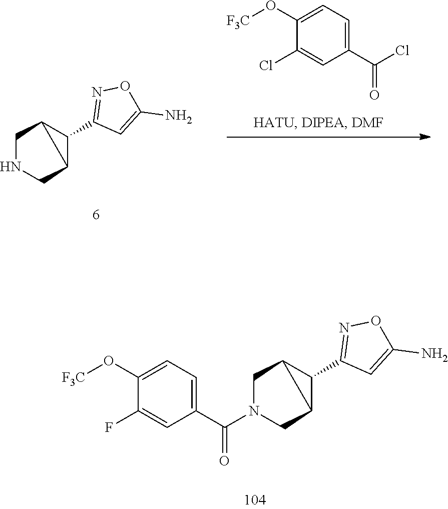 [SMILES: Nc1cc([C@H]2[C@@H]3CNC[C@@H]32)no1 ; O=C(Cl)c1ccc(OC(F)(F)F)c(Cl)c1 ; Nc1cc([C@H]2[C@@H]3CN(C(=O)c4ccc(OC(F)(F)F)c(F)c4)C[C@@H]32)no1]

Similar to the preparation of 89, purification by prep-HPLC provided the target compound 104 (30 mg, 65.82 umol, 16.40% yield) as a white solid. ¹H NMR (400 MHz, CDCl3) δ 7.50-7.40 (m, 2H), 7.28-7.21 (m, 1H), 4.88 (s, 1H), 4.58-4.20 (m, 3H), 3.80-3.55 (m, 3H), 2.02 (s, 2H), 1.65 (t, J=3.4 Hz, 1H). ESI-MS: m/z=371.8 (M+1).

### Example 105: Synthesis of [3-chloro-4-(trifluoromethoxy)phenyl]-[4-[5-(2,2,2-trifluoroethylamino)isoxazol-3-yl]-1-piperidyl]methanone (105)

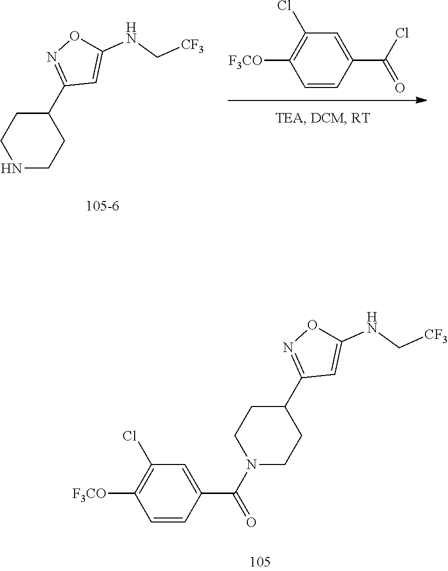 [SMILES: FC(F)(F)CNc1cc(C2CCNCC2)no1 ; O=C(Cl)c1ccc(OC(F)(F)F)c(Cl)c1 ; O=C(c1ccc(OC(F)(F)F)c(Cl)c1)N1CCC(c2cc(NCC(F)(F)F)on2)CC1]

Similar to the preparation of 101, purification by prep-HPLC provided the target compound 105 (18 mg, 38.15 umol, 9.5% yield) as a white solid. 1H NMR (400 MHz, CDCl3) δ 7.54 (s, 1H), 7.40-7.31 (m, 2H), 5.14 (s, 1H), 4.98 (d, J=15.7 Hz, 1H), 4.64 (s, 1H), 3.95-3.70 (m, 3H), 3.22-2.87 (m, 3H), 2.01 (s, 2H), 1.70 (s, 2H). ESI-MS: m/z=471.8 (M+1).

### Example 106: Synthesis of [(1S,5R)-6-(5-aminoisoxazol-3-yl)-3-azabicyclo[3.1.0]hexan-3-yl]-[3-fluoro-4-(trifluoromethoxy)phenyl]methanone (106)

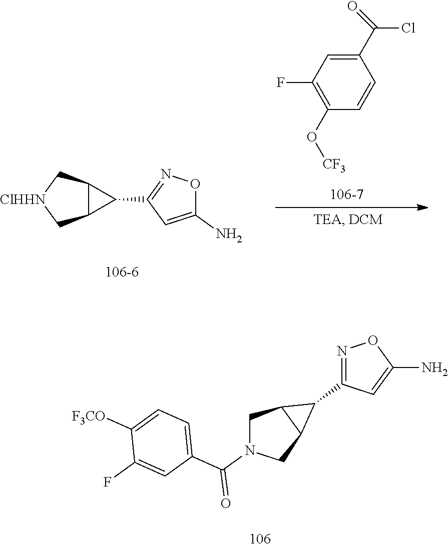

Similar to the preparation of 89, purification by prep-HPLC provided the target compound 106 (38 mg, 102.35 umol, 29.5% yield) as a white solid. ¹H NMR (400 MHz, CDCl₃) δ 7.36-7.27 (m, 3H), 4.89 (s, 1H), 4.50-4.28 (m, 3H), 3.73 (d, J=7.9 Hz, 1H), 3.67-3.57 (m, 2H), 2.03 (s, 2H), 1.64 (t, J=3.4 Hz, 1H). ESI-MS: m/z=371.8 (M+1).

### Example 107: Synthesis of [3-(5-aminoisoxazol-3-yl)pyrrolidin-1-yl]-[3-fluoro-4-(trifluoromethyl)phenyl]methanone (107)

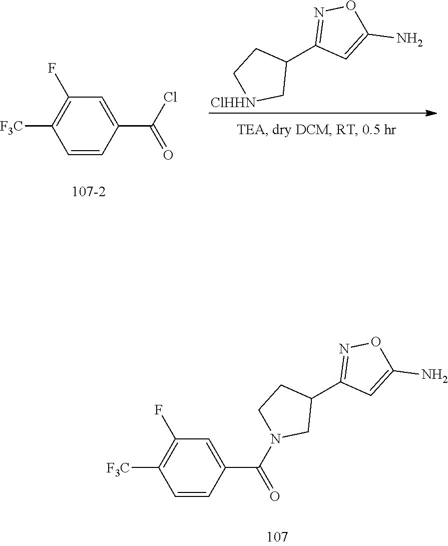

Similar to the preparation of 85, purification by prep-HPLC provided the target compound 107 (15 mg, 43.70 umol, 11.8% yield) as a white solid. ¹H NMR (400 MHz, CDCl3) major rotamer δ 7.66 (td, J=7.6, 2.9 Hz, 1H), 7.41-7.35 (m, 2H), 4.97 (s, 1H), 4.56 (s, 2H), 4.08-3.30 (m, 5H), 2.43-2.05 (m, 2H). ESI-MS: m/z=343.8 (M+1).

### Example 108: Synthesis of [3-(5-aminoisoxazol-3-yl)pyrrolidin-1-yl]-[3-chloro-4-(trifluoromethoxy)phenyl]methanone (108)

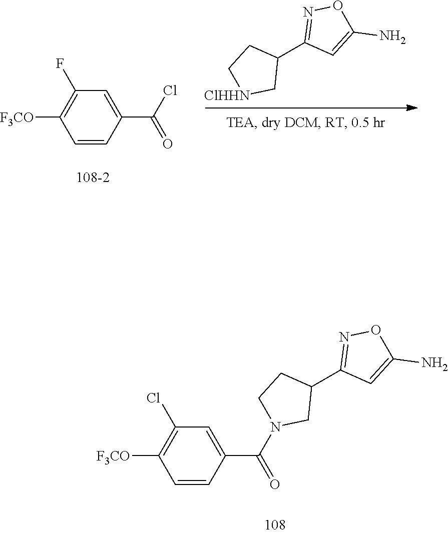

Similar to the preparation of 85, purification by prep-HPLC provided the target compound 108 (35 mg, 93.15 umol, 22.9% yield) as a white solid. ¹H NMR (400 MHz, CDCl3) major rotamer δ 7.66 (s, 1H), 7.46 (d, J=2.7 Hz, 1H), 7.36 (d, J=7.1 Hz, 1H), 4.97 (s, 1H), 4.75-4.72 (m, 2H), 4.08-3.31 (m, 6H), 2.45-2.24 (m, 1H), 2.19-2.08 (m, 1H). ESI-MS: m/z=375.8

### Example 109: Synthesis of [3-(5-aminoisoxazol-3-yl)pyrrolidin-1-yl]-[3-chloro-4-(trifluoromethyl)phenyl]methanone (109)

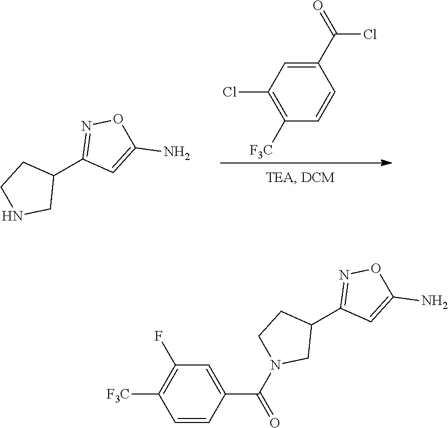 [SMILES: Nc1cc(C2CCNC2)no1 ; O=C(Cl)c1ccc(C(F)(F)F)c(Cl)c1 ; Nc1cc(C2CCN(C(=O)c3ccc(C(F)(F)F)c(F)c3)C2)no1]

Similar to the preparation of 85, purification by prep-HPLC provided the target compound 109 (15 mg, 70 umol, 10.65% yield) as a white solid. ¹H NMR (400 MHz, DMSO) major rotamer δ 7.93 (dd, J=8.1, 2.6 Hz, 1H), 7.85 (s, 1H), 7.68 (d, J=8.0 Hz, 1H), 6.60 (d, J=10.8 Hz, 2H), 4.87 (s, 1H), 3.84 (dd, J=12.0, 7.8 Hz, 1H), 3.68-3.39 (m, 3H), 3.28 (d, J=8.1 Hz, 1H), 2.26-2.10 (m, J=19.8, 15.1 Hz, 1H), 2.04-1.92 (m, 1H). ESI-MS: m/z=359.8 (M+1).

### Example 110: Synthesis of [3-(5-aminoisoxazol-3-yl)pyrrolidin-1-yl]-[3-fluoro-4-(trifluoromethoxy)phenyl]methanone (110)

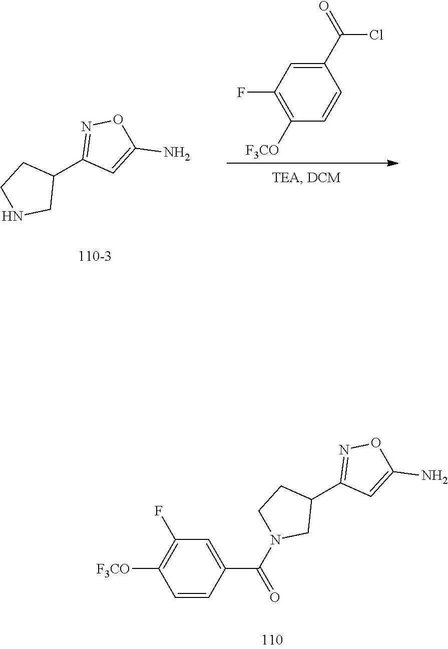 [SMILES: Nc1cc(C2CCNC2)no1 ; O=C(Cl)c1ccc(OC(F)(F)F)c(F)c1 ; Nc1cc(C2CCN(C(=O)c3ccc(OC(F)(F)F)c(F)c3)C2)no1]

Similar to the preparation of 85, purification by prep-HPLC provided the target compound 110 (38 mg, 105.77 umol, 27% yield) as a white solid. ¹H NMR (400 MHz, CDCl3) major rotamer δ 7.41 (d, J=10.3 Hz, 1H), 7.35 (s, 2H), 4.98 (s, 1H), 4.45 (s, 2H), 4.04-3.82 (m, 1H), 3.78-3.53 (m, 3H), 3.50-3.30 (m, 1H), 2.40-2.26 (m, 1H), 2.15 (ddd, J=12.7, 8.4, 4.2 Hz, 1H). ESI-MS: m/z=359.8 (M+1).

### Example 111: Synthesis of [3-fluoro-4-(trifluoromethyl)phenyl]-[4-[5-(2-methoxyethylamino)isoxazol-3-yl]-1-piperidyl]methanone (111)

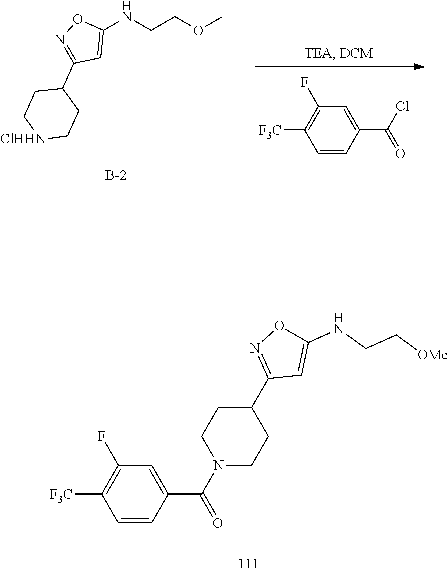

Similar to the preparation of 91, purification by prep-HPLC provided the target compound 111 (22 mg, 52.96 umol, 15% yield) as a white solid. ¹H NMR (400 MHz, CDCl3) δ 7.67 (t, J=7.5 Hz, 1H), 7.29 (s, 1H), 7.24 (s, 1H), 4.86 (s, 1H), 4.83 (d, J=5.6 Hz, 1H), 4.64 (s, 1H), 3.72 (s, 1H), 3.58-3.52 (m, 2H), 3.39 (s, 3H), 3.34 (dd, J=10.5, 5.6 Hz, 2H), 3.23-3.01 (m, 2H), 2.91 (ddd, J=15.0, 7.5, 3.9 Hz, 1H), 2.13-1.88 (m, 2H), 1.69 (s, 1H), 1.57 (s, 1H). ESI-MS: m/z=415.9 (M+1).

### Example 112: Synthesis of [3-fluoro-4-(trifluoromethoxy)phenyl]-[4-[5-(2-methoxyethylamino)isoxazol-3-yl]-1-piperidyl]methanone (112)

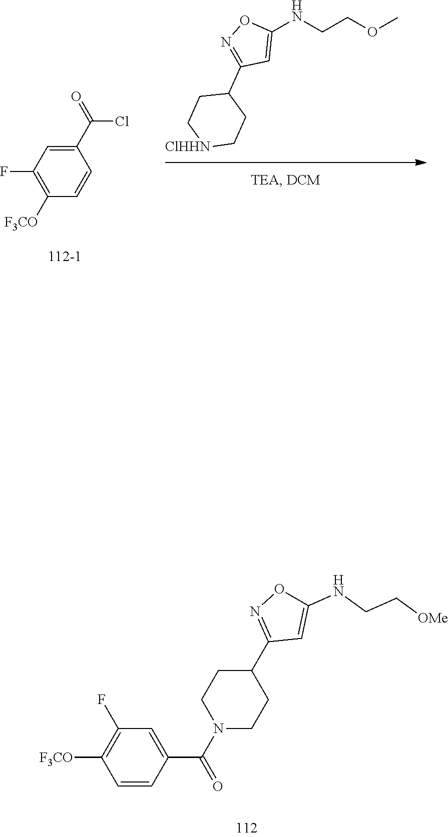

Similar to the preparation of 91, purification by prep-HPLC provided the target compound 112 (33.8 mg, 78.35 umol, 20.51% yield) as a white solid. 1H NMR (400 MHz, CDCl3) δ 7.31-7.26 (m, 1H), 7.21 (dd, J=9.8, 1.9 Hz, 1H), 7.16-7.13 (m, 1H), 4.79 (s, 1H), 4.57 (s, 1H), 3.70 (s, 1H), 3.50-3.47 (m, 2H), 3.32 (s, 3H), 3.28-3.25 (m, 2H), 3.20-2.90 (m, 2H), 2.83 (td, J=7.3, 3.6 Hz, 1H), 1.94 (s, 2H), 1.64 (s, 2H). ESI-MS: m/z=431.9 (M+1).

### Example 113: Synthesis of (3-((5-aminoisoxazol-3-yl)methyl)azetidin-1-yl)(3-fluoro-4-(trifluoromethoxy)phenyl)methanone (113)

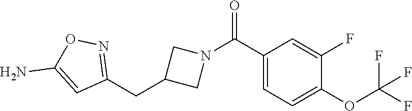 [SMILES: Nc1cc(CC2CN(C(=O)c3ccc(OC(F)(F)F)c(F)c3)C2)no1]

Similar to the preparation of 1, purification by prep-HPLC provided the target compound 113 (64.1 mg, 0.178 mmol, 99% yield) as a white solid. ¹H NMR (400 MHz, CD3SOCD3, 300 K) δ (ppm)=7.79-7.70 (m, 1H), 7.70-7.63 (m, 1H), 7.62-7.55 (m, 1H), 4.83 (s, 1H), 4.51-4.37 (m, 1H), 4.21-4.10 (m, 1H), 4.08-3.99 (m, 1H), 3.62 (qd, J=6.6, 10.5 Hz, 1H), 3.18-2.84 (m, 2H), 2.78-2.72 (m, 2H), 1.38-1.24 (m, 2H). ESI-MS: m/z=360.1 (M+1).HCl

### Example 114: Synthesis of (3-((5-aminoisoxazol-3-yl)methyl)azetidin-1-yl)(3-chloro-4-(trifluoromethyl)phenyl)methanone hydrochloride (114)

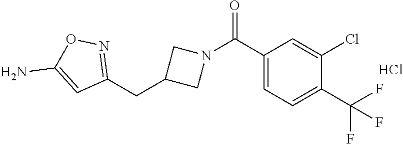 [SMILES: Nc1cc(CC2CN(C(=O)c3ccc(C(F)(F)F)c(Cl)c3)C2)no1 ; Cl]

Similar to the preparation of 1, purification by prep-HPLC provided the target compound 114 (57.2 mg, 0.159 mmol, 90% yield) as a white solid. 1H NMR (400 MHz, CDCl3, 300 K) δ (ppm)=7.68 (s, 1H), 7.66 (d, J=8.3 Hz, 1H), 7.52 (d, J=8.0 Hz, 1H), 4.90 (s, 1H), 4.42-4.22 (m, 2H), 3.96 (br dd, J=5.6, 8.2 Hz, 1H), 3.88 (br dd, J=5.9, 10.2 Hz, 1H), 3.07-2.92 (m, 1H), 2.86-2.78 (m, 2H), 1.26-1.12 (m, 1H), 0.00 (s, 1H). ESI-MS: m/z=360.1 (M+1).

### Example 115: Synthesis of [3-chloro-4-(trifluoromethoxy)phenyl]-[4-[5-(2-methoxyethylamino)isoxazol-3-yl]-1-piperidyl]methanone (115)

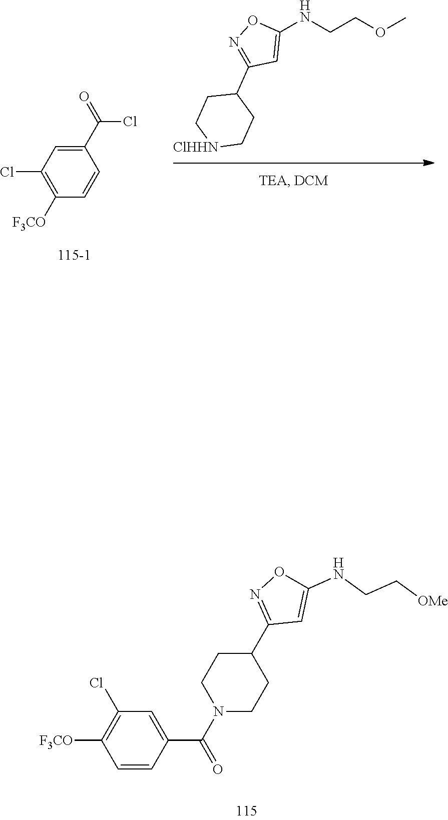

Similar to the preparation of 91, purification by prep-HPLC provided the target compound 115 (8 mg, 17.86 umol, 4.68% yield) as a white solid. 1H NMR (400 MHz, CDCl3) δ 7.47 (d, J=1.4 Hz, 1H), 7.31-7.26 (m, 2H), 4.79 (s, 1H), 4.77 (d, J=5.7 Hz, 1H), 4.55 (s, 1H), 3.69 (s, 1H), 3.50-3.47 (m, 2H), 3.32 (s, 3H), 3.29-3.25 (m, 2H), 3.15-2.93 (m, 2H), 2.84 (ddd, J=11.2, 7.3, 3.9 Hz, 1H), 1.99-1.88 (m, 2H), 1.62 (s, 2H). ESI-MS: m/z=447.9 (M+1).

### Example 116: Synthesis of (3-((5-aminoisoxazol-3-yl)methyl)pyrrolidin-1-yl)(3-chloro-4-(trifluoromethyl)phenyl)methanone (116)

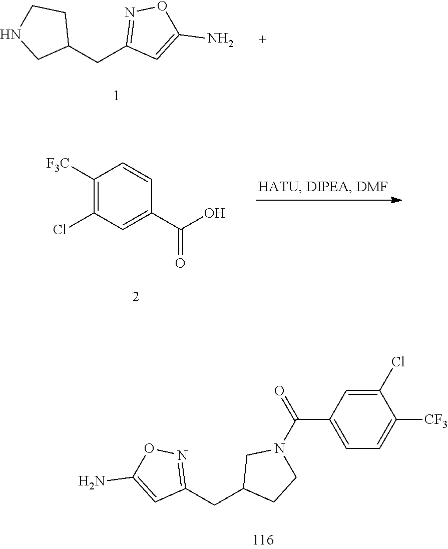 [SMILES: Nc1cc(CC2CCNC2)no1 ; O=C(O)c1ccc(C(F)(F)F)c(Cl)c1 ; Nc1cc(CC2CCN(C(=O)c3ccc(C(F)(F)F)c(Cl)c3)C2)no1]

Similar to the preparation of 1, purification by prep-HPLC provided the target compound 116 (32 mg, 85.62 μmol, 21.67% yield) as a white solid.

1H NMR (400 MHz, CDCl3) major rotamer δ 7.73 (dd, J=8.0, 1.9 Hz, 1H), 7.64 (s, 1H), 7.47 (d, J=8.0 Hz, 1H), 4.93 (s, 1H), 4.42 (s, 2H), 3.92-3.74 (m, 1H), 3.67-3.41 (m, 2H), 3.37-3.12 (m, 1H), 2.70-2.55 (m, 3H), 2.25-2.07 (m, 1H), 1.79-1.64 (m, 1H). ESI-MS: m/z=373.8 (M+1).

### Example 117: Synthesis of (3-((5-aminoisoxazol-3-yl)methyl)pyrrolidin-1-yl)(3-chloro-4-(trifluoromethoxy)phenyl)methanone (117)

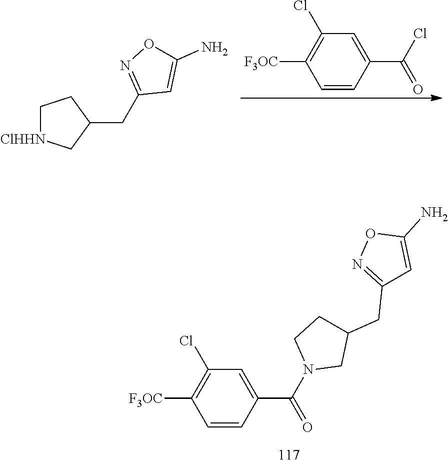

Similar to the preparation of 85, purification by prep-HPLC provided the target compound 117 (70 mg, 178.68 μmol, 42.68% yield) as a white solid. ¹H NMR (400 MHz, CDCl3) major rotamer δ 7.65 (d, J=2.0 Hz, 1H), 7.47-7.42 (m, 1H), 7.35 (d, J=8.5 Hz, 1H), 4.93 (s, 1H), 4.41 (s, 2H), 3.89-3.75 (m, 1H), 3.63-3.44 (m, 2H), 3.34-3.15 (m, 1H), 2.70-2.55 (m, 3H), 2.21-2.10 (m, 1H), 1.77-1.69 (m, 1H). ESI-MS: m/z=389.8 (M+1).

### Example 118: Synthesis of [3-[(5-aminoisoxazol-3-yl)methyl]pyrrolidin-1-yl]-[3-fluoro-4-(trifluoromethyl)phenyl]methanone (118)

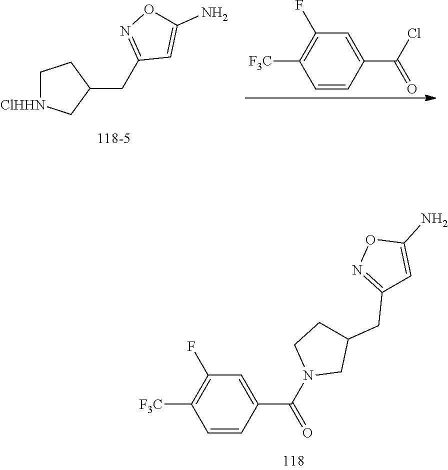

Similar to the preparation of 85, purification by prep-HPLC provided the target compound 118 (70 mg, 178.68 μmol, 42.68% yield) as a white solid. ¹H NMR (400 MHz, CDCl3) δ 7.58 (dd, J=10.4, 4.4 Hz, 1H), 7.32-7.25 (m, 2H), 4.96-4.84 (m, 1H), 4.43-4.37 (m, 2H), 3.83-3.70 (m, 1H), 3.57-3.33 (m, 2H), 3.28-3.07 (m, 1H), 2.63-2.46 (m, 3H), 2.11 (d, J=12.9 Hz, 1H), 1.71-1.64 (m, 1H). ESI-MS: 357.9 [M+H]+

### Example 119: Synthesis of (4-(5-amino-4-methylisoxazol-3-yl)piperidin-1-yl)(3-chloro-4-(trifluoromethyl)phenyl)methanone (119)

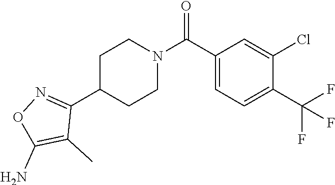 [SMILES: Cc1c(C2CCN(C(=O)c3ccc(C(F)(F)F)c(Cl)c3)CC2)noc1N]

Similar to the preparation of 1, purification by prep-HPLC provided the target compound 119 (53.3 mg, 0.075 mmol, 63% yield) as a white solid. ¹H NMR (400 MHz, CDCl3, 300 K) δ (ppm)=7.74 (d, J=8.0 Hz, 1H), 7.56 (s, 1H), 7.45-7.34 (m, 1H), 4.76-4.52 (m, 1H), 3.84-3.62 (m, 1H), 3.31-3.12 (m, 1H), 3.07 (br s, 1H), 2.82 (s, 4H), 2.74-2.71 (m, 1H), 2.15-1.96 (m, 2H), 1.87 (br d, J=14.3 Hz, 3H). ESI-MS: 388.1 [M+H]+.

### Example 120: Synthesis of (4-(5-amino-4-methylisoxazol-3-yl)piperidin-1-yl)(3-fluoro-4-(trifluoromethoxy)phenyl)methanone (120)

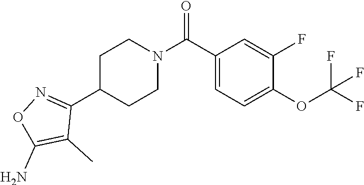 [SMILES: Cc1c(C2CCN(C(=O)c3ccc(OC(F)(F)F)c(F)c3)CC2)noc1N]

Similar to the preparation of 1, purification by prep-HPLC provided the target compound 120 (40.3 mg, 0.104 mmol, 88% yield) as a white solid. ¹H NMR (400 MHz, CDCl3, 300 K) Shift (ppm)=7.40-7.33 (m, 1H), 7.30 (dd, J=1.8, 9.8 Hz, 1H), 7.23 (td, J=1.5, 8.3 Hz, 1H), 4.64 (br s, 2H), 3.82 (br s, 2H), 3.14 (br s, 3H), 2.92-2.69 (m, 4H), 2.12-1.91 (m, 3H). ESI-MS: 388.3 [M+H]+.

### Example 121: Synthesis of N-[3-(5-aminoisoxazol-3-yl)cyclobutyl]-3-chloro-4-(trifluoromethyl)benzamide (121)

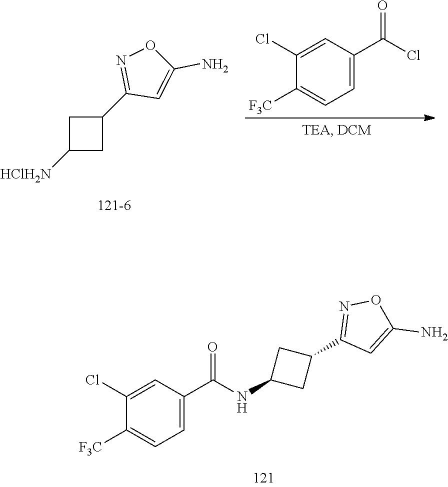

Similar to the preparation of 85, purification by prep-HPLC provided the target compound 121 (80 mg, 222.4 μmol, 52.7% yield) as a white solid. ¹H NMR (400 MHz, DMSO) δ 9.05 (d, J=7.7 Hz, 1H), 8.20 (s, 1H), 8.04 (d, J=0.7 Hz, 2H), 6.60 (s, 2H), 4.98 (s, 1H), 4.50 (d, J=7.7 Hz, 1H), 3.14-3.05 (m, 1H), 2.63 (dt, J=10.4, 7.8 Hz, 2H), 2.28 (dt, J=11.5, 9.7 Hz, 2H). ESI-MS: 359.8[M+H]+.

### Example 122: Synthesis of [3-chloro-4-(trifluoromethyl)phenyl]-[4-[5-(2-methoxyethylamino)isoxazol-3-yl]-1-piperidyl]methanone (122)

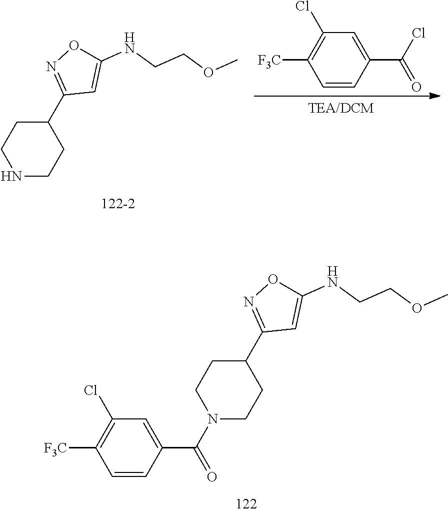

Similar to the preparation of 91, purification by prep-HPLC provided the target compound 122 (24 mg, 55.58 μmol, 12.5% yield) as a white solid. ¹H NMR (400 MHz, DMSO) δ 7.93 (d, J=8.1 Hz, 1H), 7.79 (s, 1H), 7.58 (d, J=8.5 Hz, 1H), 7.14 (s, 1H), 5.02 (s, 1H), 4.44 (d, J=12.4 Hz, 1H), 3.42 (t, J=5.6 Hz, 3H), 3.26 (s, 3H), 3.20 (dd, J=11.5, 5.7 Hz, 3H), 2.97 (t, J=11.0 Hz, 1H), 2.85-2.78 (m, 1H), 1.91 (d, J=11.9 Hz, 1H), 1.75 (d, J=11.9 Hz, 1H), 1.64-1.54 (m, 2H). ESI-MS: 431.7[M+H]+.

### Example 123: Synthesis of [3-chloro-4-(trifluoromethyl)phenyl]-[rac-(1R,5S)-6-[5-(2-methoxyethylamino)isoxazol-3-yl]-3-azabicyclo[3.1.0]hexan-3-yl]methanone (123)

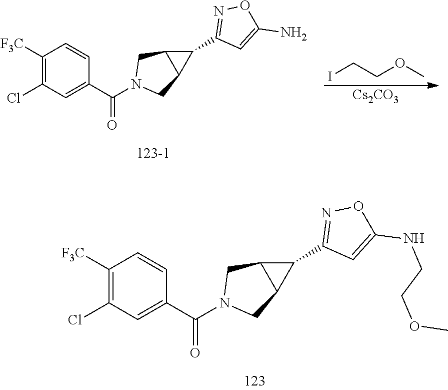

[3-chloro-4-(trifluoromethyl)phenyl]-[rac-(1R,5S)-6-(5-aminoisoxazol-3-yl)-3-azabicyclo[3.1.0]hexan-3-yl]methanone 89 (100 mg, 269.00 μmol), 1-iodo-2-methoxy-ethane (75.05 mg, 403.51 μmol) and Cs2CO3 (131.87 mg, 404.75 μmol) were dissolved in DMF (2 mL) and stirred at 100° C. for 1.5 hr. The mixture was extracted with EA (50 mL×3), washed with brine (50 mL), dried over Na2SO4. The crude product was purified by prep-HPLC to afford the [3-chloro-4-(trifluoromethyl)phenyl]-[rac-(1R,5S)-6-[5-(2-methoxyethylamino)isoxazol-3-yl]-3-azabicyclo[3.1.0]hexan-3-yl]methanone 123 (15 mg, 34.90 μmol, 12.97% yield). ¹H NMR (400 MHz, DMSO) δ 7.92 (d, J=8.1 Hz, 1H), 7.85 (s, 1H), 7.65 (d, J=8.6 Hz, 1H), 7.13 (t, J=5.9 Hz, 1H), 4.82 (s, 1H), 4.01 (d, J=12.2 Hz, 1H), 3.72 (dd, J=10.7, 4.0 Hz, 1H), 3.52 (dd, J=12.2, 4.0 Hz, 1H), 3.41 (dd, J=11.5, 5.8 Hz, 3H), 3.25 (s, 3H), 3.18 (q, J=5.7 Hz, 2H), 1.94 (ddd, J=14.9, 7.3, 3.7 Hz, 2H), 1.73 (t, J=3.4 Hz, 1H). ESI-MS: 429.8[M+H]+.

### Example 124: Synthesis of [3-fluoro-4-(trifluoromethyl)phenyl]-[4-[5-(2-hydroxyethylamino)isoxazol-3-yl]-1-piperidyl]methanone (124)

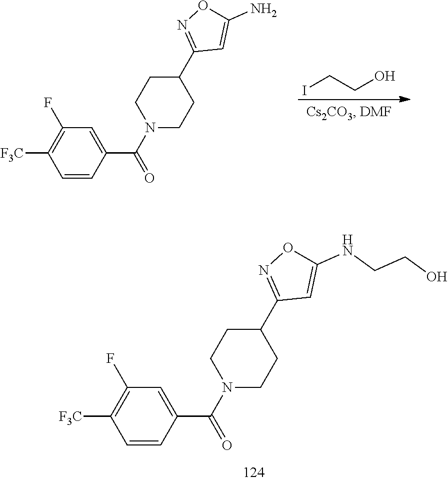

[4-(5-aminoisoxazol-3-yl)-1-piperidyl]-[3-fluoro-4-(trifluoromethyl)phenyl]methanone 63 (100 mg, 279.88 μmol), 2-iodoethanol (72.19 mg, 419.81 μmol, 32.79 μL) and Cs2CO3 (273.57 mg, 839.63 μmol) were dissolved in DMF (2 mL) and stirred at 100° C. for 5 hr. The mixture was extracted with EA (50 mL×3), washed with brine (50 mL), dried over Na₂SO₄. The crude product was purified by prep-HPLC to afford the [3-fluoro-4-(trifluoromethyl)phenyl]-[4-[5-(2-hydroxyethylamino)isoxazol-3-yl]-1-piperidyl]methanone 124 (22 mg, 54.81 μmol, 19.59% yield). ¹H NMR (400 MHz, DMSO) δ 7.87 (s, 1H), 7.61 (d, J=11.0 Hz, 1H), 7.44 (d, J=8.1 Hz, 1H), 7.05 (s, 1H), 4.99 (s, 1H), 4.72 (t, J=5.5 Hz, 1H), 4.44 (d, J=12.2 Hz, 1H), 3.49 (dd, J=11.5, 5.9 Hz, 3H), 3.16 (t, J=11.0 Hz, 1H), 3.10 (q, J=6.0 Hz, 2H), 2.95 (t, J=11.0 Hz, 1H), 2.85-2.78 (m, 1H), 1.91 (d, J=11.6 Hz, 1H), 1.75 (d, J=11.8 Hz, 1H), 1.60 (d, J=9.0 Hz, 2H). ESI-MS: 401.8[M+H]+.

### Example 125: Synthesis of ethyl (3-fluoro-4-(trifluoromethoxy)phenyl)(4-(5-(2-morpholinoethylamino)isoxazol-3-yl)piperidin-1-yl)methanone (125)

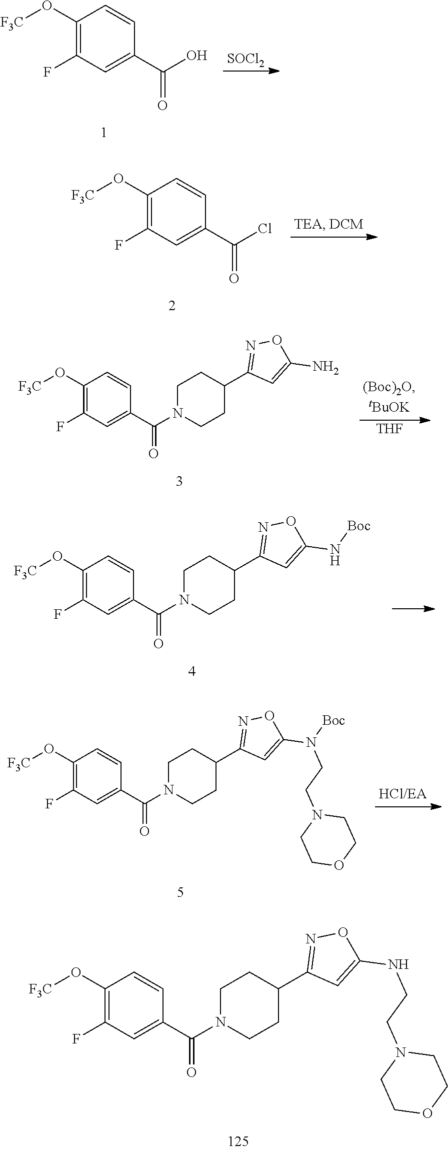

Synthesis of Compound 2 (step 1): To a solution of 3-fluoro-4-(trifluoromethoxy)benzoic acid (130 mg, 580.07 μmol) in SOCl2 (2 mL). Then the reaction was stirred at 80° C. for 2 h. TLC (EA:PE=1:1, UV, 254 nm) showed that the starting material was consumed. The reaction was directly concentrated to yield Compound 2 (140 mg, 577.19 μmol, 99.50% yield). The product was directly used in next step without further purification.

Synthesis of Compound 3 (step 2): To a solution of 3-(4-piperidyl)isoxazol-5-amine (117.56 mg, 577.19 μmol, CL) in DCM (4 mL) added N,N-diethylethanamine (175.22 mg, 1.73 mmol, 241.35 μL). Then Compound 2 (140 mg, 577.19 μmol) was added to the mixture. After the addition, the reaction was stirred at 25° C. for 2 h. The reaction was diluted with water (10 mL) and extracted with EA (20 mL*3). The organic layers were combined, washed with brine, dried over Na₂SO₄ and concentrated to give crude product. The crude product was purified with silica gel column (EA:PE=0-90%) and concentrated to yield Compound 3 (85 mg, 227.70 μmol, 39.45% yield)

Synthesis of Compound 4 (step 3): To a solution of Compound 3 (85 mg, 227.70 μmol) in THF (2 mL) added tert-butoxycarbonyl tert-butyl carbonate (74.54 mg, 341.55 μmol, 78.38 μL) and potassium 2-methylpropan-2-olate (51.10 mg, 455.40 μmol). Then the reaction was stirred at 60° C. for 2 h. LCMS showed that the major peak MS was desired product. The reaction was diluted with water (20 mL) and extracted with EA (20 mL*2). The organic layers were combined, washed with brine, dried over Na2SO4 and concentrated to give crude product. The crude product was directly purified with silica gel column (EA:PE=0-50%) and concentrated to give Compound 4 (67 mg, 141.52 μmol, 62.15% yield).

Synthesis of Compound 5 (step 4): To a solution of tert-butyl N-[3-[1-[3-fluoro-4-(trifluoromethoxy)benzoyl]-4-piperidyl]isoxazol-5-yl]carbamate (65 mg, 137.30 mol) in THE (1 mL) added sodium hydride (6.59 mg, 274.60 μmol), then the reaction was stirred at 25° C. for 30 min. After that, 4-(2-bromoethyl)morpholine (39.97 mg, 205.95 μmol) was added to the mixture. Then the reaction was stirred at 60° C. for 2 h. LCMS showed that the major peak was the desired product. The reaction was diluted with water (10 mL) and extracted with EA (20 mL*2). The organic layer was combined, washed with brine, dried over Na2SO4 and concentrated to give crude product. The crude product was purified with silica gel column (EA:PE=0 to 100%) and concentrated to give Compound 5 (71 mg, 121.04 mol, 88.16% yield).

Synthesis of 125 (step 5): To a solution of tert-butyl N-[3-[1-[3-fluoro-4-(trifluoromethyl)benzoyl]-4-piperidyl]isoxazol-5-yl]-N-(2-morpholinoethyl)carbamate (51 mg, 89.38 μmol) in 4 M HCl in EA (1 mL). Then the reaction was stirred at 25° C. for 1 h.

TLC showed that the starting material was consumed. The reaction was directly concentrated to give 125 (11 mg, 23.38 μmol, 26.16% yield). ¹H NMR (400 MHz, CDCl3) δ 7.29 (t, J=7.7 Hz, 1H), 7.23 (d, J=1.5 Hz, 1H), 7.15 (d, J=8.3 Hz, 1H), 5.11 (s, 1H), 4.77 (s, 1H), 4.55 (s, 1H), 3.78-3.55 (m, 5H), 3.21-2.90 (m, 4H), 2.84 (ddd, J=11.1, 7.5, 4.0 Hz, 1H), 2.53 (t, J=5.8 Hz, 2H), 2.41 (d, J=4.0 Hz, 4H), 1.94 (s, 2H), 1.61 (s, 2H). ESI-MS: m/z=486.8 (M+1).

### Example 126: Synthesis of [3-fluoro-4-(trifluoromethoxy)phenyl]-[4-[5-(2-hydroxyethylamino)isoxazol-3-yl]-1-piperidyl]methanone (126)

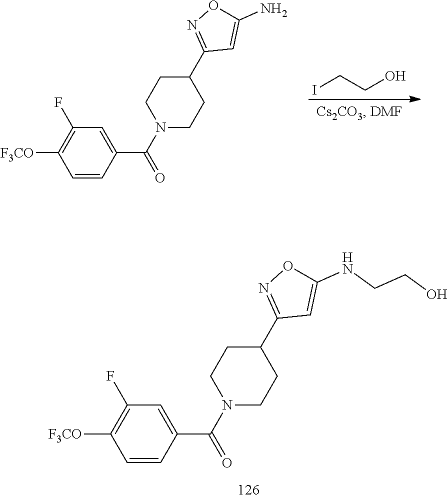

Similar to the preparation of 124, purification by prep-HPLC provided the target compound 126 (20 mg, 47.9 μmol, 17.9% yield) as a white solid. ¹H NMR (400 MHz, MeOD) δ 7.56-7.51 (m, 1H), 7.46 (dd, J=10.2, 1.9 Hz, 1H), 7.36-7.32 (m, 1H), 5.04 (s, 1H), 4.59 (brs, 1H), 3.66 (t, J=5.7 Hz, 3H), 3.26 (t, J=5.7 Hz, 3H), 3.04 (brs, 1H), 2.90 (tt, J=11.4, 3.8 Hz, 1H), 2.02 (d, J=4.5 Hz, 1H), 1.91 (d, J=20.3 Hz, 1H), 1.71 (s, 2H). ESI-MS: 417.8 [M+H]+.

### Example 127: Synthesis of [3-chloro-4-(trifluoromethoxy)phenyl]-[4-[5-(2-hydroxyethylamino)isoxazol-3-yl]-1-piperidyl]methanone (127)

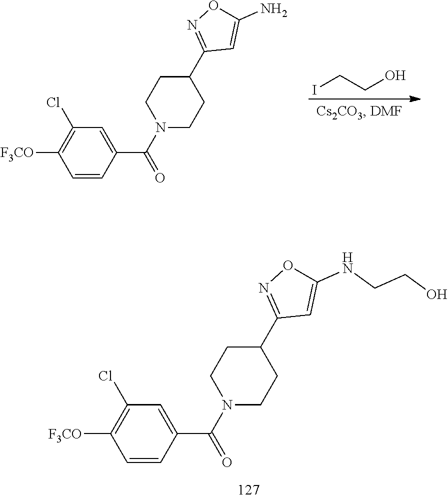

Similar to the preparation of 124, purification by prep-HPLC provided the target compound 127 (20 mg, 47.9 μmol, 17.9% yield) as a white solid. 1H NMR (400 MHz, DMSO) δ 7.77 (d, J=2.0 Hz, 1H), 7.66-7.62 (m, 1H), 7.52 (dd, J=8.4, 2.0 Hz, 1H), 7.05 (t, J=5.9 Hz, 1H), 5.00 (s, 1H), 4.72 (t, J=5.4 Hz, 1H), 4.43 (brs, 1H), 3.49 (dd, J=11.4, 5.8 Hz, 3H), 3.17 (s, 1H), 3.13-3.08 (m, 2H), 2.94 (s, 1H), 2.81 (ddd, J=11.3, 7.6, 3.8 Hz, 1H), 1.89 (s, 1H), 1.76 (s, 1H), 1.59 (dd, J=21.1, 11.4 Hz, 2H). ESI-MS: 433.8[M+H]+.

### Example 128: Synthesis of [3-chloro-4-(trifluoromethyl)phenyl]-[4-[5-(2-hydroxyethylamino)isoxazol-3-yl]-1-piperidyl]methanone (128)

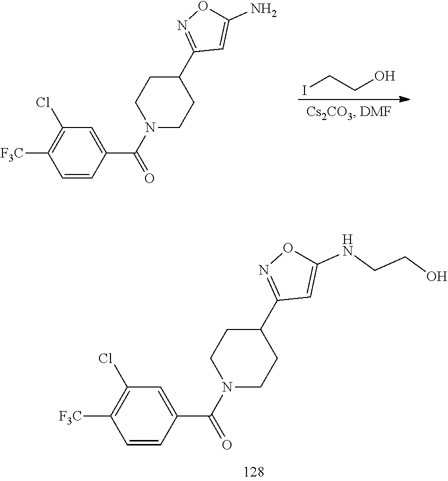

Similar to the preparation of 124, purification by prep-HPLC provided the target compound 128 (33 mg, 78.98 μmol, 29.52% yield) as a white solid. ¹H NMR (400 MHz, DMSO) δ 7.93 (d, J=8.1 Hz, 1H), 7.79 (s, 1H), 7.58 (d, J=8.5 Hz, 1H), 7.05 (t, J=5.9 Hz, 1H), 5.00 (s, 1H), 4.72 (t, J=5.4 Hz, 1H), 4.44 (d, J=12.1 Hz, 1H), 3.49 (dd, J=11.5, 5.9 Hz, 3H), 3.18 (d, J=11.5 Hz, 1H), 3.11 (q, J=6.0 Hz, 2H), 2.96 (t, J=12.1 Hz, 1H), 2.82 (ddd, J=15.1, 7.5, 3.7 Hz, 1H), 1.91 (d, J=11.8 Hz, 1H), 1.79-1.72 (m, 1H), 1.66-1.56 (m, 2H). ESI-MS: 417.8 [M+H]+.

### Example 129: Synthesis of [4-[5-(2-tert-butoxyethylamino)isoxazol-3-yl]-1-piperidyl]-[3-fluoro-4-(trifluoromethyl)phenyl]methanone (129)

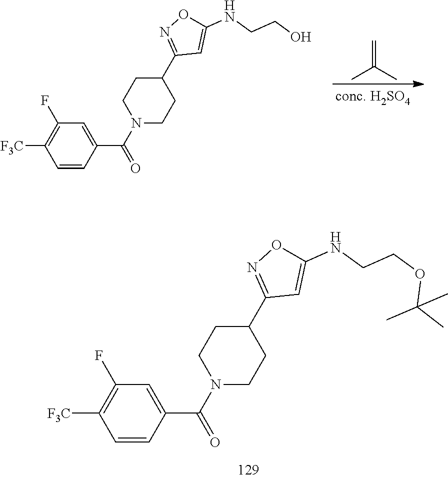

To a solution of [3-fluoro-4-(trifluoromethyl)phenyl]-[4-[5-(2-hydroxyethylamino)isoxazol-3-yl]-1-piperidyl]methanone (140 mg, 348.82 μmol) and 2-methylprop-1-ene (195.71 mg, 3.49 mmol) in DCM (3 mL) was added H2SO4 (89.05 mg, 348.82 μmol) at rt for 12 hr. The mixture was extracted with EA (50 mL×3), washed with NaHCO₃ (50 mL), dried over Na2SO4. The crude product was purified by prep-HPLC to afford the [4-[5-(2-tert-butoxyethylamino)isoxazol-3-yl]-1-piperidyl]-[3-fluoro-4-(trifluoromethyl)phenyl]methanone (13 mg, 28.45 μmol, 8.15% yield) as white solid. ¹H NMR (400 MHz, DMSO) δ 7.87 (t, J=7.7 Hz, 1H), 7.61 (d, J=11.0 Hz, 1H), 7.44 (d, J=8.0 Hz, 1H), 7.04 (t, J=6.0 Hz, 1H), 5.01 (s, 1H), 4.44 (d, J=11.7 Hz, 1H), 3.48 (d, J=13.3 Hz, 1H), 3.40 (t, J=6.0 Hz, 2H), 3.20-3.11 (m, 3H), 2.95 (t, J=11.5 Hz, 1H), 2.82 (ddd, J=11.3, 7.6, 3.8 Hz, 1H), 1.91 (d, J=11.1 Hz, 1H), 1.75 (d, J=11.7 Hz, 1H), 1.60 (d, J=8.9 Hz, 2H), 1.12 (s, 9H). ESI-MS: 457.9[M+H]+.

### Example 130: Synthesis of (3-chloro-4-(trifluoromethyl)phenyl)(4-(5-(2-morpholinoethylamino)isoxazol-3-yl)piperidin-1-yl)methanone (130)

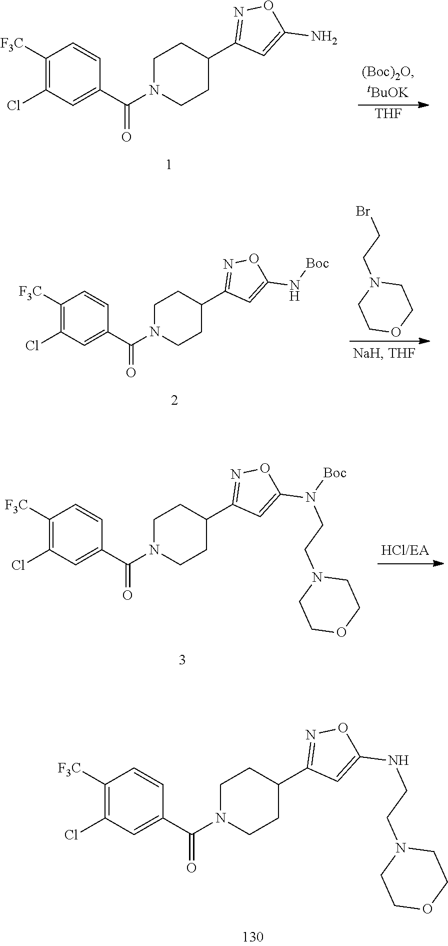 [SMILES: Nc1cc(C2CCN(C(=O)c3ccc(C(F)(F)F)c(Cl)c3)CC2)no1 ; CC(C)(C)OC(=O)Nc1cc(C2CCN(C(=O)c3ccc(C(F)(F)F)c(Cl)c3)CC2)no1 ; BrCCN1CCOCC1 ; CC(C)(C)OC(=O)N(CCN1CCOCC1)c1cc(C2CCN(C(=O)c3ccc(C(F)(F)F)c(Cl)c3)CC2)no1 ; "THF" ; O=C(c1ccc(C(F)(F)F)c(Cl)c1)N1CCC(c2cc(NCCN3CCOCC3)on2)CC1]

Similar to the preparation of 125, purification by prep-HPLC provided the target compound 130 (32 mg, 65.72 μmol, 27.56% yield) as a white solid. ¹H NMR (400 MHz, CDCl3) δ 7.74 (d, J=8.0 Hz, 1H), 7.55 (s, 1H), 7.38 (d, J=8.0 Hz, 1H), 5.17 (t, J=5.0 Hz, 1H), 4.85 (s, 1H), 4.64 (s, 1H), 3.83-3.60 (m, 5H), 3.22 (dd, J=11.5, 5.3 Hz, 2H), 3.18 (s, 1H), 3.03 (s, 1H), 2.92 (ddd, J=11.1, 7.5, 3.6 Hz, 1H), 2.64-2.57 (m, 2H), 2.52-2.39 (m, 4H), 2.06 (s, 1H), 1.94 (s, 1H), 1.80-1.60 (m, 2H). ESI-MS: m/z=486.9 (M+1).

### Example 131: Synthesis of (4-(5-(2-tert-butoxyethylamino)isoxazol-3-yl)piperidin-1-yl)(3-chloro-4-(trifluoromethoxy)phenyl)methanone (131)

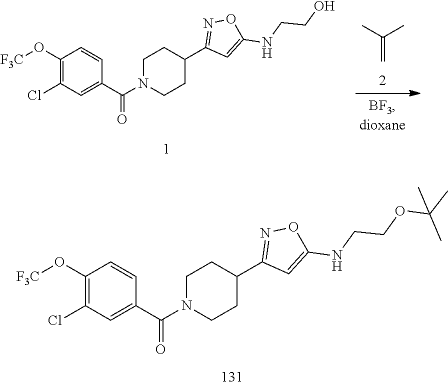

Similar to the preparation of 129, purification by prep-HPLC provided the target compound 131 (9.6 mg, 19.60 μmol, 5.45% yield) as a white solid. ¹H NMR (400 MHz, CDCl₃) δ 7.54 (d, J=1.7 Hz, 1H), 7.37-7.33 (m, 2H), 4.86 (s, 2H), 4.64 (s, 1H), 3.77 (s, 1H), 3.52 (t, J=5.2 Hz, 2H), 3.28 (dd, J=10.7, 5.6 Hz, 2H), 3.16 (s, 1H), 3.07-2.99 (m, 1H), 2.90 (ddd, J=11.1, 7.3, 3.9 Hz, 1H), 2.01 (s, 2H), 1.73 (s, 2H), 1.20 (s, 9H). ESI-MS: m/z=489.9 (M+1).

### Example 132: Synthesis of (3-fluoro-4-(trifluoromethyl)phenyl)(4-(5-(2-morpholinoethylamino)isoxazol-3-yl)piperidin-1-yl)methanone (132)

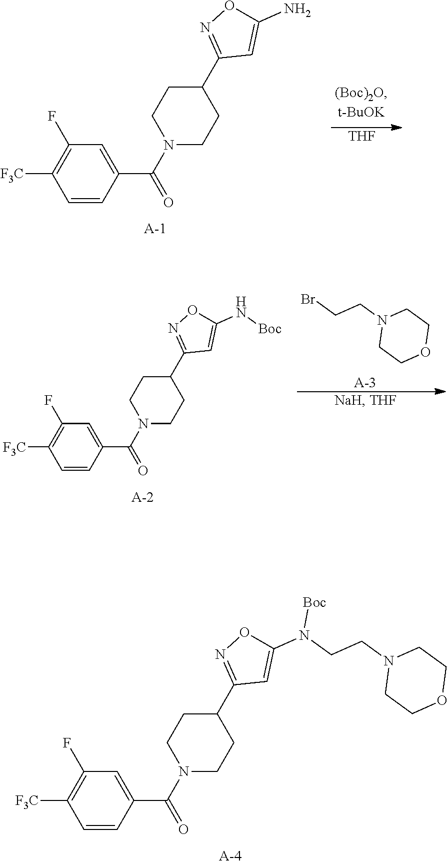

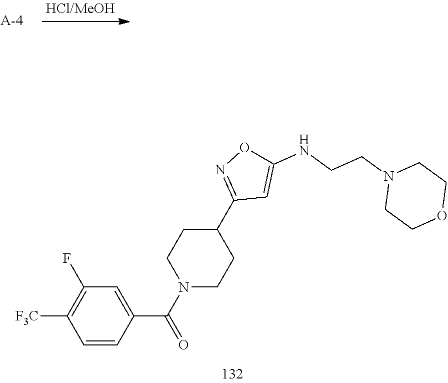 [SMILES: "A-4" ; O=C(c1ccc(C(F)(F)F)c(F)c1)N1CCC(c2cc(NCCN3CCOCC3)on2)CC1]

Similar to the preparation of 125, purification by prep-HPLC provided the target compound 132 (5.6 mg, 11.90 μmol, 7.46% yield) as a white solid. ¹H NMR (400 MHz, CDCl3) δ 7.67 (t, J=7.4 Hz, 1H), 7.29 (s, 1H), 7.24 (s, 1H), 5.19 (s, 1H), 4.85 (s, 1H), 4.75-4.55 (m, 1H), 3.73 (brs, 5H), 3.40-3.00 (m, 5H), 2.91 (tt, J=11.2, 3.8 Hz, 1H), 2.62 (s, 2H), 2.49 (s, 3H), 2.17-1.90 (m, 3H), 1.69 (s, 1H). ESI-MS: m/z=470.9 (M+1).

### Example 133: Synthesis of [3-chloro-4-(trifluoromethoxy)phenyl]-[4-[5-(2-methylsulfonylethylamino)isoxazol-3-yl]-1-piperidyl]methanone (133)

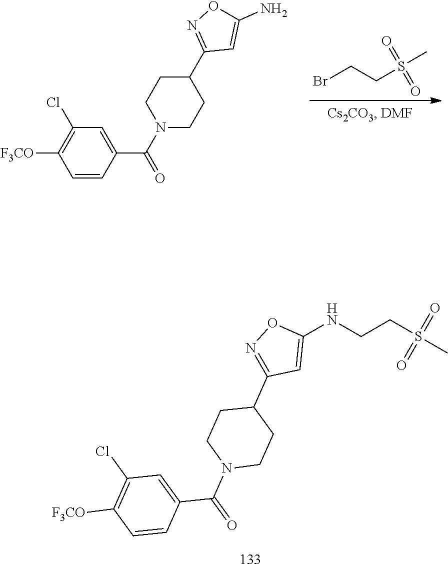

Similar to the preparation of 124, purification by prep-HPLC provided the target compound 133 (10.7 mg, 21.62 μmol, 11.52% yield) as a white solid. ¹H NMR (400 MHz, MeOD) δ 7.59 (d, J=1.9 Hz, 1H), 7.45 (dd, J=8.4, 1.3 Hz, 1H), 7.38 (dd, J=8.4, 2.0 Hz, 1H), 5.06 (s, 1H), 4.50 (s, 1H), 3.57 (t, J=6.6 Hz, 3H), 3.28 (t, J=6.5 Hz, 2H), 3.19-3.11 (m, 1H), 2.94 (d, J=11.2 Hz, 1H), 2.91 (s, 3H), 2.83 (t, J=3.8 Hz, 1H), 1.95-1.79 (m, 2H), 1.62 (s, 2H). ESI-MS: m/z=495.6[M+H]+.

### Example 134: Synthesis of (3-chloro-4-(trifluoromethyl)phenyl)(4-(5-(2-(methylsulfonyl)ethylamino)isoxazol-3-yl)piperidin-1-yl)methanone (134)

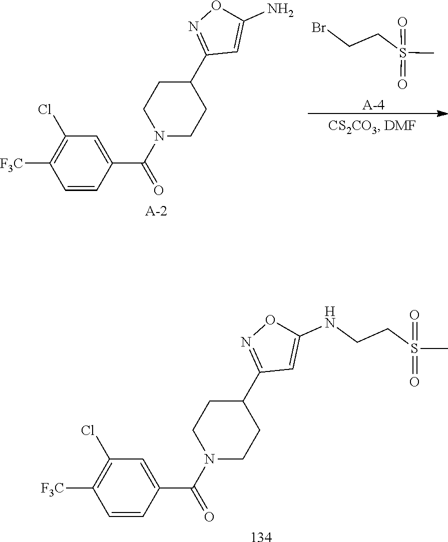

Similar to the preparation of 124, purification by prep-HPLC provided the target compound 134 (13 mg, 27.09 μmol, 5.06% yield) as a white solid. ¹H NMR (400 MHz, CDCl3) δ 7.75 (d, J=8.0 Hz, 1H), 7.55 (s, 1H), 7.39 (d, J=7.9 Hz, 1H), 5.21 (t, J=6.1 Hz, 1H), 4.97 (s, 1H), 4.65 (s, 1H), 3.79 (dd, J=11.9, 6.2 Hz, 2H), 3.71 (s, 1H), 3.38-3.26 (m, 2H), 3.17 (d, J=6.8 Hz, 1H), 3.10-2.85 (m, 5H), 2.20-1.85 (m, 2H), 1.75-1.60- (m, 2H). ESI-MS: m/z=479.8 (M+1).

### Example 135: Synthesis of (3-fluoro-4-(trifluoromethyl)phenyl)(4-(5-(2-(methylsulfonyl)ethylamino)isoxazol-3-yl)piperidin-1-yl)methanone (135)

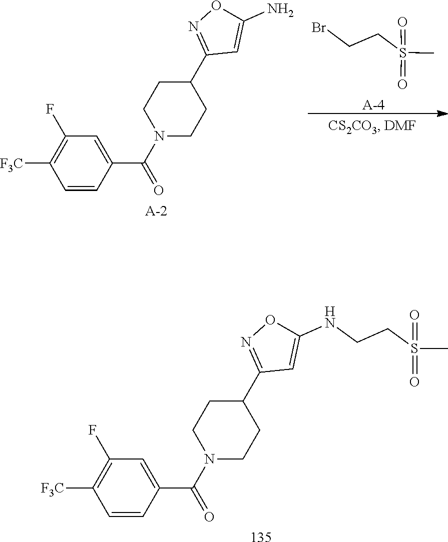

Similar to the preparation of 124, purification by prep-HPLC provided the target compound 135 (13 mg, 27.09 μmol, 5.06% yield) as a white solid. ¹H NMR (400 MHz, CDCl3) δ 7.67 (t, J=7.4 Hz, 1H), 7.29 (s, 1H), 7.24 (s, 1H), 5.20 (t, J=6.3 Hz, 1H), 4.97 (s, 1H), 4.65 (s, 1H), 3.79 (dd, J=12.0, 6.3 Hz, 2H), 3.70 (s, 1H), 3.36-3.27 (m, 2H), 3.30-3.00 (m, 2H), 2.99 (s, 3H), 2.96-2.86 (m, 1H), 2.15-1.85 (m, 2H), 1.80-1.65 (m, 2H). ESI-MS: m/z=463.8 (M+1).

### Example 136: Synthesis of (3-fluoro-4-(trifluoromethoxy)phenyl)(4-(5-(2-(methylsulfonyl)ethylamino)isoxazol-3-yl)piperidin-1-yl)methanone (136)

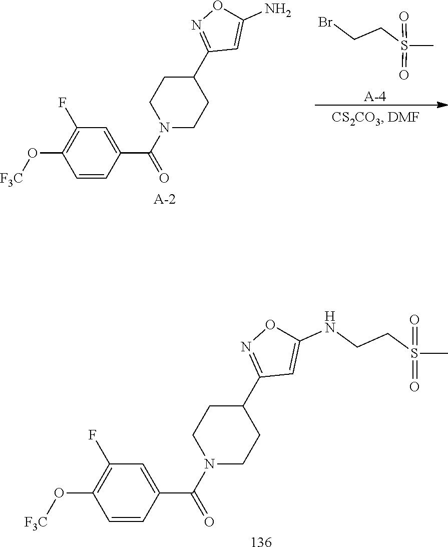

Similar to the preparation of 124, purification by prep-HPLC provided the target compound 136 (18 mg, 37.54 μmol, 12.74% yield) as a white solid. ¹H NMR (400 MHz, CDCl3) δ 7.39-7.33 (m, 1H), 7.29 (dd, J=9.8, 1.9 Hz, 1H), 7.24-7.19 (m, 1H), 5.17 (t, J=6.3 Hz, 1H), 4.97 (s, 1H), 4.69 (brs, 1H), 3.83-3.73 (m, 3H), 3.42-3.29 (m, 2H), 3.11 (d, J=44.3 Hz, 2H), 2.99 (s, 3H), 2.97-2.86 (m, 1H), 2.11-1.92 (m, 2H), 1.71 (s, 2H). ESI-MS: m/z=479.8 (M+1).

### Example 137: Synthesis of (3-chloro-4-(trifluoromethoxy)phenyl)(4-(5-(2-morpholinoethylamino)isoxazol-3-yl)piperidin-1-yl)methanone (137)

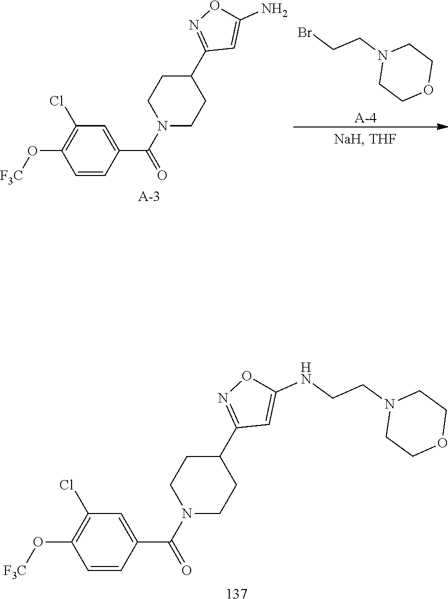

Similar to the preparation of 125, purification by prep-HPLC provided the target compound 137 (12 mg, 23.86 μmol, 4.04% yield) as a white solid. ¹H NMR (400 MHz, CDCl3) δ 7.54 (d, J=1.6 Hz, 1H), 7.41-7.31 (m, 2H), 5.19 (s, 1H), 4.85 (s, 1H), 4.63 (s, 1H), 3.73 (s, 5H), 3.23 (d, J=5.2 Hz, 2H), 3.20-2.97 (m, 2H), 2.96-2.81 (m, 1H), 2.61 (s, 2H), 2.49 (s, 4H), 2.02 (s, 2H), 1.72 (s, 2H). ESI-MS: m/z=502.8 (M+1).

### Example 138: Synthesis of (4-(5-(2-tert-butoxyethylamino)isoxazol-3-yl)piperidin-1-yl)(3-chloro-4-(trifluoromethyl)phenyl)methanone (138)

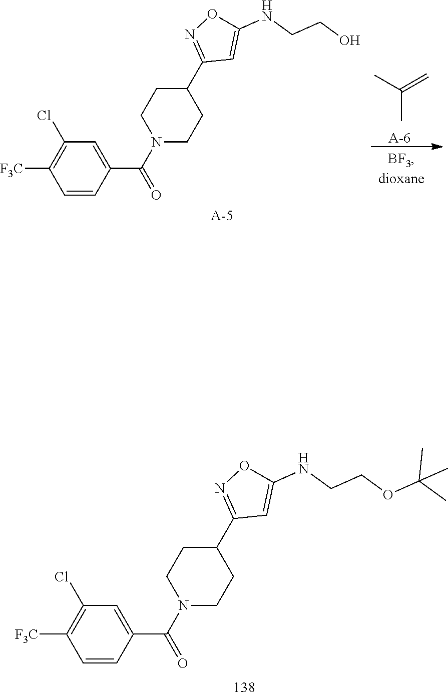

Similar to the preparation of 129, purification by prep-HPLC provided the target compound 138 (11 mg, 23.21 μmol, 8.08% yield) as a white solid. ¹H NMR (400 MHz, CDCl3) δ 7.74 (d, J=8.0 Hz, 1H), 7.55 (s, 1H), 7.38 (d, J=8.0 Hz, 1H), 4.86 (s, 2H), 4.64 (s, 1H), 3.70 (s, 1H), 3.52 (t, J=5.1 Hz, 2H), 3.28 (dd, J=10.5, 5.3 Hz, 2H), 3.17 (s, 1H), 3.03 (s, 1H), 2.91 (t, J=11.0 Hz, 1H), 2.06 (s, 1H), 1.95 (s, 1H), 1.69 (s, 2H), 1.20 (s, 9H). ESI-MS: m/z=473.9

### Example 139: Synthesis of (4-(5-(2-tert-butoxyethylamino)isoxazol-3-yl)piperidin-1-yl)(3-fluoro-4-(trifluoromethoxy)phenyl)methanone (139)

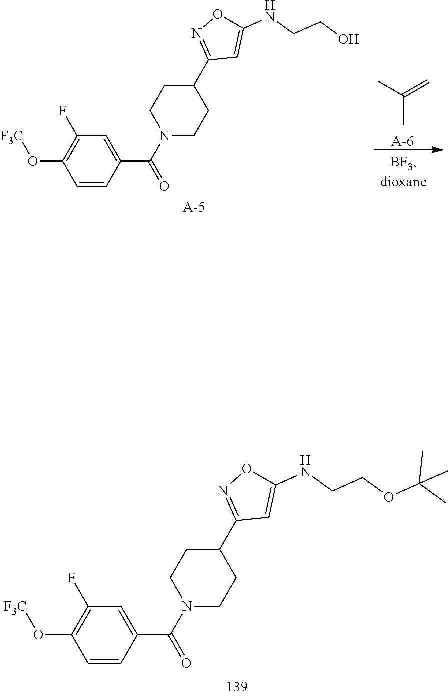

Similar to the preparation of 129, purification by prep-HPLC provided the target compound 139 (11 mg, 23.21 μmol, 8.08% yield) as a white solid. ¹H NMR (400 MHz, CDCl3) δ 7.39-7.33 (m, 1H), 7.31-7.27 (m, 1H), 7.24-7.19 (m, 1H), 4.86 (s, 2H), 4.63 (s, 1H), 3.77 (s, 1H), 3.57-3.47 (m, 2H), 3.28 (d, J=4.0 Hz, 2H), 3.05 (s, 2H), 2.91 (ddd, J=11.1, 7.4, 3.9 Hz, 1H), 2.03 (dd, J=10.4, 5.0 Hz, 2H), 1.72 (s, 2H), 1.18 (d, J=13.2 Hz, 9H). ESI-MS: m/z=473.9

### Example 140: Synthesis of [4-(5-aminoisoxazol-3-yl)-1-piperidyl]-(3-bromo-4-phenyl-phenyl)methanone (140)

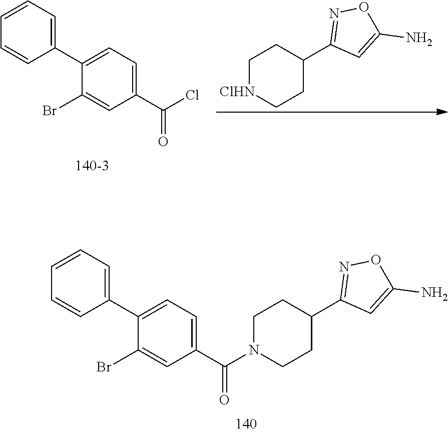

Similar to the preparation of 85, purification by prep-HPLC provided the target compound 140 (0.1 g, 234.57 μmol, 29.63% yield) as a white solid. ¹H NMR (400 MHz, CDCl3) δ 7.72 (d, J=1.3 Hz, 1H), 7.48-7.34 (m, 7H), 5.01 (s, 1H), 4.68 (s, 1H), 4.45 (s, 2H), 3.91 (s, 1H), 3.12 (d, J=65.7 Hz, 2H), 2.92 (tt, J=11.3, 3.8 Hz, 1H), 2.02 (d, J=15.9 Hz, 2H), 1.73 (s, 2H). ESI-MS: m/z=425.8 [M+1]+

### Example 141: Synthesis of [3-(5-aminoisoxazol-3-yl)azetidin-1-yl]-[3-fluoro-4-(trifluoromethyl)phenyl]methanone (141)

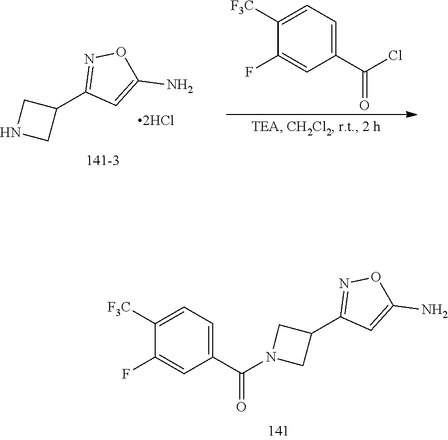 [SMILES: Nc1cc(C2CNC2)no1 ; O=C(Cl)c1ccc(C(F)(F)F)c(F)c1 ; Nc1cc(C2CN(C(=O)c3ccc(C(F)(F)F)c(F)c3)C2)no1]

Similar to the preparation of 85, purification by prep-HPLC provided the target compound 141 (150.30 mg, 456.49 μmol, 42.66% yield) as a white solid. ¹H NMR (400 MHz, DMSO) δ 7.88 (t, J=7.7 Hz, 1H), 7.74 (d, J=11.3 Hz, 1H), 7.67 (d, J=8.1 Hz, 1H), 6.69 (s, 2H), 5.05 (s, 1H), 4.61 (t, J=8.8 Hz, 1H), 4.48-4.31 (m, 2H), 4.08 (dd, J=9.9, 6.1 Hz, 1H), 3.84-3.72 (m, 1H). ESI-MS: m/z=329.8 [M+1]+

### Example 142: Synthesis of 4-(4-(5-aminoisoxazol-3-yl)piperidine-1-carbonyl)-2-fluorobenzonitrile (142)

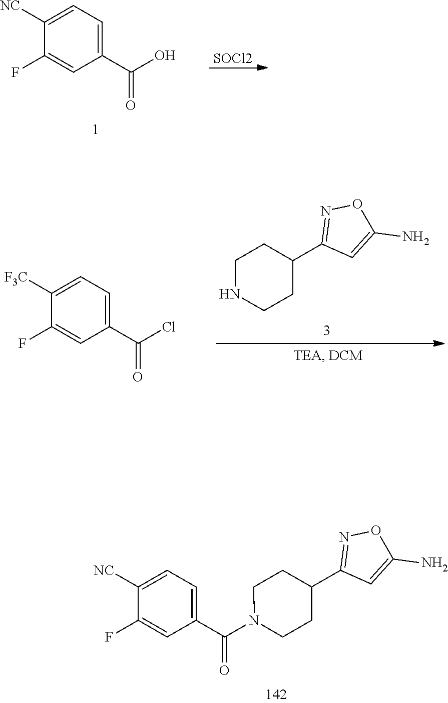

Similar to the preparation of 85, purification by prep-HPLC provided the target compound 142 (67 mg, 202.56 μmol, 36.83% yield) as a white solid. ¹H NMR (400 MHz, CDCl3) δ 7.73 (d, J=7.9 Hz, 1H), 7.55 (d, J=1.3 Hz, 1H), 7.39 (dd, J=7.9, 1.4 Hz, 1H), 5.00 (s, 1H), 4.60 (s, 1H), 4.45 (s, 2H), 3.65 (s, 1H), 3.18 (s, 1H), 3.04 (s, 1H), 2.91 (tt, J=11.1, 3.9 Hz, 1H), 2.04 (s, 1H), 1.95 (s, 1H), 1.68 (s, 2H). ESI-MS: m/z=331.1 (M+1).

### Example 143: Synthesis of [4-(5-aminoisoxazol-3-yl)-1-piperidyl]-[3-cyclopropyl-4-(trifluoromethyl)phenyl]methanone (143)

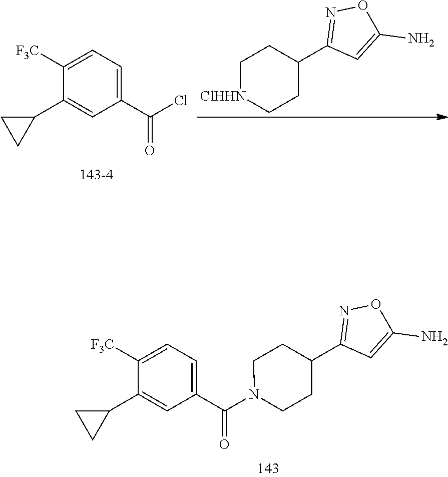

Similar to the preparation of 85, purification by prep-HPLC provided the target compound 143 (80 mg, 210.87 μmol, 48.54% yield) as a white solid. ¹H NMR (400 MHz, CDCl3) δ 7.64 (d, J=8.0 Hz, 1H), 7.23 (s, 1H), 7.06 (s, 1H), 4.99 (s, 1H), 4.65 (s, 1H), 4.45 (s, 2H), 3.68 (s, 1H), 3.19-2.94 (m, 2H), 2.89 (tt, J=11.2, 3.8 Hz, 1H), 2.22 (s, 1H), 2.04 (s, 1H), 1.89 (s, 1H), 1.69 (d, J=40.6 Hz, 2H), 1.10-1.02 (m, 2H), 0.79 (q, J=5.3 Hz, 2H). ESI-MS: m/z=380.2 (M+1)

### Example 144: Synthesis of (3-chloro-4-(trifluoromethyl)phenyl)(4-(5-(3-hydroxypropylamino)isoxazol-3-yl)piperidin-1-yl)methanone (144)

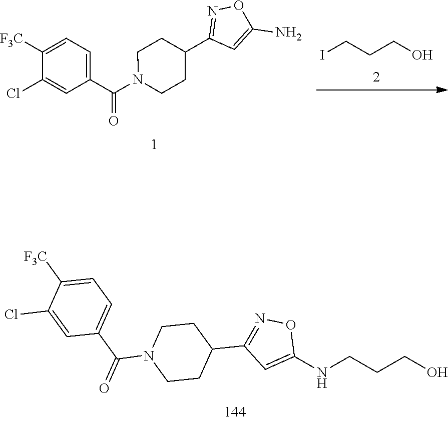 [SMILES: Nc1cc(C2CCN(C(=O)c3ccc(C(F)(F)F)c(Cl)c3)CC2)no1 ; OCCCI ; O=C(c1ccc(C(F)(F)F)c(Cl)c1)N1CCC(c2cc(NCCCO)on2)CC1]

Similar to the preparation of 124, purification by prep-HPLC provided the target compound 144 (8.3 mg, 19.22 μmol, 4.49% yield) as a white solid. ¹H NMR (400 MHz, CDCl3) δ 7.74 (d, J=8.0 Hz, 1H), 7.55 (s, 1H), 7.38 (d, J=8.0 Hz, 1H), 4.97 (t, J=5.6 Hz, 1H), 4.86 (s, 1H), 4.63 (s, 1H), 3.81 (t, J=5.3 Hz, 2H), 3.69 (s, 1H), 3.33 (q, J=6.2 Hz, 2H), 3.17 (s, 1H), 3.03 (s, 1H), 2.90 (tt, J=11.1, 3.8 Hz, 1H), 2.05 (s, 1H), 1.93 (s, 1H), 1.90-1.83 (m, 2H), 1.75 (s, 1H), 1.66 (s, 2H). ESI-MS: m/z=432.1 (M+1)

### Example 145: Synthesis of [4-(5-aminoisoxazol-3-yl)-1-piperidyl]-(4-cyclopropyl-3-phenyl-phenyl)methanone (145)

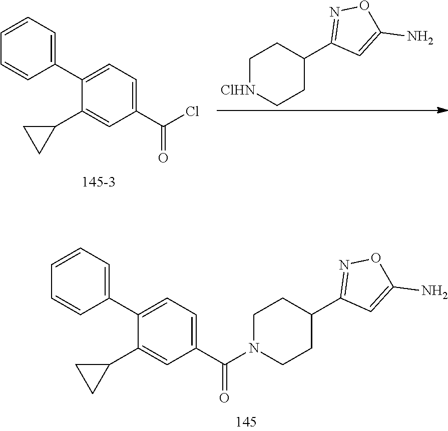

Similar to the preparation of 85, purification by prep-HPLC provided the target compound 145 (23 mg, 59.36 μmol, 14.14% yield) as a white solid. ¹H NMR (400 MHz, DMSO-d6) δ 7.44-7.49 (m, 4H), 7.38-7.41 (m, 1H), 7.24 (t, J=8.0 Hz, 2H), 6.95 (s, 1H), 6.56 (s, 2H), 4.90 (s, 1H), 4.46 (s, 1H), 3.60 (s, 1H) 3.21 (s, 1H), 2.90 (s, 1H), 2.78 (t, J=8.0 Hz, 1H), 1.82-1.86 (m, 3H), 1.56 (s, 2H), 0.86 (d, J=4.0 Hz, 2H), 0.71 (s, 2H). ESI-MS: m/z=388.2 (M+1)

### Example 146: Synthesis of 3-(3-(1-(3-chloro-4-(trifluoromethyl)benzoyl)piperidin-4-yl)isoxazol-5-ylamino)propanenitrile (146)

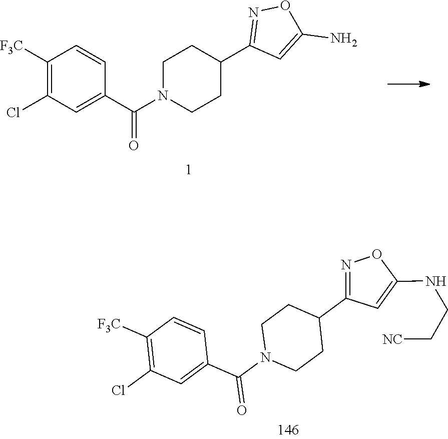 [SMILES: Nc1cc(C2CCN(C(=O)c3ccc(C(F)(F)F)c(Cl)c3)CC2)no1 ; N#CCCNc1cc(C2CCN(C(=O)c3ccc(C(F)(F)F)c(Cl)c3)CC2)no1]

Similar to the preparation of 124, purification by prep-HPLC provided the target compound 146 (8.3 mg, 19.22 μmol, 4.49% yield) as a white solid. ¹H NMR (400 MHz, CDCl3) δ 7.75 (d, J=8.0 Hz, 1H), 7.55 (s, 1H), 7.39 (d, J=8.0 Hz, 1H), 4.96 (s, 1H), 4.97 (t, J=6.5 Hz, 1H), 4.67 (s, 1H), 3.77-3.65 (m, 1H), 3.56 (q, J=6.5 Hz, 2H), 3.18 (s, 1H), 3.03 (s, 1H), 2.97-2.87 (m, 1H), 2.69 (t, J=6.4 Hz, 2H), 2.10-2.01 (m, 1H), 1.98-1.90 (m, 1H), 1.80-1.63 (m, 2H). ESI-MS: m/z=427.1 (M+1)

### Example 147: Synthesis of [4-(5-aminoisoxazol-3-yl)-1-piperidyl]-(3-chloro-4-cyclopropyl-phenyl)methanone (147)

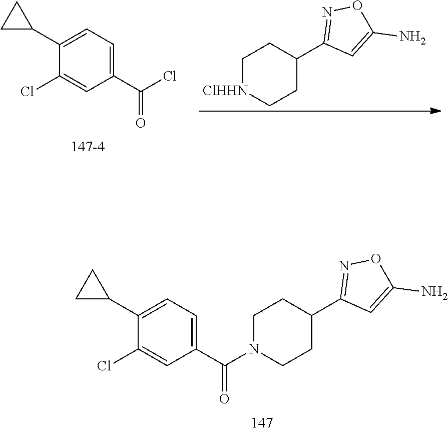

Similar to the preparation of 85, purification by prep-HPLC provided the target compound 147 (33 mg, 95.42 μmol, 18.76% yield) as a white solid. ¹H NMR (400 MHz, DMSO) δ 7.45 (d, J=1.5 Hz, 1H), 7.27 (dd, J=8.0, 1.4 Hz, 1H), 7.07 (d, J=8.0 Hz, 1H), 6.55 (s, 2H), 4.89 (s, 1H), 4.42 (s, 1H), 3.58 (s, 1H), 3.14 (s, 1H), 2.91 (s, 1H), 2.77 (ddd, J=11.4, 7.8, 3.7 Hz, 1H), 2.18 (ddd, J=13.6, 8.5, 5.3 Hz, 1H), 1.87 (s, 1H), 1.77 (s, 1H), 1.55 (d, J=10.8 Hz, 2H), 1.12-1.00 (m, 2H), 0.81-0.70 (m, 2H). ESI-MS: m/z=346.2 (M+1)

### Example 149: Synthesis of (3-chloro-4-(trifluoromethyl)phenyl)(4-(5-(2-(piperidin-1-yl)ethylamino)isoxazol-3-yl)piperidin-1-yl)methanone (149)

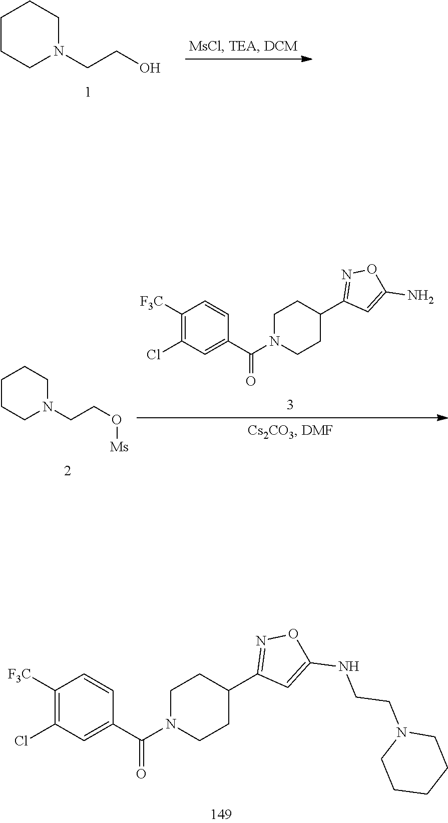 [SMILES: OCCN1CCCCC1 ; CS(=O)(=O)OCCN1CCCCC1 ; Nc1cc(C2CCN(C(=O)c3ccc(C(F)(F)F)c(Cl)c3)CC2)no1 ; O=C(c1ccc(C(F)(F)F)c(Cl)c1)N1CCC(c2cc(NCCN3CCCCC3)on2)CC1]

Synthesis of Compound 2 (step 1): To a solution of compound 1 (500 mg, 3.87 mmol, 511.77 μL) in DCM (10 mL) added N,N-diethylethanamine (587.40 mg, 5.80 mmol, 809.09 μL) and then methanesulfonyl chloride (531.97 mg, 4.64 mmol, 360.17 μL) was slowly added to the mixture. Then the reaction was stirred at 25° C. for 3 h. The reaction was diluted with water (30 mL) and extracted with DCM (40 mL*2). The organic layers were combined, washed with brine, dried over Na₂SO₄ and concentrated to give Compound 2 (630 mg, 3.04 mmol, 78.53% yield). The product was directly used in next step without further purification.

Synthesis of Compound (step 2): To a solution of Compound 2 (83.19 mg, 401.33 μmol) in DMF (3 mL) added Compound 3 (150 mg, 401.33 μmol) and cesium carbonate (196.14 mg, 602.00 μmol). Then the mixture was stirred at 90 C for 3 h. LCMS showed that the major peak was desired product. The reaction was directly purified by prep-HPLC and concentrated to give 149 (41 mg, 84.55 μmol, 21.07% yield). ¹H NMR (400 MHz, CDCl3) δ 7.74 (d, J=8.0 Hz, 1H), 7.55 (s, 1H), 7.39 (d, J=8.0 Hz, 1H), 5.31 (s, 1H), 4.84 (s, 1H), 4.64 (s, 1H), 3.69 (s, 1H), 3.20 (dd, J=11.6, 5.3 Hz, 2H), 3.02 (d, J=4.8 Hz, 1H), 2.91 (tt, J=11.1, 3.8 Hz, 1H), 2.61-2.50 (m, 2H), 2.41 (s, 3H), 2.06 (brs, 1H), 1.93 (brs, 1H), 1.80-1.40 (m, 10H). ESI-MS: m/z=485.2 (M+1)

### Examples 150 and 151: Syntheses of (4-(5-(bis(2-hydroxy-2-methylpropyl)amino)isoxazol-3-yl)piperidin-1-yl)(3-chloro-4-(trifluoromethyl)phenyl)methanone (150) & (3-chloro-4-(trifluoromethyl)phenyl)(4-(5-(2-hydroxy-2-methylpropylamino)isoxazol-3-yl)piperidin-1-yl)methanone (151)

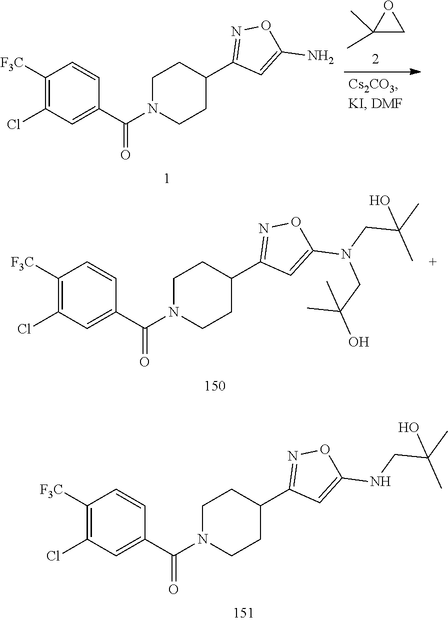

Synthesis of 150 and 151 (step 1): To a mixture of compound 1 (180 mg, 481.60 μmol) in DMF (5 mL) added 2,2-dimethyloxirane (69.45 mg, 963.19 μmol), potassium iodide (87.94 mg, 529.76 μmol, 28.19 μL) and cesium carbonate (172.60 mg, 529.76 μmol). Then the reaction was stirred at 110° C. for 12 h. LCMS showed that the major peak detected desired product MS and BP MS. The reaction was purified with prep-HPLC team to give 150 (35 mg, 78.50 μmol, 16.30% yield) and 151 (8 mg, 15.44 μmol, 3.21% yield).

150: ¹H NMR (400 MHz, CDCl3) δ 7.74 (d, J=8.0 Hz, 1H), 7.55 (s, 1H), 7.39 (d, J=8.6 Hz, 1H), 4.83 (s, 1H), 4.64 (s, 1H), 3.69 (s, 1H), 3.49 (s, 4H), 3.22-3.13 (m, 1H), 3.02 (s, 1H), 2.89 (tt, J=11.2, 3.8 Hz, 1H), 2.10-1.80 (m, 2H), 1.67 (s, 2H), 1.30-1.23 (m, 12H). ESI-MS: m/z=518.2 (M+1)

151: 1H NMR (400 MHz, CDCl3) δ 7.74 (d, J=8.0 Hz, 1H), 7.55 (s, 1H), 7.38 (d, J=8.0 Hz, 1H), 4.87 (s, 1H), 4.63 (s, 1H), 3.69 (s, 1H), 3.24-3.05 (m, 3H), 3.04 (d, J=11.2 Hz, 1H), 2.90 (ddd, J=15.0, 7.5, 4.0 Hz, 1H), 2.05 (s, 1H), 1.92 (s, 1H), 1.80-1.55 (m, 4H), 1.28 (d, J=12.0 Hz, 6H). ESI-MS: m/z=446.2 (M+1)

### Example 152: Synthesis of [4-(5-aminoisoxazol-3-yl)-1-piperidyl]-(3-fluoro-4-phenyl-phenyl)methanone (152)

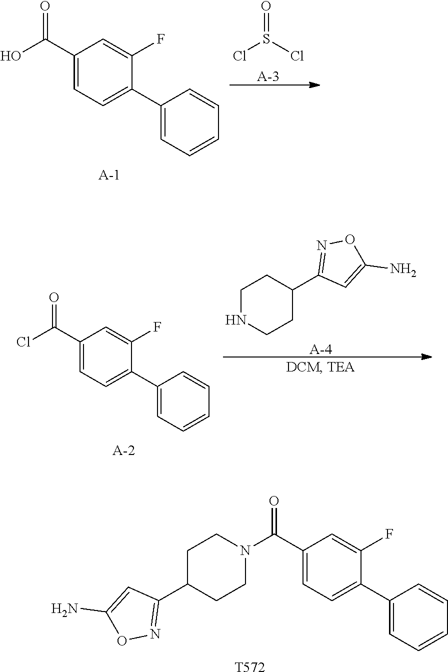

Similar to the preparation of 85, purification by prep-HPLC provided the target compound 152 (170 mg, 465.24 μmol, 50.17% yield) as a white solid. ¹H NMR (400 MHz, CDCl3) δ 7.57-7.53 (m, 2H), 7.47 (dt, J=7.6, 6.9 Hz, 3H), 7.43-7.36 (m, 1H), 7.27 (d, J=1.6 Hz, 1H), 7.22 (dd, J=10.5, 1.5 Hz, 1H), 5.01 (s, 1H), 4.68 (s, 1H), 4.44 (s, 2H), 3.90 (s, 1H), 3.17 (s, 1H), 3.03 (s, 1H), 2.92 (tt, J=11.3, 3.8 Hz, 1H), 2.01 (s, 2H), 1.73 (s, 2H). ESI-MS: m/z=366.1 (M+1)

### Example 153: Synthesis of [4-(5-aminoisoxazol-3-yl)-1-piperidyl]-(3-chloro-4-phenyl-phenyl)methanone (153)

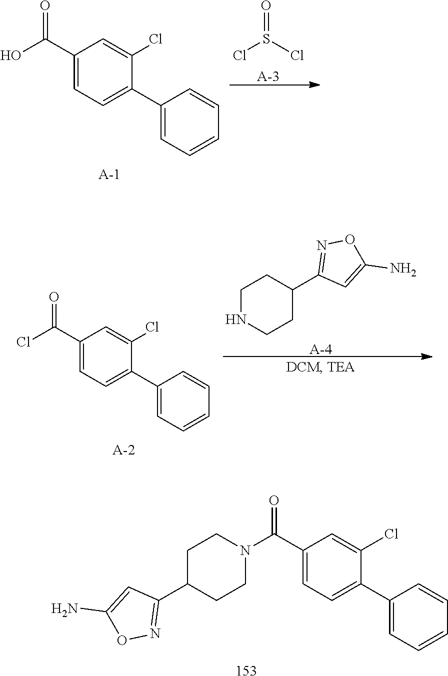

Similar to the preparation of 85, purification by prep-HPLC provided the target compound 153 (71 mg, 185.93 mol, 43.26% yield) as a white solid. ¹H NMR (400 MHz, CDCl3) δ 7.53 (d, J=1.4 Hz, 1H), 7.48-7.33 (m, 7H), 5.02 (s, 1H), 4.68 (s, 1H), 4.42 (s, 2H), 3.90 (s, 1H), 3.20 (s, 1H), 3.02 (s, 1H), 2.92 (tt, J=11.3, 3.9 Hz, 1H), 2.03 (s, 2H), 1.73 (s, 2H). ESI-MS: m/z=382.1 (M+1).

### Example 154: Synthesis of [4-(5-aminoisoxazol-3-yl)-1-piperidyl]-(4-cyclopropyl-3-fluoro-phenyl)methanone (154)

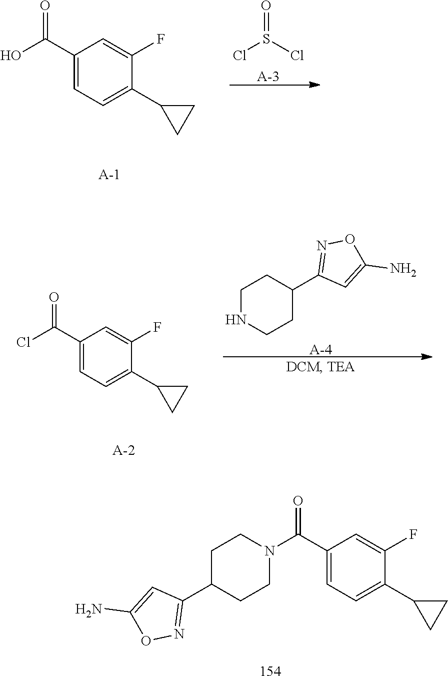

Similar to the preparation of 85, purification by prep-HPLC provided the target compound 154 (109 mg, 330.94 μmol, 59.6% yield) as a white solid. ¹H NMR (400 MHz, CDCl3) δ 7.11-7.04 (m, 2H), 6.91 (dd, J=10.2, 5.4 Hz, 1H), 5.00 (s, 1H), 4.64 (s, 1H), 4.41 (s, 2H), 3.85 (s, 1H), 3.04 (s, 2H), 2.88 (tt, J=11.4, 3.9 Hz, 1H), 2.10 (ddd, J=13.7, 8.5, 5.2 Hz, 1H), 1.96 (s, 2H), 1.69 (s, 2H), 1.02 (ddd, J=8.5, 6.5, 4.6 Hz, 2H), 0.79-0.70 (m, 2H). ESI-MS: m/z=330.1 (M+1).

### Example 155: Synthesis of (4-(5-(2,5,8,11-tetraoxatridecan-13-ylamino)isoxazol-3-yl)piperidin-1-yl)(3-chloro-4-(trifluoromethyl)phenyl)methanone (155)

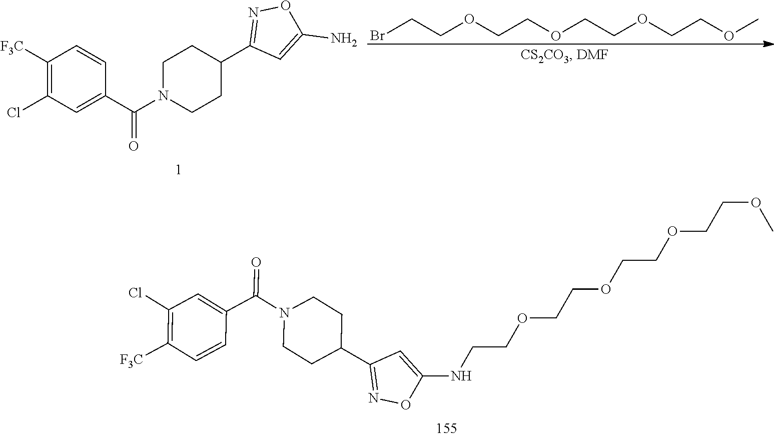 [SMILES: Nc1cc(C2CCN(C(=O)c3ccc(C(F)(F)F)c(Cl)c3)CC2)no1 ; COCCOCCOCCOCCBr ; COCCOCCOCCOCCNc1cc(C2CCN(C(=O)c3ccc(C(F)(F)F)c(Cl)c3)CC2)no1]

Similar to the preparation of 124, purification by prep-HPLC provided the target compound 155 (26 mg, 46.10 μmol, 14.36% yield) as a white solid. ¹H NMR (400 MHz, CDCl3) δ 7.74 (d, J=8.1 Hz, 1H), 7.55 (s, 1H), 7.38 (d, J=8.0 Hz, 1H), 5.44 (t, J=5.7 Hz, 1H), 4.84 (s, 1H), 4.63 (s, 1H), 3.70 (s, 1H), 3.69-3.60 (m, 12H), 3.58-3.54 (m, 2H), 3.38 (s, 3H), 3.34 (dd, J=10.3, 5.6 Hz, 2H), 3.19 (d, J=11.4 Hz, 1H), 3.05 (d, J=13.8 Hz, 1H), 2.90 (tt, J=11.0, 3.8 Hz, 1H), 2.04 (s, 1H), 1.97 (s, 1H), 1.69 (s, 2H). ESI-MS: m/z=564.2 (M+1)

### Example 156: Synthesis of (3-chloro-4-(trifluoromethyl)phenyl)(4-(5-(2-(2-(2-(2-hydroxyethoxy)ethoxy)ethoxy)ethylamino)isoxazol-3-yl)piperidin-1-yl)methanone (156)

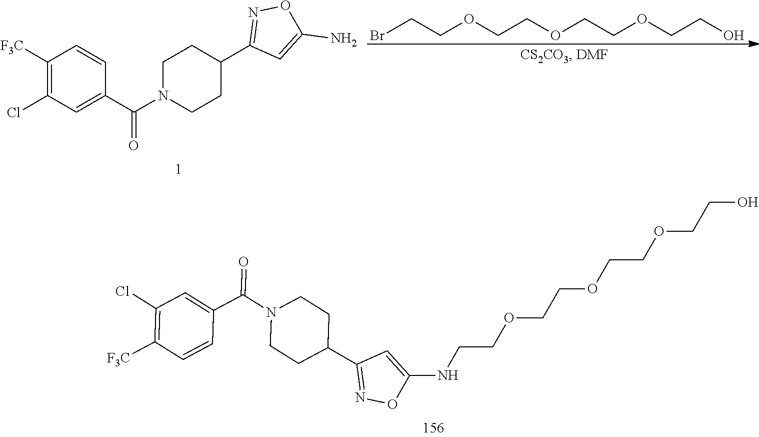 [SMILES: Nc1cc(C2CCN(C(=O)c3ccc(C(F)(F)F)c(Cl)c3)CC2)no1 ; OCCOCCOCCOCCBr ; O=C(c1ccc(C(F)(F)F)c(Cl)c1)N1CCC(c2cc(NCCOCCOCCOCCO)on2)CC1]

Similar to the preparation of 124, purification by prep-HPLC provided the target compound 156 (53 mg, 96.37 μmol, 32.74% yield) as a white solid. ¹H NMR (400 MHz, CDCl3) δ 7.74 (d, J=8.0 Hz, 1H), 7.55 (s, 1H), 7.38 (d, J=8.0 Hz, 1H), 6.07 (t, J=5.8 Hz, 1H), 4.82 (s, 1H), 4.62 (s, 1H), 3.79-3.70 (m, 4H), 3.65 (dd, J=8.8, 5.1 Hz, 10H), 3.35 (dd, J=10.0, 5.5 Hz, 2H), 3.29 (s, 1H), 3.17 (s, 1H), 3.03 (s, 1H), 2.90 (tt, J=11.0, 3.8 Hz, 1H), 2.15-1.90 (m, 2H), 1.76 (s, 2H). ESI-MS: m/z=550.2 (M+1)

### Example 157: Synthesis of (3-chloro-4-(trifluoromethyl)phenyl)(4-(5-(2,3-dihydroxypropylamino)isoxazol-3-yl)piperidin-1-yl)methanone (157)

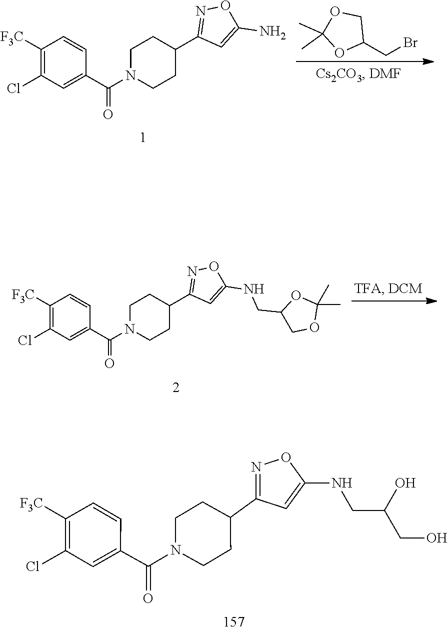

Synthesis of Compound 2 (step 1): To a solution of Compound 1 (150 mg, 401.33 μmol) in DMF (2.5 mL) added 4-(bromomethyl)-2,2-dimethyl-1,3-dioxolane (78.28 mg, 401.33 μmol) and cesium carbonate (196.14 mg, 602.00 μmol). Then the reaction was stirred at 90° C. for 2 h. LCMS showed that the major peak was desired product. The reaction was diluted with water (20 mL) and extracted with EA (20 mL*3). The organic layers were combined, washed with brine, dried over Na₂SO₄ and concentrated to give crude product. The crude product was purified with silica gel column (EA:PE=0 to 100%) and concentrated to give Compound 2 (110 mg, 225.46 μmol, 56.18% yield).

Synthesis of 157 (step 2): To a solution of Compound 2 (110 mg, 225.46 mol) in DCM (4 mL) added TFA (2 mL). Then the reaction was stirred at 25° C. for 2 h. The reaction was directly purified with prep-HPLC to yield 157 (21 mg, 46.89 μmol, 20.80% yield). ¹H NMR (400 MHz, CDCl3) δ 7.74 (d, J=8.1 Hz, 1H), 7.54 (s, 1H), 7.38 (d, J=8.0 Hz, 1H), 5.27 (t, J=5.9 Hz, 1H), 4.89 (s, 1H), 4.61 (s, 1H), 3.98-3.85 (m, 1H), 3.72 (dd, J=11.2, 3.7 Hz, 1H), 3.65 (s, 1H), 3.60 (dd, J=11.2, 5.9 Hz, 1H), 3.31 (ddd, J=13.1, 6.7, 4.3 Hz, 1H), 3.27-3.11 (m, 2H), 3.00 (s, 1H), 2.89 (tt, J=11.1, 3.7 Hz, 1H), 2.11-1.90 (m, 3H), 1.73 (s, 1H), 1.66 (s, 1H). ESI-MS: m/z=448.1 (M+1)

### Example 158: Synthesis of 4-(3-(1-(3-chloro-4-(trifluoromethyl)benzoyl)piperidin-4-yl)isoxazol-5-ylamino)butanenitrile (158)

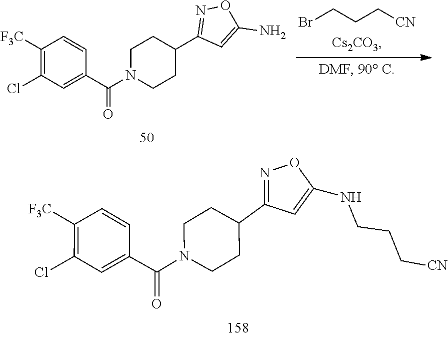

Similar to the preparation of 124, purification by prep-HPLC provided the target compound 158 (16.5 mg, 37.4 mol, 6.99% yield) as a white solid. ¹H NMR (400 MHz, CDCl3) δ 7.74 (d, J=8.0 Hz, 1H), 7.55 (s, 1H), 7.39 (d, J=8.0 Hz, 1H), 4.93 (s, 1H), 4.65 (s, 1H), 4.61 (t, J=6.2 Hz, 1H), 3.70 (s, 1H), 3.38 (q, J=6.5 Hz, 2H), 3.18 (s, 1H), 3.03 (s, 1H), 2.91 (tt, J=11.1, 3.9 Hz, 1H), 2.48 (t, J=7.0 Hz, 2H), 2.05 (s, 1H), 2.03-1.92 (m, 3H), 1.75 (s, 1H) 1.68 (s, 1H). ESI-MS: m/z=440.8 (M+1)

### Example 159: Synthesis of (4-(5-amino-4-chloroisoxazol-3-yl)piperidin-1-yl)(3-chloro-4-(trifluoromethyl)phenyl)methanone (159)

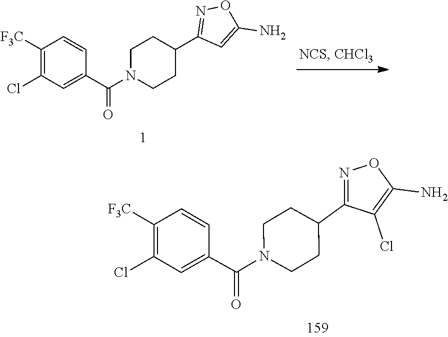

Synthesis of 159 (step 1): To a solution of Compound 1 (100 mg, 267.55 mol) in CHCl3 (2 mL) added 1-chloropyrrolidine-2,5-dione (35.73 mg, 267.55 μmol, 21.65 L). Then the reaction was stirred at 0° C. for 2 h. LCMS showed that the major peak was desired product. The reaction was diluted with water (20 mL) and extracted with DCM (20 mL*2). The organic layer was combined, washed with brine, dried over Na₂SO₄ and concentrated to give crude product. The crude product was purified with prep-HPLC to yield 159 (19 mg, 46.55 μmol, 17.40% yield). ¹H NMR (400 MHz, CDCl3) δ 7.75 (d, J=8.0 Hz, 1H), 7.55 (s, 1H), 7.42-7.36 (m, 1H), 4.64 (s, 1H), 4.58 (s, 2H), 3.73 (s, 1H), 3.18 (s, 1H), 3.07 (s, 1H), 2.96 (tt, J=10.9, 4.0 Hz, 1H), 2.10 (s, 1H), 1.87 (s, 3H). ESI-MS: m/z=408.1 (M+1)

### Example 160: Synthesis of (4-(5-amino-4-bromoisoxazol-3-yl)piperidin-1-yl)(3-chloro-4-(trifluoromethyl)phenyl)methanone (160)

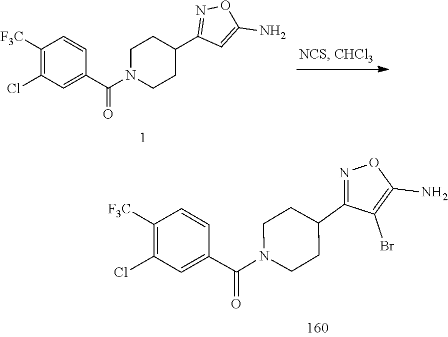

Synthesis of 160 (step 1): To a solution of Compound 1 (100 mg, 267.55 mol) in CDCl3 (2.00 mL) added NBS (47.62 mg, 267.55 μmol, 22.70 μL). Then the reaction was stirred at 0° C. for 2 h. LCMS showed that the major peak was desired product. The reaction was diluted with water (20 mL) and extracted with DCM (20 mL*2). The organic layer was combined, washed with brine, dried over Na2SO4 and concentrated to give crude product. The crude product was purified with prep-HPLC to yield 160 (48 mg, 106.04 μmol, 39.63% yield). ¹H NMR (400 MHz, CDCl3) δ 7.74 (d, J=8.0 Hz, 1H), 7.55 (s, 1H), 7.39 (d, J=8.0 Hz, 1H), 4.63 (s, 3H), 3.72 (s, 1H), 3.18 (s, 1H), 3.07 (s, 1H), 2.92 (tt, J=10.9, 3.9 Hz, 1H), 2.10 (s, 1H), 1.86 (s, 3H). ESI-MS: m/z=451.5 (M+1)

### Example 161: Synthesis of (3-chloro-4-(trifluoromethyl)phenyl)(4-(5-(oxetan-3-ylamino)isoxazol-3-yl)piperidin-1-yl)methanone (161)

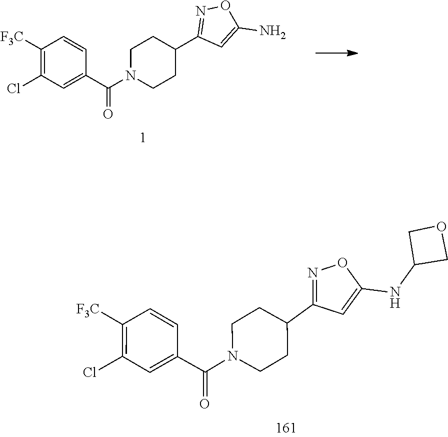

Similar to the preparation of 124, purification by prep-HPLC provided the target compound 161 (5.5 mg, 12.80 μmol, 2.39% yield) as a white solid. ¹H NMR (400 MHz, CDCl₃) δ 7.74 (d, J=8.1 Hz, 1H), 7.55 (s, 1H), 7.38 (d, J=8.0 Hz, 1H), 5.10-4.92 (m, 3H), 4.89 (s, 1H), 4.75-4.55 (m, 4H), 3.72 (s, 1H), 3.17 (s, 1H), 3.02 (s, 1H), 2.98-2.85 (m, 1H), 2.20-1.90 (m, 2H), 1.80-1.75 (s, 2H). ESI-MS: m/z=429.7 (M+1)

### Example 162: Synthesis of [4-(5-aminoisoxazol-3-yl)-1-piperidyl]-[2-bromo-4-(trifluoromethyl)phenyl]methanone (162)

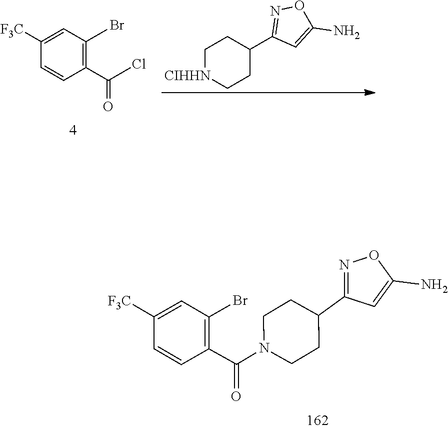

Similar to the preparation of 85, purification by prep-HPLC provided the target compound 162 (190 mg, 454.32 μmol, 65.30% yield) as a white solid. ¹H NMR (400 MHz, CDCl3) δ 7.86 (d, J=5.5 Hz, 1H), 7.63 (d, J=7.9 Hz, 1H), 7.39 (dd, J=19.9, 7.9 Hz, 1H), 4.99 (d, J=2.9 Hz, 1H), 4.71 (dd, J=25.5, 13.7 Hz, 1H), 4.44 (s, 2H), 3.44 (t, J=13.7 Hz, 1H), 3.26-2.99 (m, 2H), 2.90 (ddd, J=14.6, 7.4, 3.7 Hz, 1H), 2.07 (dd, J=17.6, 7.2 Hz, 1H), 1.97-1.69 (m, 3H). ESI-MS: m/z=417.6[M+H]+.

### Example 163: Synthesis of (4-(5-amino-4-methoxyisoxazol-3-yl)piperidin-1-yl)(3-chloro-4-(trifluoromethyl)phenyl)methanone (163)

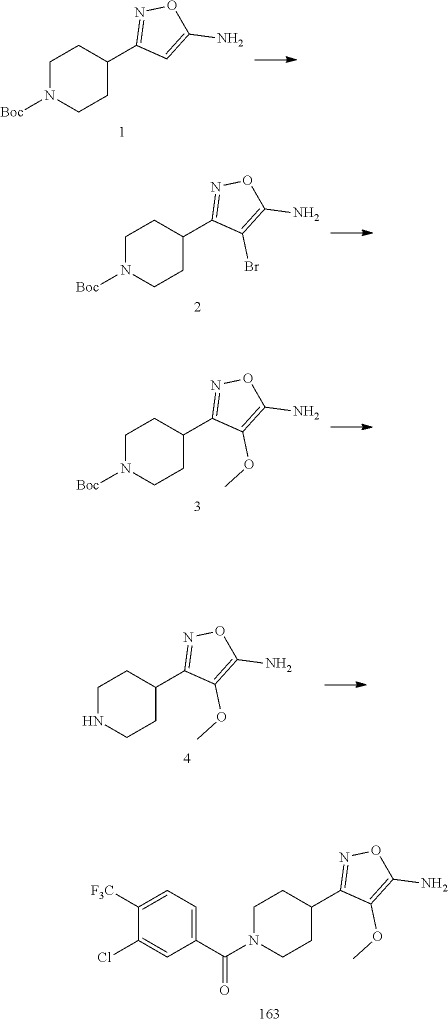 [SMILES: CC(C)(C)OC(=O)N1CCC(c2cc(N)on2)CC1 ; CC(C)(C)OC(=O)N1CCC(c2noc(N)c2Br)CC1 ; COc1c(C2CCN(C(=O)OC(C)(C)C)CC2)noc1N ; COc1c(C2CCNCC2)noc1N ; COc1c(C2CCN(C(=O)c3ccc(C(F)(F)F)c(Cl)c3)CC2)noc1N]

Synthesis of Compound 2 (step 1): A solution of Compound 1 (400 mg, 1.50 mmol) in chloroform (4 mL) added 1-bromopyrrolidine-2,5-dione (266.31 mg, 1.50 mmol, 126.94 μL). Then the reaction was stirred at 25° C. for 2 h. When the LCMS showed that the major peak was desired product, the reaction was diluted with water (20 mL) and extracted with EA (20 M*2). The organic layer was combined, washed with brine, dried over Na2SO4 and concentrated to give crude product. The crude product was purified with silica gel column (EA:EP=0 to 60%) and concentrated to give Compound 2 (460 mg, 1.33 mmol, 88.79% yield).

Synthesis of Compound 3 (step 2): To a solution of Compound 2 (150 mg, 433.25 μmol) in NaOCH3 (3.01 mL). Then the reaction was stirred at 50° C. for 2 h. LCMS showed that the major peak was desired product. The reaction was quenched with aq. NH4Cl (20 mL) and extracted with EA (20 mL). The organic layer was combined, washed with brined, dried over Na2SO4 and concentrated to give Compound 3 (136 mg, 457.37 μmol, >99% yield). The crude product was directly used at next step without further purified.

Synthesis of Compound 4 (step 3): A mixture of Compound 3 (130 mg, 437.20 μmol) in 4 M HCl in dioxane (1 mL). Then the reaction was stirred at 25° C. for 2 h. TLC (EA:PE=1:1, UV, 254 nm) showed that the start material was consumed and new spot was detected.

The reaction was directly concentrated to give Compound 4 (89 mg, 380.84 mol, 87.11% yield). The product was directly concentrated to use at next step without further purified.

Synthesis of 163 (step 4): A mixture of Compound 4 (72.23 mg, 309.10 mol, CL) in DMF (1 mL) and DCM (2 mL) added N,N-diethylethanamine (111.18 mg, 1.10 mmol, 153.14 μL). Then 3-chloro-4-(trifluoromethyl)benzoyl chloride (89 mg, 366.24 μmol) in DCM (1 mL) was added to the mixture. The reaction was stirred at 25° C. for 2 h. When the LCMS showed the desired product MS and the reaction was concentrated, then purified with HPLC team to give 163 (2.14 mg, 5.30 μmol, 1.45% yield). ¹H NMR (400 MHz, CDCl3) δ 7.74 (d, J=8.0 Hz, 1H), 7.55 (s, 1H), 7.39 (d, J=8.1 Hz, 1H), 4.62 (s, 1H), 4.24 (s, 2H), 3.73 (s, 1H). 3.71 (s, 3H), 3.25-3.01 (m, 2H), 2.97 (tt, J=10.9, 3.9 Hz, 1H), 2.10 (s, 1H), 2.01-1.80 (m, 3H). ESI-MS: m/z=404.1 [M+H]+.

### Example 164: Synthesis of 2-[4-(5-aminoisoxazol-3-yl)piperidine-1-carbonyl]-5-(trifluoromethyl)benzonitrile (164)

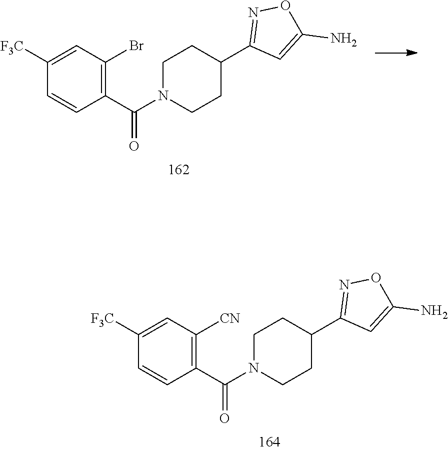 [SMILES: Nc1cc(C2CCN(C(=O)c3ccc(C(F)(F)F)cc3Br)CC2)no1 ; N#Cc1cc(C(F)(F)F)ccc1C(=O)N1CCC(c2cc(N)on2)CC1]

To a solution of [4-(5-aminoisoxazol-3-yl)-1-piperidyl]-[2-bromo-4-(trifluoromethyl)phenyl]methanone (70 mg, 167.38 μmol) in DMF (5 mL) was added zinc dicyanide (78.62 mg, 669.52 μmol, 42.45 μL), Pd₂(dba)₃ (30.65 mg, 33.48 μmol) and dicyclohexyl-[2-(2,6-dimethoxyphenyl)phenyl]phosphane (27.49 mg, 66.95 μmol). The resulting mixture was stirred at 120° C. under microwave for 1 hour. LCMS showed the starting material was consumed and the desired product was generated. The reaction was concentrated and purified by prep-HPLC provided 2-[4-(5-aminoisoxazol-3-yl)piperidine-1-carbonyl]-5-(trifluoromethyl)benzonitrile (17 mg, 46.7 μmol, 27.9% yield) as a white solid. ¹H NMR (400 MHz, CDCl3) δ 7.98 (s, 1H), 7.92 (d, J=8.6 Hz, 1H), 7.62 (d, J=8.1 Hz, 1H), 5.03 (s, 1H), 4.70 (d, J=13.2 Hz, 1H), 4.44 (s, 2H), 3.47 (d, J=13.6 Hz, 1H), 3.25 (t, J=11.4 Hz, 1H), 3.11 (t, J=11.7 Hz, 1H), 2.94 (tt, J=11.0, 3.8 Hz, 1H), 2.08 (d, J=11.3 Hz, 1H), 1.93 (d, J=11.1 Hz, 1H), 1.89-1.73 (m, 2H). ESI-MS: m/z=364.7[M+H]+.

### Example 165: Synthesis of 2-[2-[[3-[1-[3-chloro-4-(trifluoromethyl)benzoyl]-4-piperidyl]isoxazol-5-yl]amino]ethyl]isoindoline-1,3-dione (165)

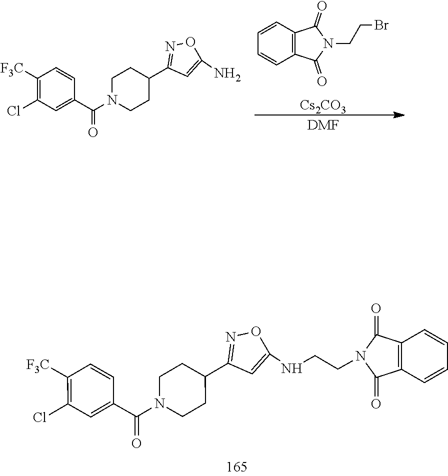 [SMILES: Nc1cc(C2CCN(C(=O)c3ccc(C(F)(F)F)c(Cl)c3)CC2)no1 ; O=C1c2ccccc2C(=O)N1CCBr ; O=C(c1ccc(C(F)(F)F)c(Cl)c1)N1CCC(c2cc(NCCN3C(=O)c4ccccc4C3=O)on2)CC1]

Similar to the preparation of 124, purification by prep-HPLC provided the target compound 165 (184 mg, 336.43 μmol, 83.83% yield) as a white solid. ¹H NMR (400 MHz, DMSO) δ 11.93 (s, 1H), 7.94 (d, J=8.1 Hz, 1H), 7.84 (s, 2H), 7.69-7.51 (m, 4H), 6.40 (s, 1H), 4.50 (d, J=11.4 Hz, 1H), 4.31 (t, J=9.5 Hz, 2H), 3.88 (t, J=9.5 Hz, 2H), 3.51 (d, J=12.7 Hz, 1H), 3.21 (t, J=11.4 Hz, 1H), 3.08-2.92 (m, 2H), 2.00 (d, J=11.4 Hz, 1H), 1.81 (s, 1H), 1.72 (dt, J=12.0, 8.6 Hz, 2H).

### Biological Methods and Assays

Tissue culture. THP-1 Lucia ISG cells (cat. no. thpl-isg) and TREX1 KO THP-1 Lucia ISG cells (cat. no. thpd-kotrex), which express luciferase reporter gene under the control of a promoter containing five tandem repeats of the interferon stimulated response element (ISRE), were purchased from Invivogen and maintained in growth media consisting of RPMI 1640, 2 mM L-glutamine, 25 mM HEPES, 10% heat-inactivated fetal bovine serum (FBS), 1,000 units/ml penicillin, 1,000 μg/ml streptomycin, 0.25 μg/ml Amphotericin B, and 100 μg/ml zeocin unless otherwise stated.

ISRE-luciferase assay. THP-1 Lucia ISG cells were resuspended in low-serum growth media (2% FBS) at a density of 4×10⁵ cells/ml and were transfected with VACV-70-LyoVec (cat. no. tlrl-vav70c, Invivogen) at a concentration of 2 μg/mL). 100 nL of compound or vehicle was spotted onto a 384-well white greiner plate using an acoustic echo liquid handler (Labycte). 50 μL of cells were seeded into each well and incubated for 48 hours. To evaluate expression of the luciferase reporter, 30 μl of Quanti-luc (cat. no. rep-qlcl, Invivogen) detection reagent was added to each well and luminescence was read using an Envision plate reader (Perkin Elmer) set with an integration time of 0.1 seconds. Results are shown in Table 2 below.

Viability assay. THP-1 Lucia ISG cells were resuspended in low-serum growth media (2% FBS) at a density of 4×10⁵ cells/ml and were transfected with VACV-70-LyoVec (cat. no. tlrl-vav70c, Invivogen) at a concentration of 2 μg/mL). 100 nL of compound or vehicle was spotted onto a 384-well white greiner plate using an acoustic echo liquid handler (Labycte). 50 μL of cells were seeded into each well and incubated for 48 hours. To evaluate ATP levels as a measurement of metabolically active cells, 30 μl of CellTiter-Glo (cat. no. G7570, Promega) detection reagent was added to each well and luminescence was detected using an Envision Plate Reader set with an integration time of 0.1 seconds.

2′3′-cGAMP ELISA. THP-1 Lucia ISG cells were resuspended in low-serum growth media (2% FBS) at a density of 4×10⁵ cells/ml and were transfected with VACV-70-LyoVec (cat. no. tlrl-vav70c, Invivogen) at a concentration of 2 μg/mL) or left untreated (UT). 2 mL of transfected cells were treated with compound (dissolved in DMSO) or DMSO for a final dilution of DMSO of 1000×. To determine basal levels of 2′3′-cGAMP, UT cells were treated with DMSO. Cells were incubated for 48 hours before lysing the cells in M-PER protein extraction buffer (cat. no. 78501, ThermoFisher) following manufacturer's instructions. 2′3′-cGAMP levels were quantified using a 2′3′-cGAMP ELISA (cat. no. 501700, Cayman Chemicals) and normalized to protein concentration which was quantified using a BCA protein assay (cat. no. 23225, Thermo Fisher).

TABLE 2
Formula (I) Compound Analytical Data and Inhibition of cGAS

| | | Activity | |
|---|---|---|---|
| Cmpd. | Structure | Score | Analytical Data |
| 1 | 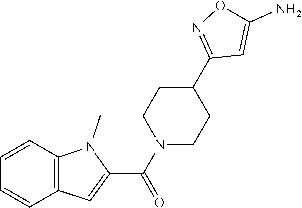 [SMILES: Cn1c(C(=O)N2CCC(c3cc(N)on3)CC2)cc2ccccc21] | ++ | ¹HNMR (400 MHz, DMSO-d₆) δ 7.59 (d, J = 7.6 Hz, 1H), 7.49 (d, J = 8.4 Hz, 1H), 7.23 (t, J = 7.6 Hz, 1H), 7.08 (t, J = 7.6 Hz, 1H), 6.64 (s, 1H), 6.53 (s, 2H), 4.88 (s, 1H), 3.90-4.60 (m, 2H), 3.74 (s, 3H), 3.01-3.20 (m, 2H), 2.78-2.84 (m, 1H), 1.81-1.92 (m, 2H), 1.52-1.62 (m, 2H). ESI-MS: m/z = 325.2 (M + 1). |
| 2 | 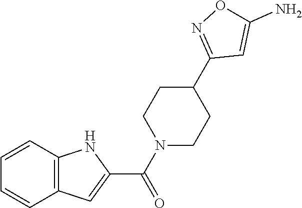 [SMILES: Nc1cc(C2CCN(C(=O)c3cc4ccccc4[nH]3)CC2)no1] | ++ | ¹HNMR (400 MHz, MeOD-d₄) δ 7.60 (d, J = 8.0 Hz, 1H), 7.42 (d, J = 8.0 Hz, 1H), 7.20 (t, J = 8.0 Hz, 1H), 7.05 (t, J = 8.0 Hz, 1H), 6.81 (s, 1H), 4.99 (s, 1H), 4.80-4.90 (m, 2H), 4.58-4.61 (m, 2H), 2.90-3.00 (m, 1H), 2.00-2.03 (m, 2H), 1.71-1.75 (m, 2H). ESI-MS: m/z 311.3 (M + 1). |
| 3 | 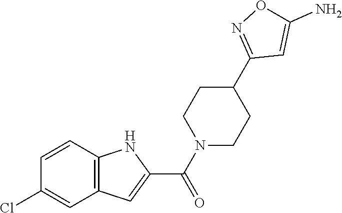 [SMILES: Nc1cc(C2CCN(C(=O)c3cc4cc(Cl)ccc4[nH]3)CC2)no1] | +++ | ¹HNMR (400 MHz, DMSO-d₆) δ 11.75 (brs, 1H), 7.63 (d, J = 2.0 Hz, 1H), 7.40 (d, J = 8.8 Hz, 1H), 7.15 (dd, J = 8.8, 2.0 Hz, 1H), 6.75 (s, 1H), 6.53 (s, 2H), 4.86 (s, 1H), 4.38-4.41 (m, 2H), 3.14-3.20 (m, 2H), 2.80-2.85 (m, 1H), 1.87-1.90 (m, 2H), 1.52-1.62 (m, 2H). ESI-MS: m/z = 345.2 (M + 1). |
| 4 | 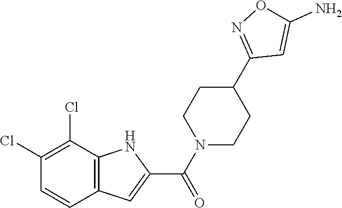 [SMILES: Nc1cc(C2CCN(C(=O)c3cc4ccc(Cl)c(Cl)c4[nH]3)CC2)no1] | +++ | ¹HNMR (400 MHz, DMSO-d₆) δ 11.90 (brs, 1H), 7.53-7.55 (m, 1H), 7.05-7.18 (m, 1H), 6.75-6.80 (m, 1H), 6.53 (s, 2H), 4.86 (s, 1H), 4.32-4.56 (m, 2H), 2.93-3.22 (m, 2H), 2.79-2.93 (m, 1H), 1.80-1.92 (m, 2H), 1.51-1.61 (m, 2H). ESI-MS: m/z 379.2 (M + 1). |
| 5 | 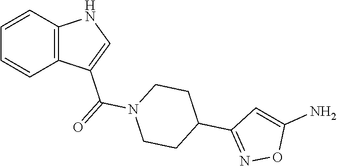 [SMILES: Nc1cc(C2CCN(C(=O)c3c[nH]c4ccccc34)CC2)no1] | + | ¹H NMR (400 MHz, DMSO-d₆) δ 11.56 (s, 1 H), 7.61-7.67 (m, 2 H), 7.41-7.44 (m, 1 H), 7.05-7.14 (m, 2 H), 6.51 (s, 2 H), 4.86 (s, 1 H), 4.26 (d, J = 13.2 Hz, 2 H), 3.01-3.07 (m, 2 H), 2.73-2.79 (m, 1 H), 1.83 (d, J = 14.0 Hz, 2 H), 1.48-1.59 (m, 2 H). ESI-MS: m/z = 311.4 (M + 1). |
| 6 | 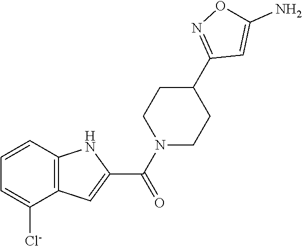 | +++ | ¹H NMR (400 MHz, DMSO-d₆) δ 11.97 (s, 1 H), 7.37 (d, J = 8.0 Hz, 1 H), 7.07-7.13 (m, 2 H), 6.72 (s, 1 H), 6.52 (s, 2 H), 4.87 (s, 1 H), 4.44 (d, J = 10.8 Hz, 2 H), 2.78-2.87 (m, 1 H), 2.39-2.42 (m, 2 H), 1.88 (d, J = 13.2 Hz, 2 H), 1.52-1.62 (m, 2 H). ESI-MS: m/z = 345.3 (M + 1). |
| 7 | 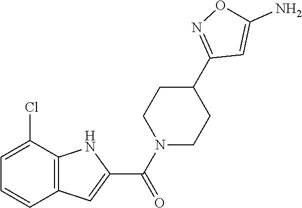 [SMILES: Nc1cc(C2CCN(C(=O)c3cc4cccc(Cl)c4[nH]3)CC2)no1] | +++ | ¹H NMR (400 MHz, DMSO-d₆) δ 11.79 (s, 1 H), 7.55 (d, J = 7.6 Hz, 1 H), 7.23 (d, J = 7.6 Hz, 1 H), 7.03 (t, J = 8.0 Hz, 1 H), 6.77 (s, 1 H), 6.53 (s, 2 H), 4.85 (s, 1 H), 4.26 (bs, 2 H), 3.17-3.23 (m, 1 H), 2.78-2.86 (m, 2 H), 1.82-1.90 (m, 2 H), 1.52-1.64 (m, 2 H). ESI-MS: m/z = 345.4 (M + 1). |
| 8 | 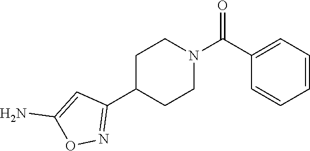 [SMILES: Nc1cc(C2CCN(C(=O)c3ccccc3)CC2)no1] | − | ¹HNMR (400 MHz, DMSO-d₆) δ 7.41-7.44 (m, 3H), 7.36-7.39 (m, 2H), 6.51 (s, 2H), 4.86 (s, 1H), 4.38-4.50 (m, 1H), 3.49-3.55 (m, 1H), 3.03-3.18 (m, 1H), 2.84-2.96 (m, 1H), 2.72-2.79 (m, 1H), 1.69-1.92 (m, 2H), 1.46-1.59 (m, 2H). ESI-MS: m/z = 272.2 (M + 1). |
| 9 | 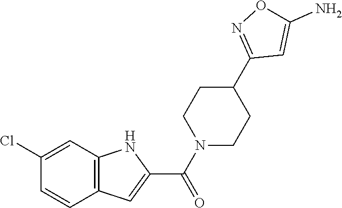 [SMILES: Nc1cc(C2CCN(C(=O)c3cc4ccc(Cl)cc4[nH]3)CC2)no1] | +++ | ¹HNMR (400 MHz, DMSO-d₆) δ 11.71 (brs, 1H), 7.62 (d, J = 8.4 Hz, 1H), 7.42 (s, 1H), 7.05 (dd, J = 8.4, 2.0 Hz, 1H), 6.82 (s, 1H), 6.54 (s, 2H), 4.88 (s, 1H), 4.42-4.45 (m, 2H), 3.00- 3.20 (m, 2H), 2.82-2.87 (m, 1H), 1.89-1.92 (m, 2H), 1.58-1.63 (m, 2H). ESI-MS: m/z = 345.2 (M + 1). |
| 10 | 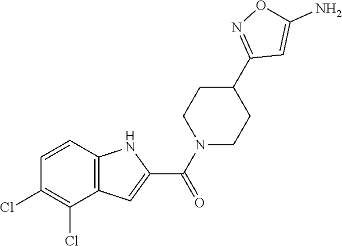 [SMILES: Nc1cc(C2CCN(C(=O)c3cc4c(Cl)c(Cl)ccc4[nH]3)CC2)no1] | ++ | ¹H NMR (400 MHz, DMSO-d₆) δ 12.09 (s, 1 H), 7.32 (d, J = 8.4 Hz, 1 H), 7.14 (s, 1 H), 6.69 (s, 1 H), 6.51 (s, 2 H), 4.85 (s, 1 H), 4.49 (bs, 2 H), 3.24 (s, 2 H), 2.76-2.80 (m, 1 H), 1.86 (d, J = 12.0 Hz, 2 H), 1.53-1.56 (m, 2 H). ESI-MS: m/z = 379.2 (M + 1). |
| 11 | 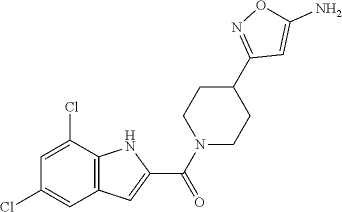 [SMILES: Nc1cc(C2CCN(C(=O)c3cc4cc(Cl)cc(Cl)c4[nH]3)CC2)no1] | ++++ | 1H NMR (400 MHz, DMSO-d6) δ 12.04 (s, 1 H), 7.59 (s, 1 H), 7.26 (s, 1 H), 6.73 (s, 1 H), 6.52 (s, 2 H), 4.85 (s, 1 H), 4.45 (bs, 2 H), 3.21 (s, 2 H), 2.78-2.83 (m, 1 H), 1.84 (s, 2 H), 1.52-1.59 (m, 2 H). ESI-MS: m/z = 379.2 (M + 1). |
| 12 | 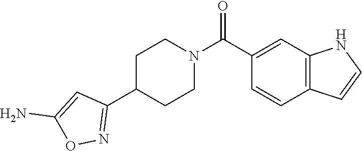 [SMILES: Nc1cc(C2CCN(C(=O)c3ccc4cc[nH]c4c3)CC2)no1] | + | ¹H NMR (400 MHz, DMSO-d₆) δ 11.25 (s, 1 H), 7.55 (d, J = 8.0 Hz, 1 H), 7.43 (s, 2 H), 6.99-7.02 (m, 1 H), 6.52 (s, 2 H), 6.45 (d, J = 3.2 Hz, 1 H), 4.87 (s, 1 H), 4.15 (bs, 2 H), 2.99 (s, 2 H), 2.70-2.79 (m, 1 H), 1.80 (s, 2 H), 1.51-1.55 (m, 2 H). ESI-MS: m/z = 311.4 (M + 1). |
| 13 | 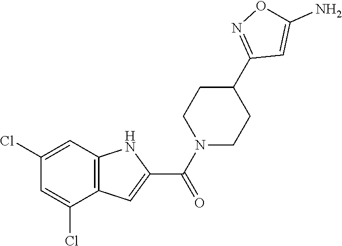 [SMILES: Nc1cc(C2CCN(C(=O)c3cc4c(Cl)cc(Cl)cc4[nH]3)CC2)no1] | +++ | ¹HNMR (400 MHz, DMSO-d₆) δ 12.10 (brs, 1H), 7.39 (s, 1H), 7.15 (s, 1H), 6.76 (s, 1H), 6.54 (s, 2H), 4.88 (s, 1H), 4.47-4.51 (m, 2H), 2.90- 3.20 (m, 2H), 2.79-2.86 (m, 1H), 1.88- 1.91 (m, 2H), 1.54-1.62 (m, 2H). ESI- MS: m/z = 379.1 (M + 1). |
| 14 | 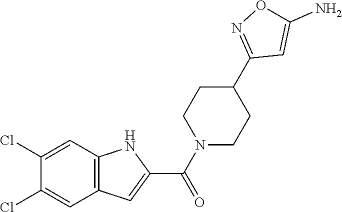 [SMILES: Nc1cc(C2CCN(C(=O)c3cc4cc(Cl)c(Cl)cc4[nH]3)CC2)no1] | +++ | ¹H NMR (400 MHz, DMSO-d₆) δ 11.87 (s, 1 H), 7.83 (s, 1 H), 7.58 (s, 1 H), 6.77 (s, 1 H), 6.52 (s, 2 H), 4.86 (s, 1 H), 4.42 (d, J = 12.8 Hz, 2 H), 3.23 (s, 2 H), 2.78-2.86 (m, 1 H), 1.89 (d, J = 13.2 Hz, 2 H), 1.50-1.61 (m, 2 H). ESI-MS: m/z = 379.3 (M + 1). |
| 15 | 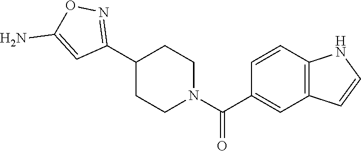 [SMILES: Nc1cc(C2CCN(C(=O)c3ccc4[nH]ccc4c3)CC2)no1] | + | ¹H NMR (400 MHz, DMSO-d₆) δ 11.28 (s, 1H), 7.59 (s, 1H), 7.39- 7.41 (m, 2H), 7.11 (dd, J = 8.4, 1.6 Hz, 1H), 6.51 (s, 2H), 6.47 (d, J = 3.2 Hz, 1H), 4.87 (s, 1H), 3.90-4.30 (brs, 2H), 2.90-3.10 (m, 2H), 2.74-2.76 (m, 1H), 1.78-1.81 (m, 2H), 1.51-1.55 (m, 2H). ESI-MS: m/z = 311.3 (M + 1). |
| 16 | 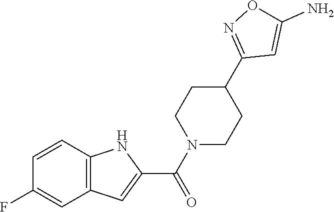 [SMILES: Nc1cc(C2CCN(C(=O)c3cc4cc(F)ccc4[nH]3)CC2)no1] | ++ | ¹H NMR (400 MHz, DMSO-d₆) δ 11.65 (s, 1 H), 7.32-7.40 (m, 2 H), 6.98-7.04 (m, 1 H), 6.74 (s, 1 H), 6.53 (s, 2 H), 4.86 (s, 1 H), 4.40 (d, J = 12.4 Hz, 2 H), 3.22 (s, 2 H), 2.79- 2.86 (m, 1 H), 1.89 (d, J = 16.0 Hz, 2 H), 1.51-1.62 (m, 2 H). ESI-MS: m/z = 329.4 (M + 1). |
| 17 | 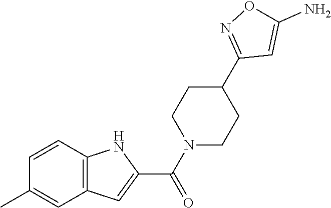 [SMILES: Cc1ccc2[nH]c(C(=O)N3CCC(c4cc(N)on4)CC3)cc2c1] | ++ | ¹H NMR (400 MHz, DMSO-d₆) δ 11.40 (s, 1H), 7.35 (s, 1H), 7.28 (d, J = 8.4 Hz, 1H), 6.98 (dd, J = 8.0, 1.6 Hz, 1H), 6.66 (s, 1H), 6.52 (s, 2H), 4.86 (s, 1H), 4.40-4.43 (m, 2H), 3.10- 3.20 (m, 2H), 2.81-2.83 (m, 1H), 2.34 (s, 3H), 1.87-1.90 (m, 2H), 1.55-1.58 (m, 2H). ESI-MS: m/z = 325.3 (M + 1). |
| 18 | 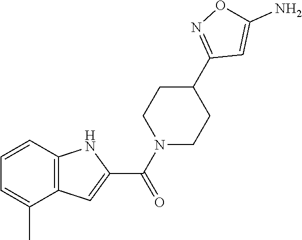 [SMILES: Cc1cccc2[nH]c(C(=O)N3CCC(c4cc(N)on4)CC3)cc12] | ++ | ¹H NMR (400 MHz, DMSO-d₆) δ 11.50 (s, 1H), 7.21 (d, J = 8.4 Hz, 1H), 7.05 (t, J = 6.8 Hz, 1H), 6.81 (d, J = 6.8 Hz, 1H), 6.77 (s, 1H), 6.52 (s, 2H), 4.87 (s, 1H), 4.43-4.46 (m, 2H), 3.10-3.20 (m, 2H), 2.82-2.83 (m, 1H), 2.46 (s, 3H), 1.88- 1.91 (m, 2H), 1.56-1.59 (m, 2H). ESI-MS: m/z = 325.3 (M + 1). |
| 19 | 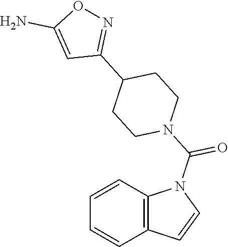 [SMILES: Nc1cc(C2CCN(C(=O)n3ccc4ccccc43)CC2)no1] | +++ | ¹HNMR (400 MHz, DMSO-d₆) δ 7.60 (t, J = 7.6 Hz, 2H), 7.53 (d, J = 3.6 Hz, 1H), 7.24 (t, J = 8.0 Hz, 1H), 7.15 (t, J = 8.0 Hz, 1H), 6.64 (d, J = 3.2 Hz, 1H), 6.53 (s, 2H), 4.88 (s, 1H), 3.87-3.90 (m, 2H), 3.15- 3.22 (m, 2H), 2.77-2.79 (m, 1H), 1.84-1.88 (m, 2H), 1.63-1.69 (m, 2H). ESI-MS: m/z = 311.3 (M + 1). |
| 20 | 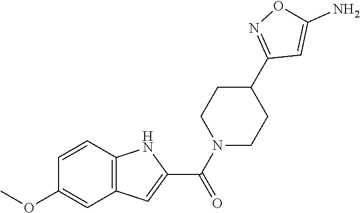 [SMILES: COc1ccc2[nH]c(C(=O)N3CCC(c4cc(N)on4)CC3)cc2c1] | ++ | 1H NMR (400 MHz, DMSO-d6) δ 11.38 (s, 1 H), 7.28 (d, J = 8.8 Hz, 1 H), 7.05 (d, J = 2.4 Hz, 1 H), 6.81 (dd, J = 9.2 Hz, J = 2.8 Hz, 1 H), 6.66 (s, 1 H), 6.52 (s, 2 H), 4.86 (s, 1 H), 4.41 (d, J = 13.2 Hz, 2 H), 3.72 (s, 3 H), 3.01-3.25 (m, 2 H), 2.75-2.90 (m, 1 H), 1.86-1.89 (m, 2 H), 1.55-1.57 (m, 2 H). ESI-MS: m/z = 341.4 (M + 1). |
| 21 | 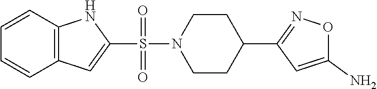 [SMILES: Nc1cc(C2CCN(S(=O)(=O)c3cc4ccccc4[nH]3)CC2)no1] | ++ | ¹HNMR (400 MHz, DMSO-d₆) δ 12.15 (s, 1H), 7.70 (d, J = 8.0 Hz, 1H), 7.48 (dd, J = 8.4, 0.8 Hz, 1H), 7.28-7.33 (m, 1H), 7.13 (dt, J = 8.0, 0.8 Hz, 1H), 7.01 (d, J = 1.2 Hz, 1H), 6.51 (s, 2H), 4.78 (s, 1H), 3.67-3.70 |
| | | | (m, 2H), 3.30-3.31 (m, 2H), 2.51-2.52 |
| | | | (m, 1H), 1.86-1.90 (m, 2H), 1.57-1.61 |
| | | | (m, 2H). ESI-MS: m/z = 347.2 |
| | | | (M + 1). |
| 22 | 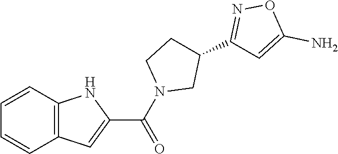 [SMILES: Nc1cc([C@H]2CCN(C(=O)c3cc4ccccc4[nH]3)C2)no1] | ++ | ¹H NMR (400 MHz, DMSO-d₆) δ 11.56 (s, 1 H), 7.63 (d, J = 8.0 Hz, 1 H), 7.44 (d, J = 8.0 Hz, 1 H), 7.19 (t, J = 7.6 Hz, 1 H), 7.04 (t, J = 7.6 Hz, 1 H), 6.98 (d, J = 5.6 Hz, 1 H), 6.62 (s, 2 H), 4.95 (d, J = 4.4 Hz, 1 H), 4.15- 4.17 (m, 1 H), 3.87-3.98 (m, 2 H), 3.41-3.71 (m, 2 H), 2.00-2.24 (m, 2 H). ESI-MS: m/z = 297.4 (M + 1). |
| 23 | 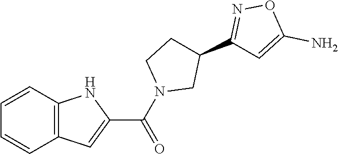 [SMILES: Nc1cc([C@@H]2CCN(C(=O)c3cc4ccccc4[nH]3)C2)no1] | ++ | ¹H NMR (400 MHz, DMSO-d₆) δ 11.56 (s, 1 H), 7.63 (d, J = 8.0 Hz, 1 H), 7.44 (d, J = 8.0 Hz, 1 H), 7.19 (t, J = 7.6 Hz, 1 H), 7.04 (t, J = 7.6 Hz, 1 H), 6.98 (d, J = 5.6 Hz, 1 H), 6.62 (s, 2 H), 4.95 (d, J = 4.4 Hz, 1 H), 4.15-4.17 (m, 1 H), 3.87-3.98 (m, 2 H), 3.41-3.71 (m, 2 H), 2.00-2.24 (m, 2 H). ESI-MS: m/z = 297.4 (M + 1). |
| 24 | 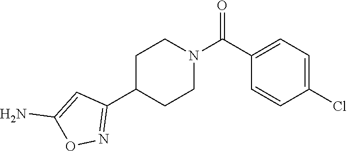 [SMILES: Nc1cc(C2CCN(C(=O)c3ccc(Cl)cc3)CC2)no1] | ++ | ¹HNMR (400 MHz, DMSO-d₆) δ 7.40-7.50 (m, 4H), 6.52 (s, 2H), 4.86 (s, 1H), 4.38-4.46 (m, 1H), 3.51- 3.59 (m, 1H), 3.08-3.16 (m, 1H), 2.88-2.94 (m, 1H), 2.72-2.79 (m, 1H), 1.70-1.89 (m, 2H), 1.48-1.56 (m, 2H). ESI-MS: m/z = 306.0 (M + 1). |
| 25 | 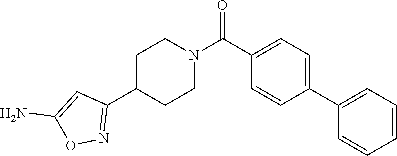 [SMILES: Nc1cc(C2CCN(C(=O)c3ccc(-c4ccccc4)cc3)CC2)no1] | +++ | ¹HNMR (400 MHz, DMSO-d₆): δ 7.68-7.72 (m, 4H), 7.45-7.48 (m, 4H), 7.36-7.40 (m, 1H), 6.52 (s, 2H), 4.87 (s, 1H), 4.42-4.52 (m, 1H), 3.60- 3.72 (m, 1H), 2.90-3.21 (m, 2H), 2.72-2.81 (m, 1H), 1.73-1.91 (m, 2H), 1.49-1.61 (m, 2H). ESI-MS: m/z = 348.3 (M + 1). |
| 26 | 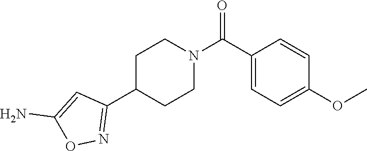 [SMILES: COc1ccc(C(=O)N2CCC(c3cc(N)on3)CC2)cc1] | − | ¹HNMR (400 MHz, DMSO-d₆): δ 7.32-7.36 (m, 2H), 6.94-6.97 (m, 2H), 6.51 (s, 2H), 4.86 (s, 1H), 4.19- 4.51 (m, 1H), 3.72-3.78 (m, 4H), 2.91- 3.11 (m, 2H), 2.72-2.77 (m, 1H), 1.74- 1.85 (m, 2H), 1.46-1.55 (m, 2H). ESI-MS: m/z = 302.2 (M + 1). |
| 27 | 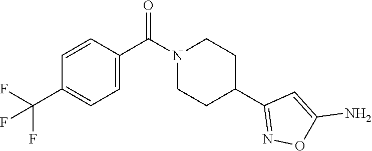 [SMILES: Nc1cc(C2CCN(C(=O)c3ccc(C(F)(F)F)cc3)CC2)no1] | ++ | ¹H NMR (400 MHz, DMSO-d₆) δ 7.80 (d, J = 8.0 Hz, 2 H), 7.63 (d, J = 8.4 Hz, 2 H), 6.54 (s, 2 H), 6.54 (s, 2 H), 4.88 (s, 1 H), 4.46 (d, J = 11.2 Hz, 1 H), 3.48 (d, J = 12.0 Hz, 1 H), 3.14 (t, J = 15.6 Hz, 1 H), 2.87-3.00 (m, 1H), 2.75-2.83 (m, 1 H), 1.90 (d, J = 12.8 Hz, 1 H), 1.74 (d, J = 16.0 Hz, 1 H), 1.48-1.62 (m, 2 H). ESI-MS: m/z 340.3 (M + 1). |
| 28 | 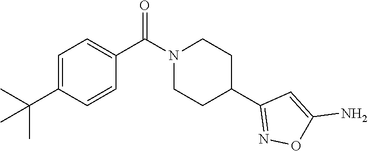 [SMILES: CC(C)(C)c1ccc(C(=O)N2CCC(c3cc(N)on3)CC2)cc1] | +++ | ¹H NMR (400 MHz, DMSO-d₆) δ 7.43 (d, J = 8.4 Hz, 2H), 7.3 (d, J = 8.4 Hz, 2H), 6.50 (s, 2H), 4.85 (s, 1H), 4.48-4.52 (m, 1H), 3.53-3.75 (m, 1H), 2.85-3.12 (m, 2H), 2.71-2.78 (m, 1H), 1.86-1.73 (m, 2H), 1.50 (d, J = 12.8H z, 2H), 1.27 (s, 9H). ESI-MS: m/z = 328.4 (M + 1). |
| 29 | 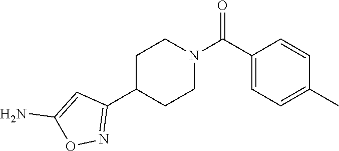 [SMILES: Cc1ccc(C(=O)N2CCC(c3cc(N)on3)CC2)cc1] | ++ | ¹HNMR (400 MHz, DMSO) δ 7.28 (d, J = 8 Hz, 2H), 7.24 (d, J = 7.6 Hz, 2H), 6.54 (s, 2H), 4.87 (s, 1H), 4.73 (brs, 1H), 3.64-3.59 (m, 2H), 3.05-2.88 (m, 2H), 2.80~2.75 (m, 1H), 1.87-1.73 (m, 4H), 1.52 (s, 2H). ESI-MS: m/z = 286.2 (M + 1). |
| 30 | 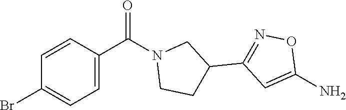 [SMILES: Nc1cc(C2CCN(C(=O)c3ccc(Br)cc3)C2)no1] | + | ¹H NMR (400 MHz, DMSO-d₆) δ 7.64 (t, J = 4.2 Hz, 2 H), 7.49 (d, J = 8.4 Hz, 2 H), 6.62 (d, J = 9.2 Hz, 2 H), 4.85-4.94 (m, 1 H), 3.45-3.83 (m, 4 H), 3.24-3.31 (m, 1 H), 2.14-2.27 (m, 1 H), 1.89-2.04 (m, 1 H). ESI-MS: m/z = 338.3 (M + 1). |
| 31 | 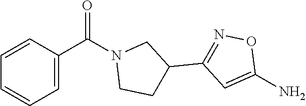 [SMILES: Nc1cc(C2CCN(C(=O)c3ccccc3)C2)no1] | − | ¹H NMR (400 MHz, DMSO-d₆) δ 7.44-7.53 (m, 5 H), 6.62 (d, J = 10.8 Hz, 2 H), 4.84-4.94 (m, 1 H), 3.49- 3.83 (m, 4 H), 3.21-3.28 (m, 1 H), 2.12-2.26 (m, 1 H), 1.87-2.03 (m, 1 H). ESI-MS: m/z = 258.4 (M + 1). |
| 32 | 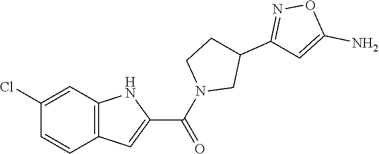 [SMILES: Nc1cc(C2CCN(C(=O)c3cc4ccc(Cl)cc4[nH]3)C2)no1] | +++ | ¹H NMR (400 MHz, DMSO-d₆) δ 11.71 (s, 1 H), 7.66 (d, J = 8.4 Hz, 1 H), 7.45 (s, 1 H), 7.02-7.07 (m, 2 H), 6.63 (s, 2 H), 4.95 (d, J = 6.8 Hz, 1 H), 3.41-4.19 (m, 5 H), 1.97-2.24 (m, 2 H). ESI-MS: m/z = 331.1 (M + 1) |
| 33 | 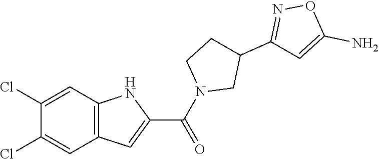 [SMILES: Nc1cc(C2CCN(C(=O)c3cc4cc(Cl)c(Cl)cc4[nH]3)C2)no1] | +++ | ¹H NMR (400 MHz, DMSO-d₆) δ 11.88 (s, 1 H), 7.92 (s, 1 H), 7.63 (s, 1 H), 7.01 (s, 1 H), 6.64 (d, J = 3.6 Hz, 2 H), 4.95 (d, J = 6.4 Hz, 1 H), 3.62-4.20 (m, 5 H), 1.94-2.24 (m, 2 H). ESI-MS: m/z = 365.2 (M + 1) |
| 34 | 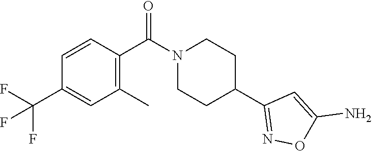 [SMILES: Cc1cc(C(F)(F)F)ccc1C(=O)N1CCC(c2cc(N)on2)CC1] | +++ | ¹HNMR (400 MHz, DMSO-d₆): δ 7.68 (s, 1H), 7.60-7.62 (m, 1H), 7.47-7.49 (m, 1H), 7.38-7.40 (m, 1H), 6.55 (s, 2H), 4.85-4.89 (m, 1H), 4.50- 4.53 (m, 1H), 3.21-3.28 (m, 1H), 3.07- 3.13 (m, 1H), 2.91-3.02 (m, 1H), 2.68- 2.81 (m, 1H), 2.28-2.33 (m, 3H), 1.79-1.96 (m, 1H), 1.37-1.77 (m, 3H). ESI-MS: m/z = 354.3 (M + 1) |
| 35 | 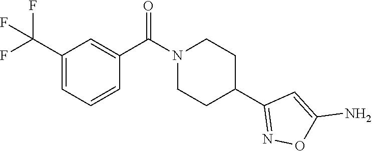 [SMILES: Nc1cc(C2CCN(C(=O)c3cccc(C(F)(F)F)c3)CC2)no1] | ++ | ¹H NMR (400 MHz, DMSO-d₆) δ 7.83 (d, J = 7.2 Hz, 1 H), 7.67-7.76 (m, 3 H), 6.55 (s, 2 H), 4.89 (s, 1 H), 4.46 (d, J = 9.2 Hz, 1 H), 3.49 (t, J = 5.6 Hz, 1 H), 3.19 (s, 1 H), 2.94 (s, 1 H), 2.75-2.82 (m, 1 H), 1.83-1.91 (m, 1 H), 1.73 (t, J = 11.2 Hz, 1 H), 1.58 (s, 2 H). ESI-MS: m/z = 340.1 (M + 1). |
| 36 | 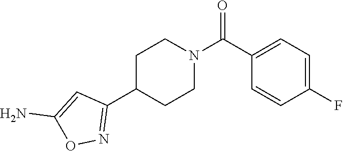 [SMILES: Nc1cc(C2CCN(C(=O)c3ccc(F)cc3)CC2)no1] | + | ¹H NMR (400 MHz, DMSO-d₆) δ 7.46-7.49 (m, 2 H), 7.23-7.30 (m, 2 H), 6.55 (s, 2 H), 4.88 (s, 1 H), 4.43 (bs, 1 H), 3.58 (bs, 1 H), 3.13 (bs, 1 H), 2.75-2.92 (m, 2 H), 1.76- 1.86 (m, 2 H), 1.50-1.58 (m, 2 H). ESI-MS: m/z = 290.2 (M + 1). |
| 37 | 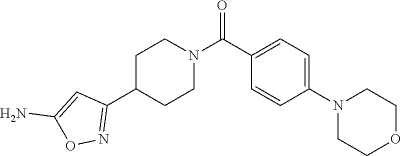 [SMILES: Nc1cc(C2CCN(C(=O)c3ccc(N4CCOCC4)cc3)CC2)no1] | − | ¹HNMR (400 MHz, DMSO-d₆) δ 7.29 (d, J = 8.8 Hz, 2H), 6.96 (d, J = 8.8 Hz, 2H), 6.54 (s, 2H), 4.88 (s, 1H), 3.93-4.38 (m, 2H), 3.74 (t, J = 5.2 Hz, 4H), 3.17 (t, J = 5.2 Hz, 4H), 2.94-3.08 (m, 2H), 2.72-2.80 (m, 1H), 1.77-1.86 (m, 2H), 1.46-1.57 (m, 2H). ESI-MS: m/z = 357.3 (M + 1). |
| 38 | 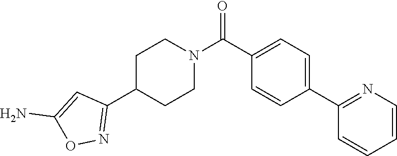 [SMILES: Nc1cc(C2CCN(C(=O)c3ccc(-c4ccccn4)cc3)CC2)no1] | + | ¹HNMR (400 MHz, DMSO-d₆) δ 8.69 (d, J = 4 Hz, 1H), 8.15 (d, J = 8 Hz, 2H), 8.02 (d, J = 8 Hz, 1H), 7.93-7.89 (m, 1H), 7.52 (d, J = 8.4 Hz, 2H), 7.40-7.37 (m, 1H), 6.55 (s, 2H), 4.90 (s, 1H), 4.52-4.45 (m, 1H), 3.69-3.62 (m, 1H), 3.21-3.12 (m, 1H), 2.96-2.75 (m, 1H), 1.90-1.78 (m, 2H), 1.57 (d, J = 10.8 Hz, 2H). ESI-MS: m/z = 349.1 (M + 1). |
| 39 | 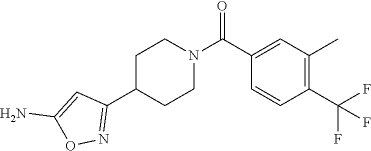 [SMILES: Cc1cc(C(=O)N2CCC(c3cc(N)on3)CC2)ccc1C(F)(F)F] | ++++ | ¹HNMR (400 MHz, DMSO-d₆) δ 7.73 (d, J = 8 Hz, 1H), 7.47 (s, 1H), 7.40 (d, J = 8 Hz, 1H), 6.54 (s, 2H), 4.88 (s, 1H), 4.45 (d, J = 12 Hz, 1H), 3.49 (d, J = 12.8 Hz, 1H), 3.14 (t, J = 12.6 Hz, 1H), 2.99-2.87 (m, 1H), 2.81-2.75 (m, 1H), 2.47 (s, 3H), 1.90 (d, J = 12.4 Hz, 1H), 1.74 (d, J = 12 Hz, 1H), 1.55 (t, J = 11.6 Hz, 2H). ESI-MS: m/z = 354.1 (M + 1). |
| 40 | 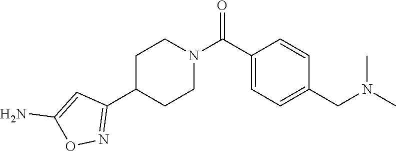 [SMILES: CN(C)Cc1ccc(C(=O)N2CCC(c3cc(N)on3)CC2)cc1] | − | ¹HNMR (400 MHz, DMSO-d₆) δ 7.35 (s, 4H), 6.55 (s, 2H), 4.88 (s, 1 H), 4.38-4.55 (m, 1H), 3.53-3.65 (m, 1H), 3.43 (s, 2 H), 3.06-3.21 (m, 1 H), 2.85- 2.96 (m, 1H), 2.73-2.80 (m, 1H), 2.16 (s, 6H), 1.70-1.73 (m, 2H), 1.46-1.60 (m, 2H). ESI-MS: m/z = 329.4 (M + 1). |
| 41 | 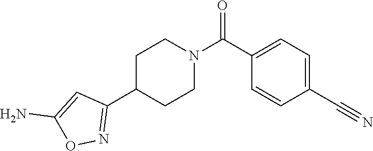 [SMILES: N#Cc1ccc(C(=O)N2CCC(c3cc(N)on3)CC2)cc1] | − | ¹H NMR (400 MHz, DMSO-d₆) δ 8.33 (d, J = 8.4 Hz, 2 H), 8.10 (d, J = 8.4 Hz, 2 H), 6.40 (s, 2 H), 5.47 (s, 1 H), 5.03 (bs, 1 H), 4.11 (bs, 1 H), 3.68 (bs, 1 H), 3.45 (bs, 1 H), 3.27- 3.38 (m, 1 H), 2.12-2.42 (m, 4 H). ESI-MS: m/z = 297.3 (M + 1). |
| 42 | 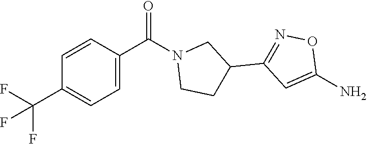 [SMILES: Nc1cc(C2CCN(C(=O)c3ccc(C(F)(F)F)cc3)C2)no1] | − | ¹H NMR (400 MHz, DMSO-d₆) δ 8.21-8.27 (m, 4 H), 6.48 (d, J = 13.2 Hz, 2 H), 5.45-5.54 (m, 1 H), 3.79-4.40 (m, 5 H), 2.69-2.83 (m, 1 H), 1.76 (s, 1 H). ESI-MS: m/z = 326.3 (M + 1) |
| 43 | 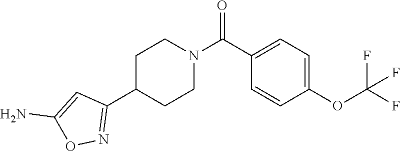 [SMILES: Nc1cc(C2CCN(C(=O)c3ccc(OC(F)(F)F)cc3)CC2)no1] | ++ | ¹H NMR (400 MHz, DMSO-d₆) δ 7.54-7.56 (m, 2 H), 7.44 (d, J = 7.6 Hz, 2 H), 6.55 (s, 2 H), 4.88 (s, 1 H), 4.62 (bs, 1 H), 3.55 (bs, 1 H), 3.15 (bs, 1 H), 2.92 (bs, 1 H), 2.74-2.82 (m, 1 H), 1.88 (bs, 1 H), 1.76 (bs, 1 H), 1.55 (d, J = 15.6 Hz, 2 H). ESI-MS: m/z = 356.1 (M + 1). |
| 44 | 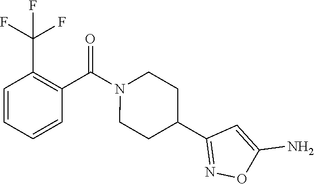 [SMILES: Nc1cc(C2CCN(C(=O)c3ccccc3C(F)(F)F)CC2)no1] | + | ¹H NMR (400 MHz, DMSO-d₆) δ 7.80-7.83 (m, 1 H), 7.55 (t, J = 7.4 Hz, 1 H), 7.65 (t, J = 7.6 Hz, 1 H), 7.44- 7.56 (m, 1 H), 6.55 (s, 2 H), 4.79- 4.89 (m, 1 H), 4.48 (d, J = 13.2 Hz, 1 H), 2.86-3.22 (m, 3 H), 2.76-2.82 (m, 1 H), 1.91 (t, J = 12.4 Hz, 1 H), 1.71 (t, J = 14.8 Hz, 1 H), 1.31-1.62 (m, 2 H). ESI-MS: m/z = 340.2 (M + 1). |
| 45 | 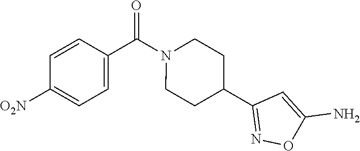 [SMILES: Nc1cc(C2CCN(C(=O)c3ccc([N+](=O)[O-])cc3)CC2)no1] | − | ¹H NMR (400 MHz, DMSO-d₆) δ 8.29 (d, J = 8.8 Hz, 2 H), 7.69 (d, J = 8.8 Hz, 2 H), 6.55 (s, 2 H), 4.89 (s, 1 H), 4.46 (d, J = 13.2 Hz, 1 H), 3.45 (d, J = 13.2 Hz, 1 H), 3.16 (t, J = 13.4 Hz, 1 H), 2.96 (t, J = 12.2 Hz, 1 H), 2.77- 2.84 (m, 1 H), 1.92 (d, J = 16.0 Hz, 1 H), 1.74 (d, J = 13.6 Hz, 1 H), 1.51- 1.64 (m, 2 H). ESI-MS: m/z = 317.3 (M + 1). |
| 46 | 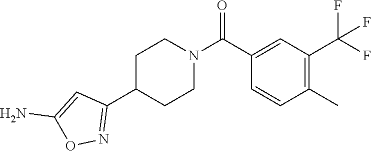 [SMILES: Cc1ccc(C(=O)N2CCC(c3cc(N)on3)CC2)cc1C(F)(F)F] | +++ | ¹H NMR (400 MHz, DMSO-d₆) δ 7.67 (s, 1 H), 7.61 (d, J = 7.6 Hz, 1 H), 7.52 (d, J = 7.6 Hz, 1 H), 6.54 (s, 2 H), 4.87 (s, 1 H), 4.43 (s, 1 H), 3.54 (s, 1 H), 3.16 (s, 1 H), 2.94 (s, 1 H), 2.75-2.81 (m, 1 H), 2.48-2.51 (m, 3 H), 1.88 (s, 1 H), 1.75 (s, 1 H), 1.56 (s, 2 H). ESI-MS: m/z = 354.3 (M + 1). |
| 47 | 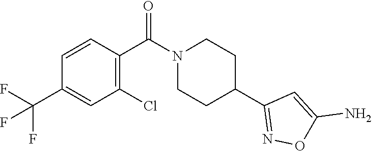 [SMILES: Nc1cc(C2CCN(C(=O)c3ccc(C(F)(F)F)cc3Cl)CC2)no1] | +++ | ¹H NMR (400 MHz, DMSO-d₆) δ 7.99 (s, 1 H), 7.81 (d, J = 7.6 Hz, 1 H), 7.60-7.73 (m, 1 H), 6.55 (s, 2 H), 4.86 (d, J = 18 Hz, 1 H), 4.48 (d, J = 13.2 Hz, 1 H), 2.23-3.27 (m, 1 H), 3.08- 3.18 (m, 1 H), 2.93-3.02 (m, 1 H), 2.77-2.84 (m, 1 H), 1.93 (t, J = 14.8 Hz, 1 H), 1.70-1.82 (m, 1 H), 1.47- 1.62 (m, 2 H). ESI-MS: m/z = 374.2 (M + 1). |
| 48 | 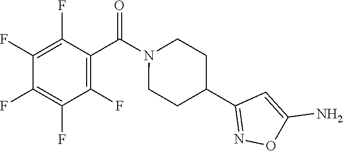 [SMILES: Nc1cc(C2CCN(C(=O)c3c(F)c(F)c(F)c(F)c3F)CC2)no1] | ++ | ¹H NMR (400 MHz, DMSO-d₆) δ 6.56 (s, 2 H), 4.86 (s, 1 H), 4.45 (d, J = 13.2 Hz, 1 H), 3.59 (d, J = 13.6 Hz, 1 H), 3.19-3.26 (m, 1 H), 3.00-3.07 (m, 1 H), 2.79-2.87 (m, 1 H), 1.96 (d, J = 13.2 Hz, 1 H), 1.82 (d, J = 13.2 Hz, 1 H), 1.38-1.55 (m, 2 H). |
| 49 | 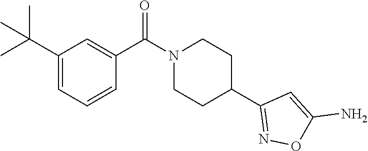 [SMILES: CC(C)(C)c1cccc(C(=O)N2CCC(c3cc(N)on3)CC2)c1] | ++ | ¹H NMR (400 MHz, DMSO-d₆) δ 7.46-7.49 (m, 1 H), 7.35-7.38 (m, 2 H), 7.18-7.21 (m, 1 H), 6.54 (s, 2 H), 4.89 (s, 1 H), 4.46 (s, 1 H), 3.58 (s, 1 H), 3.11 (s, 1 H), 2.94 (s, 1 H), 2.74- 2.80 (m, 1 H), 1.88 (s, 2 H), 1.75 (s, 2 H), 1.29 (s, 9 H). ESI-MS: m/z = 328.4 (M + 1). |
| 50 | 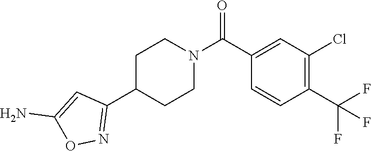 [SMILES: Nc1cc(C2CCN(C(=O)c3ccc(C(F)(F)F)c(Cl)c3)CC2)no1] | ++++ | ¹HNMR (400 MHz, DMSO) δ 7.93 (d, J = 8.4 Hz, 1H), 7.80 (s, 1H), 7.57 (d, J = 8 Hz, 1H), 6.54 (s, 2H), 4.88 (s, 1H), 4.44 (d, J = 13.6 Hz, 1H), 3.48-3.44 (m, 1H), 3.18-3.11 (m, 1H), 2.97-2.90 (m, 1H), 2.81-2.75 (m, 1H), 1.91-1.88 (m, 1H), 1.76-1.72 (m, 1H), 1.60-1.55 (m, 2H). ESI-MS: m/z = 374.1 (M + 1). |
| 51 | 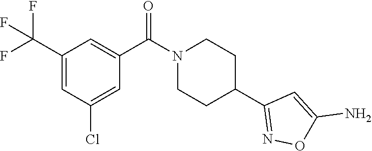 [SMILES: Nc1cc(C2CCN(C(=O)c3cc(Cl)cc(C(F)(F)F)c3)CC2)no1] | ++ | ¹HNMR (400 MHz, DMSO-d₆) δ 8.20 (s, 1H), 8.15 (s, 2H), 6.54 (s, 2H), 4.89 (s, 1H), 4.47-4.44 (m, 1H), 3.45-3.39 (m, 1H), 3.22-3.15 (m, 1H), 2.99-2.93 (m, 1H), 2.82-2.76 (m, 1H), 1.95-1.85 (m, 1H), 1.77-1.57 (m, 3H). ESI-MS: m/z = 374.1 (M + 1). |
| 52 | 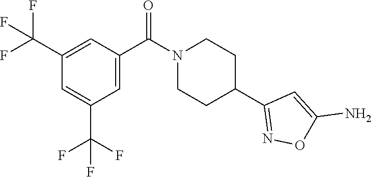 [SMILES: Nc1cc(C2CCN(C(=O)c3cc(C(F)(F)F)cc(C(F)(F)F)c3)CC2)no1] | ++ | ¹HNMR (400 MHz, DMSO) δ 7.96 (s, 1H), 7.85 (s, 1H), 7.77 (s, 1H), 6.54 (s, 2H), 4.89 (s, 1H), 4.45-4.42 (m, 1H), 3.47-3.42 (m, 1H), 3.20-3.14 (m, 1H), 2.96-2.91 (m, 1H), 2.81-2.74 (m, 1H), 1.90-1.87 (m, 1H), 1.74-1.71 (m, 1H), 1.62-1.57 (m, 2H). ESI-MS: m/z = 408.1 (M + 1). |
| 53 | 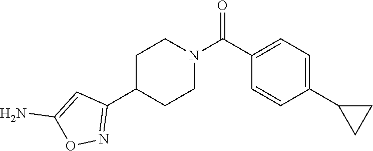 [SMILES: Nc1cc(C2CCN(C(=O)c3ccc(C4CC4)cc3)CC2)no1] | ++ | ¹HNMR (400 MHz, DMSO-d₆): δ 7.27 (d, J = 8.4 Hz, 2H), 7.12 (d, J = 8.4 Hz, 2H), 6.54 (s, 2H), 4.86 (s, 1H), 4.37-4.48 (m, 1H), 3.60-3.68 (m, 1H), 2.89-3.15 (m, 2H), 2.73-2.79 (m, 1H), 1.92-1.97 (m, 1H), 1.75- 1.84 (m, 2H), 1.50-1.56 (m, 2H), 0.96- 1.00 (m, 2H), 0.69-0.73 (m, 2H). ESI-MS: m/z = 312.3 (M + 1). |
| 54 | 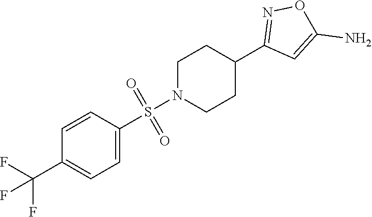 [SMILES: Nc1cc(C2CCN(S(=O)(=O)c3ccc(C(F)(F)F)cc3)CC2)no1] | ++ | ¹HNMR (400 MHz, DMSO-d₆) δ 7.97-8.06 (m, 4H), 6.53 (s, 2H), 4.81 (s, 1H), 3.65-3.68 (m, 2H), 2.42-2.55 (m, 3H), 1.85-1.89 (m, 2H), 1.54-1.64 (m, 2H). ESI-MS: m/z = 376.2 (M + 1). |
| 55 | 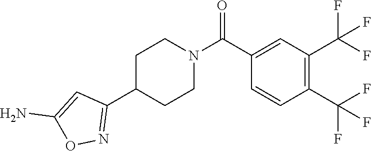 [SMILES: Nc1cc(C2CCN(C(=O)c3ccc(C(F)(F)F)c(C(F)(F)F)c3)CC2)no1] | ++ | ¹H NMR (400 MHz, DMSO-d₆) δ 8.13 (d, J = 8.4 Hz, 1 H), 8.07 (s, 1 H), 7.96 (d, J = 8.0 Hz, 1 H), 6.55 (s, 2 H), 4.89 (s, 1 H), 4.46 (d, J = 12.0 Hz, 1 H), 3.46 (d, J = 13.6 Hz, 1 H), 3.18 (t, J = 12.2 Hz, 1 H), 2.97 (t, J = 11.6 Hz, 1 H), 2.76-2.83 (m, 1 H), 1.91 (d, J = 12.8 Hz, 1 H), 1.74 (d, J = 12.0 Hz, 1 H), 1.57-1.64 (m, 2 H). ESI-MS: m/z 407.8 (M + 1). |
| 56 | 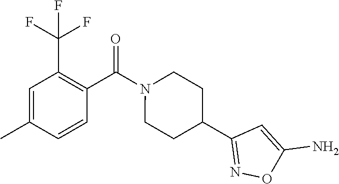 [SMILES: Cc1ccc(C(=O)N2CCC(c3cc(N)on3)CC2)c(C(F)(F)F)c1] | + | ¹HNMR (400 MHz, DMSO-d₆) δ 7.62-7.63 (m, 1H), 7.54 (d, J = 7.6 Hz, 1H), 7.32-7.42 (m, 1H), 6.55 (s, 2H), 4.78-4.89 (m, 1H), 4.47 (d, J = 13.2 Hz, 1H), 2.88-3.27 (m, 3H), 2.75-2.81 (m, 1H), 2.41 (s, 3H), 1.88-1.93 (m, 1H), 1.66-1.73 (m, 1H), 1.30-1.60 (m, 2H). ESI-MS: m/z = 354.2 (M + 1). |
| 57 | 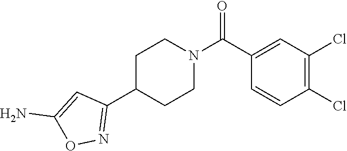 [SMILES: Nc1cc(C2CCN(C(=O)c3ccc(Cl)c(Cl)c3)CC2)no1] | ++++ | ¹HNMR (400 MHz, DMSO-d₆) δ 7.72-7.70 (m, 2H), 7.41-7.39 (m, 1H), 6.54 (s, 2H), 4.88 (s, 1H), 4.45-4.37 (m, 1H), 3.53-3.49 (m, 1H), 3.23- 2.59 (m, 3H), 1.86 (s, 1H), 1.73 (s, 1H), 1.60-1.50 (m, 2H). ESI-MS: m/z = 340.2 (M + 1). |
| 58 | 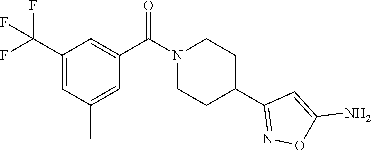 [SMILES: Cc1cc(C(=O)N2CCC(c3cc(N)on3)CC2)cc(C(F)(F)F)c1] | ++ | ¹HNMR (400 MHz, DMSO-d₆) δ 7.64 (s, 1H), 7.53 (s, 2H), 6.54 (s, 2H), 4.89 (s, 1H), 4.50-4.40 (m, 1H), 3.54- 3.43 (m, 1H), 3.25-3.09 (m, 1H), 3.01- 2.63 (m, 2H), 2.43 (s, 3H), 1.93-1.83 (m, 1H), 1.78-1.71 (m, 1H), 1.61-1.49 (m, 2H). ESI-MS: m/z = 354.1 (M + 1). |
| 59 | 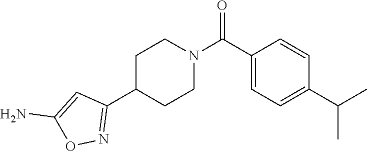 [SMILES: CC(C)c1ccc(C(=O)N2CCC(c3cc(N)on3)CC2)cc1] | +++ | ¹HNMR (400 MHz, DMSO-d₆) δ 7.31 (s, 4H), 6.54 (s, 2H), 4.87 (s, 1H), 4.53-4.34 (m, 1H), 3.73-3.53 (m, 1H), 3.21-3.02 (m, 1H), 2.96-2.63 (m, 3H), 1.95-1.73 (m, 2H), 1.58-1.48 (m, 2H), 1.21 (d, J = 6.8 Hz, 6H). ESI-MS: m/z = 314.3 (M + 1). |
| 60 | 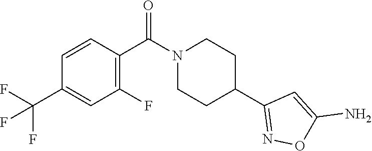 [SMILES: Nc1cc(C2CCN(C(=O)c3ccc(C(F)(F)F)cc3F)CC2)no1] | + | ¹HNMR (400 MHz, DMSO-d₆) δ 7.83 (d, J = 9.6 Hz, 1H), 7.69 (s, 2H), 6.55 (s, 2H), 4.86 (s, 1H), 4.48 (d, J = 12.4 Hz, 1H), 3.41-3.35 (m, 1H), 3.20- 3.13 (m, 1H), 3.00-2.94 (m, 1H), 2.83- 2.76 (m, 1H), 1.93-1.88 (m, 1H), 1.77- 1.73 (m, 1H), 1.58-1.43 (m, 2H). ESI-MS: m/z = 358.1 (M + 1). |
| 61 | 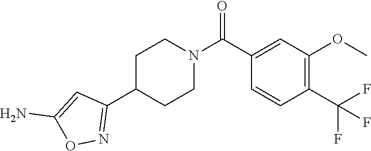 [SMILES: COc1cc(C(=O)N2CCC(c3cc(N)on3)CC2)ccc1C(F)(F)F] | ++ | ¹H NMR (400 MHz, DMSO-d₆) δ 7.67 (d, J = 8 Hz, 1H), 7.26 (s, 1H), 7.09 (d, J = 8.4 Hz, 1H), 6.54 (s, 2H), 4.88 (s, 1H), 4.47-4.44 (m, 1H), 3.91 (s, 3H), 3.51-3.48 (m, 1H), 3.17-3.10 (m, 1H), 2.97-2.90 (m, 1H), 2.83-2.75 (m, 1H), 1.94-1.87 (m, 1H), 1.77-1.71 (m, 1H), 1.64-1.51 (m, 2H). ESI-MS: m/z = 370.1 (M + 1). |
| 62 | 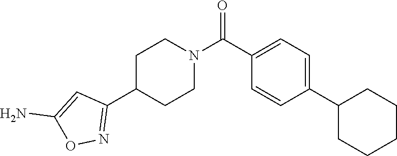 [SMILES: Nc1cc(C2CCN(C(=O)c3ccc(C4CCCCC4)cc3)CC2)no1] | +++ | ¹H NMR (400 MHz, DMSO-d₆) δ 7.27-7.32 (m, 4 H), 6.54 (s, 2 H), 4.88 (s, 1 H), 4.44 (s, 1 H), 3.62 (s, 1 H), 2.73-2.90 (m, 3 H), 2.50-2.56 (m, 1 H), 1.69-1.80 (m, 7 H), 1.22-1.57 (m, 7 H). ESI-MS: m/z = 354.3 (M + 1). |
| 63 | 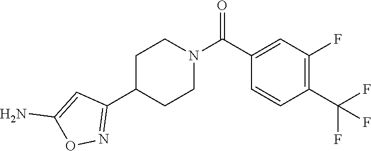 [SMILES: Nc1cc(C2CCN(C(=O)c3ccc(C(F)(F)F)c(F)c3)CC2)no1] | +++ | ¹H NMR (400 MHz, DMSO-d₆) δ 7.87 (t, J = 7.8 Hz, 1 H), 7.63 (d, J = 11.2 Hz, 1 H), 7.44 (d, J = 8.4 Hz, 1 H), 6.55 (s, 2 H), 4.89 (s, 1 H), 4.44 (d, J = 13.6 Hz, 1 H), 3.47 (d, J = 12.4 Hz, 1 H), 3.15 (t, J = 12.0 Hz, 1 H), 2.94 (t, J = 11.6 Hz, 1 H), 2.75 2.82 (m, 1 H), 1.90 (d, J = 13.6 Hz, 1 H), 1.73 (d, J = 11.2 Hz, 1 H), 1.55- |
| | | | 1.63 (m, 2 H). ESI-MS: m/z = 358.3 |
| | | | (M + 1). |
| 64 | 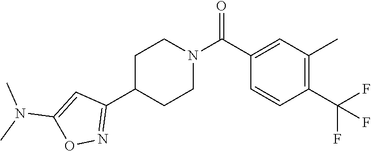 [SMILES: Cc1cc(C(=O)N2CCC(c3cc(N(C)C)on3)CC2)ccc1C(F)(F)F] | +++ | ¹H NMR (400 MHz, DMSO-d₆) δ 7.73 (d, J = 8.0 Hz, 1 H), 7.47 (s, 1 H), 7.40 (d, J = 8.0 Hz, 1 H), 5.14 (s, 1 H), 4.47 (d, J = 12.0 Hz, 1 H), 3.51 (d, J = 15.6 Hz, 1 H), 3.16 (t, J = 12.0 Hz, 1 H), 2.91-2.88 (m, 1 H), 2.88 (s, 6 H), 2.81-2.86 (m, 1 H), 2.48 (s, 3 H), 1.92 (d, J = 12.0 Hz, 1 H), 1.75 (d, J = 16.0 Hz, 1 H), 1.57 (t, J = 13.4 Hz, 2 H). ESI-MS: m/z = 382.1 (M + 1). |
| 65 | 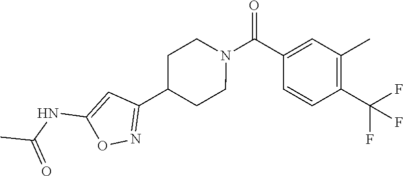 [SMILES: CC(=O)Nc1cc(C2CCN(C(=O)c3ccc(C(F)(F)F)c(C)c3)CC2)no1] | + | ¹HNMR (400 MHz, DMSO-d₆) δ 11.55 (s, 1H), 7.73 (d, J = 8.0 Hz, 1H), 7.50 (s, 1H), 7.43 (d, J = 8.0 Hz, 1H), 6.23 (s, 1H), 4.49 (d, J = 10.8 Hz, 1H), 3.51 (d, J = 11.6 Hz, 1H), 3.16 (t, J = 11.2 Hz, 1H), 2.92-3.00 (m, 2H), 2.48 (s, 3H), 2.08 (s, 3H), 1.96-1.97 (m, 1H), 1.78-1.81 (m, 1H), 1.59- 1.68 (m, 2H). ESI-MS: m/z = 396.2 (M + 1). |
| 66 | 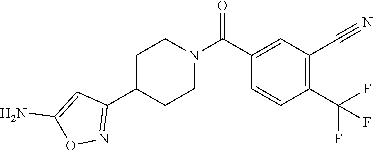 [SMILES: N#Cc1cc(C(=O)N2CCC(c3cc(N)on3)CC2)ccc1C(F)(F)F] | + | ¹H NMR (400 MHz, DMSO-d₆) δ 8.27 (s, 1 H), 8.08 (d, J = 8.4 Hz, 1 H), 7.96 (d, J = 8.4 Hz, 1 H), 6.55 (s, 2 H), 4.89 (s, 1 H), 4.45 (d, J = 11.2 Hz, 1 H), 3.45 (d, J = 14.8 Hz, 1 H), 3.16 (t, J = 12.0 Hz, 1 H), 2.96 (t, J = 12.4 Hz, 1 H), 2.76-2.83 (m, 1 H), 1.91 (d, J = 12.4 Hz, 1 H), 1.73 (d, J = 16.0 Hz, 1 H), 1.55-1.65 (m, 2 H). ESI-MS: m/z = 365.2 (M + 1). |
| 67 | 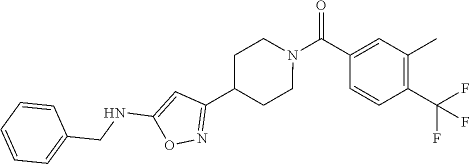 [SMILES: Cc1cc(C(=O)N2CCC(c3cc(NCc4ccccc4)on3)CC2)ccc1C(F)(F)F] | ++ | ¹HNMR (400 MHz, DMSO-d₆) δ 7.68-7.74 (m, 2H), 7.47 (s, 1H), 7.39 (d, J = 8.0 Hz, 1H), 7.33-7.34 (m, 4H), 7.23-7.28 (m, 1H), 5.02 (s, 1H), 4.43- 4.47 (m, 1H), 4.25 (d, J = 6.0 Hz, 2H), 3.47-3.50 (m, 1H), 3.10-3.16 (m, 1H), 2.91-2.94 (m, 1H), 2.77-2.82 (m, 1H), 2.47 (s, 3H), 1.88-1.91 (m, 1H), 1.72- 1.75 (m, 1H), 1.48-1.56 (m, 2H). ESI-MS: m/z = 444.3 (M + 1). |
| 68 | 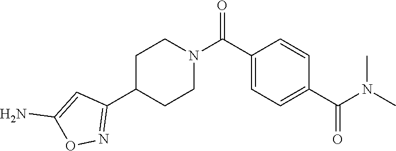 [SMILES: CN(C)C(=O)c1ccc(C(=O)N2CCC(c3cc(N)on3)CC2)cc1] | − | ¹H NMR (400 MHz, DMSO-d₆) δ 7.45 (s, 4 H), 6.54 (s, 2 H), 4.89 (s, 1 H), 4.47 (s, 1 H), 3.56 (s, 1 H), 3.31 (s, 1 H), 3.13 (s, 1 H), 2.99 (s, 3 H), 2.91 (s, 3 H), 2.76-2.81 (m, 1 H), 1.91 (s, 1 H), 1.72 (s, 1 H), 1.55 (s, 2 H). ESI-MS: m/z = 343.2 (M + 1). |
| 69 | 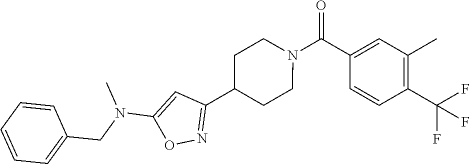 [SMILES: Cc1cc(C(=O)N2CCC(c3cc(N(C)Cc4ccccc4)on3)CC2)ccc1C(F)(F)F] | ++ | ¹H NMR (400 MHz, DMSO-d₆) δ 7.31 (d, J = 8.0 Hz, 1 H), 7.47 (s, 1 H), 7.34-7.41 (m, 3 H), 7.25-7.31 (m, 3 H), 5.22 (s, 1 H), 4.46 (s, 3 H), 3.51 (d, J = 12.0 Hz, 1 H), 3.16 (t, J = 11.2 Hz, 1 H), 2.95 (d, J = 12.4 Hz, 1 H), 2.89 (s, 3 H), 2.81-2.87 (m, 1 H), 2.48 (s, 3 H), 1.93 (d, J = 14.0 Hz, 1 H), 1.77 (d, J = 11.2 Hz, 1 H), 1.58 (t, J = 11.8 Hz, 2 H). ESI-MS: m/z = 458.2 (M + 1). |
| 70 | 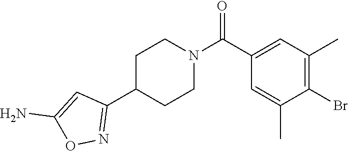 [SMILES: Cc1cc(C(=O)N2CCC(c3cc(N)on3)CC2)cc(C)c1Br] | ++ | ¹H NMR (400 MHz, DMSO-d₆) δ 7.66 (s, 2 H), 6.39 (s, 2 H), 5.46 (s, 1 H), 4.99 (bs, 1 H), 4.23 (bs, 1 H), 3.30-3.45 (m, 3 H), 2.89 (s, 6 H), 2.35-2.37 (m, 2 H), 2.05-2.14 (m, 2 H). ESI-MS: m/z = 380.3 (M + 1). |
| 71 | 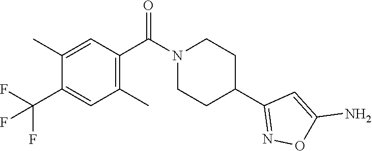 [SMILES: Cc1cc(C(F)(F)F)c(C)cc1C(=O)N1CCC(c2cc(N)on2)CC1] | + | ¹HNMR (400 MHz, DMSO-d₆) δ 7.58 (s, 1H), 7.24-7.33 (m, 1H), 6.54 (s, 2H), 4.84-4.89 (m, 1H), 4.49-4.52 (m, 1H), 3.23-3.27 (m, 1H), 3.07-3.13 (m, 1H), 2.92-2.95 (m, 1H), 2.75-2.81 (m, 1H), 2.41 (s, 3H), 2.21-2.27 (m, 3H), 1.89-1.95 (m, 1H), 1.69-1.77 (m, 1H), 1.34-1.58 (m, 2H). ESI-MS: m/z = 368.2 (M + 1). |
| 72 | 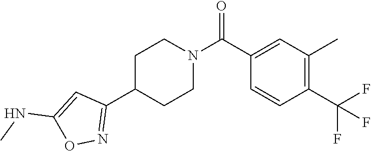 [SMILES: CNc1cc(C2CCN(C(=O)c3ccc(C(F)(F)F)c(C)c3)CC2)no1] | ++++ | ¹H NMR (400 MHz, CDCl₃) δ 7.59 (t, J = 7.4 Hz, 1H), 7.21 (d, J = 11.9 Hz, 2H), 4.78 (s, 1H), 4.61 (s, 1H), 4.43 (d, J = 4.4 Hz, 1H), 3.67 (d, J = 8.3 Hz, 1H), 2.90-2.74 (m, 4H), 2.45 (s, 3H), 1.91 (d, J = 58.2 Hz, 2H), 1.64 (d, J = 37.7 Hz, 3H), 1.19 (s, 1H). M + H calc’d 367.2. M + H found 368.2. |
| 73 | 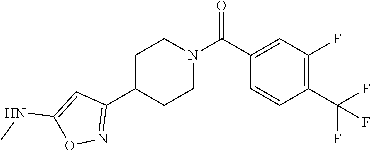 [SMILES: CNc1cc(C2CCN(C(=O)c3ccc(C(F)(F)F)c(F)c3)CC2)no1] | +++ | ¹H NMR (400 MHz, CDCl₃) δ 7.67 (t, J = 7.4 Hz, 1H), 7.29 (s, 1H), 7.24 (s, 1H), 4.84 (s, 1H), 4.73-4.50 (m, 2H), 3.70 (s, 1H), 3.17 (s, 1H), 3.10- 2.98 (m, 1H), 2.97-2.90 (m, 1H), 2.89 (d, J = 5.2 Hz, 3H), 2.00 (dd, J = 29.1, 23.3 Hz, 2H), 1.76 (s, 2H). ESI-MS: m/z = 371.9 (M + 1). |
| 74 | 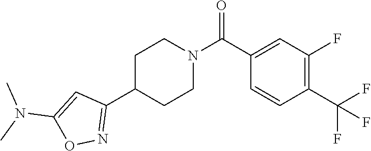 [SMILES: CN(C)c1cc(C2CCN(C(=O)c3ccc(C(F)(F)F)c(F)c3)CC2)no1] | ++ | ¹H NMR (400 MHz, CDCl₃) δ 7.67 (t, J = 7.4 Hz, 1H), 7.28 (d, J = 6.6 Hz, 1H), 7.24 (s, 1H), 4.76 (s, 1H), 4.63 (s, 1H), 3.70 (dd, J = 10.2, 3.9 Hz, 1H), 3.26-3.00 (m, 2H), 2.97 (s, 6H), 2.91 (tt, J = 11.1, 3.9 Hz, 1H), 2.13-1.91 (m, 2H), 1.78 (s, 2H). ESI-MS: m/z = 385.9 (M + 1) |
| 75 | 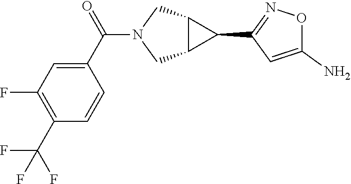 [SMILES: Nc1cc([C@H]2[C@@H]3CN(C(=O)c4ccc(C(F)(F)F)c(F)c4)C[C@@H]32)no1] | ++ | ¹H NMR (400 MHz, CDCl₃) δ 7.67 (t, J = 7.4 Hz, 1H), 7.31 (t, J = 9.9 Hz, 2H), 4.88 (s, 1H), 4.40 (s, 2H), 4.30 (d, J = 12.5 Hz, 1H), 3.72 (dd, J = 10.8, 3.7 Hz, 1H), 3.67-3.51 (m, 2H), 2.10- 1.98 (m, 2H), 1.65 (t, J = 3.4 Hz, 1H). ESI-MS: m/z = 356.09 (M + 1). |
| 76 | 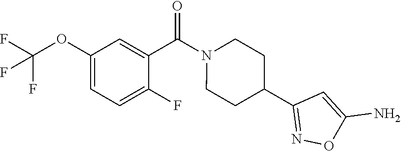 [SMILES: Nc1cc(C2CCN(C(=O)c3cc(OC(F)(F)F)ccc3F)CC2)no1] | ++ | ¹H NMR (400 MHz, CDCl₃) δ 7.48- 7.32 (m, 1H), 7.25 (d, J = 4.1 Hz, 1H), 7.17-7.10 (m, 1H), 5.00 (s, 1H), 4.69 (d, J = 13.4 Hz, 1H), 4.49 (s, 2H), 3.60 (d, J = 13.6 Hz, 1H), 3.18 (s, 1H), 3.02 (t, J = 11.9 Hz, 1H), 2.90 (tt, J = 11.2, 3.8 Hz, 1H), 2.05 (dt, J = 13.7, 7.0 Hz, 1H), 1.98-1.88 (m, 1H), 1.84- 1.71 (m, 2H). ESI-MS: m/z = 373.8 (M + 1). |
| 77 | 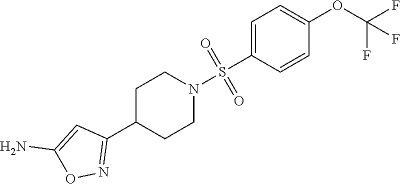 [SMILES: Nc1cc(C2CCN(S(=O)(=O)c3ccc(OC(F)(F)F)cc3)CC2)no1] | ++ | ¹H NMR (400 MHz, CDCl₃) δ 7.85- 7.79 (m, 2H), 7.43-7.33 (m, 2H), 4.95 (s, 1H), 4.40 (s, 2H), 3.81 (dt, J = 11.8, 3.0 Hz, 2H), 2.59 (tt, J = 11.3, 3.9 Hz, 1H), 2.49 (td, J = 11.7, 2.7 Hz, 2H), 1.99 (dd, J = 13.4, 2.9 Hz, 2H), 1.90- 1.75 (m, 2H). ESI-MS: m/z = 391.8 (M + 1). |
| 78 | 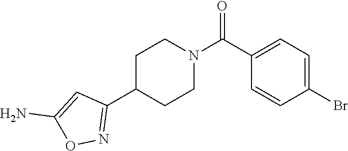 [SMILES: Nc1cc(C2CCN(C(=O)c3ccc(Br)cc3)CC2)no1] | ++ | ¹H NMR (400 MHz, CDCl₃) δ 7.57- 7.53 (m, 2H), 7.32-7.27 (m, 2H), 4.99 (s, 1H), 4.66 (s, 1H), 4.43 (s, 2H), 3.79 (s, 1H), 3.03 (s, 2H), 2.88 (ddd, J = 11.2, 7.6, 3.7 Hz, 1H), 2.01 (d, J = 5.6 Hz, 2H), 1.69 (s, 2H). ESI-MS: m/z = 351.8 (M + 1). |
| 79 | 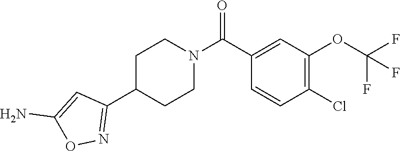 [SMILES: Nc1cc(C2CCN(C(=O)c3ccc(Cl)c(OC(F)(F)F)c3)CC2)no1] | ++ | ¹H NMR (400 MHz, CDCl₃) δ 7.53 (d, J = 8.2 Hz, 1H), 7.39 (s, 1H), 7.31 (d, J = 8.2 Hz, 1H), 5.00 (s, 1H), 4.62 (s, 1H), 4.48 (s, 2H), 3.76 (s, 1H), 3.05 (s, 2H), 2.97-2.81 (m, 1H), 2.01 (s, 2H), 1.73 (s, 2H). ESI-MS: m/z = 389.8 (M + 1). |
| 80 | 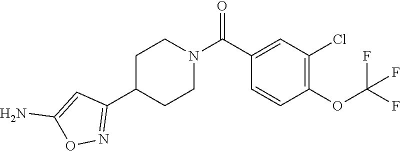 [SMILES: Nc1cc(C2CCN(C(=O)c3ccc(OC(F)(F)F)c(Cl)c3)CC2)no1] | ++++ | ¹H NMR (400 MHz, CDCl₃) δ 7.54 (t, J = 4.2 Hz, 1H), 7.40-7.31 (m, 2H), 5.00 (s, 1H), 4.62 (s, 1H), 4.46 (s, 2H), 3.77 (s, 1H), 3.10 (d, J = 44.8 Hz, 2H), 2.90 (tt, J = 11.2, 3.7 Hz, 1H), 2.01 (d, J = 4.9 Hz, 2H), 1.69 (s, 2H). ESI-MS: m/z = 389.8 (M + 1). |
| 81 | 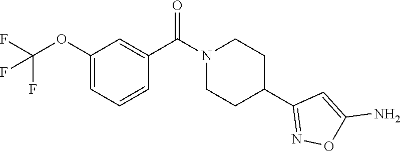 [SMILES: Nc1cc(C2CCN(C(=O)c3cccc(OC(F)(F)F)c3)CC2)no1] | + | ¹H NMR (400 MHz, CDCl₃) δ 7.49- 7.28 (m, 2H), 7.27-7.11 (m, 2H), 4.99 (s, 1H), 4.69 (d, J = 13.0 Hz, 1H), 4.52 (s, 2H), 3.80-3.56 (m, 1H), 3.15 (s, 1H), 3.09-2.97 (m, 1H), 2.90 (tt, J = 11.2, 3.8 Hz, 1H), 2.10-2.01 (m, 1H), 1.93 (d, J = 11.4 Hz, 1H), 1.74 (ddd, J = 25.0, 11.6, 4.2 Hz, 2H). ESI-MS: m/z = 355.9 (M + 1). |
| 82 | 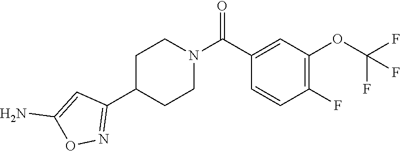 [SMILES: Nc1cc(C2CCN(C(=O)c3ccc(F)c(OC(F)(F)F)c3)CC2)no1] | +++ | 1H NMR (400 MHz, CDCl3) δ 7.43- 7.34 (m, 2H), 7.28 (s, 1H), 7.26-7.22 (m, 1H), 5.00 (s, 1H), 4.60 (s, 1H), 4.44 (s, 2H), 3.83 (d, J = 37.5 Hz, 1H), 3.10 (s, 2H), 2.90 (tt, J = 11.2, 3.9 Hz, 1H), 1.99 (s, 2H), 1.76 (m, 1H). ESI- MS: m/z = 373.8 (M + 1). |
| 83 | 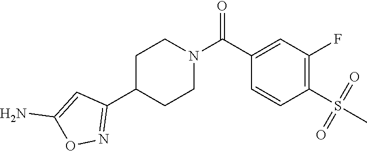 [SMILES: CS(=O)(=O)c1ccc(C(=O)N2CCC(c3cc(N)on3)CC2)cc1F] | − | ¹H NMR (400 MHz, MeOD) δ 7.92 (t, J = 7.5 Hz, 1H), 7.42-7.34 (m, 2H), 4.90 (s, 1H), 4.51 (d, J = 12.8 Hz, 1H), 3.55 (d, J = 12.8 Hz, 1H), 3.19 (s, 3H), 3.14 (d, J = 14.8 Hz, 1H), 2.97 (dd, J = 22.9, 10.3 Hz, 1H), 2.79 (tt, J = 11.4, 3.8 Hz, 1H), 1.94 (t, J = 6.2 Hz, 1H), 1.78 (d, J = 10.3 Hz, 1H), 1.70-1.49 (m, 2H). ESI-MS: m/z = 367.8 (M + 1) |
| 84 | 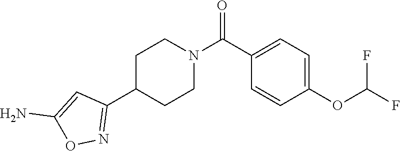 [SMILES: Nc1cc(C2CCN(C(=O)c3ccc(OC(F)F)cc3)CC2)no1] | + | ¹H NMR (400 MHz, CDCl₃) δ 7.47- 7.40 (m, 2H), 7.16 (d, J = 8.6 Hz, 2H), 6.54 (t, J = 73.4 Hz, 1H), 4.99 (s, 1H), 4.66 (s, 1H), 4.45 (s, 2H), 3.82 (s, 1H), 3.06 (s, 2H), 2.89 (ddd, J = 15.2, 7.6, 3.9 Hz, 1H), 2.08-1.89 (m, 2H), 1.69 (s, 1H). ESI-MS: m/z = 337.9 (M + 1) |
| 85 | 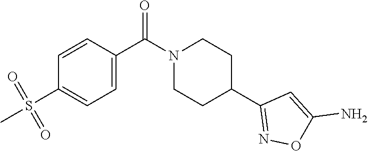 [SMILES: CS(=O)(=O)c1ccc(C(=O)N2CCC(c3cc(N)on3)CC2)cc1] | − | ¹H NMR (400 MHz, CDCl₃) δ 8.03- 7.98 (m, 2H), 7.62-7.57 (m, 2H), 5.00 (s, 1H), 4.66 (s, 1H), 4.44 (s, 2H), 3.66 (s, 1H), 3.17 (s, 1H), 3.08 (d, J = 2.3 Hz, 4H), 2.91 (tt, J = 11.0, 3.8 Hz, 1H), 2.06 (s, 1H), 1.91 (s, 1H), 1.76 (s, 1H), 1.67 (s, 1H). ESI-MS: m/z = 349.8 (M + 1). |
| 86 | 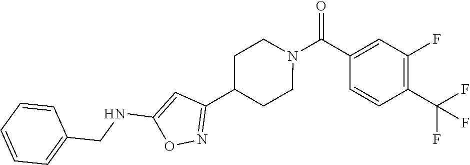 [SMILES: O=C(c1ccc(C(F)(F)F)c(F)c1)N1CCC(c2cc(NCc3ccccc3)on2)CC1] | +++ | ¹H NMR (400 MHz, CDCl₃) δ 7.65 (dd, J = 8.5, 2.7 Hz, 1H), 7.37-7.30 (m, 5H), 7.27 (s, 1H), 7.24 (d, J = 10.4 Hz, 1H), 4.95 (t, J = 5.8 Hz, 1H), 4.84 (s, 1H), 4.63 (t, J = 6.0 Hz, 1H), 4.35 (d, J = 5.9 Hz, 2H), 3.68 (s, 1H), 3.24- 2.96 (m, 2H), 2.89 (tt, J = 11.1, 3.8 Hz, 1H), 2.14-1.90 (m, 2H), 1.67 (s, 2H). ESI-MS: m/z = 447.9 (M + 1). |
| 87 | 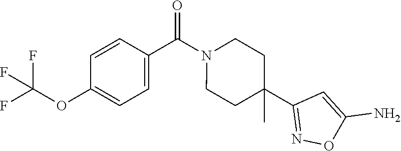 [SMILES: CC1(c2cc(N)on2)CCN(C(=O)c2ccc(OC(F)(F)F)cc2)CC1] | − | ¹H NMR (400 MHz, CDCl₃) δ 7.46- 7.40 (m, 2H), 7.25-7.21 (m, 2H), 5.00 (s, 1H), 4.42 (s, 2H), 4.25 (s, 1H), 3.49 (s, 1H), 3.39 (s, 1H), 3.28 (s, 1H), 2.09 (d, J = 13.0 Hz, 2H), 1.67 (s, 1H), 1.45 (s, 1H), 1.29 (s, 3H). ESI-MS: m/z = 369.9 (M + 1). |
| 88 | 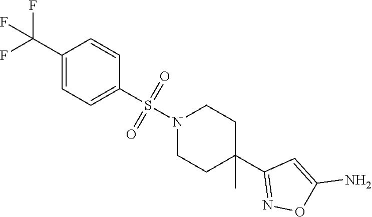 [SMILES: CC1(c2cc(N)on2)CCN(S(=O)(=O)c2ccc(C(F)(F)F)cc2)CC1] | + | ¹H NMR (400 MHz, CDCl₃) δ 7.85 (d, J = 8.2 Hz, 2H), 7.77 (d, J = 8.3 Hz, 2H), 4.90 (s, 1H), 4.38 (s, 2H), 3.51 (dt, J = 12.0, 3.8 Hz, 2H), 2.76 (td, J = 11.9, 2.7 Hz, 2H), 2.13 (d, J = 13.7 Hz, 2H), 1.80-1.67 (m, 2H), 1.21 (s, 3H). ESI-MS: m/z = 389.8 (M + 1) |
| 89 | 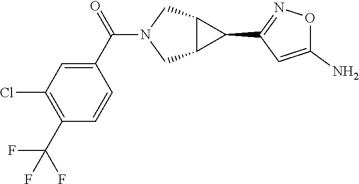 [SMILES: Nc1cc([C@H]2[C@@H]3CN(C(=O)c4ccc(C(F)(F)F)c(Cl)c4)C[C@@H]32)no1] | ++++ | ¹H NMR (400 MHz, CDCl₃) δ 7.74 (d, J = 8.1 Hz, 1H), 7.59 (s, 1H), 7.43 (d, J = 8.1 Hz, 1H), 4.89 (s, 1H), 4.40 (s, 2H), 4.29 (d, J = 12.5 Hz, 1H), 3.72 (dd, J = 10.9, 3.7 Hz, 1H), 3.67- 3.55 (m, 2H), 2.09-1.96 (m, 2H), 1.66 (t, J = 3.4 Hz, 1H). ESI-MS: m/z = 371.8 (M + 1). |
| 90 | 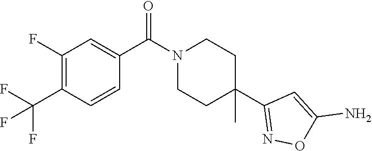 [SMILES: CC1(c2cc(N)on2)CCN(C(=O)c2ccc(C(F)(F)F)c(F)c2)CC1] | + | ¹H NMR (400 MHz, CDCl₃) δ 7.65 (t, J = 7.6 Hz, 1H), 7.25-7.20 (m, 2H), 5.00 (s, 1H), 4.43 (s, 2H), 4.32-4.24 (m, 1H), 3.50-3.35 (m, 2H), 3.30-3.20 (m, 1H), 2.10 (s, 2H), 1.64 (s, 2H), 1.30 (s, 3H). ESI-MS: m/z = 372.13 (M + 1). |
| 91 | 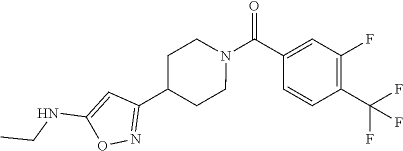 [SMILES: CCNc1cc(C2CCN(C(=O)c3ccc(C(F)(F)F)c(F)c3)CC2)no1] | ++++ | ¹H NMR (400 MHz, CDCl₃) δ 7.67 (t, J = 7.5 Hz, 1H), 7.29 (s, 1H), 7.24 (s, 1H), 4.84 (s, 1H), 4.64 (s, 1H), 4.48 (s, 1H), 3.70 (s, 1H), 3.28-3.12 (m, 3H), 3.04 (s, 1H), 2.91 (tt, J = 11.0, 3.7 Hz, 1H), 2.16-1.88 (m, 2H), 1.76 (s, 2H), 1.25 (d, J = 7.2 Hz, 3H). ESI-MS: m/z = 385.9 (M + 1). |
| 92 | 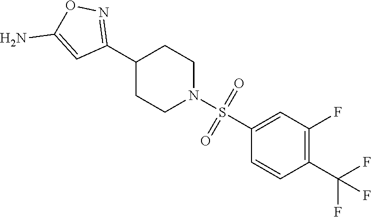 [SMILES: Nc1cc(C2CCN(S(=O)(=O)c3ccc(C(F)(F)F)c(F)c3)CC2)no1] | +++ | ¹H NMR (400 MHz, CDCl₃) δ 7.81 (t, J = 7.3 Hz, 1H), 7.69-7.60 (m, 2H), 4.96 (s, 1H), 4.43 (s, 2H), 3.80 (d, J = 11.9 Hz, 2H), 2.71-2.53 (m, 3H), 2.05- 1.99 (m, 2H), 1.88-1.76 (m, 2H). ESI- MS: m/z = 393.8 (M + 1) |
| 93 | 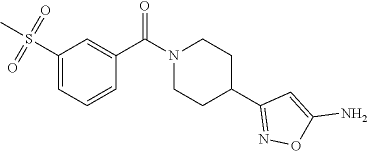 | − | ″1H NMR (400 MHz, CD3SOCD3, 299K) δ (ppm) = 8.53-8.36 (m, 1H), 8.34-8.22 (m, 1H), 8.18 (br d, J = 7.5 Hz, 1H), 8.01-7.76 (m, 1H), 6.54 (s, 2H), 4.90 (s, 1H), 4.52-4.36 (m, 1H), 3.59-3.47 (m, 1H), 3.35-3.30 (m, 3H), 3.16-3.12 (m, 1H), 3.00-2.90 (m, 1H), 2.87-2.75 (m, 1H), 2.03-1.85 (m, 1H), 1.82-1.70 (m, 1H), 1.67-1.50 (m, 2H). ESI-MS: m/z = |
| | | | 349.9 (M + 1).″ |
| 94 | 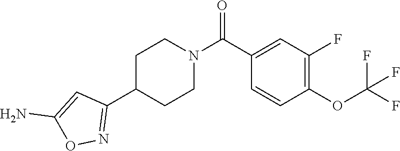 [SMILES: Nc1cc(C2CCN(C(=O)c3ccc(OC(F)(F)F)c(F)c3)CC2)no1] | +++ | 1H NMR (400 MHz, CD3SOCD3, 299K) Shift (ppm = 7.70-7.57 (m, 2H), 7.45-7.30 (m, 1H), 6.54 (s, 2H), 4.90 (s, 1H), 4.52-4.36 (m, 1H), 3.59-3.47 (m, 1H), 3.16- 3.12 (m, 1H), 3.00-2.90 (m, 1H), 2.87- 2.75 (m, 1H), 2.03-1.85 (m, 1H), 1.82- 1.70 (m, 1H), 1.67-1.50 (m, 2H). ESI- MS: m/z = 373.9 (M + 1). |
| 95 | 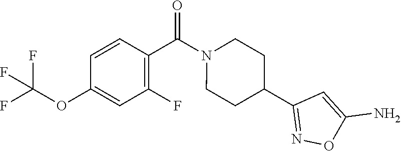 [SMILES: Nc1cc(C2CCN(C(=O)c3ccc(OC(F)(F)F)cc3F)CC2)no1] | ++ | 1H NMR (400 MHz, CD3SOCD3, 299K) Shift (ppm) = 7.67-7.56 (m, 1H), 7.56-7.49 (m, 1H), 7.35 (br d, J = 9.3 Hz, 1H), 6.55 (s, 2H), 4.87 (s, 1H), 4.56-4.38 (m, 1H), 3.41 (br d, J = 13.3 Hz, 1H), 3.14 (br d, J = 7.3 Hz, 1H), 2.97 (dt, J = 2.5, 12.5 Hz, 1H), 2.81 (tt, J = 3.7, 11.4 Hz, 1H), 1.94 (br d, J = 12.3 Hz, 1H), 1.80 (br d, J = 13.1 Hz, 1H), 1.67-1.39 (m, 2H). ESI-MS: |
| | | | m/z = 373.9 (M + 1). |
| 96 | 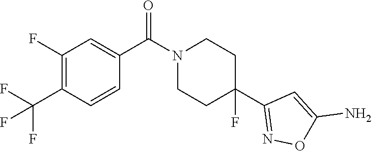 [SMILES: Nc1cc(C2(F)CCN(C(=O)c3ccc(C(F)(F)F)c(F)c3)CC2)no1] | + | ¹H NMR (400 MHz, CDCl₃) δ 7.68 (t, J = 7.5 Hz, 1H), 7.32-7.26 (m, 2H), 5.22 (s, 1H), 4.55 (s, 3H), 3.60 (s, 1H), 3.50 (s, 1H), 3.37 (s, 1H), 2.37-1.99 (m, 4H). ESI-MS: m/z = 375.9 (M + 1) |
| 97 | 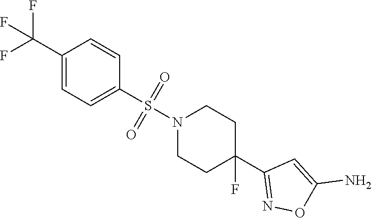 [SMILES: Nc1cc(C2(F)CCN(S(=O)(=O)c3ccc(C(F)(F)F)cc3)CC2)no1] | ++ | ¹H NMR (400 MHz, CDCl₃) δ 7.92 (d, J = 8.2 Hz, 2H), 7.82 (d, J = 8.3 Hz, 2H), 5.15 (s, 1H), 4.50 (s, 2H), 3.77-3.67 (m, 2H), 2.83 (ddd, J = 12.0, 7.6, 3.3 Hz, 2H), 2.23-2.16 (m, 3H), 2.06-1.96 (m, 1H). ESI-MS: m/z = 393.8 (M + 1) |
| 98 | 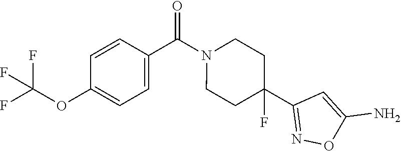 [SMILES: Nc1cc(C2(F)CCN(C(=O)c3ccc(OC(F)(F)F)cc3)CC2)no1] | + | ¹H NMR (400 MHz, CDCl₃) δ 7.47 (d, J = 8.0 Hz, 2H), 7.27 (d, J = 8.0 Hz, 2H), 5.22 (s, 1H), 4.53 (s, 3H), 3.69 (s, 1H), 3.43 (s, 2H), 2.22-2.09 (m, 4H). ESI-MS: m/z = 373.9 (M + 1) |
| 99 | 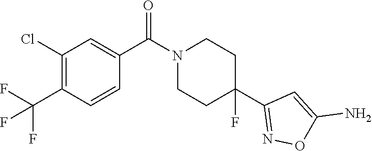 [SMILES: Nc1cc(C2(F)CCN(C(=O)c3ccc(C(F)(F)F)c(Cl)c3)CC2)no1] | ++ | ¹H NMR (400 MHz, CDCl₃) δ 7.75 (d, J = 8.0 Hz, 1H), 7.56 (s, 1H), 7.40 (dd, J = 8.0, 0.7 Hz, 1H), 5.23 (s, 1H), 4.54 (s, 3H), 3.66-3.31 (m, 3H), 2.28-2.06 (m, 4H). ESI-MS: m/z = 391.8 (M + 1). |
| 100 | 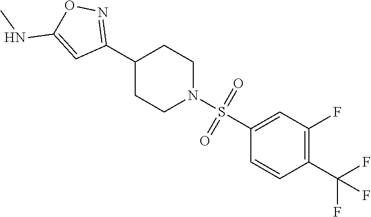 [SMILES: CNc1cc(C2CCN(S(=O)(=O)c3ccc(C(F)(F)F)c(F)c3)CC2)no1] | ++ | ¹H NMR (400 MHz, CDCl₃) δ 7.83- 7.78 (m, 1H), 7.66 (d, J = 8.9 Hz, 1H), 7.64 (d, J = 8.9 Hz, 1H), 4.80 (s, 1H), 4.41 (d, J = 4.7 Hz, 1H), 3.81 (dt, J = 12.0, 3.3 Hz, 2H), 2.88 (d, J = 5.2 Hz, 2H), 2.68-2.55 (m, 3H), 2.02 (dd, J = 13.7, 3.1 Hz, 2H), 1.91-1.80 (m, 2H). ESI-MS: m/z = 407.8 (M + 1). |
| 101 | 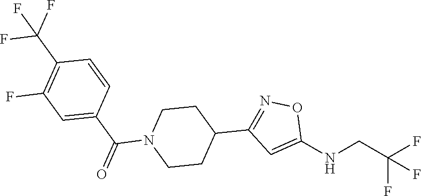 [SMILES: O=C(c1ccc(C(F)(F)F)c(F)c1)N1CCC(c2cc(NCC(F)(F)F)on2)CC1] | +++ | ¹H NMR (400 MHz, CDCl₃) δ 7.67 (t, J = 7.4 Hz, 1H), 7.29 (s, 1H), 7.24 (s, 1H), 5.01 (s, 1H), 4.83 (t, J = 6.7 Hz, 1H), 4.64 (s, 1H), 3.80 (tt, J = 28.1, 14.1 Hz, 2H), 3.77 (s, 1H), 3.16 (s, 1H), 3.06 (s, 1H), 2.93 (tt, J = 11.0, 3.7 Hz, 1H), 2.12-1.90 (m, 2H), 1.66 (s, 2H). ESI-MS: m/z = 439.8 (M + 1). |
| 102 | 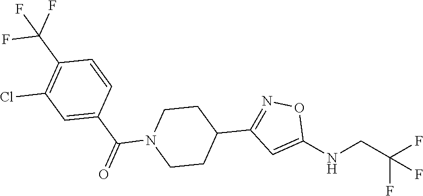 [SMILES: O=C(c1ccc(C(F)(F)F)c(Cl)c1)N1CCC(c2cc(NCC(F)(F)F)on2)CC1] | +++ | ¹H NMR (400 MHz, CDCl₃) δ 7.75 (d, J = 8.0 Hz, 1H), 7.55 (s, 1H), 7.39 (d, J = 7.9 Hz, 1H), 5.13-4.97 (m, 2H), 4.64 (s, 1H), 3.82-3.70 (m, 3H), 3.27- 2.85 (m, 3H), 2.06 (s, 1H), 1.96 (s, 1H), 1.68 (s, 2H). ESI-MS: m/z = 455.8 (M + 1). |
| 103 | 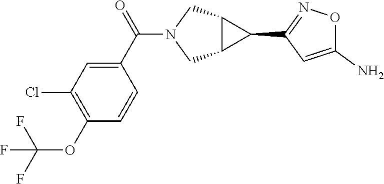 [SMILES: Nc1cc([C@H]2[C@@H]3CN(C(=O)c4ccc(OC(F)(F)F)c(Cl)c4)C[C@@H]32)no1] | ++++ | ¹H NMR (400 MHz, CDCl₃) δ 7.60 (d, J = 1.8 Hz, 1H), 7.42-7.33 (m, 2H), 4.89 (s, 1H), 4.70-4.20 (m, 3H), 3.74 (d, J = 8.8 Hz, 1H), 3.68-3.55 (m, 2H), 2.06-1.99 (m, 2H), 1.65 (t, J = 3.4 Hz, 1H). ESI-MS: m/z = 387.8 (M + 1). |
| 104 | 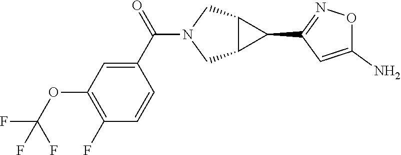 [SMILES: Nc1cc([C@H]2[C@@H]3CN(C(=O)c4ccc(F)c(OC(F)(F)F)c4)C[C@@H]32)no1] | +++ | ¹H NMR (400 MHz, CDCl₃) δ 7.50- 7.40 (m, 2H), 7.28-7.21 (m, 1H), 4.88 (s, 1H), 4.58-4.20 (m, 3H), 3.80-3.55 (m, 3H), 2.02 (s, 2H), 1.65 (t, J = 3.4 Hz, 1H). ESI-MS: m/z = 371.8 (M + 1). |
| 105 | 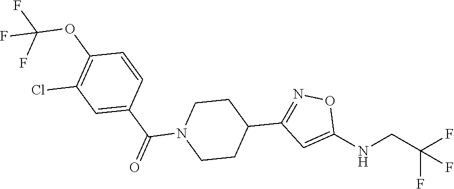 [SMILES: O=C(c1ccc(OC(F)(F)F)c(Cl)c1)N1CCC(c2cc(NCC(F)(F)F)on2)CC1] | +++ | ¹H NMR (400 MHz, CDCl₃) δ 7.54 (s, 1H), 7.40-7.31 (m, 2H), 5.14 (s, 1H), 4.98 (d, J = 15.7 Hz, 1H), 4.64 (s, 1H), 3.95-3.70 (m, 3H), 3.22-2.87 (m, 3H), 2.01 (s, 2H), 1.70 (s, 2H). ESI-MS: m/z = 471.8 (M + 1). |
| 106 | 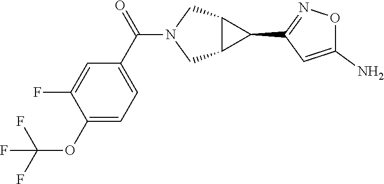 [SMILES: Nc1cc([C@H]2[C@@H]3CN(C(=O)c4ccc(OC(F)(F)F)c(F)c4)C[C@@H]32)no1] | +++ | ¹H NMR (400 MHz, CDCl₃) δ 7.36- 7.27 (m, 3H), 4.89 (s, 1H), 4.50-4.28 (m, 3H), 3.73 (d, J = 7.9 Hz, 1H), 3.67-3.57 (m, 2H), 2.03 (s, 2H), 1.64 (t, J = 3.4 Hz, 1H). ESI-MS: m/z = 371.8 (M + 1). |
| 107 | 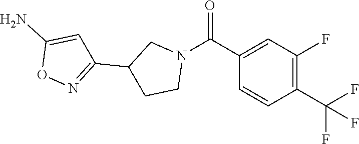 [SMILES: Nc1cc(C2CCN(C(=O)c3ccc(C(F)(F)F)c(F)c3)C2)no1] | ++ | ¹H NMR (400 MHz, CDCl₃) major rotamer δ 7.66 (td, J = 7.6, 2.9 Hz, 1H), 7.41-7.35 (m, 2H), 4.97 (s, 1H), 4.56 (s, 2H), 4.08-3.30 (m, 5H), 2.43-2.05 (m, 2H). ESI-MS: m/z = 343.8 (M + 1). |
| 108 | 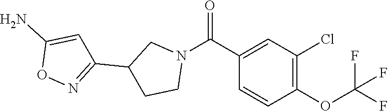 [SMILES: Nc1cc(C2CCN(C(=O)c3ccc(OC(F)(F)F)c(Cl)c3)C2)no1] | +++ | ¹H NMR (400 MHz, CDCl₃) major rotamer δ 7.66 (s, 1H), 7.46 (d, J = 2.7 Hz, 1H), 7.36 (d, J = 7.1 Hz, 1H), 4.97 (s, 1H), 4.75-4.72 (m, 2H), 4.08-3.31 (m, 6H), 2.45-2.24 (m, 1H), 2.19-2.08 (m, 1H). ESI-MS: m/z = 375.8 |
| 109 | 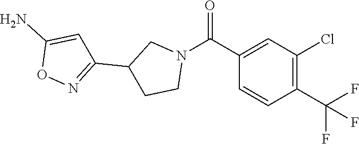 [SMILES: Nc1cc(C2CCN(C(=O)c3ccc(C(F)(F)F)c(Cl)c3)C2)no1] | +++ | ¹H NMR (400 MHz, DMSO) major rotamer δ 7.93 (dd, J = 8.1, 2.6 Hz, 1H), 7.85 (s, 1H), 7.68 (d, J = 8.0 Hz, 1H), 6.60 (d, J = 10.8 Hz, 2H), 4.87 (s, 1H), 3.84 (dd, J = 12.0, 7.8 Hz, 1H), 3.68-3.39 (m, 3H), 3.28 (d, J = 8.1 Hz, 1H), 2.26-2.10 (m, J = 19.8, 15.1 Hz, 1H), 2.04-1.92 (m, 1H). ESI-MS: m/z = 359.8 (M + 1). |
| 110 | 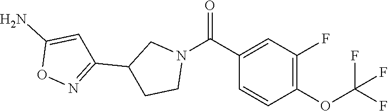 [SMILES: Nc1cc(C2CCN(C(=O)c3ccc(OC(F)(F)F)c(F)c3)C2)no1] | ++ | ¹H NMR (400 MHz, CDCl₃) major rotamer δ 7.41 (d, J = 10.3 Hz, 1H), 7.35 (s, 2H), 4.98 (s, 1H), 4.45 (s, 2H), 4.04-3.82 (m, 1H), 3.78-3.53 (m, 3H), 3.50-3.30 (m, 1H), 2.40-2.26 (m, 1H), 2.15 (ddd, J = 12.7, 8.4, 4.2 Hz, 1H). ESI-MS: m/z = 359.8 (M + 1). |
| 111 | 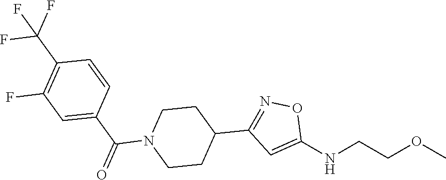 [SMILES: COCCNc1cc(C2CCN(C(=O)c3ccc(C(F)(F)F)c(F)c3)CC2)no1] | ++++ | ¹H NMR (400 MHz, CDCl₃) δ 7.67 (t, J = 7.5 Hz, 1H), 7.29 (s, 1H), 7.24 (s, 1H), 4.86 (s, 1H), 4.83 (d, J = 5.6 Hz, 1H), 4.64 (s, 1H), 3.72 (s, 1H), 3.58-3.52 (m, 2H), 3.39 (s, 3H), 3.34 (dd, J = 10.5, 5.6 Hz, 2H), 3.23-3.01 (m, 2H), 2.91 (ddd, J = 15.0, 7.5, 3.9 Hz, 1H), 2.13-1.88 (m, 2H), 1.69 (s, 1H), 1.57 (s, 1H). ESI-MS: m/z = 415.9 (M + 1). |
| 112 | 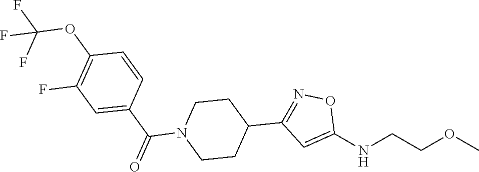 [SMILES: COCCNc1cc(C2CCN(C(=O)c3ccc(OC(F)(F)F)c(F)c3)CC2)no1] | ++ | ¹H NMR (400 MHz, CDCl₃) δ 7.31- 7.26 (m, 1H), 7.21 (dd, J = 9.8, 1.9 Hz, 1H), 7.16-7.13 (m, 1H), 4.79 (s, 1H), 4.57 (s, 1H), 3.70 (s, 1H), 3.50- 3.47 (m, 2H), 3.32 (s, 3H), 3.28-3.25 (m, 2H), 3.20-2.90 (m, 2H), 2.83 (td, J = 7.3, 3.6 Hz, 1H), 1.94 (s, 2H), 1.64 (s, 2H). ESI-MS: m/z = 431.9 (M + 1). |
| 113 | 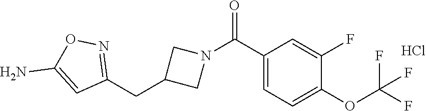 [SMILES: Nc1cc(CC2CN(C(=O)c3ccc(OC(F)(F)F)c(F)c3)C2)no1 ; Cl] | ++ | ¹H NMR (400 MHz, CD3SOCD3, 300K) δ (ppm) = 7.79-7.70 (m, 1H), 7.70-7.63 (m, 1H), 7.62-7.55 (m, 1H), 4.83 (s, 1H), 4.51-4.37 (m, 1H), 4.21- 4.10 (m, 1H), 4.08-3.99 (m, 1H), 3.62 (qd, J = 6.6, 10.5 Hz, 1H), 3.18-2.84 (m, 2H), 2.78-2.72 (m, 2H), 1.38-1.24 |
| | | | (m, 2H). ESI-MS: m/z = 360.1 (M + 1). |
| 114 | 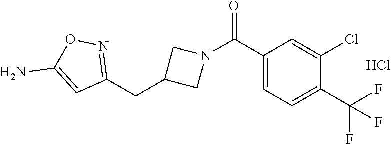 [SMILES: Nc1cc(CC2CN(C(=O)c3ccc(C(F)(F)F)c(Cl)c3)C2)no1 ; Cl] | ++ | ¹H NMR (400 MHz, CDCl3, 300K) δ (ppm) = 7.68 (s, 1H), 7.66 (d, J = 8.3 Hz, 1H), 7.52 (d, J = 8.0 Hz, 1H), 4.90 (s, 1H), 4.42-4.22 (m, 2H), 3.96 (br dd, J = 5.6, 8.2 Hz, 1H), 3.88 (br dd, J = 5.9, 10.2 Hz, 1H), 3.07-2.92 (m, 1H), 2.86-2.78 (m, 2H), 1.26-1.12 (m, 1H), 0.00 (s, 1H). ESI-MS: m/z = 360.1 (M + 1). |
| 115 | 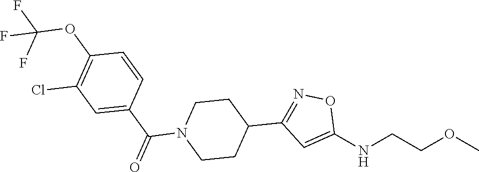 [SMILES: COCCNc1cc(C2CCN(C(=O)c3ccc(OC(F)(F)F)c(Cl)c3)CC2)no1] | +++ | ¹H NMR (400 MHz, CDCl₃) δ 7.47 (d, J = 1.4 Hz, 1H), 7.31-7.26 (m, 2H), 4.79 (s, 1H), 4.77 (d, J = 5.7 Hz, 1H), 4.55 (s, 1H), 3.69 (s, 1H), 3.50-3.47 (m, 2H), 3.32 (s, 3H), 3.29-3.25 (m, 2H), 3.15-2.93 (m, 2H), 2.84 (ddd, J = 11.2, 7.3, 3.9 Hz, 1H), 1.99-1.88 (m, 2H), 1.62 (s, 2H). ESI-MS: m/z = 447.9 (M + 1). |
| 116 | 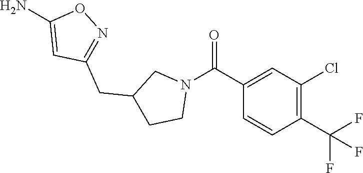 [SMILES: Nc1cc(CC2CCN(C(=O)c3ccc(C(F)(F)F)c(Cl)c3)C2)no1] | +++ | ¹H NMR (400 MHz, CDCl₃) major rotamer δ 7.73 (dd, J = 8.0, 1.9 Hz, 1H), 7.64 (s, 1H), 7.47 (d, J = 8.0 Hz, 1H), 4.93 (s, 1H), 4.42 (s, 2H), 3.92- 3.74 (m, 1H), 3.67-3.41 (m, 2H), 3.37- 3.12 (m, 1H), 2.70-2.55 (m, 3H), 2.25- 2.07 (m, 1H), 1.79-1.64 (m, 1H). ESI- MS: m/z = 373.8 (M + 1). |
| 117 | 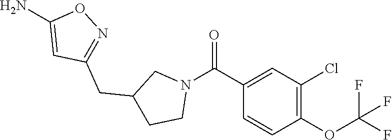 [SMILES: Nc1cc(CC2CCN(C(=O)c3ccc(OC(F)(F)F)c(Cl)c3)C2)no1] | ++++ | ¹H NMR (400 MHz, CDCl₃) major rotamer δ 7.65 (d, J = 2.0 Hz, 1H), 7.47-7.42 (m, 1H), 7.35 (d, J = 8.5 Hz, 1H), 4.93 (s, 1H), 4.41 (s, 2H), 3.89-3.75 (m, 1H), 3.63-3.44 (m, 2H), 3.34-3.15 (m, 1H), 2.70-2.55 (m, 3H), 2.21-2.10 (m, 1H), 1.77-1.69 (m, 1H). ESI-MS: m/z = 389.8 (M + 1). |
| 118 | 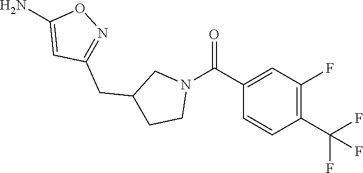 [SMILES: Nc1cc(CC2CCN(C(=O)c3ccc(C(F)(F)F)c(F)c3)C2)no1] | ++ | ¹H NMR (400 MHz, CDCl₃) δ 7.58 (dd, J = 10.4, 4.4 Hz, 1H), 7.32-7.25 (m, 2H), 4.96-4.84 (m, 1H), 4.43-4.37 (m, 2H), 3.83-3.70 (m, 1H), 3.57-3.33 (m, 2H), 3.28-3.07 (m, 1H), 2.63-2.46 (m, 3H), 2.11 (d, J = 12.9 Hz, 1H), 1.71-1.64 (m, 1H).; ESI-MS: 357.9 [M + 1]+. |
| 119 | 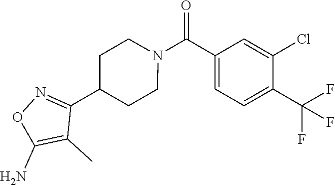 | + | ¹H NMR (400 MHz, CDCl₃) δ 7.41- 7.36 (m, 1H), 7.32 (d, J = 7.9 Hz, 2H), 5.03-4.91 (m, 1H), 4.41 (s, 2H), 3.91- 3.75 (m, 1H), 3.66-3.43 (m, 2H), 3.37- 3.18 (m, 1H), 2.62 (ddd, J = 20.6, 16.6, 7.9 Hz, 3H), 2.17 (dd, J = 16.0, 11.7 Hz, 1H), 1.77-1.69 (m, 1H). ESI-MS: 373.9 [M + 1]+. |
| 120 | 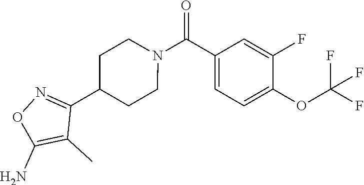 [SMILES: Cc1c(C2CCN(C(=O)c3ccc(OC(F)(F)F)c(F)c3)CC2)noc1N] | + | 1H NMR (400 MHz, CDCl3, 300K) Shift (ppm) = 7.40-7.33 (m, 1H), 7.30 (dd, J = 1.8, 9.8 Hz, 1H), 7.23 (td, J = 1.5, 8.3 Hz, 1H), 4.64 (br s, 2H), 3.82 (br s, 2H), 3.14 (br s, 3H), 2.92-2.69 (m, 4H), 2.12-1.91 (m, 3H). ESI-MS: 388.3 [M + 1]+. |
| 121 | 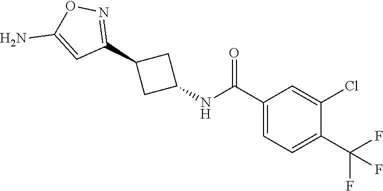 [SMILES: Nc1cc([C@H]2C[C@H](NC(=O)c3ccc(C(F)(F)F)c(Cl)c3)C2)no1] | + | ¹H NMR (400 MHz, DMSO) δ 9.05 (d, J = 7.7 Hz, 1H), 8.20 (s, 1H), 8.04 (d, J = 0.7 Hz, 2H), 6.60 (s, 2H), 4.98 (s, 1H), 4.50 (d, J = 7.7 Hz, 1H), 3.14- 3.05 (m, 1H), 2.63 (dt, J = 10.4, 7.8 Hz, 2H), 2.28 (dt, J = 11.5, 9.7 Hz, 2H). ESI-MS: 359.8 [M + 1]+. |
| 122 | 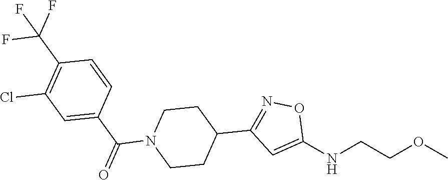 [SMILES: COCCNc1cc(C2CCN(C(=O)c3ccc(C(F)(F)F)c(Cl)c3)CC2)no1] | ++++ | ¹H NMR (400 MHz, DMSO) δ 7.39 (d, J = 8.1 Hz, 1H), 7.79 (s, 1H), 7.58 (d, J = 8.5 Hz, 1H), 7.14 (s, 1H), 5.02 (s, 1H), 4.44 (d, J = 12.4 Hz, 1H), 3.42 (t, J = 5.6 Hz, 3H), 3.26 (s, 3H), 3.20 (dd, J = 11.5, 5.7 Hz, 3H), 2.97 (t, J = 11.0 Hz, 1H), 2.85-2.78 (m, 1H), 1.91 (d, J = 11.9 Hz, 1H), 1.75 (d, J = 11.9 Hz, 1H), 1.64-1.54 (m, 2H). ESI-MS: 431.7 [M + 1]+. |
| 123 | 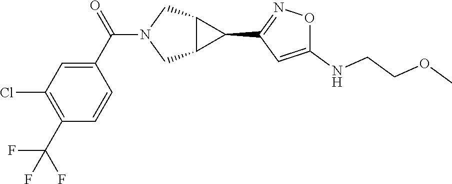 [SMILES: COCCNc1cc([C@H]2[C@@H]3CN(C(=O)c4ccc(C(F)(F)F)c(Cl)c4)C[C@@H]32)no1] | ++ | ¹H NMR (400 MHz, DMSO) δ 7.92 (d, J = 8.1 Hz, 1H), 7.85 (s, 1H), 7.65 (d, J = 8.6 Hz, 1H), 7.13 (t, J = 5.9 Hz, 1H), 4.82 (s, 1H), 4.01 (d, J = 12.2 Hz, 1H), 3.72 (dd, J = 10.7, 4.0 Hz, 1H), 3.52 (dd, J = 12.2, 4.0 Hz, 1H), 3.41 (dd, J = 11.5, 5.8 Hz, 3H), 3.25 (s, 3H), 3.18 (q, J = 5.7 Hz, 2H), 1.94 (ddd, J = 14.9, 7.3, 3.7 Hz, 2H), 1.73 (t, J = 3.4 Hz, 1H). ESI-MS: 429.8 [M + 1]+. |
| 124 | 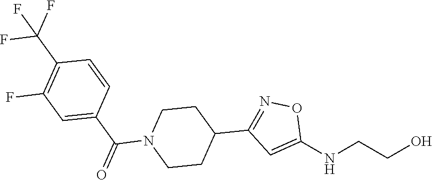 [SMILES: O=C(c1ccc(C(F)(F)F)c(F)c1)N1CCC(c2cc(NCCO)on2)CC1] | +++ | ¹H NMR (400 MHz, DMSO) δ 7.87 (s, 1H), 7.61 (d, J = 11.0 Hz, 1H), 7.44 (d, J = 8.1 Hz, 1H), 7.05 (s, 1H), 4.99 (s, 1H), 4.72 (t, J = 5.5 Hz, 1H), 4.44 (d, J = 12.2 Hz, 1H), 3.49 (dd, J = 11.5, 5.9 Hz, 3H), 3.16 (t, J = 11.0 Hz, 1H), 3.10 (q, J = 6.0 Hz, 2H), 2.95 (t, J = 11.0 Hz, 1H), 2.85-2.78 (m, 1H), 1.91 (d, J = 11.6 Hz, 1H), 1.75 (d, J = 11.8 Hz, 1H), 1.60 (d, J = 9.0 Hz, 2H). ESI-MS: 401.8 [M + 1]+. |
| 125 | 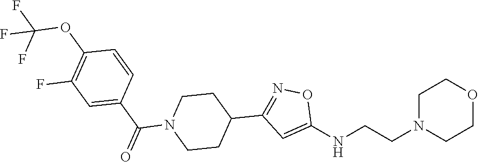 [SMILES: O=C(c1ccc(OC(F)(F)F)c(F)c1)N1CCC(c2cc(NCCN3CCOCC3)on2)CC1] | +++ | ¹H NMR (400 MHz, CDCl₃) δ 7.29 (t, J = 7.7 Hz, 1H), 7.23 (d, J = 1.5 Hz, 1H), 7.15 (d, J = 8.3 Hz, 1H), 5.11 (s, 1H), 4.77 (s, 1H), 4.55 (s, 1H), 3.78- 3.55 (m, 5H), 3.21-2.90 (m, 4H), 2.84 (ddd, J = 11.1, 7.5, 4.0 Hz, 1H), 2.53 (t, J = 5.8 Hz, 2H), 2.41 (d, J = 4.0 Hz, 4H), 1.94 (s, 2H), 1.61 (s, 2H). ESI- MS: m/z = 486.8 (M + 1). |
| 126 | 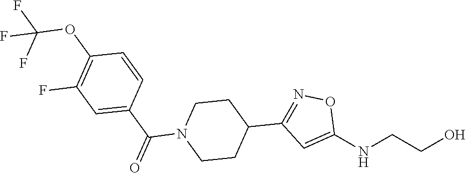 [SMILES: O=C(c1ccc(OC(F)(F)F)c(F)c1)N1CCC(c2cc(NCCO)on2)CC1] | +++ | ¹H NMR (400 MHz, MeOD) δ 7.56- 7.51 (m, 1H), 7.46 (dd, J = 10.2, 1.9 Hz, 1H), 7.36-7.32 (m, 1H), 5.04 (s, 1H), 4.59 (brs, 1H), 3.66 (t, J = 5.7 Hz, 3H), 3.26 (t, J = 5.7 Hz, 3H), 3.04 (brs, 1H), 2.90 (tt, J = 11.4, 3.8 Hz, 1H), 2.02 (d, J = 4.5 Hz, 1H), 1.91 (d, J = 20.3 Hz, 1H), 1.71 (s, 2H). ESI-MS: 417.8 [M + 1]+. |
| 127 | 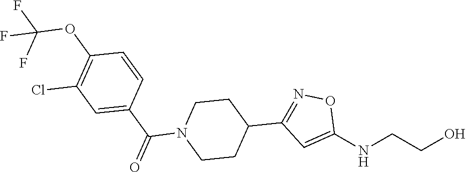 [SMILES: O=C(c1ccc(OC(F)(F)F)c(Cl)c1)N1CCC(c2cc(NCCO)on2)CC1] | ++++ | ¹H NMR (400 MHz, DMSO) δ 7.77 (d, J = 2.0 Hz, 1H), 7.66-7.62 (m, 1H), 7.52 (dd, J = 8.4, 2.0 Hz, 1H), 7.05 (t, J = 5.9 Hz, 1H), 5.00 (s, 1H), 4.72 (t, J = 5.4 Hz, 1H), 4.43 (brs, 1H), 3.49 (dd, J = 11.4, 5.8 Hz, 3H), 3.17 (s, 1H), 3.13-3.08 (m, 2H), 2.94 (s, 1H), 2.81 (ddd, J = 11.3, 7.6, 3.8 Hz, 1H), 1.89 (s, 1H), 1.76 (s, 1H), 1.59 (dd, J = 21.1, 11.4 Hz, 2H). ESI-MS: 433.8 [M + 1]+. |
| 128 | 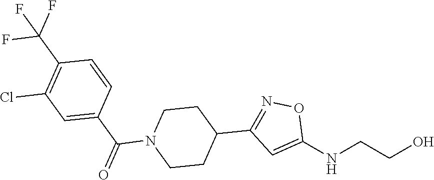 [SMILES: O=C(c1ccc(C(F)(F)F)c(Cl)c1)N1CCC(c2cc(NCCO)on2)CC1] | ++++ | ¹H NMR (400 MHz, DMSO) δ 7.93 (d, J = 8.1 Hz, 1H), 7.79 (s, 1H), 7.58 (d, J = 8.5 Hz, 1H), 7.05 (t, J = 5.9 Hz, 1H), 5.00 (s, 1H), 4.72 (t, J = 5.4 Hz, 1H), 4.44 (d, J = 12.1 Hz, 1H), 3.49 (dd, J = 11.5, 5.9 Hz, 3H), 3.18 (d, J = 11.5 Hz, 1H), 3.11 (q, J = 6.0 Hz, 2H), 2.96 (t, J = 12.1 Hz, 1H), 2.82 (ddd, J = 15.1, 7.5, 3.7 Hz, 1H), 1.91 (d, J = 11.8 Hz, 1H), 1.79-1.72 (m, 1H), 1.66- 1.56 (m, 2H). ESI-MS: 417.8 [M + 1]+. |
| 129 | 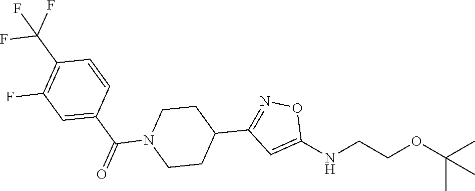 [SMILES: CC(C)(C)OCCNc1cc(C2CCN(C(=O)c3ccc(C(F)(F)F)c(F)c3)CC2)no1] | +++ | ¹H NMR (400 MHz, DMSO) δ 7.87 (t, J = 7.7 Hz, 1H), 7.61 (d, J = 11.0 Hz, 1H), 7.44 (d, J = 8.0 Hz, 1H), 7.04 (t, J = 6.0 Hz, 1H), 5.01 (s, 1H), 4.44 (d, J = 11.7 Hz, 1H), 3.48 (d, J = 13.3 Hz, 1H), 3.40 (t, J = 6.0 Hz, 2H), 3.20- 3.11 (m, 3H), 2.95 (t, J = 11.5 Hz, 1H), 2.82 (ddd, J = 11.3, 7.6, 3.8 Hz, 1H), 1.91 (d, J = 11.1 Hz, 1H), 1.75 (d, J = 11.7 Hz, 1H), 1.60 (d, J = 8.9 Hz, 2H), 1.12 (s, 9H). ESI-MS: 457.9 [M + 1]+. |
| 130 | 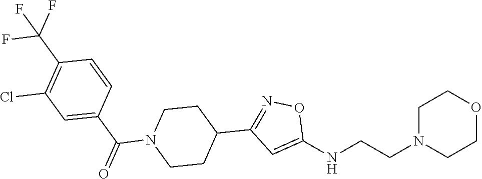 [SMILES: O=C(c1ccc(C(F)(F)F)c(Cl)c1)N1CCC(c2cc(NCCN3CCOCC3)on2)CC1] | ++++ | ¹H NMR (400 MHz, CDCl₃) δ 7.74 (d, J = 8.0 Hz, 1H), 7.55 (s, 1H), 7.38 (d, J = 8.0 Hz, 1H), 5.17 (t, J = 5.0 Hz, 1H), 4.85 (s, 1H), 4.64 (s, 1H), 3.83- 3.60 (m, 5H), 3.22 (dd, J = 11.5, 5.3 Hz, 2H), 3.18 (s, 1H), 3.03 (s, 1H), 2.92 (ddd, J = 11.1, 7.5, 3.6 Hz, 1H), 2.64-2.57 (m, 2H), 2.52-2.39 (m, 4H), 2.06 (s, 1H), 1.94 (s, 1H), 1.80-1.60 (m, 2H). ESI-MS: m/z = 486.9 (M + 1). |
| 131 | 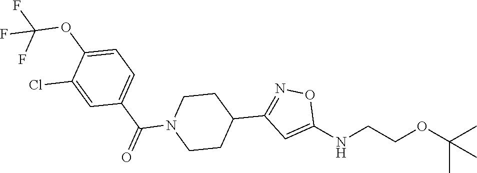 [SMILES: CC(C)(C)OCCNc1cc(C2CCN(C(=O)c3ccc(OC(F)(F)F)c(Cl)c3)CC2)no1] | ++ | ¹H NMR (400 MHz, CDCl₃) δ 7.54 (d, J = 1.7 Hz, 1H), 7.37-7.33 (m, 2H), 4.86 (s, 2H), 4.64 (s, 1H), 3.77 (s, 1H), 3.52 (t, J = 5.2 Hz, 2H), 3.28 (dd, J = 10.7, 5.6 Hz, 2H), 3.16 (s, 1H), 3.07- 2.99 (m, 1H), 2.90 (ddd, J = 11.1, 7.3, 3.9 Hz, 1H), 2.01 (s, 2H), 1.73 (s, 2H), 1.20 (s, 9H). ESI-MS: m/z = 489.9 (M + 1). |
| 132 | 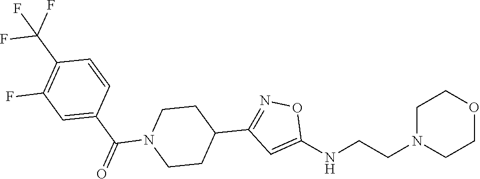 [SMILES: O=C(c1ccc(C(F)(F)F)c(F)c1)N1CCC(c2cc(NCCN3CCOCC3)on2)CC1] | +++ | 1H NMR (400 MHz, CDCl3) δ 7.67 (t, J = 7.4 Hz, 1H), 7.29 (s, 1H), 7.24 (s, 1H), 5.19 (s, 1H), 4.85 (s, 1H), 4.75- 4.55 (m, 1H), 3.73 (brs, 5H), 3.40-3.00 (m, 5H), 2.91 (tt, J = 11.2, 3.8 Hz, 1H), 2.62 (s, 2H), 2.49 (s, 3H), 2.17-1.90 (m, 3H), 1.69 (s, 1H). ESI-MS: m/z = 470.9 (M + 1). |
| 133 | 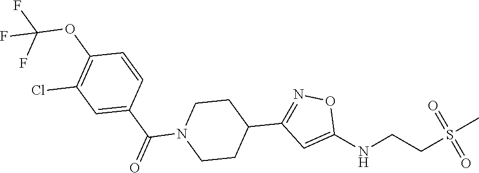 [SMILES: CS(=O)(=O)CCNc1cc(C2CCN(C(=O)c3ccc(OC(F)(F)F)c(Cl)c3)CC2)no1] | ++++ | ¹H NMR (400 MHz, MeOD) δ 7.59 (d, J = 1.9 Hz, 1H), 7.45 (dd, J = 8.4, 1.3 Hz, 1H), 7.38 (dd, J = 8.4, 2.0 Hz, 1H), 5.06 (s, 1H), 4.50 (s, 1H), 3.57 (t, J = 6.6 Hz, 3H), 3.28 (t, J = 6.5 Hz, 2H), 3.19-3.11 (m, 1H), 2.94 (d, J = 11.2 Hz, 1H), 2.91 (s, 3H), 2.83 (t, J = 3.8 Hz, 1H), 1.95-1.79 (m, 2H), 1.62 (s, 2H). ESI-MS: m/z = 495.6 [M + 1]+. |
| 134 | 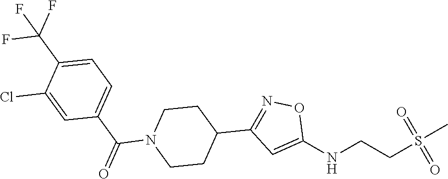 [SMILES: CS(=O)(=O)CCNc1cc(C2CCN(C(=O)c3ccc(C(F)(F)F)c(Cl)c3)CC2)no1] | ++++ | ¹H NMR (400 MHz, CDCl₃) δ 7.75 (d, J = 8.0 Hz, 1H), 7.55 (s, 1H), 7.39 (d, J = 7.9 Hz, 1H), 5.21 (t, J = 6.1 Hz, 1H), 4.97 (s, 1H), 4.65 (s, 1H), 3.79 (dd, J = 11.9, 6.2 Hz, 2H), 3.71 (s, 1H), 3.38-3.26 (m, 2H), 3.17 (d, J = 6.8 Hz, 1H), 3.10-2.85 (m, 5H), 2.20-1.85 (m, 2H), 1.75-1.60- (m, 2H). ESI-MS: m/z = 479.8 (M + 1). |
| 135 | 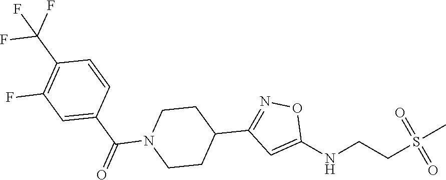 [SMILES: CS(=O)(=O)CCNc1cc(C2CCN(C(=O)c3ccc(C(F)(F)F)c(F)c3)CC2)no1] | +++ | ¹H NMR (400 MHz, CDCl₃) δ 7.67 (t, J = 7.4 Hz, 1H), 7.29 (s, 1H), 7.24 (s, 1H), 5.20 (t, J = 6.3 Hz, 1H), 4.97 (s, 1H), 4.65 (s, 1H), 3.79 (dd, J = 12.0, 6.3 Hz, 2H), 3.70 (s, 1H), 3.36-3.27 (m, 2H), 3.30-3.00 (m, 2H), 2.99 (s, 3H), 2.96-2.86 (m, 1H), 2.15-1.85 (m, 2H), 1.80-1.65 (m, 2H). ESI-MS: m/z = 463.8 (M + 1). |
| 136 | 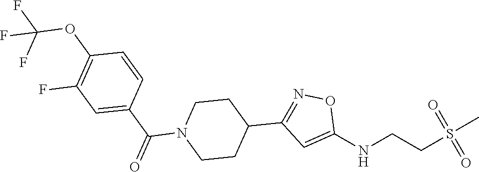 [SMILES: CS(=O)(=O)CCNc1cc(C2CCN(C(=O)c3ccc(OC(F)(F)F)c(F)c3)CC2)no1] | ++ | ¹H NMR (400 MHz, CDCl₃) δ 7.39- 7.33 (m, 1H), 7.29 (dd, J = 9.8, 1.9 Hz, 1H), 7.24-7.19 (m, 1H), 5.17 (t, J = 6.3 Hz, 1H), 4.97 (s, 1H), 4.69 (brs, 1H), 3.83-3.73 (m, 3H), 3.42-3.29 (m, 2H), 3.11 (d, J = 44.3, Hz, 2H), 2.99 (s, 3H), 2.97-2.86 (m, 1H), 2.11-1.92 (m, 2H), 1.71 (s, 2H). ESI-MS: m/z = 479.8 (M + 1). |
| 137 | 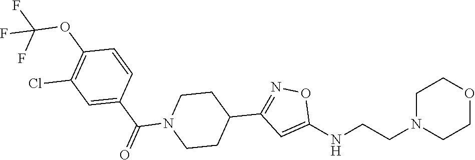 [SMILES: O=C(c1ccc(OC(F)(F)F)c(Cl)c1)N1CCC(c2cc(NCCN3CCOCC3)on2)CC1] | ++++ | ¹H NMR (400 MHz, CDCl₃) δ 7.54 (d, J = 1.6 Hz, 1H), 7.41-7.31 (m, 2H), 5.19 (s, 1H), 4.85 (s, 1H), 4.63 (s, 1H), 3.73 (s, 5H), 3.23 (d, J = 5.2 Hz, 2H), 3.20-2.97 (m, 2H), 2.96-2.81 (m, 1H), 2.61 (s, 2H), 2.49 (s, 4H), 2.02 (s, 2H), 1.72 (s, 2H). ESI-MS: m/z = 502.8 (M + 1). |
| 138 | 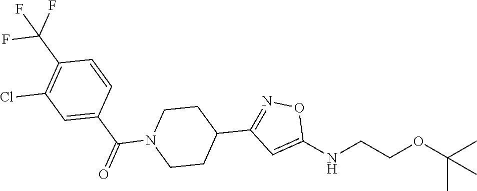 [SMILES: CC(C)(C)OCCNc1cc(C2CCN(C(=O)c3ccc(C(F)(F)F)c(Cl)c3)CC2)no1] | ++ | ¹H NMR (400 MHz, CDCl₃) δ 7.74 (d, J = 8.0 Hz, 1H), 7.55 (s, 1H), 7.38 (d, J = 8.0 Hz, 1H), 4.86 (s, 2H), 4.64 (s, 1H), 3.70 (s, 1H), 3.52 (t, J = 5.1 Hz, 2H), 3.28 (dd, J = 10.5, 5.3 Hz, 2H), 3.17 (s, 1H), 3.03 (s, 1H), 2.91 (t, J = 11.0 Hz, 1H), 2.06 (s, 1H), 1.95 (s, 1H), 1.69 (s, 2H), 1.20 (s, 9H). ESI- MS: m/z = 473.9 |
| 139 | 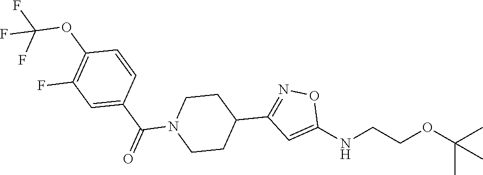 [SMILES: CC(C)(C)OCCNc1cc(C2CCN(C(=O)c3ccc(OC(F)(F)F)c(F)c3)CC2)no1] | ++ | ¹H NMR (400 MHz, CDCl₃) δ 7.39- 7.33 (m, 1H), 7.31-7.27 (m, 1H), 7.24- 7.19 (m, 1H), 4.86 (s, 2H), 4.63 (s, 1H), 3.77 (s, 1H), 3.57-3.47 (m, 2H), 3.28 (d, J = 4.0 Hz, 2H), 3.05 (s, 2H), 2.91 (ddd, J = 11.1, 7.4, 3.9 Hz, 1H), 2.03 (dd, J = 10.4, 5.0 Hz, 2H), 1.72 (s, 2H), 1.18 (d, J = 13.2 Hz, 9H). ESI- MS: m/z = 473.9 |
| 140 | 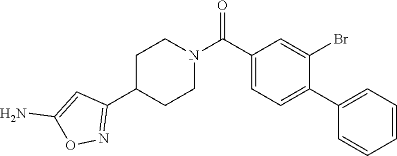 [SMILES: Nc1cc(C2CCN(C(=O)c3ccc(-c4ccccc4)c(Br)c3)CC2)no1] | +++ | ¹H NMR (400 MHz, CDCl₃) δ 7.72 (d, J = 1.3 Hz, 1H), 7.48-7.34 (m, 7H), 5.01 (s, 1H), 4.68 (s, 1H), 4.45 (s, 2H), 3.91 (s, 1H), 3.12 (d, J = 65.7 Hz, 2H), 2.92 (tt, J = 11.3, 3.8 Hz, 1H), 2.02 (d, J = 15.9 Hz, 2H), 1.73 (s, 2H). ESI- MS: m/z = 425.8 [M + 1]+ |
| 141 | 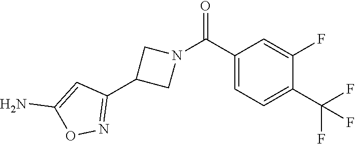 [SMILES: Nc1cc(C2CN(C(=O)c3ccc(C(F)(F)F)c(F)c3)C2)no1] | + | 1H NMR (400 MHz, DMSO) δ 7.88 (t, J = 7.7 Hz, 1H), 7.74 (d, J = 11.3 Hz, 1H), 7.67 (d, J = 8.1 Hz, 1H), 6.69 (s, 2H), 5.05 (s, 1H), 4.61 (t, J = 8.8 Hz, 1H), 4.48-4.31 (m, 2H), 4.08 (dd, J = 9.9, 6.1 Hz, 1H), 3.84-3.72 (m, 1H). ESI-MS: m/z = 329.8 [M + 1]+ |
| 142 | 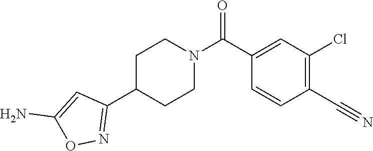 [SMILES: N#Cc1ccc(C(=O)N2CCC(c3cc(N)on3)CC2)cc1Cl] | ++ | ¹H NMR (400 MHz, CDCl₃) δ 7.73 (d, J = 7.9 Hz, 1H), 7.55 (d, J = 1.3 Hz, 1H), 7.39 (dd, J = 7.9, 1.4 Hz, 1H), 5.00 (s, 1H), 4.60 (s, 1H), 4.45 (s, 2H), 3.65 (s, 1H), 3.18 (s, 1H), 3.04 (s, 1H), 2.91 (tt, J = 11.1, 3.9 Hz, 1H), 2.04 (s, 1H), 1.95 (s, 1H), 1.68 (s, 2H). ESI- MS: m/z = 331.1 (M + 1) |
| 143 | 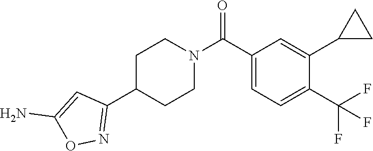 [SMILES: Nc1cc(C2CCN(C(=O)c3ccc(C(F)(F)F)c(C4CC4)c3)CC2)no1] | ++ | ¹H NMR (400 MHz, CDCl₃) δ 7.64 (d, J = 8.0 Hz, 1H), 7.23 (s, 1H), 7.06 (s, 1H), 4.99 (s, 1H), 4.65 (s, 1H), 4.45 (s, 2H), 3.68 (s, 1H), 3.19-2.94 (m, 2H), 2.89 (tt, J = 11.2, 3.8 Hz, 1H), 2.22 (s, 1H), 2.04 (s, 1H), 1.89 (s, 1H), 1.69 (d, J = 40.6 Hz, 2H), 1.10-1.02 (m, 2H), 0.79 (q, J = 5.3 Hz, 2H). ESI-MS: m/z = 380.2 (M + 1) |
| 144 | 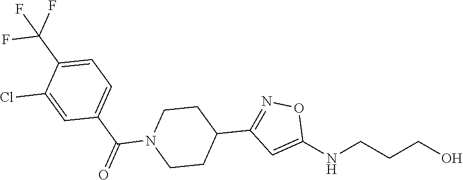 [SMILES: O=C(c1ccc(C(F)(F)F)c(Cl)c1)N1CCC(c2cc(NCCCO)on2)CC1] | ++++ | ¹H NMR (400 MHz, CDCl₃) δ 7.74 (d, J = 8.0 Hz, 1H), 7.55 (s, 1H), 7.38 (d, J = 8.0 Hz, 1H), 4.97 (t, J = 5.6 Hz, 1H), 4.86 (s, 1H), 4.63 (s, 1H), 3.81 (t, J = 5.3 Hz, 2H), 3.69 (s, 1H), 3.33 (q, J = 6.2 Hz, 2H), 3.17 (s, 1H), 3.03 (s, 1H), 2.90 (tt, J = 11.1, 3.8 Hz, 1H), 2.05 (s, 1H), 1.93 (s, 1H), 1.90-1.83 (m, 2H), 1.75 (s, 1H), 1.66 (s, 2H). ESI-MS: m/z = 432.1 (M + 1) |
| 145 | 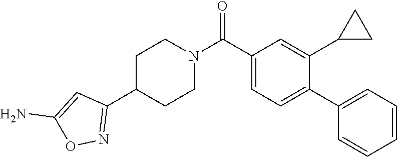 [SMILES: Nc1cc(C2CCN(C(=O)c3ccc(-c4ccccc4)c(C4CC4)c3)CC2)no1] | +++ | ¹H NMR (400 MHz, DMSO-d6) δ 7.44-7.49 (m, 4H), 7.38-7.41 (m, 1H), 7.24 (t, J = 8.0 Hz, 2H), 6.95 (s, 1H), 6.56 (s, 2H), 4.90 (s, 1H), 4.46 (s, 1H), 3.60 (s, 1H) 3.21 (s, 1H), 2.90 (s, 1H), 2.78 (t, J = 8.0 Hz, 1H), 1.82-1.86 (m, 3H), 1.56 (s, 2H), 0.86 (d, J = 4.0 Hz, 2H), 0.71 (s, 2H). ESI-MS: m/z = 388.2 (M + 1) |
| 146 | 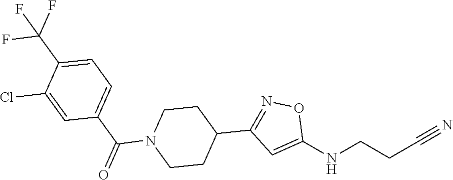 [SMILES: N#CCCNc1cc(C2CCN(C(=O)c3ccc(C(F)(F)F)c(Cl)c3)CC2)no1] | + | ¹H NMR (400 MHz, CDCl₃) δ 7.75 (d, J = 8.0 Hz, 1H), 7.55 (s, 1H), 7.39 (d, J = 8.0 Hz, 1H), 4.96 (s, 1H), 4.97 (t, J = 6.5 Hz, 1H), 4.67 (s, 1H), 3.77-3.65 (m, 1H), 3.56 (q, J = 6.5 Hz, 2H), 3.18 (s, 1H), 3.03 (s, 1H), 2.97-2.87 (m, 1H), 2.69 (t, J = 6.4 Hz, 2H), 2.10-2.01 (m, 1H), 1.98-1.90 (m, 1H), 1.80-1.63 (m, 2H). ESI-MS: m/z = 427.1 (M + 1) |
| 147 | 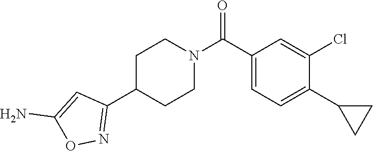 [SMILES: Nc1cc(C2CCN(C(=O)c3ccc(C4CC4)c(Cl)c3)CC2)no1] | − | ¹H NMR (400 MHz, DMSO) δ 7.45 (d, J = 1.5 Hz, 1H), 7.27 (dd, J = 8.0, 1.4 Hz, 1H), 7.07 (d, J = 8.0 Hz, 1H), 6.55 (s, 2H), 4.89 (s, 1H), 4.42 (s, 1H), 3.58 (s, 1H), 3.14 (s, 1H), 2.91 (s, 1H), 2.77 (ddd, J = 11.4, 7.8, 3.7 Hz, 1H), 2.18 (ddd, J = 13.6, 8.5, 5.3 Hz, 1H), 1.87 (s, 1H), 1.77 (s, 1H), 1.55 (d, J = 10.8 Hz, 2H), 1.12-1.00 (m, 2H), 0.81- 0.70 (m, 2H). ESI-MS: m/z = 346.2 (M + 1) |
| 149 | 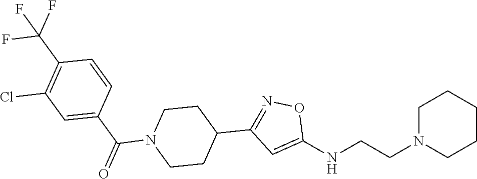 [SMILES: O=C(c1ccc(C(F)(F)F)c(Cl)c1)N1CCC(c2cc(NCCN3CCCCC3)on2)CC1] | +++ | ¹H NMR (400 MHz, CDCl₃) δ 7.74 (d, J = 8.0 Hz, 1H), 7.55 (s, 1H), 7.39 (d, J = 8.0 Hz, 1H), 5.31 (s, 1H), 4.84 (s, 1H), 4.64 (s, 1H), 3.69 (s, 1H), 3.20 (dd, J = 11.6, 5.3 Hz, 2H), 3.02 (d, J = 4.8 Hz, 1H), 2.91 (tt, J = 11.1, 3.8 Hz, 1H), 2.61-2.50 (m, 2H), 2.41 (s, 3H), 2.06 (brs, 1H), 1.93 (brs, 1H), 1.80- 1.40 (m, 10H). ESI-MS: m/z = 485.2 (M + 1) |
| 150 | 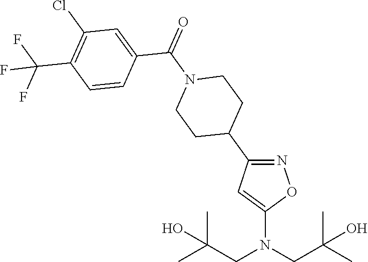 [SMILES: CC(C)(O)CN(CC(C)(C)O)c1cc(C2CCN(C(=O)c3ccc(C(F)(F)F)c(Cl)c3)CC2)no1] | ++++ | ¹H NMR (400 MHz, CDCl₃) δ 7.74 (d, J = 8.0 Hz, 1H), 7.55 (s, 1H), 7.39 (d, J = 8.6 Hz, 1H), 4.83 (s, 1H), 4.64 (s, 1H), 3.69 (s, 1H), 3.49 (s, 4H), 3.22- 3.13 (m, 1H), 3.02 (s, 1H), 2.89 (tt, J = 11.2, 3.8 Hz, 1H), 2.10-1.80 (m, 2H), 1.67 (s, 2H), 1.30-1.23 (m, 12H). ESI-MS: m/z = 518.2 (M + 1) |
| 151 | 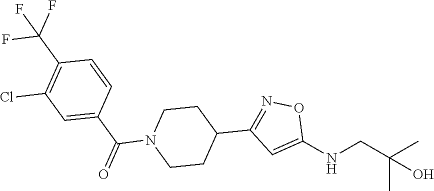 [SMILES: CC(C)(O)CNc1cc(C2CCN(C(=O)c3ccc(C(F)(F)F)c(Cl)c3)CC2)no1] | +++ | ¹H NMR (400 MHz, CDCl₃) δ 7.74 (d, J = 8.0 Hz, 1H), 7.55 (s, 1H), 7.38 (d, J = 8.0 Hz, 1H), 4.87 (s, 1H), 4.63 (s, 1H), 3.69 (s, 1H), 3.24-3.05 (m, 3H), 3.04 (d, J = 11.2 Hz, 1H), 2.90 (ddd, J = 15.0, 7.5, 4.0 Hz, 1H), 2.05 (s, 1H), 1.92 (s, 1H), 1.80-1.55 (m, 4H), 1.28 (d, J = 12.0 Hz, 6H). ESI-MS: m/z = 446.2 (M + 1) |
| 152 | 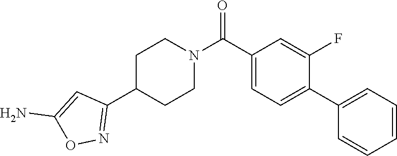 [SMILES: Nc1cc(C2CCN(C(=O)c3ccc(-c4ccccc4)c(F)c3)CC2)no1] | ++++ | ¹H NMR (400 MHz, CDCl₃) δ 7.57- 7.53 (m, 2H), 7.47 (dt, J = 7.6, 6.9 Hz, 3H), 7.43-7.36 (m, 1H), 7.27 (d, J = 1.6 Hz, 1H), 7.22 (dd, J = 10.5, 1.5 Hz, 1H), 5.01 (s, 1H), 4.68 (s, 1H), 4.44 (s, 2H), 3.90 (s, 1H), 3.17 (s, 1H), 3.03 (s, 1H), 2.92 (tt, J = 11.3, 3.8 Hz, 1H), 2.01 (s, 2H), 1.73 (s, 2H). ESI-MS: m/z = 366.1 (M + 1) |
| 153 | 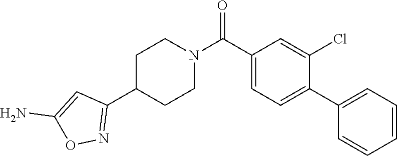 [SMILES: Nc1cc(C2CCN(C(=O)c3ccc(-c4ccccc4)c(Cl)c3)CC2)no1] | +++ | ¹H NMR (400 MHz, CDCl₃) δ 7.53 (d, J = 1.4 Hz, 1H), 7.48-7.33 (m, 7H), 5.02 (s, 1H), 4.68 (s, 1H), 4.42 (s, 2H), 3.90 (s, 1H), 3.20 (s, 1H), 3.02 (s, 1H), 2.92 (tt, J = 11.3, 3.9 Hz, 1H), 2.03 (s, 2H), 1.73 (s, 2H). ESI-MS: m/z = 382.1 (M + 1). |
| 154 | 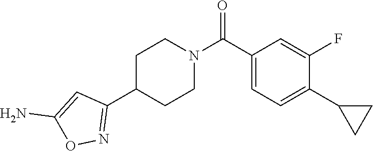 [SMILES: Nc1cc(C2CCN(C(=O)c3ccc(C4CC4)c(F)c3)CC2)no1] | − | ¹H NMR (400 MHz, CDCl₃) δ 7.11- 7.04 (m, 2H), 6.91 (dd, J = 10.2, 5.4 Hz, 1H), 5.00 (s, 1H), 4.64 (s, 1H), 4.41 (s, 2H), 3.85 (s, 1H), 3.04 (s, 2H), 2.88 (tt, J = 11.4, 3.9 Hz, 1H), 2.10 (ddd, J = 13.7, 8.5, 5.2 Hz, 1H), 1.96 (s, 2H), 1.69 (s, 2H), 1.02 (ddd, J = 8.5, 6.5, 4.6 Hz, 2H), 0.79-0.70 (m, 2H). ESI-MS: m/z = 330.1 (M + 1). |
| 155 | 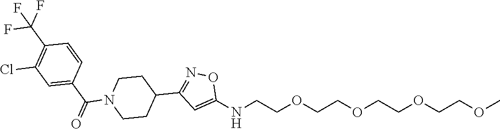 [SMILES: COCCOCCOCCOCCNc1cc(C2CCN(C(=O)c3ccc(C(F)(F)F)c(Cl)c3)CC2)no1] | ++++ | ¹H NMR (400 MHz, CDCl₃) δ 7.74 (d, J = 8.1 Hz, 1H), 7.55 (s, 1H), 7.38 (d, J = 8.0 Hz, 1H), 5.44 (t, J = 5.7 Hz, 1H), 4.84 (s, 1H), 4.63 (s, 1H), 3.70 (s, 1H), 3.69-3.60 (m, 12H), 3.58-3.54 (m, 2H), 3.38 (s, 3H), 3.34 (dd, J = 10.3, 5.6 Hz, 2H), 3.19 (d, J = 11.4 Hz, 1H), 3.05 (d, J = 13.8 Hz, 1H), 2.90 (tt, J = |
| | | | 11.0, 3.8 Hz, 1H), 2.04 (s, 1H), 1.97 |
| | | | (s, 1H), 1.69 (s, 2H). ESI-MS: m/z = |
| | | | 564.2 (M + 1) |
| 156 | 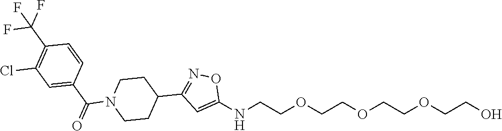 [SMILES: O=C(c1ccc(C(F)(F)F)c(Cl)c1)N1CCC(c2cc(NCCOCCOCCOCCO)on2)CC1] | ++++ | ¹H NMR (400 MHz, CDCl₃) δ 7.74 (d, J = 8.0 Hz, 1H), 7.55 (s, 1H), 7.38 (d, J = 8.0 Hz, 1H), 6.07 (t, J = 5.8 Hz, 1H), 4.82 (s, 1H), 4.62 (s, 1H), 3.79- 3.70 (m, 4H), 3.65 (dd, J = 8.8, 5.1 Hz, 10H), 3.35 (dd, J = 10.0, 5.5 Hz, 2H), 3.29 (s, 1H), 3.17 (s, 1H), 3.03 (s, 1H), 2.90 (tt, J = 11.0, 3.8 Hz, 1H), 2.15- |
| | | | 1.90 (m, 2H), 1.76 (s, 2H). ESI-MS: |
| | | | m/z = 550.2 (M + 1) |
| 157 | 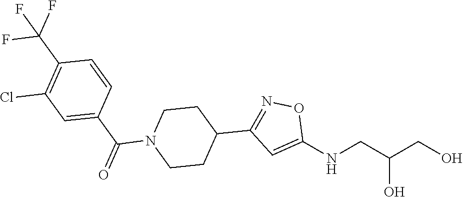 [SMILES: O=C(c1ccc(C(F)(F)F)c(Cl)c1)N1CCC(c2cc(NCC(O)CO)on2)CC1] | +++ | ¹H NMR (400 MHz, CDCl₃) δ 7.74 (d, J = 8.1 Hz, 1H), 7.54 (s, 1H), 7.38 (d, J = 8.0 Hz, 1H), 5.27 (t, J = 5.9 Hz, 1H), 4.89 (s, 1H), 4.61 (s, 1H), 3.98- 3.85 (m, 1H), 3.72 (dd, J = 11.2, 3.7 Hz, 1H), 3.65 (s, 1H), 3.60 (dd, J = 11.2, 5.9 Hz, 1H), 3.31 (ddd, J = 13.1, 6.7, 4.3 Hz, 1H), 3.27-3.11 (m, 2H), 3.00 (s, 1H), 2.89 (tt, J = 11.1, 3.7 Hz, 1H), 2.11-1.90 (m, 3H), 1.73 (s, 1H), 1.66 (s, 1H). ESI- MS: m/z = 448.1 (M + 1) |
| 158 | 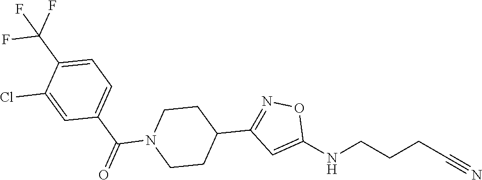 [SMILES: N#CCCCNc1cc(C2CCN(C(=O)c3ccc(C(F)(F)F)c(Cl)c3)CC2)no1] | ++++ | ¹H NMR (400 MHz, CDCl₃) δ 7.74 (d, J = 8.0 Hz, 1H), 7.55 (s, 1H), 7.39 (d, J = 8.0 Hz, 1H), 4.93 (s, 1H), 4.65 (s, 1H), 4.61 (t, J = 6.2 Hz, 1H), 3.70 (s, 1H), 3.38 (q, J = 6.5 Hz, 2H), 3.18 (s, 1H), 3.03 (s, 1H), 2.91 (tt, J = 11.1, 3.9 Hz, 1H), 2.48 (t, J = 7.0 Hz, 2H), 2.05 (s, 1H), 2.03-1.92 (m, 3H), 1.75 (s, 1H) 1.68 (s, 1H). ESI-MS: m/z = 440.8 (M + 1) |
| 159 | 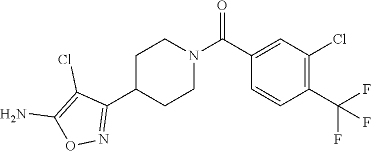 [SMILES: Nc1onc(C2CCN(C(=O)c3ccc(C(F)(F)F)c(Cl)c3)CC2)c1Cl] | ++++ | ¹H NMR (400 MHz, CDCl₃) δ 7.75 (d, J = 8.0 Hz, 1H), 7.55 (s, 1H), 7.42-7.36 (m, 1H), 4.64 (s, 1H), 4.58 (s, 2H), 3.73 (s, 1H), 3.18 (s, 1H), 3.07 (s, 1H), 2.96 (tt, J = 10.9, 4.0 Hz, 1H), 2.10 (s, 1H), 1.87 (s, 3H). ESI-MS: m/z = 408.1 (M + 1) |
| 160 | 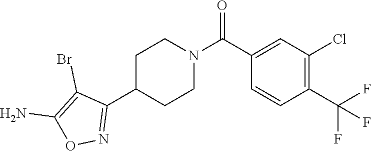 [SMILES: Nc1onc(C2CCN(C(=O)c3ccc(C(F)(F)F)c(Cl)c3)CC2)c1Br] | ++++ | ¹H NMR (400 MHz, CDCl₃) δ 7.74 (d, J = 8.0 Hz, 1H), 7.55 (s, 1H), 7.39 (d, J = 8.0 Hz, 1H), 4.63 (s, 3H), 3.72 (s, 1H), 3.18 (s, 1H), 3.07 (s, 1H), 2.92 (tt, J = 10.9, 3.9 Hz, 1H), 2.10 (s, 1H), 1.86 (s, 3H). ESI-MS: m/z = 451.5 (M + 1) |
| 161 | 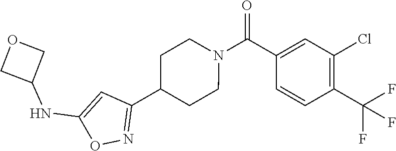 [SMILES: O=C(c1ccc(C(F)(F)F)c(Cl)c1)N1CCC(c2cc(NC3COC3)on2)CC1] | ++++ | 1H NMR (400 MHz, CDCl3) δ 7.74 (d, J = 8.1 Hz, 1H), 7.55 (s, 1H), 7.38 (d, J = 8.0 Hz, 1H), 5.10-4.92 (m, 3H), 4.89 (s, 1H), 4.75-4.55 (m, 4H), 3.72 (s, 1H), 3.17 (s, 1H), 3.02 (s, 1H), 2.98-2.85 (m, 1H), 2.20-1.90 (m, 2H), 1.80-1.75 (s, 2H). ESI-MS: m/z = 429.7 (M + 1) |
| 162 | 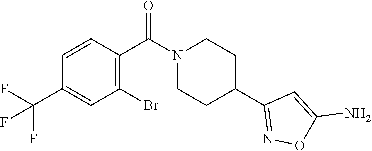 [SMILES: Nc1cc(C2CCN(C(=O)c3ccc(C(F)(F)F)cc3Br)CC2)no1] | ++++ | 1H NMR (400 MHz, CDCl3) δ 7.86 (d, J = 5.5 Hz, 1H), 7.63 (d, J = 7.9 Hz, 1H), 7.39 (dd, J = 19.9, 7.9 Hz, 1H), 4.99 (d, J = 2.9 Hz, 1H), 4.71 (dd, J = 25.5, 13.7 Hz, 1H), 4.44 (s, 2H), 3.44 (t, J = 13.7 Hz, 1H), 3.26-2.99 (m, 2H), 2.90 (ddd, J = 14.6, 7.4, 3.7 Hz, 1H), 2.07 (dd, J = 17.6, 7.2 Hz, 1H), 1.97-1.69 (m, 3H). ESI-MS: m/z = 417.6 [M + H]⁺. |
| 163 | 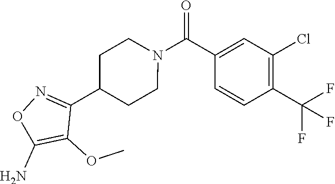 [SMILES: COc1c(C2CCN(C(=O)c3ccc(C(F)(F)F)c(Cl)c3)CC2)noc1N] | ++++ | 1H NMR (400 MHz, CDCl3) δ 7.74 (d, J = 8.0 Hz, 1H), 7.55 (s, 1H), 7.39 (d, J = 8.1 Hz, 1H), 4.62 (s, 1H), 4.24 (s, 2H), 3.73 (s, 1H). 3.71 (s, 3H), 3.25- 3.01 (m, 2H), 2.97 (tt, J = 10.9, 3.9 Hz, 1H), 2.10 (s, 1H), 2.01-1.80 (m, 3H). ESI-MS: m/z = 404.1 [M + H]+. |
| 164 | 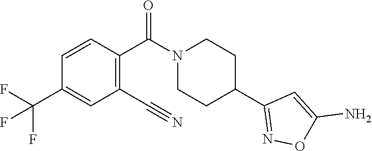 [SMILES: N#Cc1cc(C(F)(F)F)ccc1C(=O)N1CCC(c2cc(N)on2)CC1] | + | 1H NMR (400 MHz, CDCl3) δ 7.98 (s, 1H), 7.92 (d, J = 8.6 Hz, 1H), 7.62 (d, J = 8.1 Hz, 1H), 5.03 (s, 1H), 4.70 (d, J = 13.2 Hz, 1H), 4.44 (s, 2H), 3.47 (d, J = 13.6 Hz, 1H), 3.25 (t, J = 11.4 Hz, 1H), 3.11 (t, J = 11.7 Hz, 1H), 2.94 (tt, J = 11.0, 3.8 Hz, 1H), 2.08 (d, J = 11.3 Hz, 1H), 1.93 (d, J = 11.1 Hz, 1H), 1.89-1.73 (m, 2H). ESI-MS: m/z = 364.7 [M + H]⁺. |
| 165 | 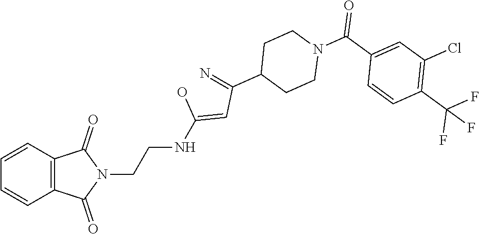 [SMILES: N=C(/C=C(\O)NCCN1C(=O)c2ccccc2C1=O)C1CCN(C(=O)c2ccc(C(F)(F)F)c(Cl)c2)CC1] | ++++ | 1H NMR (400 MHz, DMSO) Î′ 11.93 (s, 1H), 7.94 (d, J = 8.1 Hz, 1H), 7.84 (s, 2H), 7.69 â€“ 7.51 (m, 4H), 6.40 (s, 1H), 4.50 (d, J = 11.4 Hz, 1H), 4.31 (t, J = 9.5 Hz, 2H), 3.88 (t, J = 9.5 Hz, 2H), 3.51 (d, J = 12.7 Hz, 1H), 3.21 (t, J = 11.4 Hz, 1H), 3.08 â€“ 2.92 (m, 2H), 2.00 (d, J = 11.4 Hz, 1H), 1.81 (s, 1H), 1.72 (dt, J = 12.0, 8.6 Hz, 2H). |

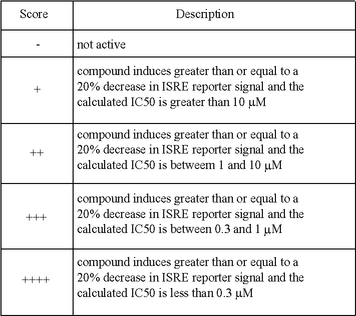

### Example 166: Inhibition of cGAMP Formation in Response to Cytosolic Viral dsDNA

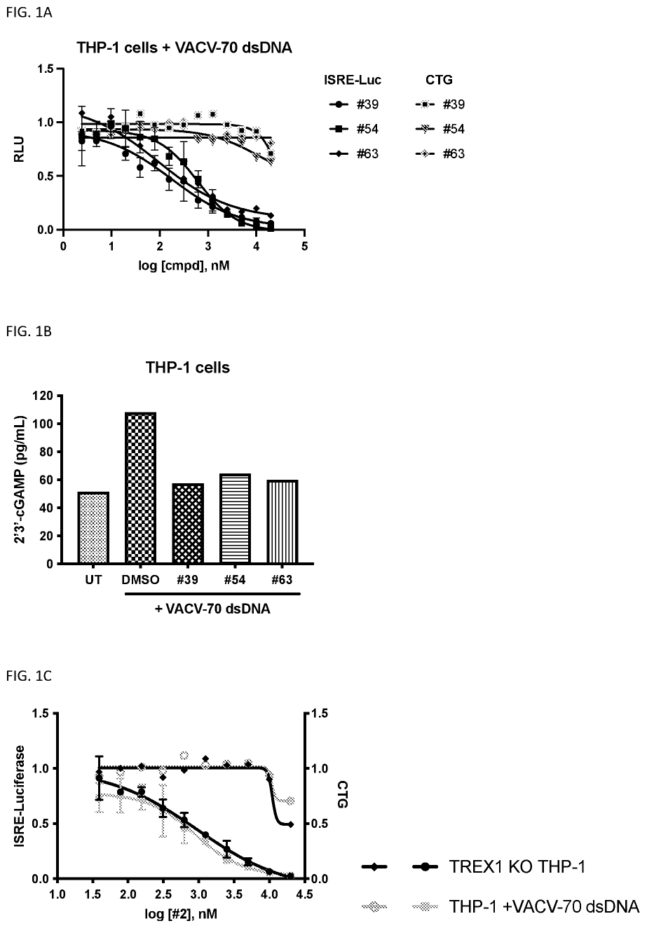

THP-1 ISG Luc cells were transiently transfected with VACV-70 dsDNA and treated with increasing concentrations of compounds 39, 54, and 63, respectively, for 48 h. Luminescence was detected after adding Quanti-Luc reading reagent for luciferase activity or Cell-Titer Glo (CTG) for viability. Relative luminescence units (RLU) were calculated by dividing test article luminescence by DMSO luminescence, and a dose response was graphed using a 4-parameter fit with a variable slope (FIG. 1A).

In addition, THP-1 ISG Luc cells were transiently transfected with VACV-70 dsDNA and treated with either compound 39 [180 nM], compound 54 [600 nM], compound 63 [90 nM], or DMSO for 48 h. Intracellular 2′3′-cGAMP levels were quantified using a 2′3′-cGAMP ELISA and standard curve (FIG. 1B).

TREX1 KO THP-1 ISG Luc cells or VACV-70 transfected (WT) THP-1 ISG Luc cells were treated with increasing concentrations of compound 2 for 48 h. Luminescence was detected after adding Quanti-Luc reading reagent for luciferase activity or Cell-Titer Glo (CTG) for viability. Relative luminescence units (RLU) were calculated by dividing test article luminescence by DMSO luminescence and a dose response was graphed using a 4-parameter fit with a variable slope (FIG. 1C). Calculated concentration of compound 2 is for 50% inhibition of ISRE-luciferase activity or cell viability in TREX1 KO THP-1 ISG Luc cells or THP-1 ISG Luc cells transiently transfected with VACV-70 dsDNA (Table 3).

TABLE 3
Administration of Compound 2.

| | ISRE-Luc IC₅₀ [μM] | CTG IC₅₀ [μM] |
|---|---|---|
| TREX1 KO | 0.95 | >10 |
| +VACV-70 dsDNA | 0.89 | >10 |

### NUMBERED REFERENCES IN THE PRESENT DISCLOSURE ARE AS FOLLOWS AND ARE HEREIN INCORPORATED IN THEIR ENTIRETIES

1. Decout, A., et al. The cGAS-STING pathway as a therapeutic target in inflammatory diseases. Nat. Rev. Immunol. (2021).
2. Crow, Y. J. et al. Aicardi-Goutieres syndrome and the type I interferonopathies. Nat. Rev. Immunol. 15, 429-440 (2015).
3. Uggenti, C. et al. Self-awareness: nucleic acid-driven inflammation and the type I interferonopathies. Annu. Rev. Immunol. 37, 247-267 (2019).
4. An, J. et al. Expression of cyclic GMP-AMP synthase in patients with systemic lupus erythematosus. Arthritis Rheumatol. 69, 800-807 (2017).
5. Kato, Y. et al. Apoptosis-derived membrane vesicles drive the cGAS-STING pathway and enhance type I IFN production in systemic lupus erythematosus. Ann. Rheum. Dis. 77, 1507-1515 (2018).
6. Thim-uam, A. et al. STING mediates lupus via the activation of conventional dendritic cell maturation and plasmacytoid dendritic cell differentiation. iScience 23, 101530 (2020).
7. J. Wang et al., Accumulation of cytosolic dsDNA contributes to fibroblast-like synoviocytes-mediated rheumatoid arthritis synovial inflammation, International Immunopharmacology (76) (2019) 105791.
8. Li, Q. et al. Inhibition of double-strand DNA-sensing cGAS ameliorates brain injury after ischemic stroke. EMBO Mol. Med. 12, e11002 (2020).
9. Sliter, D. A. et al. Parkin and PINK1 mitigate STING-induced inflammation. Nature 561, 258-262 (2018).
10. Nazmi, A. et al. Chronic neurodegeneration induces type I interferon synthesis via STING, shaping microglial phenotype and accelerating disease progression. Glia 67, 1254-1276 (2019).
11. Sharma, M., Rajendrarao, S., Shahani, N., Ramirez-Jarquin, U. N. & Subramaniam, S. Cyclic GMP-AMP synthase promotes the inflammatory and autophagy responses in Huntington disease. Proc. Natl Acad. Sci. USA 117, 15989-15999 (2020).
12. Yu, C. H. et al. TDP-43 triggers mitochondrial DNA release via mPTP to activate cGAS/STING in ALS. Cell 183, 636-649 (2020).
13. McCauley, M. E. et al. C9orf72 in myeloid cells suppresses STING-induced inflammation. Nature 585, 96-101 (2020).
14. Abdullah, A. et al. STING-mediated type-I interferons contribute to the neuroinflammatory process and detrimental effects following traumatic brain injury. J. Neuroinflammation 15, 323 (2018).
15. Glück, S. et al. Innate immune sensing of cytosolic chromatin fragments through cGAS promotes senescence. Nat. Cell Biol. 19, 1061-1070 (2017).
16. Dou, Z. et al. Cytoplasmic chromatin triggers inflammation in senescence and cancer. Nature 550, 402-406 (2017).
17. Yang, H., Wang, H., Ren, J., Chen, Q. & Chen, Z. J. cGAS is essential for cellular senescence. Proc. Natl Acad. Sci. USA 114, E4612-E4620 (2017).
18. Kerur, N. et al. cGAS drives noncanonical-inflammasome activation in age-related macular degeneration. Nat. Med. 24, 50-61 (2018).
19. Lu, G.-L. et al. Synergistic inflammatory signaling by cGAS may be involved in the development of atherosclerosis, Aging 13(4), 5650 (2021).

We claim:

1. A method of treating an inflammatory disease or condition in a subject suffering therefrom, comprising administering to the subject a compound of Formula (I): 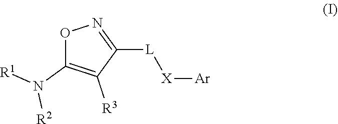
wherein
R¹ and R² are independently selected from the group consisting of H, C₁-C₆-alkyl (optionally substituted by one to five substituents independently selected from halo, CN, and OH), —C(O)C₁-C₆-alkyl, —C(O)H, —C₁-C₆-alkyl-(C₆-C₁₀-aryl), —C₁-C₆-alkyl-(C₁-C₆-alkoxy), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S), and —(C₁-C₆-alkyl)-SO₂—(C₁-C₆-alkyl), —(CH₂CH₂O)ₙ—R (wherein n is an integer selected from 2, 3, 4, 5, 6, 7, 8, 9, and 10, and R is H or C₁-C₆-alkyl);
R³ is selected from the group consisting of H, halo, C₁-C₆-alkyl, C₁-C₆-haloalkyl, and C₁-C₆-alkoxy;
L is a moiety selected from the group consisting of: 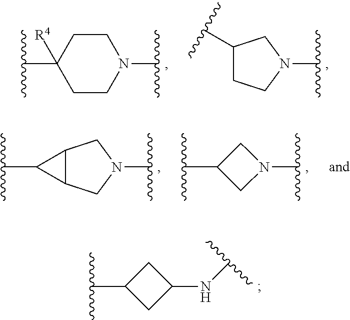
R⁴ is selected from the group consisting of H, halo, and C₁-C₆-alkyl;
X is —C(O)— or —SO₂—; and
Ar is phenyl or indolyl, wherein Ar is optionally substituted with one to five substituents selected from the group consisting of halo, C₁-C₆-alkyl, C₁-C₆-haloalkyl, C₁-C₆-alkoxy, C₁-C₆-haloalkoxy, CN, OH, NO₂, —NRR′ (wherein R and R′ are independently selected from H and C₁-C₆-alkyl), —(C₁-C₆-alkyl)NRR′, —C(O)NRR′, —SO₂R, C₆-C₁₀-aryl, 3- to 6-membered heterocycloalkyl (wherein 1-4 ring members are independently selected from N, O, and S), 5- to 10-membered heteroaryl (wherein 1-4 heteroaryl members are independently selected from N, O, and S), and C₃-C₁₀-cycloalkyl;
or a pharmaceutically acceptable salt thereof.

2. The method according to claim 1, wherein Ar is optionally substituted phenyl.

3. The method according to claim 1 or 2, wherein Ar is substituted with one to three substituents selected from the group consisting of halo, C₁-C₆-alkyl, C₁-C₆-haloalkyl, C₁-C₆-alkoxy, C₁-C₆-haloalkoxy.

4. The method according to any one of claims 1 to 3, wherein Ar is substituted with one to three substituents selected from the group consisting of halo, C₁-C₆-haloalkyl, and C₁-C₆-haloalkoxy.

5. The method according to any one of claims 1 to 4, wherein Ar is substituted with at least one of —CF₃ and —OCF₃.

6. The method according to any one of claims 1 to 5, wherein L is a moiety selected from:

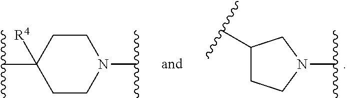

7. The method according to any one of claims 1 to 6, wherein L is

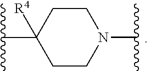

8. The method according to any one of claims 1 to 7, wherein R⁴ is H.

9. The method according to any one of claims 1 to 8, wherein X is —C(O)—.

10. The method according to any one of claims 1 to 8, wherein X is —SO₂—.

11. The method according to any one of claims 1 to 10, wherein R¹ and R² are independently selected from the group consisting of H and optionally substituted C₁-C₆-alkyl.

12. The method according to any one of claims 1 to 11, wherein each of R¹ and R² is H.

13. The method according to any one of claims 1 to 12, wherein R³ is H.

14. The method according to claim 1, wherein
each of R¹, R², R³, and R⁴ is H;
L is 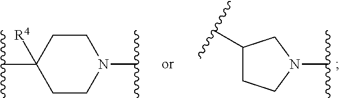
X is —C(O)—; and
Ar is phenyl substituted with one to three substituents independently selected from halo, C₁-C₆-haloalkyl, and C₁-C₆-haloalkoxy.

15. The method according to claim 1, wherein the compound or pharmaceutically acceptable salt thereof is one selected from the following table:

| | |
|---|---|
| 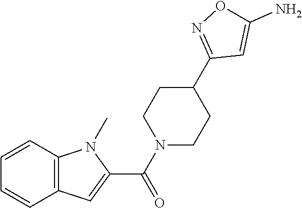 [SMILES: Cn1c(C(=O)N2CCC(c3cc(N)on3)CC2)cc2ccccc21] | 1 |
| 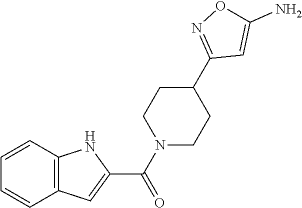 [SMILES: Nc1cc(C2CCN(C(=O)c3cc4ccccc4[nH]3)CC2)no1] | 2 |
| 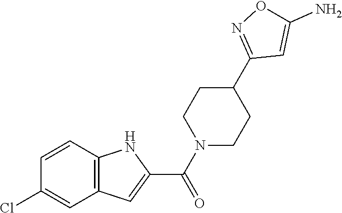 [SMILES: Nc1cc(C2CCN(C(=O)c3cc4cc(Cl)ccc4[nH]3)CC2)no1] | 3 |
| 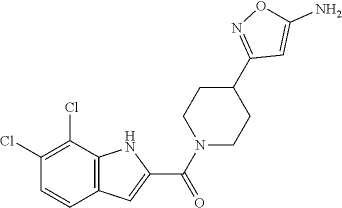 [SMILES: Nc1cc(C2CCN(C(=O)c3cc4ccc(Cl)c(Cl)c4[nH]3)CC2)no1] | 4 |
| 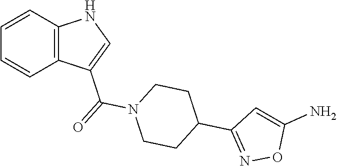 [SMILES: Nc1cc(C2CCN(C(=O)c3c[nH]c4ccccc34)CC2)no1] | 5 |
| 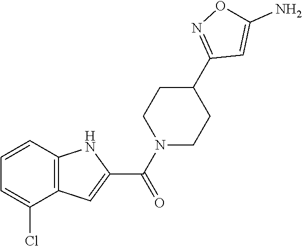 [SMILES: Nc1cc(C2CCN(C(=O)c3cc4c(Cl)cccc4[nH]3)CC2)no1] | 6 |
| 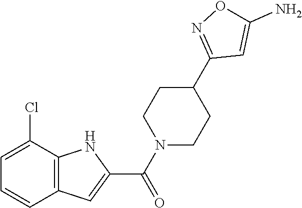 [SMILES: Nc1cc(C2CCN(C(=O)c3cc4cccc(Cl)c4[nH]3)CC2)no1] | 7 |
| 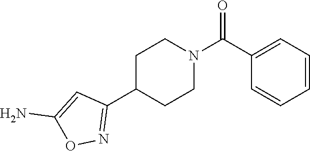 [SMILES: Nc1cc(C2CCN(C(=O)c3ccccc3)CC2)no1] | 8 |
| 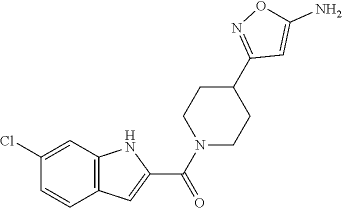 [SMILES: Nc1cc(C2CCN(C(=O)c3cc4ccc(Cl)cc4[nH]3)CC2)no1] | 9 |
| 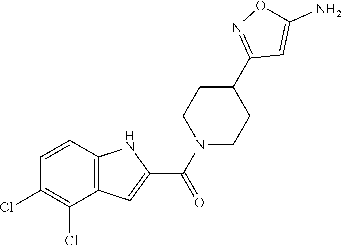 [SMILES: Nc1cc(C2CCN(C(=O)c3cc4c(Cl)c(Cl)ccc4[nH]3)CC2)no1] | 10 |
| 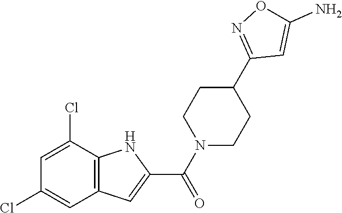 [SMILES: Nc1cc(C2CCN(C(=O)c3cc4cc(Cl)cc(Cl)c4[nH]3)CC2)no1] | 11 |
| 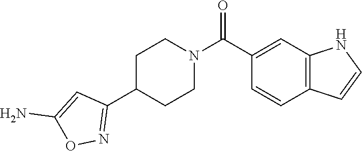 [SMILES: Nc1cc(C2CCN(C(=O)c3ccc4cc[nH]c4c3)CC2)no1] | 12 |
| 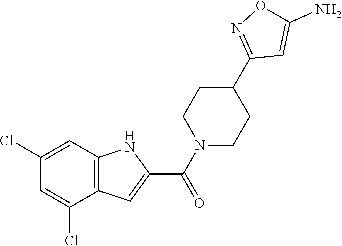 [SMILES: Nc1cc(C2CCN(C(=O)c3cc4c(Cl)cc(Cl)cc4[nH]3)CC2)no1] | 13 |
| 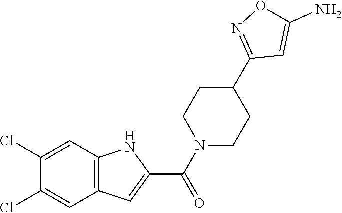 [SMILES: Nc1cc(C2CCN(C(=O)c3cc4cc(Cl)c(Cl)cc4[nH]3)CC2)no1] | 14 |
| 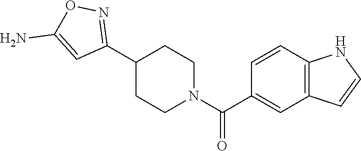 [SMILES: Nc1cc(C2CCN(C(=O)c3ccc4[nH]ccc4c3)CC2)no1] | 15 |
| 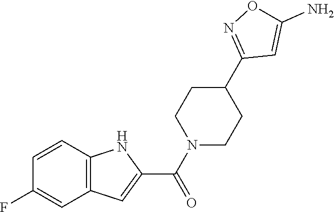 [SMILES: Nc1cc(C2CCN(C(=O)c3cc4cc(F)ccc4[nH]3)CC2)no1] | 16 |
| 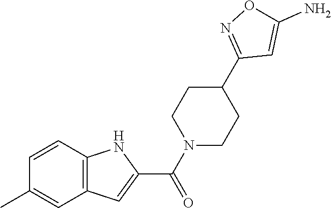 [SMILES: Cc1ccc2[nH]c(C(=O)N3CCC(c4cc(N)on4)CC3)cc2c1] | 17 |
| 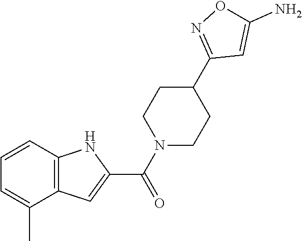 [SMILES: Cc1cccc2[nH]c(C(=O)N3CCC(c4cc(N)on4)CC3)cc12] | 18 |
| 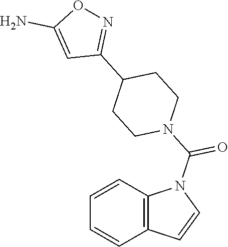 [SMILES: Nc1cc(C2CCN(C(=O)n3ccc4ccccc43)CC2)no1] | 19 |
| 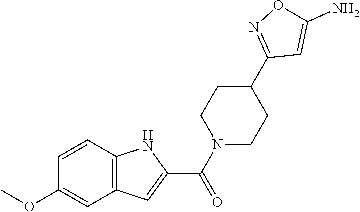 [SMILES: COc1ccc2[nH]c(C(=O)N3CCC(c4cc(N)on4)CC3)cc2c1] | 20 |
| 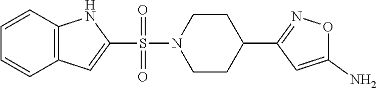 [SMILES: Nc1cc(C2CCN(S(=O)(=O)c3cc4ccccc4[nH]3)CC2)no1] | 21 |
| 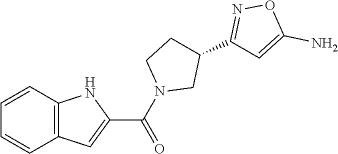 [SMILES: Nc1cc([C@H]2CCN(C(=O)c3cc4ccccc4[nH]3)C2)no1] | 22 |
| 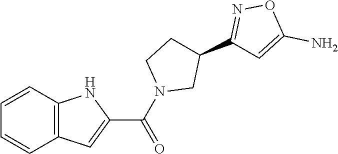 [SMILES: Nc1cc([C@@H]2CCN(C(=O)c3cc4ccccc4[nH]3)C2)no1] | 23 |
| 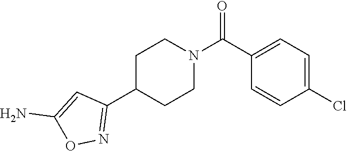 [SMILES: Nc1cc(C2CCN(C(=O)c3ccc(Cl)cc3)CC2)no1] | 24 |
| 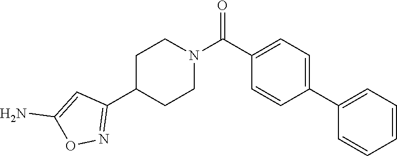 [SMILES: Nc1cc(C2CCN(C(=O)c3ccc(-c4ccccc4)cc3)CC2)no1] | 25 |
| 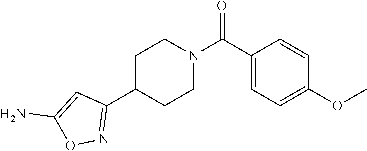 [SMILES: COc1ccc(C(=O)N2CCC(c3cc(N)on3)CC2)cc1] | 26 |
| 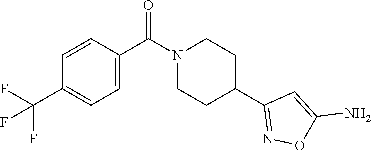 [SMILES: Nc1cc(C2CCN(C(=O)c3ccc(C(F)(F)F)cc3)CC2)no1] | 27 |
| 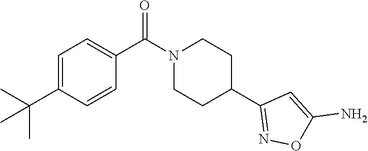 [SMILES: CC(C)(C)c1ccc(C(=O)N2CCC(c3cc(N)on3)CC2)cc1] | 28 |
| 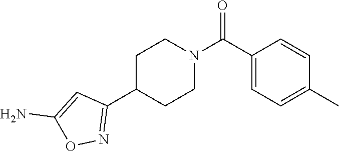 [SMILES: Cc1ccc(C(=O)N2CCC(c3cc(N)on3)CC2)cc1] | 29 |
| 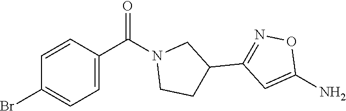 [SMILES: Nc1cc(C2CCN(C(=O)c3ccc(Br)cc3)C2)no1] | 30 |
| 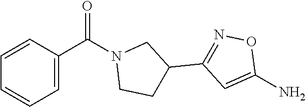 [SMILES: Nc1cc(C2CCN(C(=O)c3ccccc3)C2)no1] | 31 |
| 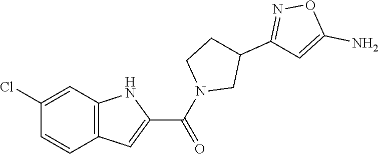 [SMILES: Nc1cc(C2CCN(C(=O)c3cc4ccc(Cl)cc4[nH]3)C2)no1] | 32 |
| 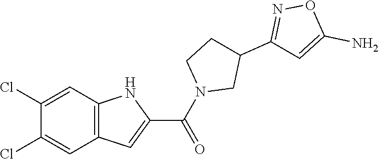 [SMILES: Nc1cc(C2CCN(C(=O)c3cc4cc(Cl)c(Cl)cc4[nH]3)C2)no1] | 33 |
| 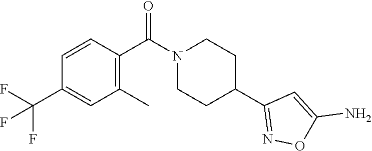 [SMILES: Cc1cc(C(F)(F)F)ccc1C(=O)N1CCC(c2cc(N)on2)CC1] | 34 |
| 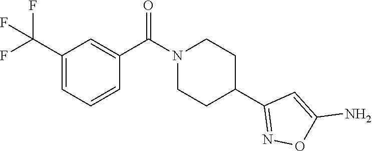 [SMILES: Nc1cc(C2CCN(C(=O)c3cccc(C(F)(F)F)c3)CC2)no1] | 35 |
| 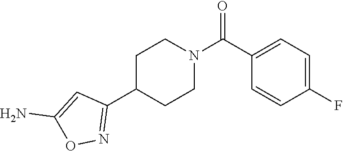 [SMILES: Nc1cc(C2CCN(C(=O)c3ccc(F)cc3)CC2)no1] | 36 |
| 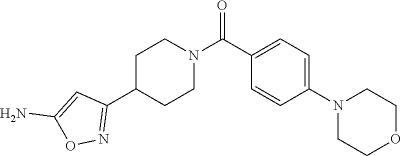 [SMILES: Nc1cc(C2CCN(C(=O)c3ccc(N4CCOCC4)cc3)CC2)no1] | 37 |
| 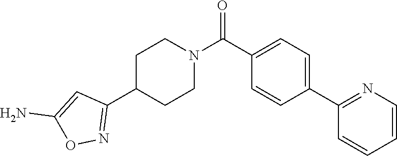 [SMILES: Nc1cc(C2CCN(C(=O)c3ccc(-c4ccccn4)cc3)CC2)no1] | 38 |
| 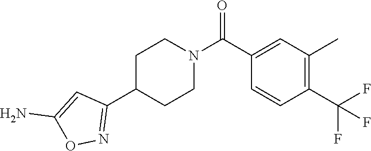 [SMILES: Cc1cc(C(=O)N2CCC(c3cc(N)on3)CC2)ccc1C(F)(F)F] | 39 |
| 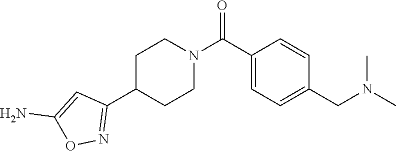 [SMILES: CN(C)Cc1ccc(C(=O)N2CCC(c3cc(N)on3)CC2)cc1] | 40 |
| 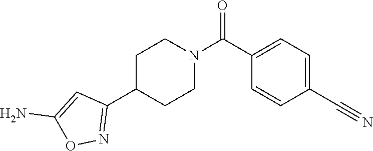 [SMILES: N#Cc1ccc(C(=O)N2CCC(c3cc(N)on3)CC2)cc1] | 41 |
| 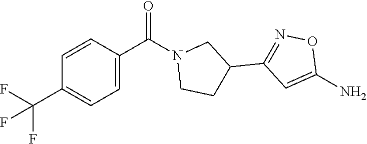 [SMILES: Nc1cc(C2CCN(C(=O)c3ccc(C(F)(F)F)cc3)C2)no1] | 42 |
| 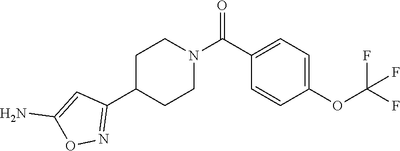 [SMILES: Nc1cc(C2CCN(C(=O)c3ccc(OC(F)(F)F)cc3)CC2)no1] | 43 |
| 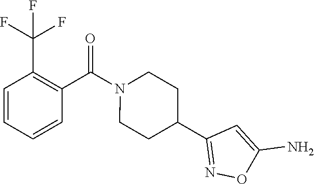 [SMILES: Nc1cc(C2CCN(C(=O)c3ccccc3C(F)(F)F)CC2)no1] | 44 |
| 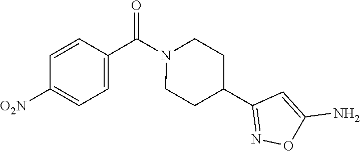 [SMILES: Nc1cc(C2CCN(C(=O)c3ccc([N+](=O)[O-])cc3)CC2)no1] | 45 |
| 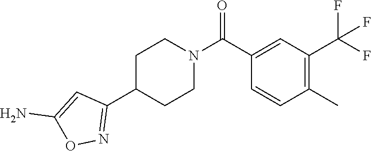 [SMILES: Cc1ccc(C(=O)N2CCC(c3cc(N)on3)CC2)cc1C(F)(F)F] | 46 |
| 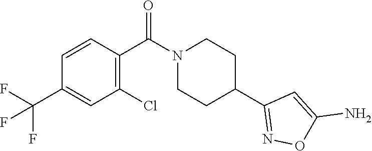 [SMILES: Nc1cc(C2CCN(C(=O)c3ccc(C(F)(F)F)cc3Cl)CC2)no1] | 47 |
| 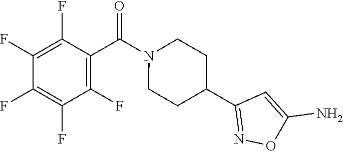 [SMILES: Nc1cc(C2CCN(C(=O)c3c(F)c(F)c(F)c(F)c3F)CC2)no1] | 48 |
| 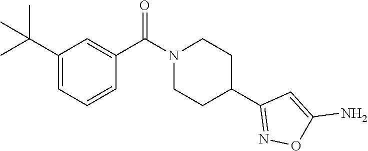 [SMILES: CC(C)(C)c1cccc(C(=O)N2CCC(c3cc(N)on3)CC2)c1] | 49 |
| 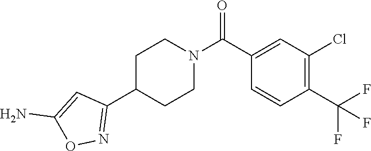 [SMILES: Nc1cc(C2CCN(C(=O)c3ccc(C(F)(F)F)c(Cl)c3)CC2)no1] | 50 |
| 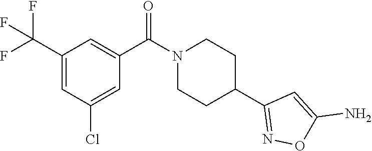 [SMILES: Nc1cc(C2CCN(C(=O)c3cc(Cl)cc(C(F)(F)F)c3)CC2)no1] | 51 |
| 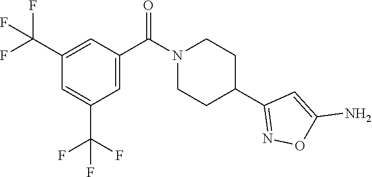 [SMILES: Nc1cc(C2CCN(C(=O)c3cc(C(F)(F)F)cc(C(F)(F)F)c3)CC2)no1] | 52 |
| 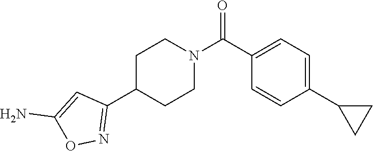 [SMILES: Nc1cc(C2CCN(C(=O)c3ccc(C4CC4)cc3)CC2)no1] | 53 |
| 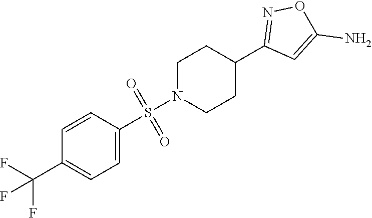 [SMILES: Nc1cc(C2CCN(S(=O)(=O)c3ccc(C(F)(F)F)cc3)CC2)no1] | 54 |
| 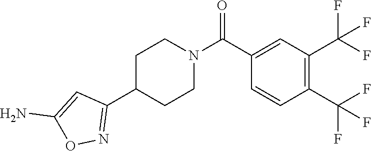 [SMILES: Nc1cc(C2CCN(C(=O)c3ccc(C(F)(F)F)c(C(F)(F)F)c3)CC2)no1] | 55 |
| 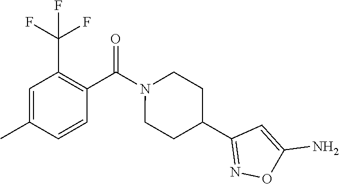 [SMILES: Cc1ccc(C(=O)N2CCC(c3cc(N)on3)CC2)c(C(F)(F)F)c1] | 56 |
| 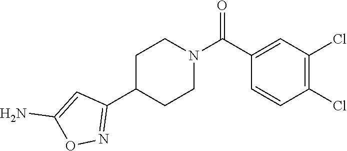 [SMILES: Nc1cc(C2CCN(C(=O)c3ccc(Cl)c(Cl)c3)CC2)no1] | 57 |
| 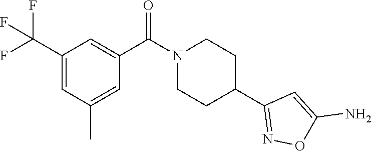 [SMILES: Cc1cc(C(=O)N2CCC(c3cc(N)on3)CC2)cc(C(F)(F)F)c1] | 58 |
| 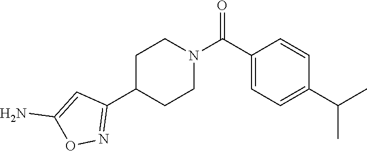 [SMILES: CC(C)c1ccc(C(=O)N2CCC(c3cc(N)on3)CC2)cc1] | 59 |
| 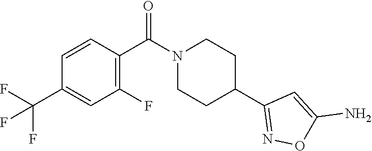 [SMILES: Nc1cc(C2CCN(C(=O)c3ccc(C(F)(F)F)cc3F)CC2)no1] | 60 |
| 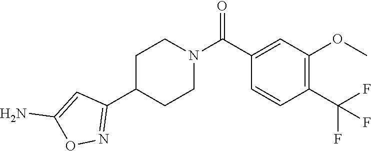 [SMILES: COc1cc(C(=O)N2CCC(c3cc(N)on3)CC2)ccc1C(F)(F)F] | 61 |
| 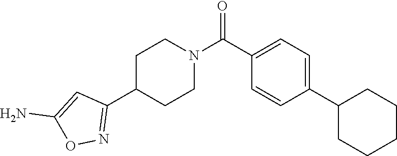 [SMILES: Nc1cc(C2CCN(C(=O)c3ccc(C4CCCCC4)cc3)CC2)no1] | 62 |
| 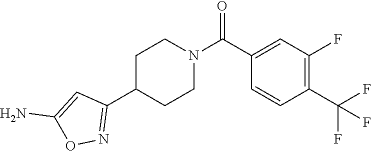 [SMILES: Nc1cc(C2CCN(C(=O)c3ccc(C(F)(F)F)c(F)c3)CC2)no1] | 63 |
| 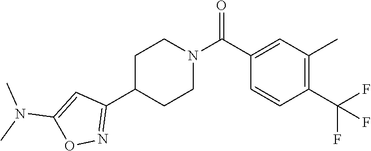 [SMILES: Cc1cc(C(=O)N2CCC(c3cc(N(C)C)on3)CC2)ccc1C(F)(F)F] | 64 |
| 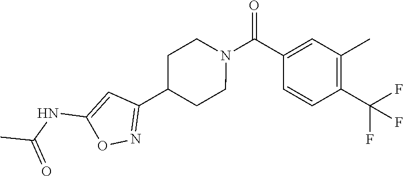 [SMILES: CC(=O)Nc1cc(C2CCN(C(=O)c3ccc(C(F)(F)F)c(C)c3)CC2)no1] | 65 |
| 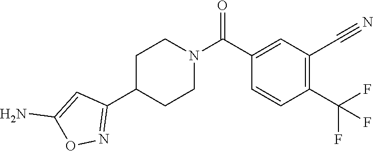 [SMILES: N#Cc1cc(C(=O)N2CCC(c3cc(N)on3)CC2)ccc1C(F)(F)F] | 66 |
| 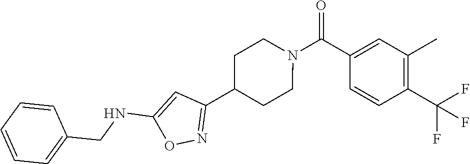 [SMILES: Cc1cc(C(=O)N2CCC(c3cc(NCc4ccccc4)on3)CC2)ccc1C(F)(F)F] | 67 |
| 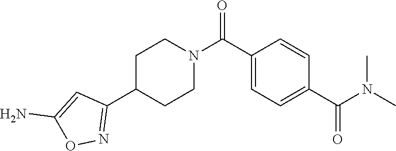 [SMILES: CN(C)C(=O)c1ccc(C(=O)N2CCC(c3cc(N)on3)CC2)cc1] | 68 |
| 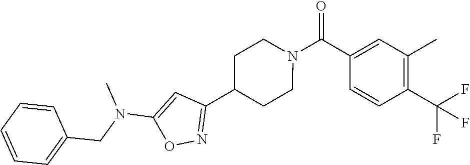 [SMILES: Cc1cc(C(=O)N2CCC(c3cc(N(C)Cc4ccccc4)on3)CC2)ccc1C(F)(F)F] | 69 |
| 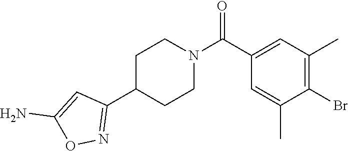 [SMILES: Cc1cc(C(=O)N2CCC(c3cc(N)on3)CC2)cc(C)c1Br] | 70 |
| 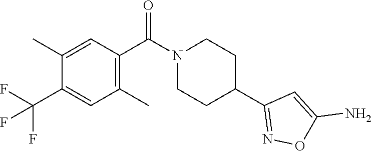 [SMILES: Cc1cc(C(F)(F)F)c(C)cc1C(=O)N1CCC(c2cc(N)on2)CC1] | 71 |
| 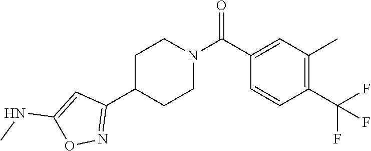 [SMILES: CNc1cc(C2CCN(C(=O)c3ccc(C(F)(F)F)c(C)c3)CC2)no1] | 72 |
| 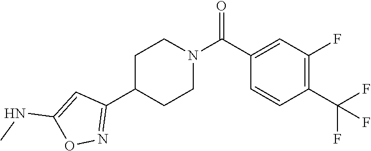 [SMILES: CNc1cc(C2CCN(C(=O)c3ccc(C(F)(F)F)c(F)c3)CC2)no1] | 73 |
| 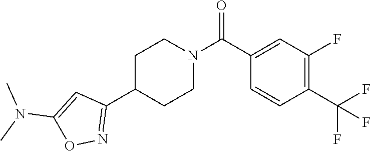 [SMILES: CN(C)c1cc(C2CCN(C(=O)c3ccc(C(F)(F)F)c(F)c3)CC2)no1] | 74 |
| 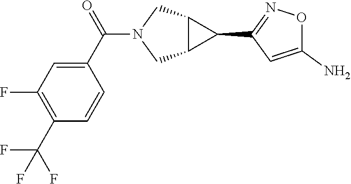 [SMILES: Nc1cc([C@H]2[C@@H]3CN(C(=O)c4ccc(C(F)(F)F)c(F)c4)C[C@@H]32)no1] | 75 |
| 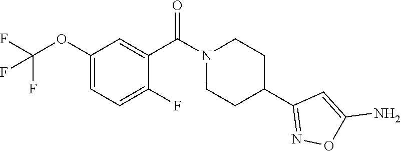 [SMILES: Nc1cc(C2CCN(C(=O)c3cc(OC(F)(F)F)ccc3F)CC2)no1] | 76 |
| 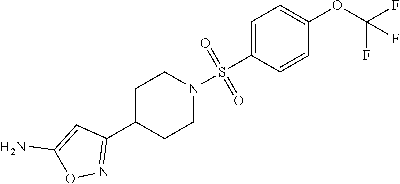 [SMILES: Nc1cc(C2CCN(S(=O)(=O)c3ccc(OC(F)(F)F)cc3)CC2)no1] | 77 |
| 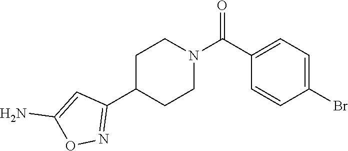 [SMILES: Nc1cc(C2CCN(C(=O)c3ccc(Br)cc3)CC2)no1] | 78 |
| 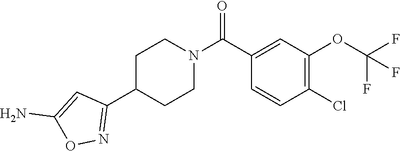 [SMILES: Nc1cc(C2CCN(C(=O)c3ccc(Cl)c(OC(F)(F)F)c3)CC2)no1] | 79 |
| 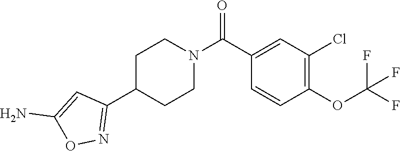 [SMILES: Nc1cc(C2CCN(C(=O)c3ccc(OC(F)(F)F)c(Cl)c3)CC2)no1] | 80 |
| 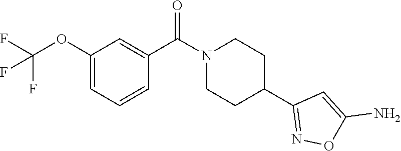 [SMILES: Nc1cc(C2CCN(C(=O)c3cccc(OC(F)(F)F)c3)CC2)no1] | 81 |
| 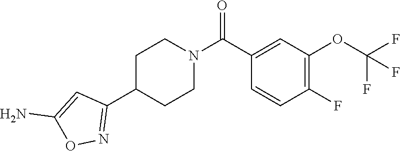 [SMILES: Nc1cc(C2CCN(C(=O)c3ccc(F)c(OC(F)(F)F)c3)CC2)no1] | 82 |
| 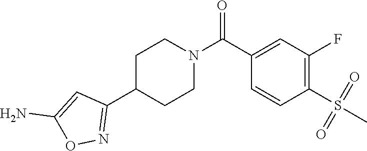 [SMILES: CS(=O)(=O)c1ccc(C(=O)N2CCC(c3cc(N)on3)CC2)cc1F] | 83 |
| 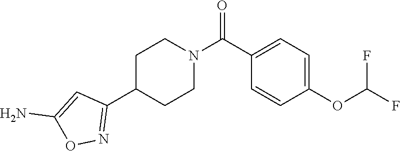 [SMILES: Nc1cc(C2CCN(C(=O)c3ccc(OC(F)F)cc3)CC2)no1] | 84 |
| 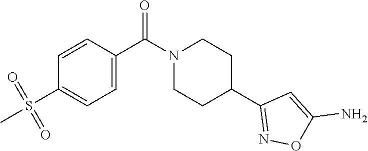 [SMILES: CS(=O)(=O)c1ccc(C(=O)N2CCC(c3cc(N)on3)CC2)cc1] | 85 |
| 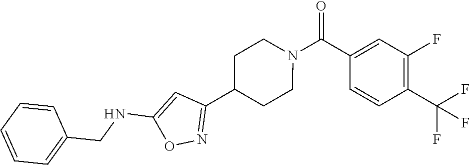 [SMILES: O=C(c1ccc(C(F)(F)F)c(F)c1)N1CCC(c2cc(NCc3ccccc3)on2)CC1] | 86 |
| 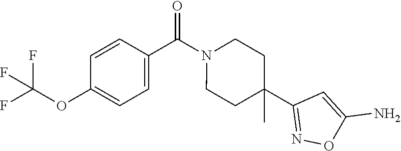 [SMILES: CC1(c2cc(N)on2)CCN(C(=O)c2ccc(OC(F)(F)F)cc2)CC1] | 87 |
| 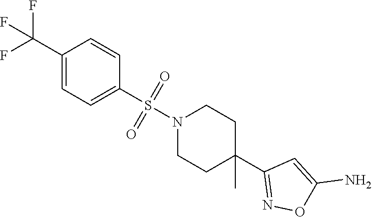 [SMILES: CC1(c2cc(N)on2)CCN(S(=O)(=O)c2ccc(C(F)(F)F)cc2)CC1] | 88 |
| 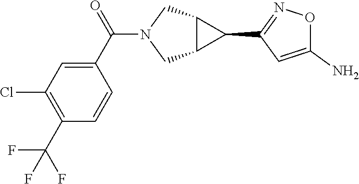 [SMILES: Nc1cc([C@H]2[C@@H]3CN(C(=O)c4ccc(C(F)(F)F)c(Cl)c4)C[C@@H]32)no1] | 89 |
| 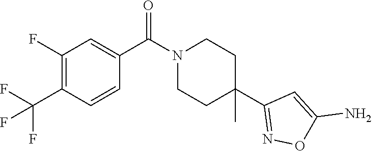 [SMILES: CC1(c2cc(N)on2)CCN(C(=O)c2ccc(C(F)(F)F)c(F)c2)CC1] | 90 |
| 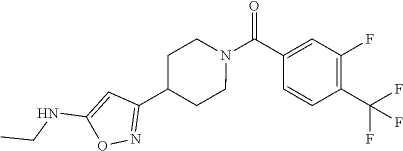 [SMILES: CCNc1cc(C2CCN(C(=O)c3ccc(C(F)(F)F)c(F)c3)CC2)no1] | 91 |
| 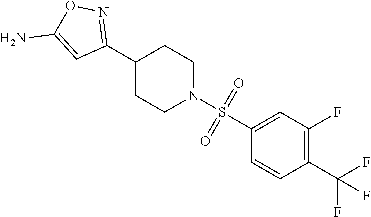 [SMILES: Nc1cc(C2CCN(S(=O)(=O)c3ccc(C(F)(F)F)c(F)c3)CC2)no1] | 92 |
| 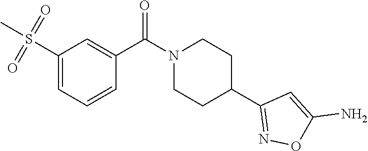 [SMILES: CS(=O)(=O)c1cccc(C(=O)N2CCC(c3cc(N)on3)CC2)c1] | 93 |
| 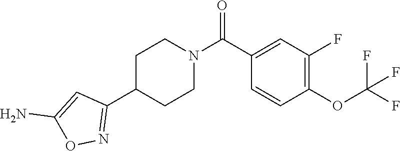 [SMILES: Nc1cc(C2CCN(C(=O)c3ccc(OC(F)(F)F)c(F)c3)CC2)no1] | 94 |
| 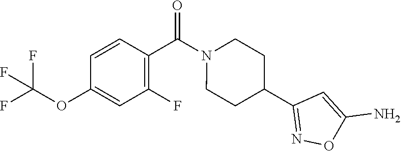 [SMILES: Nc1cc(C2CCN(C(=O)c3ccc(OC(F)(F)F)cc3F)CC2)no1] | 95 |
| 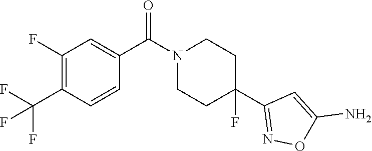 [SMILES: Nc1cc(C2(F)CCN(C(=O)c3ccc(C(F)(F)F)c(F)c3)CC2)no1] | 96 |
| 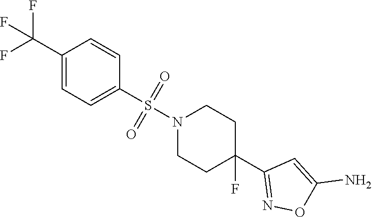 [SMILES: Nc1cc(C2(F)CCN(S(=O)(=O)c3ccc(C(F)(F)F)cc3)CC2)no1] | 97 |
| 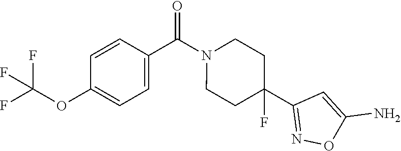 [SMILES: Nc1cc(C2(F)CCN(C(=O)c3ccc(OC(F)(F)F)cc3)CC2)no1] | 98 |
| 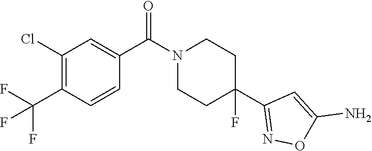 [SMILES: Nc1cc(C2(F)CCN(C(=O)c3ccc(C(F)(F)F)c(Cl)c3)CC2)no1] | 99 |
| 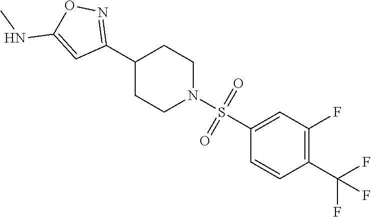 [SMILES: CNc1cc(C2CCN(S(=O)(=O)c3ccc(C(F)(F)F)c(F)c3)CC2)no1] | 100 |
| 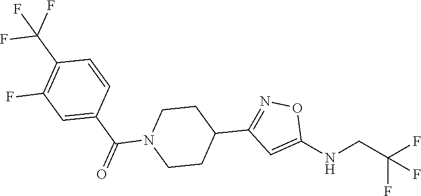 [SMILES: O=C(c1ccc(C(F)(F)F)c(F)c1)N1CCC(c2cc(NCC(F)(F)F)on2)CC1] | 101 |
| 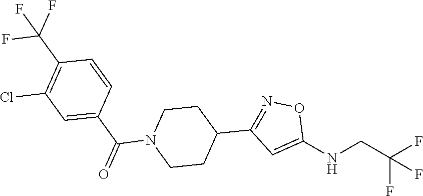 [SMILES: O=C(c1ccc(C(F)(F)F)c(Cl)c1)N1CCC(c2cc(NCC(F)(F)F)on2)CC1] | 102 |
| 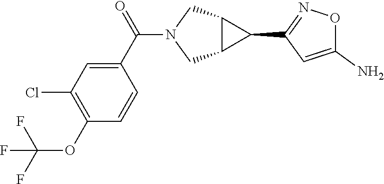 [SMILES: Nc1cc([C@H]2[C@@H]3CN(C(=O)c4ccc(OC(F)(F)F)c(Cl)c4)C[C@@H]32)no1] | 103 |
| 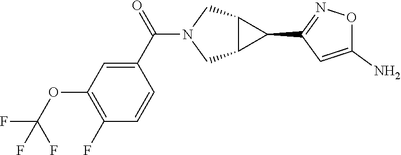 [SMILES: Nc1cc([C@H]2[C@@H]3CN(C(=O)c4ccc(F)c(OC(F)(F)F)c4)C[C@@H]32)no1] | 104 |
| 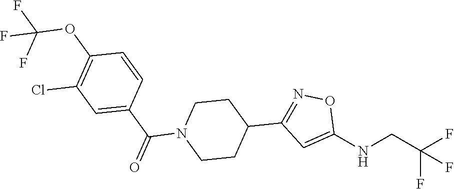 [SMILES: O=C(c1ccc(OC(F)(F)F)c(Cl)c1)N1CCC(c2cc(NCC(F)(F)F)on2)CC1] | 105 |
| 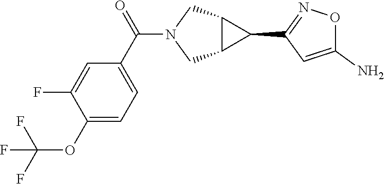 [SMILES: Nc1cc([C@H]2[C@@H]3CN(C(=O)c4ccc(OC(F)(F)F)c(F)c4)C[C@@H]32)no1] | 106 |
| 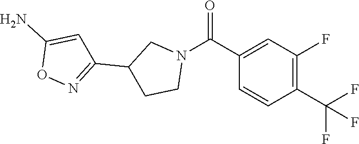 [SMILES: Nc1cc(C2CCN(C(=O)c3ccc(C(F)(F)F)c(F)c3)C2)no1] | 107 |
| 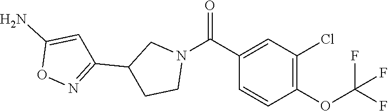 [SMILES: Nc1cc(C2CCN(C(=O)c3ccc(OC(F)(F)F)c(Cl)c3)C2)no1] | 108 |
| 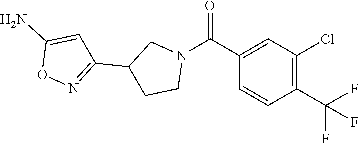 [SMILES: Nc1cc(C2CCN(C(=O)c3ccc(C(F)(F)F)c(Cl)c3)C2)no1] | 109 |
| 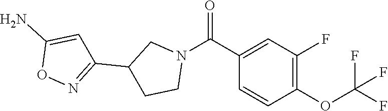 [SMILES: Nc1cc(C2CCN(C(=O)c3ccc(OC(F)(F)F)c(F)c3)C2)no1] | 110 |
| 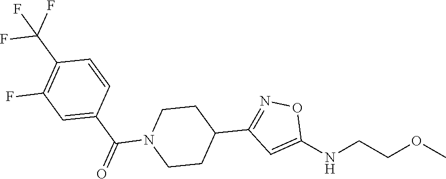 [SMILES: COCCNc1cc(C2CCN(C(=O)c3ccc(C(F)(F)F)c(F)c3)CC2)no1] | 111 |
| 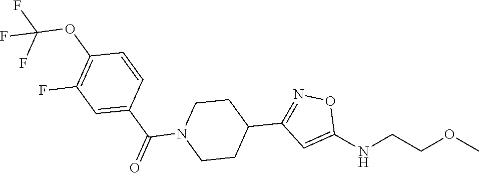 [SMILES: COCCNc1cc(C2CCN(C(=O)c3ccc(OC(F)(F)F)c(F)c3)CC2)no1] | 112 |
| 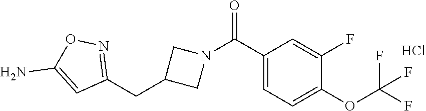 [SMILES: Nc1cc(CC2CN(C(=O)c3ccc(OC(F)(F)F)c(F)c3)C2)no1 ; Cl] | 113 |
| 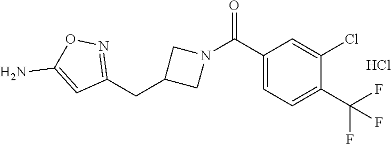 [SMILES: Nc1cc(CC2CN(C(=O)c3ccc(C(F)(F)F)c(Cl)c3)C2)no1 ; Cl] | 114 |
| 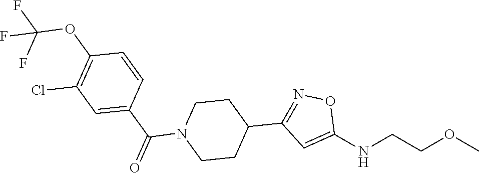 [SMILES: COCCNc1cc(C2CCN(C(=O)c3ccc(OC(F)(F)F)c(Cl)c3)CC2)no1] | 115 |
| 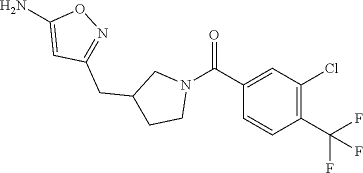 [SMILES: Nc1cc(CC2CCN(C(=O)c3ccc(C(F)(F)F)c(Cl)c3)C2)no1] | 116 |
| 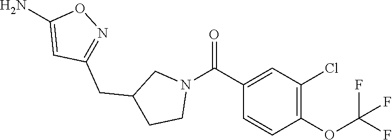 [SMILES: Nc1cc(CC2CCN(C(=O)c3ccc(OC(F)(F)F)c(Cl)c3)C2)no1] | 117 |
| 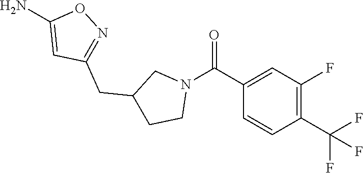 [SMILES: Nc1cc(CC2CCN(C(=O)c3ccc(C(F)(F)F)c(F)c3)C2)no1] | 118 |
| 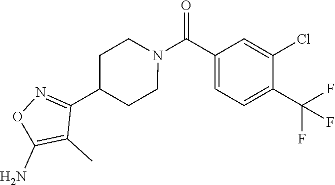 [SMILES: Cc1c(C2CCN(C(=O)c3ccc(C(F)(F)F)c(Cl)c3)CC2)noc1N] | 119 |
| 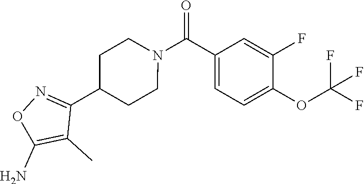 [SMILES: Cc1c(C2CCN(C(=O)c3ccc(OC(F)(F)F)c(F)c3)CC2)noc1N] | 120 |
| 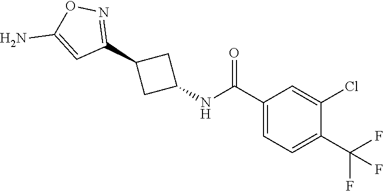 [SMILES: Nc1cc([C@H]2C[C@H](NC(=O)c3ccc(C(F)(F)F)c(Cl)c3)C2)no1] | 121 |
| 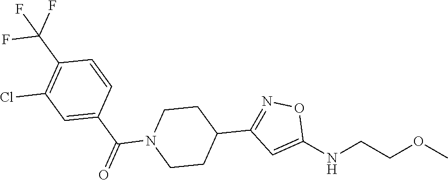 [SMILES: COCCNc1cc(C2CCN(C(=O)c3ccc(C(F)(F)F)c(Cl)c3)CC2)no1] | 122 |
| 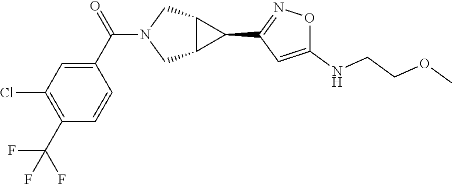 [SMILES: COCCNc1cc([C@H]2[C@@H]3CN(C(=O)c4ccc(C(F)(F)F)c(Cl)c4)C[C@@H]32)no1] | 123 |
| 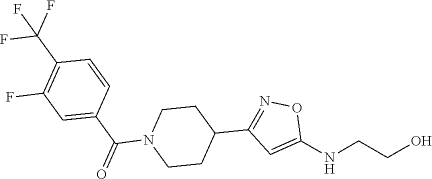 [SMILES: O=C(c1ccc(C(F)(F)F)c(F)c1)N1CCC(c2cc(NCCO)on2)CC1] | 124 |
| 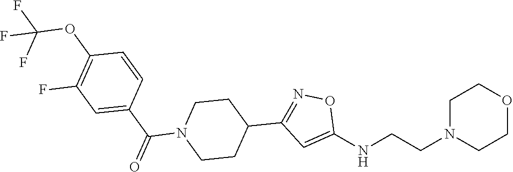 [SMILES: O=C(c1ccc(OC(F)(F)F)c(F)c1)N1CCC(c2cc(NCCN3CCOCC3)on2)CC1] | 125 |
| 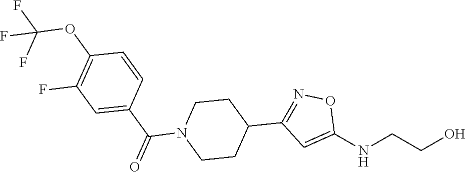 [SMILES: O=C(c1ccc(OC(F)(F)F)c(F)c1)N1CCC(c2cc(NCCO)on2)CC1] | 126 |
| 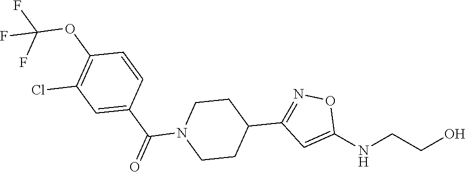 [SMILES: O=C(c1ccc(OC(F)(F)F)c(Cl)c1)N1CCC(c2cc(NCCO)on2)CC1] | 127 |
| 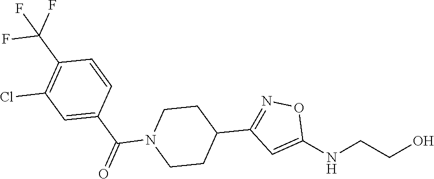 [SMILES: O=C(c1ccc(C(F)(F)F)c(Cl)c1)N1CCC(c2cc(NCCO)on2)CC1] | 128 |
| 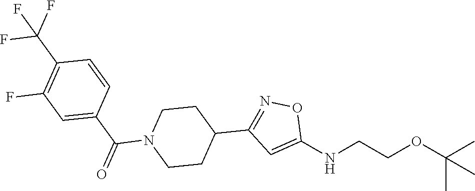 [SMILES: CC(C)(C)OCCNc1cc(C2CCN(C(=O)c3ccc(C(F)(F)F)c(F)c3)CC2)no1] | 129 |
| 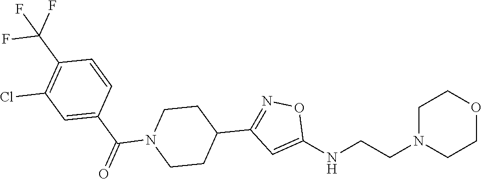 [SMILES: O=C(c1ccc(C(F)(F)F)c(Cl)c1)N1CCC(c2cc(NCCN3CCOCC3)on2)CC1] | 130 |
| 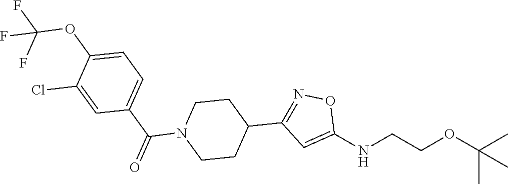 [SMILES: CC(C)(C)OCCNc1cc(C2CCN(C(=O)c3ccc(OC(F)(F)F)c(Cl)c3)CC2)no1] | 131 |
| 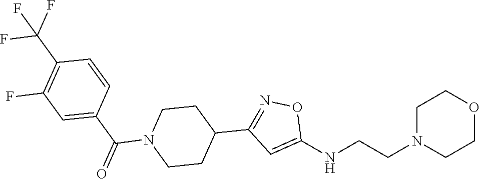 [SMILES: O=C(c1ccc(C(F)(F)F)c(F)c1)N1CCC(c2cc(NCCN3CCOCC3)on2)CC1] | 132 |
| 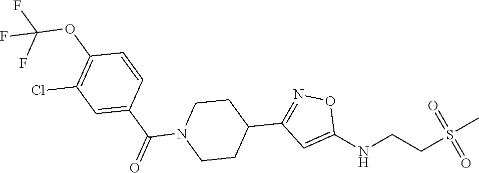 [SMILES: CS(=O)(=O)CCNc1cc(C2CCN(C(=O)c3ccc(OC(F)(F)F)c(Cl)c3)CC2)no1] | 133 |
| 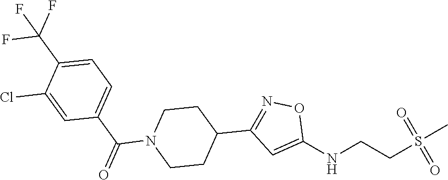 [SMILES: CS(=O)(=O)CCNc1cc(C2CCN(C(=O)c3ccc(C(F)(F)F)c(Cl)c3)CC2)no1] | 134 |
| 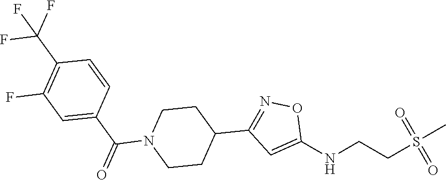 [SMILES: CS(=O)(=O)CCNc1cc(C2CCN(C(=O)c3ccc(C(F)(F)F)c(F)c3)CC2)no1] | 135 |
| 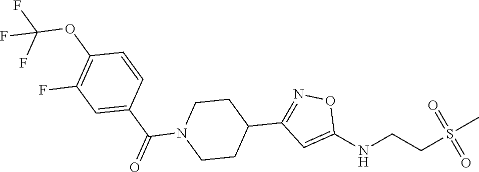 [SMILES: CS(=O)(=O)CCNc1cc(C2CCN(C(=O)c3ccc(OC(F)(F)F)c(F)c3)CC2)no1] | 136 |
| 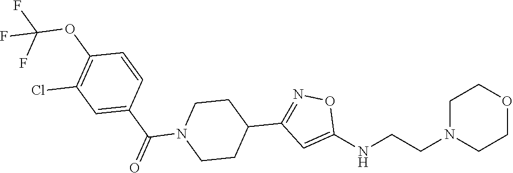 [SMILES: O=C(c1ccc(OC(F)(F)F)c(Cl)c1)N1CCC(c2cc(NCCN3CCOCC3)on2)CC1] | 137 |
| 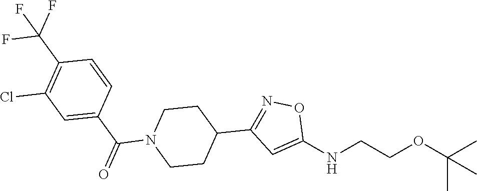 [SMILES: CC(C)(C)OCCNc1cc(C2CCN(C(=O)c3ccc(C(F)(F)F)c(Cl)c3)CC2)no1] | 138 |
| 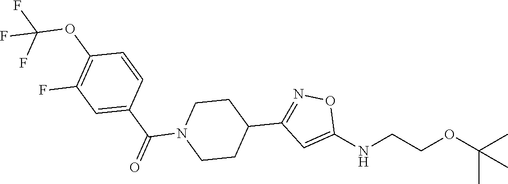 [SMILES: CC(C)(C)OCCNc1cc(C2CCN(C(=O)c3ccc(OC(F)(F)F)c(F)c3)CC2)no1] | 139 |
| 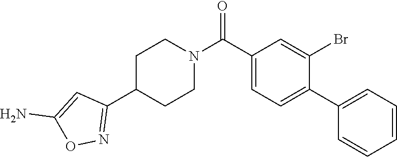 [SMILES: Nc1cc(C2CCN(C(=O)c3ccc(-c4ccccc4)c(Br)c3)CC2)no1] | 140 |
| 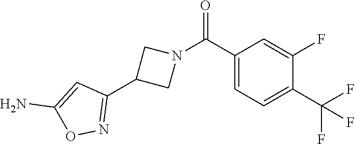 [SMILES: Nc1cc(C2CN(C(=O)c3ccc(C(F)(F)F)c(F)c3)C2)no1] | 141 |
| 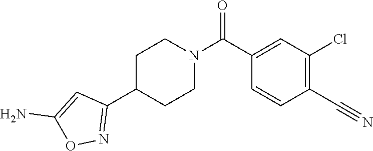 [SMILES: N#Cc1ccc(C(=O)N2CCC(c3cc(N)on3)CC2)cc1Cl] | 142 |
| 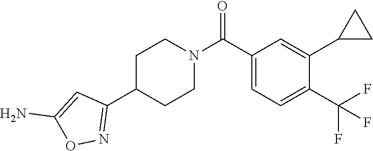 [SMILES: Nc1cc(C2CCN(C(=O)c3ccc(C(F)(F)F)c(C4CC4)c3)CC2)no1] | 143 |
| 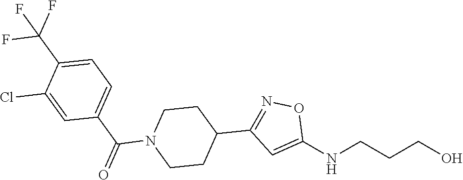 [SMILES: O=C(c1ccc(C(F)(F)F)c(Cl)c1)N1CCC(c2cc(NCCCO)on2)CC1] | 144 |
| 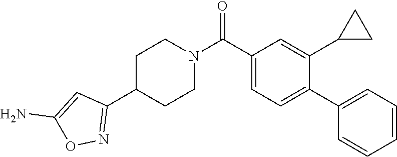 [SMILES: Nc1cc(C2CCN(C(=O)c3ccc(-c4ccccc4)c(C4CC4)c3)CC2)no1] | 145 |
| 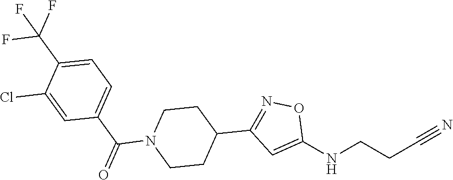 [SMILES: N#CCCNc1cc(C2CCN(C(=O)c3ccc(C(F)(F)F)c(Cl)c3)CC2)no1] | 146 |
| 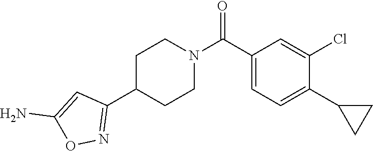 [SMILES: Nc1cc(C2CCN(C(=O)c3ccc(C4CC4)c(Cl)c3)CC2)no1] | 147 |
| 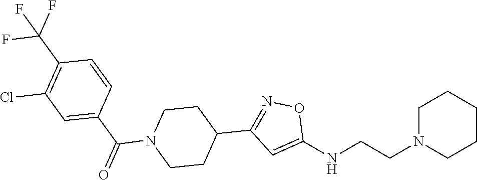 [SMILES: O=C(c1ccc(C(F)(F)F)c(Cl)c1)N1CCC(c2cc(NCCN3CCCCC3)on2)CC1] | 149 |
| 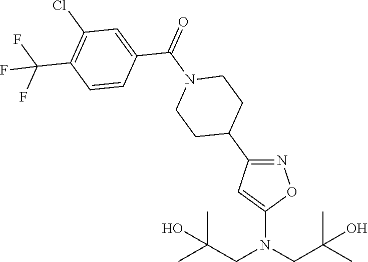 [SMILES: CC(C)(O)CN(CC(C)(C)O)c1cc(C2CCN(C(=O)c3ccc(C(F)(F)F)c(Cl)c3)CC2)no1] | 150 |
| 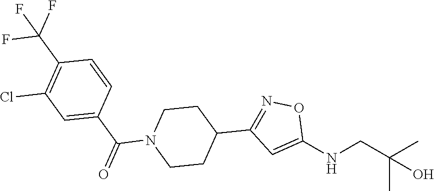 [SMILES: CC(C)(O)CNc1cc(C2CCN(C(=O)c3ccc(C(F)(F)F)c(Cl)c3)CC2)no1] | 151 |
| 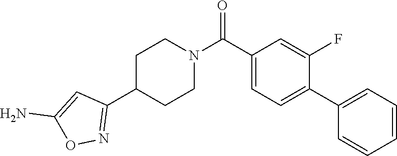 [SMILES: Nc1cc(C2CCN(C(=O)c3ccc(-c4ccccc4)c(F)c3)CC2)no1] | 152 |
| 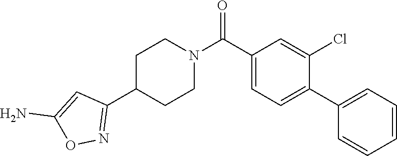 [SMILES: Nc1cc(C2CCN(C(=O)c3ccc(-c4ccccc4)c(Cl)c3)CC2)no1] | 153 |
| 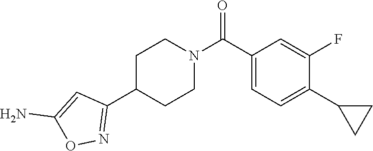 [SMILES: Nc1cc(C2CCN(C(=O)c3ccc(C4CC4)c(F)c3)CC2)no1] | 154 |
| 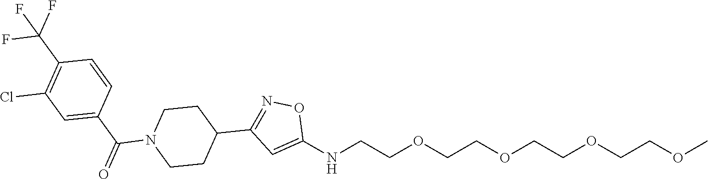 [SMILES: COCCOCCOCCOCCNc1cc(C2CCN(C(=O)c3ccc(C(F)(F)F)c(Cl)c3)CC2)no1] | 155 |
| 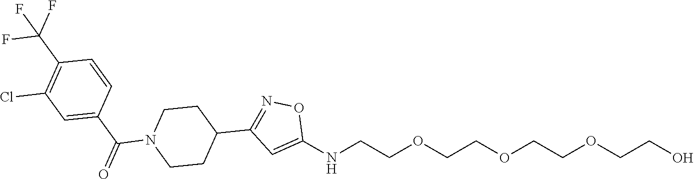 [SMILES: O=C(c1ccc(C(F)(F)F)c(Cl)c1)N1CCC(c2cc(NCCOCCOCCOCCO)on2)CC1] | 156 |
| 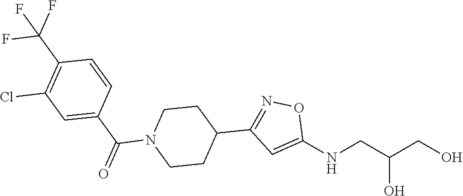 [SMILES: O=C(c1ccc(C(F)(F)F)c(Cl)c1)N1CCC(c2cc(NCC(O)CO)on2)CC1] | 157 |
| 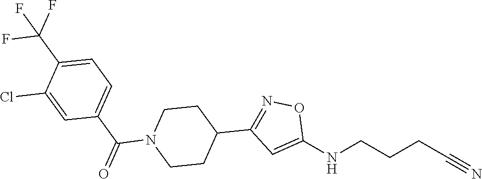 [SMILES: N#CCCCNc1cc(C2CCN(C(=O)c3ccc(C(F)(F)F)c(Cl)c3)CC2)no1] | 158 |
| 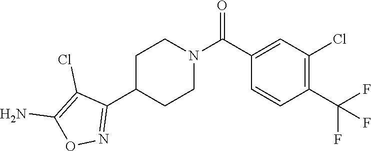 [SMILES: Nc1onc(C2CCN(C(=O)c3ccc(C(F)(F)F)c(Cl)c3)CC2)c1Cl] | 159 |
| 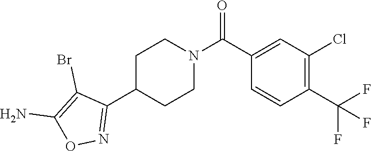 [SMILES: Nc1onc(C2CCN(C(=O)c3ccc(C(F)(F)F)c(Cl)c3)CC2)c1Br] | 160 |
| 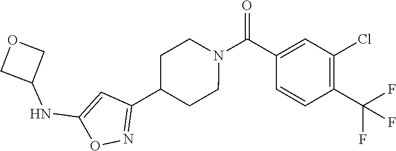 [SMILES: O=C(c1ccc(C(F)(F)F)c(Cl)c1)N1CCC(c2cc(NC3COC3)on2)CC1] | 161 |
| 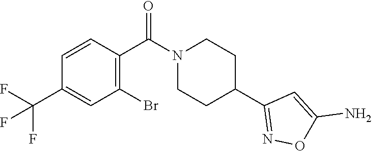 [SMILES: Nc1cc(C2CCN(C(=O)c3ccc(C(F)(F)F)cc3Br)CC2)no1] | 162 |
| 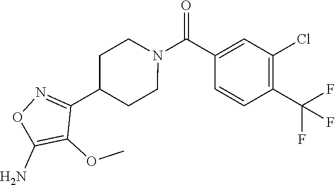 [SMILES: COc1c(C2CCN(C(=O)c3ccc(C(F)(F)F)c(Cl)c3)CC2)noc1N] | 163 |
| 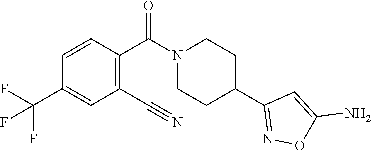 [SMILES: N#Cc1cc(C(F)(F)F)ccc1C(=O)N1CCC(c2cc(N)on2)CC1] | 164 |
| 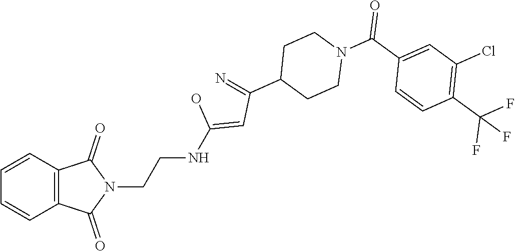 [SMILES: N=C(/C=C(\O)NCCN1C(=O)c2ccccc2C1=O)C1CCN(C(=O)c2ccc(C(F)(F)F)c(Cl)c2)CC1] | 165 |

16. The method according to any one of claims 1 to 15, wherein the inflammatory disease is selected from Type I interferonopathies, autoimmune diseases, neurological disorders, silica-induced fibrosis, and senescence-associated inflammatory diseases or disorders.

17. The method according to any one of claims 1 to 16, wherein the inflammatory disease is a Type I interferonopathy.

18. The method according to claim 17, wherein the Type I interferonopathy is chosen from Aicardi-Goutieres syndrome, spondyloenchondro-dysplasia with immune dysregulation, stimulator of interferon genes-associated vasculopathy with onset in infancy, X-linked reticulate pigmentary disorder, ubiquitin-specific peptidase 18 deficiency, chronic atypical neutrophilic dermatitis with lipodystrophy, Singleton-Merten syndrome, interferon-stimulated gene 15 deficiency, and DNAse II deficiency.

19. The method according to any one of claims 1 to 16, wherein the inflammatory disease is an autoimmune disease.

20. The method according to claim 19, wherein the autoimmune disease is chosen from systemic lupus erythematosus and rheumatoid arthritis.

21. The method according to any one of claims 1 to 16, wherein the inflammatory disease is a neurological disorder.

22. The method according to claim 21, wherein the neurological disorder is chosen from ischaemic brain injury, Parkinson's disease, general neurodegeneration, Huntington's disease, amyotrophic lateral sclerosis, frontotemporal dementia, and traumatic brain injury.

23. The method according to any one of claims 1 to 16, wherein the inflammatory disease is a senescence-associated inflammatory disease or disorder.

24. The method according to claim 23, wherein the senescence-associated inflammatory disease or disorder is chosen from age-dependent macular degeneration, atherosclerosis, and osteoarthritis.

25. A compound of Formula (I): 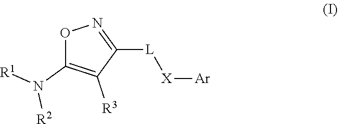
wherein
R¹ and R² are independently selected from the group consisting of H, C₁-C₆-alkyl (optionally substituted by one to five substituents independently selected from halo, CN, and OH), —C(O)C₁-C₆-alkyl, —C(O)H, —C₁-C₆-alkyl-(C₆-C₁₀-aryl), —C₁-C₆-alkyl-(C₁-C₆-alkoxy), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S), and —(C₁-C₆-alkyl)-SO₂—(C₁-C₆-alkyl), —(CH₂CH₂O)ₙ—R (wherein n is an integer selected from 2, 3, 4, 5, 6, 7, 8, 9, and 10, and R is H or C₁-C₆-alkyl);
R³ is selected from the group consisting of H, halo, C₁-C₆-alkyl, C₁-C₆-haloalkyl, and C₁-C₆-alkoxy;
L is a moiety selected from the group consisting of: 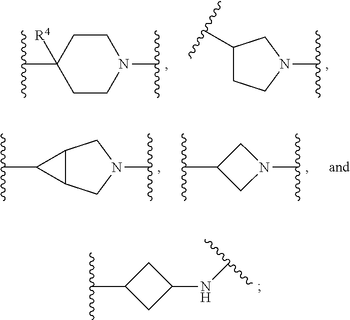
R⁴ is selected from the group consisting of H, halo, and C₁-C₆-alkyl;
X is —C(O)— or —SO₂—; and
Ar is: 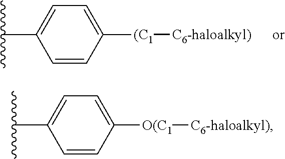
wherein Ar is optionally substituted with one to four substituents selected from the group consisting of halo, C₁-C₆-alkyl, C₁-C₆-alkoxy, CN, OH, NO₂, —NRR′ (wherein R and R′ are independently selected from H and C₁-C₆-alkyl), —(C₁-C₆-alkyl)NRR′, —C(O)NRR′, —SO₂R, C₆-C₁₀-aryl, 3- to 6-membered heterocycloalkyl (wherein 1-4 ring members are independently selected from N, O, and S), 5- to 10-membered heteroaryl (wherein 1-4 heteroaryl members are independently selected from N, O, and S), and C₃-C₁₀-cycloalkyl;
or a pharmaceutically acceptable salt thereof.

26. The compound or pharmaceutically acceptable salt thereof according to claim 25, wherein Ar is optionally substituted

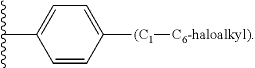

27. The compound or pharmaceutically acceptable salt thereof according to claim 25, wherein Ar is optionally substituted

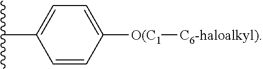

28. The compound or pharmaceutically acceptable salt thereof according to claim 25 or 26, wherein the —(C₁-C₆-haloalkyl) substituent on Ar is —CF₃.

29. The compound or pharmaceutically acceptable salt thereof according to claim 25 or 27, wherein the —O(C₁-C₆-haloalkyl) substituent on Ar is —OCF₃.

30. The compound or pharmaceutically acceptable salt thereof according to any one of claims 25 to 29, wherein Ar is substituted with one or two substituents selected from halo and C₁-C₆-alkyl.

31. The compound or pharmaceutically acceptable salt thereof according to any one of claims 25 to 30, wherein L is a moiety selected from:

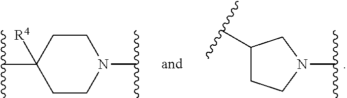

32. The compound or pharmaceutically acceptable salt thereof according to any one of claims 25 to 31, wherein L is

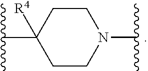

33. The compound or pharmaceutically acceptable salt thereof according to any one of claims 25 to 32, wherein R⁴ is H.

34. The compound or pharmaceutically acceptable salt thereof according to any one of claims 25 to 33, wherein X is —C(O)—.

35. The compound or pharmaceutically acceptable salt thereof according to any one of claims 25 to 33, wherein X is —SO₂—.

36. The compound or pharmaceutically acceptable salt thereof according to any one of claims 25 to 35, wherein R¹ and R² are independently selected from the group consisting of H and optionally substituted C₁-C₆-alkyl.

37. The compound or pharmaceutically acceptable salt thereof according to any one of claims 25 to 36, wherein each of R¹ and R² is H.

38. The compound or pharmaceutically acceptable salt thereof according to any one of claims 25 to 37, wherein R³ is H.

39. The compound or pharmaceutically acceptable salt thereof according to claim 25, wherein:
each of R¹, R², R³, and R⁴ is H;
L is 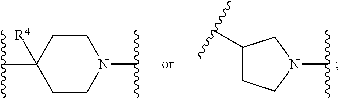
and
X is —C(O)—.

40. A compound or pharmaceutically acceptable salt thereof, wherein the compound is one selected from the following table:

| | |
|---|---|
| 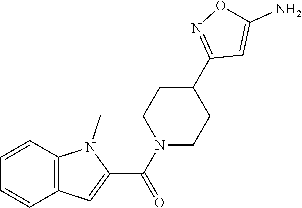 [SMILES: Cn1c(C(=O)N2CCC(c3cc(N)on3)CC2)cc2ccccc21] | 1 |
| 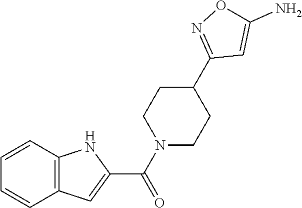 [SMILES: Nc1cc(C2CCN(C(=O)c3cc4ccccc4[nH]3)CC2)no1] | 2 |
| 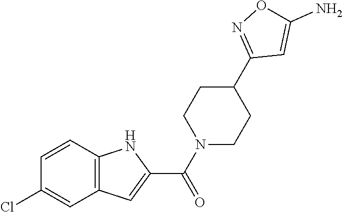 [SMILES: Nc1cc(C2CCN(C(=O)c3cc4cc(Cl)ccc4[nH]3)CC2)no1] | 3 |
| 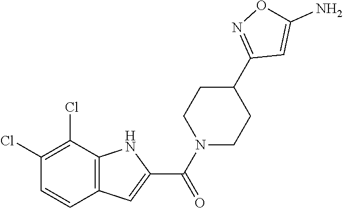 [SMILES: Nc1cc(C2CCN(C(=O)c3cc4ccc(Cl)c(Cl)c4[nH]3)CC2)no1] | 4 |
| 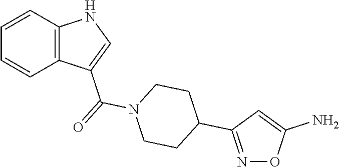 [SMILES: Nc1cc(C2CCN(C(=O)c3c[nH]c4ccccc34)CC2)no1] | 5 |
| 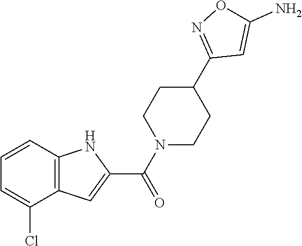 [SMILES: Nc1cc(C2CCN(C(=O)c3cc4c(Cl)cccc4[nH]3)CC2)no1] | 6 |
| 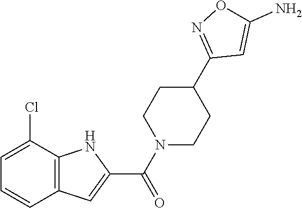 [SMILES: Nc1cc(C2CCN(C(=O)c3cc4cccc(Cl)c4[nH]3)CC2)no1] | 7 |
| 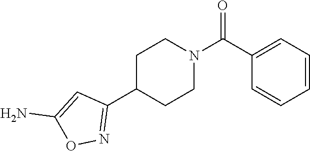 [SMILES: Nc1cc(C2CCN(C(=O)c3ccccc3)CC2)no1] | 8 |
| 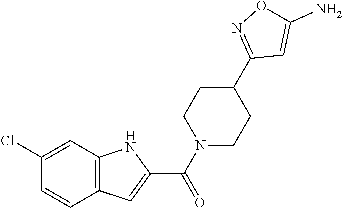 [SMILES: Nc1cc(C2CCN(C(=O)c3cc4ccc(Cl)cc4[nH]3)CC2)no1] | 9 |
| 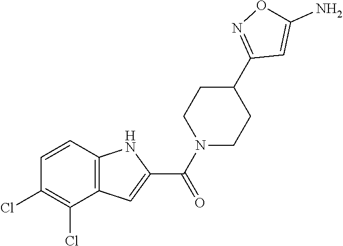 [SMILES: Nc1cc(C2CCN(C(=O)c3cc4c(Cl)c(Cl)ccc4[nH]3)CC2)no1] | 10 |
| 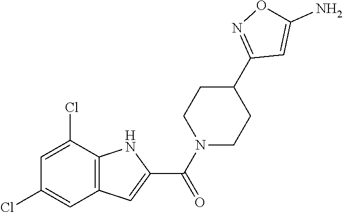 [SMILES: Nc1cc(C2CCN(C(=O)c3cc4cc(Cl)cc(Cl)c4[nH]3)CC2)no1] | 11 |
| 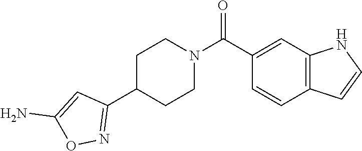 [SMILES: Nc1cc(C2CCN(C(=O)c3ccc4cc[nH]c4c3)CC2)no1] | 12 |
| 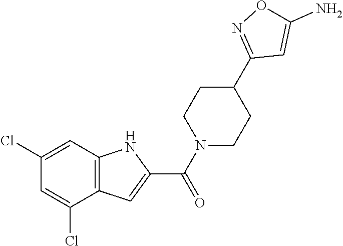 [SMILES: Nc1cc(C2CCN(C(=O)c3cc4c(Cl)cc(Cl)cc4[nH]3)CC2)no1] | 13 |
| 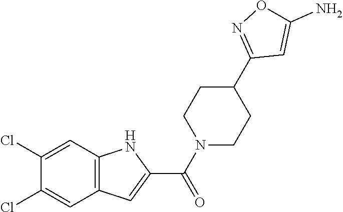 [SMILES: Nc1cc(C2CCN(C(=O)c3cc4cc(Cl)c(Cl)cc4[nH]3)CC2)no1] | 14 |
| 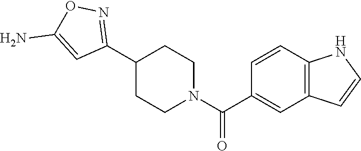 [SMILES: Nc1cc(C2CCN(C(=O)c3ccc4[nH]ccc4c3)CC2)no1] | 15 |
| 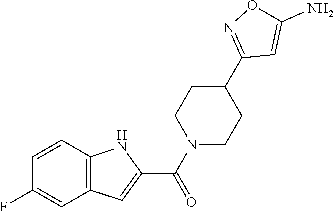 [SMILES: Nc1cc(C2CCN(C(=O)c3cc4cc(F)ccc4[nH]3)CC2)no1] | 16 |
| 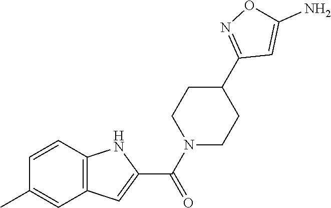 [SMILES: Cc1ccc2[nH]c(C(=O)N3CCC(c4cc(N)on4)CC3)cc2c1] | 17 |
| 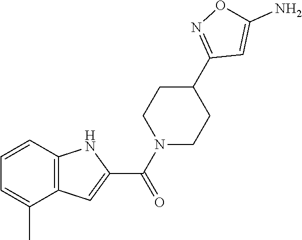 [SMILES: Cc1cccc2[nH]c(C(=O)N3CCC(c4cc(N)on4)CC3)cc12] | 18 |
| 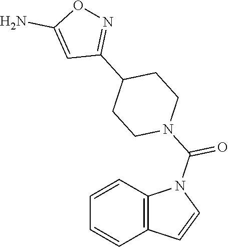 [SMILES: Nc1cc(C2CCN(C(=O)n3ccc4ccccc43)CC2)no1] | 19 |
| 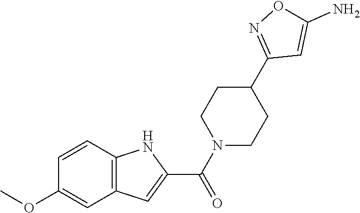 [SMILES: COc1ccc2[nH]c(C(=O)N3CCC(c4cc(N)on4)CC3)cc2c1] | 20 |
| 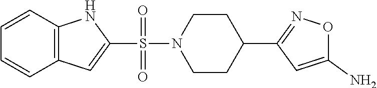 [SMILES: Nc1cc(C2CCN(S(=O)(=O)c3cc4ccccc4[nH]3)CC2)no1] | 21 |
| 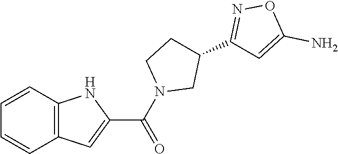 [SMILES: Nc1cc([C@H]2CCN(C(=O)c3cc4ccccc4[nH]3)C2)no1] | 22 |
| 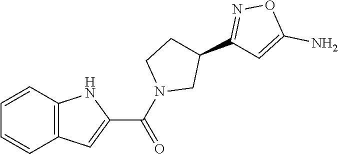 [SMILES: Nc1cc([C@@H]2CCN(C(=O)c3cc4ccccc4[nH]3)C2)no1] | 23 |
| 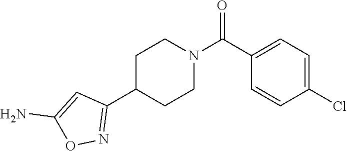 [SMILES: Nc1cc(C2CCN(C(=O)c3ccc(Cl)cc3)CC2)no1] | 24 |
| 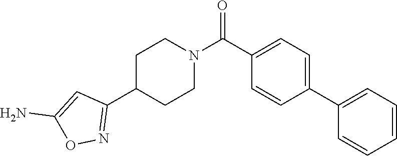 [SMILES: Nc1cc(C2CCN(C(=O)c3ccc(-c4ccccc4)cc3)CC2)no1] | 25 |
| 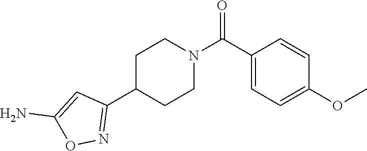 [SMILES: COc1ccc(C(=O)N2CCC(c3cc(N)on3)CC2)cc1] | 26 |
| 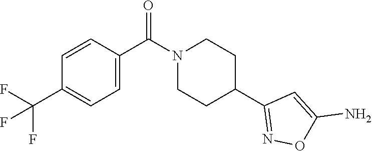 [SMILES: Nc1cc(C2CCN(C(=O)c3ccc(C(F)(F)F)cc3)CC2)no1] | 27 |
| 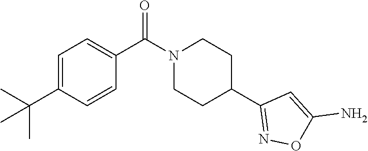 [SMILES: CC(C)(C)c1ccc(C(=O)N2CCC(c3cc(N)on3)CC2)cc1] | 28 |
| 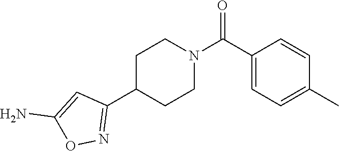 [SMILES: Cc1ccc(C(=O)N2CCC(c3cc(N)on3)CC2)cc1] | 29 |
| 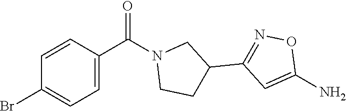 [SMILES: Nc1cc(C2CCN(C(=O)c3ccc(Br)cc3)C2)no1] | 30 |
| 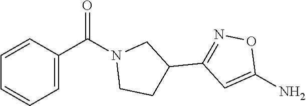 [SMILES: Nc1cc(C2CCN(C(=O)c3ccccc3)C2)no1] | 31 |
| 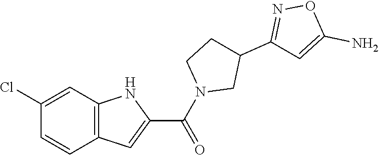 [SMILES: Nc1cc(C2CCN(C(=O)c3cc4ccc(Cl)cc4[nH]3)C2)no1] | 32 |
| 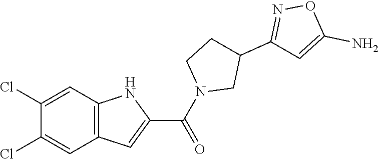 [SMILES: Nc1cc(C2CCN(C(=O)c3cc4cc(Cl)c(Cl)cc4[nH]3)C2)no1] | 33 |
| 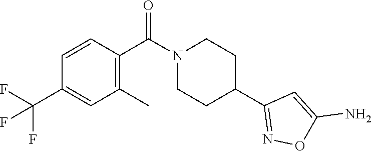 [SMILES: Cc1cc(C(F)(F)F)ccc1C(=O)N1CCC(c2cc(N)on2)CC1] | 34 |
| 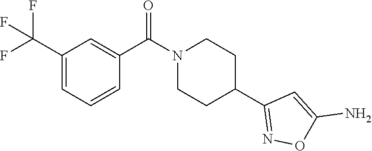 [SMILES: Nc1cc(C2CCN(C(=O)c3cccc(C(F)(F)F)c3)CC2)no1] | 35 |
| 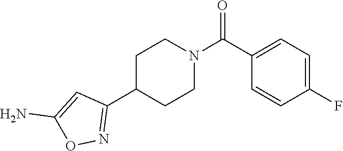 [SMILES: Nc1cc(C2CCN(C(=O)c3ccc(F)cc3)CC2)no1] | 36 |
| 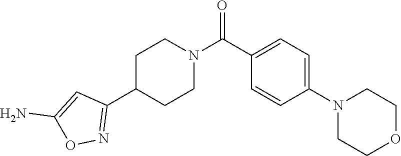 [SMILES: Nc1cc(C2CCN(C(=O)c3ccc(N4CCOCC4)cc3)CC2)no1] | 37 |
| 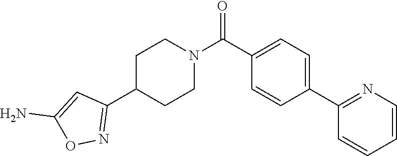 [SMILES: Nc1cc(C2CCN(C(=O)c3ccc(-c4ccccn4)cc3)CC2)no1] | 38 |
| 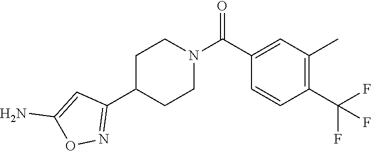 [SMILES: Cc1cc(C(=O)N2CCC(c3cc(N)on3)CC2)ccc1C(F)(F)F] | 39 |
| 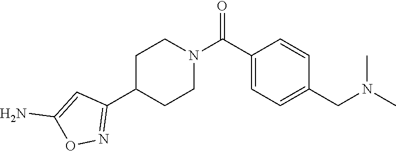 [SMILES: CN(C)Cc1ccc(C(=O)N2CCC(c3cc(N)on3)CC2)cc1] | 40 |
| 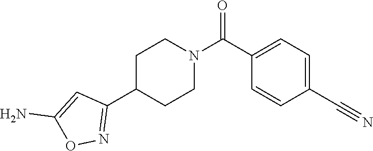 [SMILES: N#Cc1ccc(C(=O)N2CCC(c3cc(N)on3)CC2)cc1] | 41 |
| 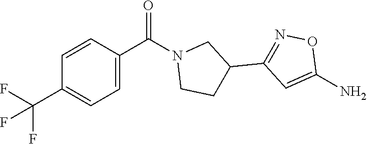 [SMILES: Nc1cc(C2CCN(C(=O)c3ccc(C(F)(F)F)cc3)C2)no1] | 42 |
| 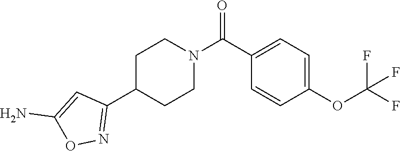 [SMILES: Nc1cc(C2CCN(C(=O)c3ccc(OC(F)(F)F)cc3)CC2)no1] | 43 |
| 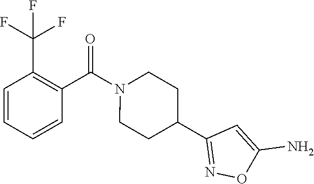 [SMILES: Nc1cc(C2CCN(C(=O)c3ccccc3C(F)(F)F)CC2)no1] | 44 |
| 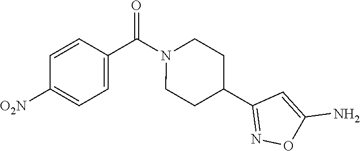 [SMILES: Nc1cc(C2CCN(C(=O)c3ccc([N+](=O)[O-])cc3)CC2)no1] | 45 |
| 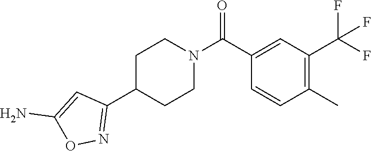 [SMILES: Cc1ccc(C(=O)N2CCC(c3cc(N)on3)CC2)cc1C(F)(F)F] | 46 |
| 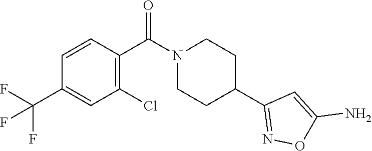 [SMILES: Nc1cc(C2CCN(C(=O)c3ccc(C(F)(F)F)cc3Cl)CC2)no1] | 47 |
| 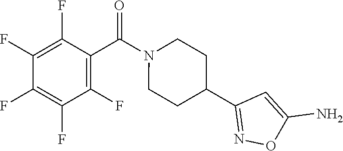 [SMILES: Nc1cc(C2CCN(C(=O)c3c(F)c(F)c(F)c(F)c3F)CC2)no1] | 48 |
| 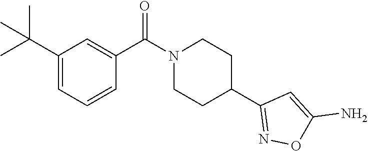 [SMILES: CC(C)(C)c1cccc(C(=O)N2CCC(c3cc(N)on3)CC2)c1] | 49 |
| 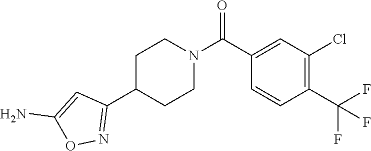 [SMILES: Nc1cc(C2CCN(C(=O)c3ccc(C(F)(F)F)c(Cl)c3)CC2)no1] | 50 |
| 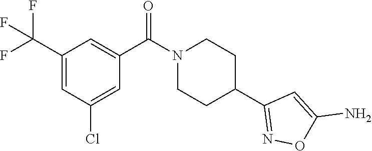 [SMILES: Nc1cc(C2CCN(C(=O)c3cc(Cl)cc(C(F)(F)F)c3)CC2)no1] | 51 |
| 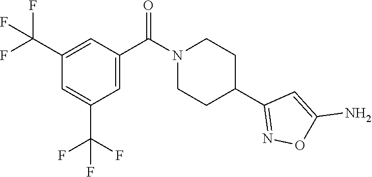 [SMILES: Nc1cc(C2CCN(C(=O)c3cc(C(F)(F)F)cc(C(F)(F)F)c3)CC2)no1] | 52 |
| 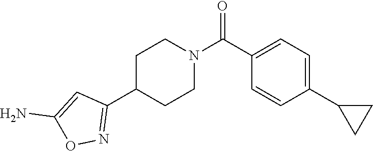 [SMILES: Nc1cc(C2CCN(C(=O)c3ccc(C4CC4)cc3)CC2)no1] | 53 |
| 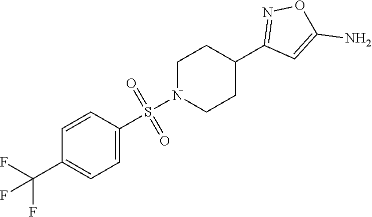 [SMILES: Nc1cc(C2CCN(S(=O)(=O)c3ccc(C(F)(F)F)cc3)CC2)no1] | 54 |
| 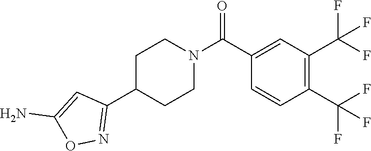 [SMILES: Nc1cc(C2CCN(C(=O)c3ccc(C(F)(F)F)c(C(F)(F)F)c3)CC2)no1] | 55 |
| 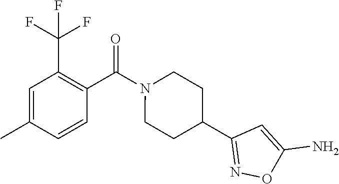 [SMILES: Cc1ccc(C(=O)N2CCC(c3cc(N)on3)CC2)c(C(F)(F)F)c1] | 56 |
| 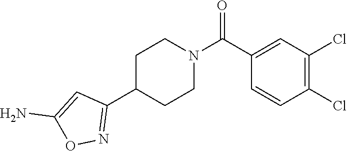 [SMILES: Nc1cc(C2CCN(C(=O)c3ccc(Cl)c(Cl)c3)CC2)no1] | 57 |
| 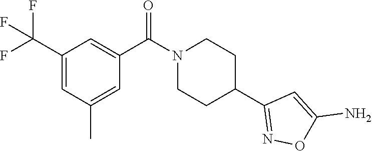 [SMILES: Cc1cc(C(=O)N2CCC(c3cc(N)on3)CC2)cc(C(F)(F)F)c1] | 58 |
| 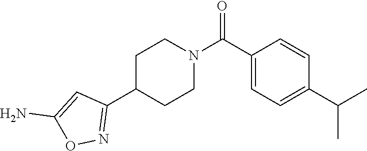 [SMILES: CC(C)c1ccc(C(=O)N2CCC(c3cc(N)on3)CC2)cc1] | 59 |
| 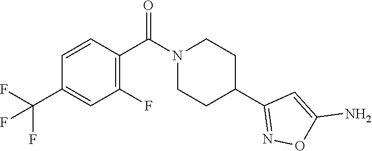 [SMILES: Nc1cc(C2CCN(C(=O)c3ccc(C(F)(F)F)cc3F)CC2)no1] | 60 |
| 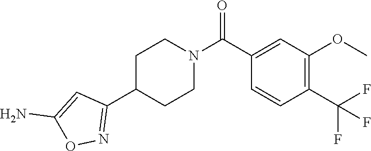 [SMILES: COc1cc(C(=O)N2CCC(c3cc(N)on3)CC2)ccc1C(F)(F)F] | 61 |
| 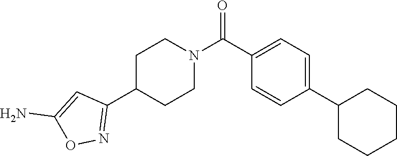 [SMILES: Nc1cc(C2CCN(C(=O)c3ccc(C4CCCCC4)cc3)CC2)no1] | 62 |
| 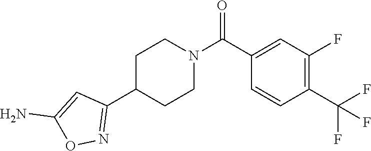 [SMILES: Nc1cc(C2CCN(C(=O)c3ccc(C(F)(F)F)c(F)c3)CC2)no1] | 63 |
| 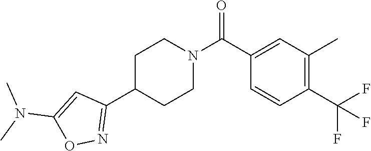 [SMILES: Cc1cc(C(=O)N2CCC(c3cc(N(C)C)on3)CC2)ccc1C(F)(F)F] | 64 |
| 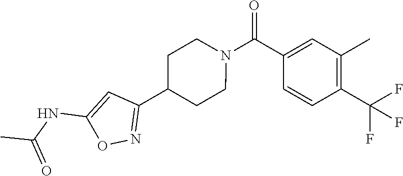 [SMILES: CC(=O)Nc1cc(C2CCN(C(=O)c3ccc(C(F)(F)F)c(C)c3)CC2)no1] | 65 |
| 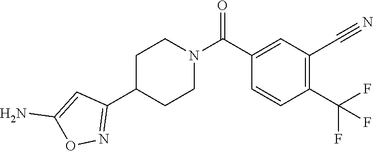 [SMILES: N#Cc1cc(C(=O)N2CCC(c3cc(N)on3)CC2)ccc1C(F)(F)F] | 66 |
| 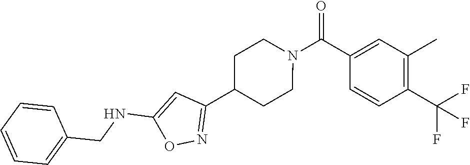 [SMILES: Cc1cc(C(=O)N2CCC(c3cc(NCc4ccccc4)on3)CC2)ccc1C(F)(F)F] | 67 |
| 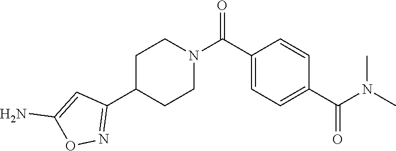 [SMILES: CN(C)C(=O)c1ccc(C(=O)N2CCC(c3cc(N)on3)CC2)cc1] | 68 |
| 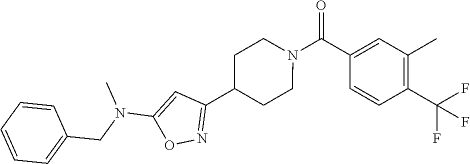 [SMILES: Cc1cc(C(=O)N2CCC(c3cc(N(C)Cc4ccccc4)on3)CC2)ccc1C(F)(F)F] | 69 |
| 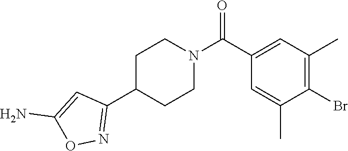 [SMILES: Cc1cc(C(=O)N2CCC(c3cc(N)on3)CC2)cc(C)c1Br] | 70 |
| 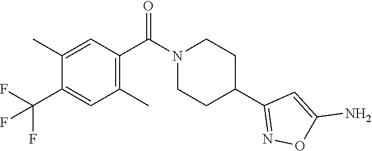 [SMILES: Cc1cc(C(F)(F)F)c(C)cc1C(=O)N1CCC(c2cc(N)on2)CC1] | 71 |
| 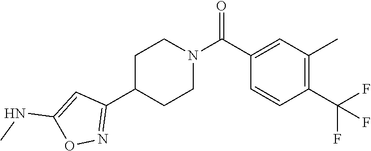 [SMILES: CNc1cc(C2CCN(C(=O)c3ccc(C(F)(F)F)c(C)c3)CC2)no1] | 72 |
| 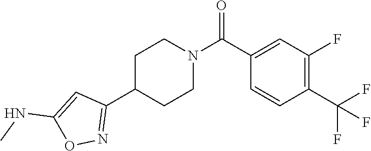 [SMILES: CNc1cc(C2CCN(C(=O)c3ccc(C(F)(F)F)c(F)c3)CC2)no1] | 73 |
| 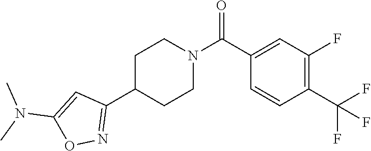 [SMILES: CN(C)c1cc(C2CCN(C(=O)c3ccc(C(F)(F)F)c(F)c3)CC2)no1] | 74 |
| 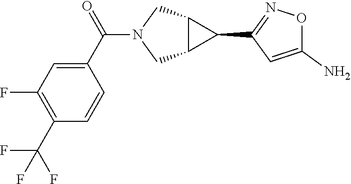 [SMILES: Nc1cc([C@H]2[C@@H]3CN(C(=O)c4ccc(C(F)(F)F)c(F)c4)C[C@@H]32)no1] | 75 |
| 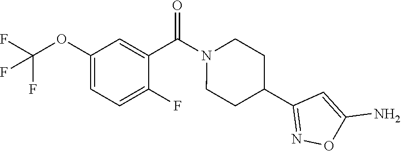 [SMILES: Nc1cc(C2CCN(C(=O)c3cc(OC(F)(F)F)ccc3F)CC2)no1] | 76 |
| 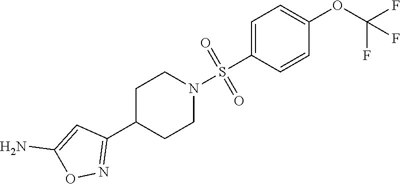 [SMILES: Nc1cc(C2CCN(S(=O)(=O)c3ccc(OC(F)(F)F)cc3)CC2)no1] | 77 |
| 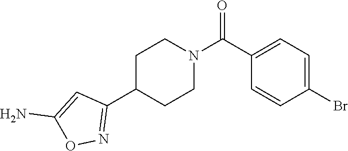 [SMILES: Nc1cc(C2CCN(C(=O)c3ccc(Br)cc3)CC2)no1] | 78 |
| 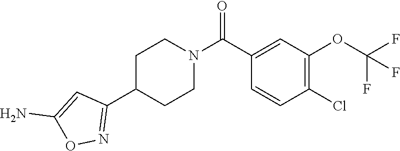 [SMILES: Nc1cc(C2CCN(C(=O)c3ccc(Cl)c(OC(F)(F)F)c3)CC2)no1] | 79 |
| 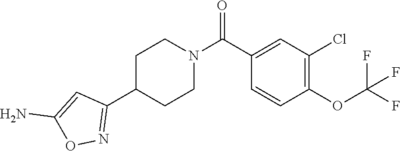 [SMILES: Nc1cc(C2CCN(C(=O)c3ccc(OC(F)(F)F)c(Cl)c3)CC2)no1] | 80 |
| 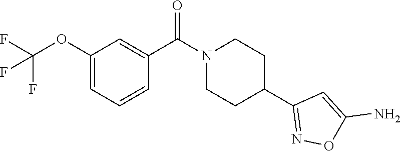 [SMILES: Nc1cc(C2CCN(C(=O)c3cccc(OC(F)(F)F)c3)CC2)no1] | 81 |
| 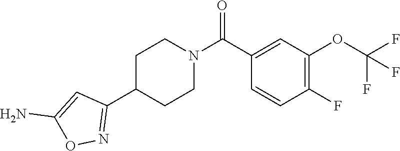 [SMILES: Nc1cc(C2CCN(C(=O)c3ccc(F)c(OC(F)(F)F)c3)CC2)no1] | 82 |
| 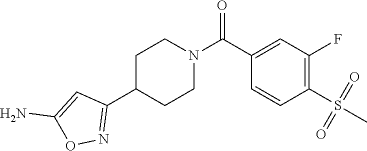 [SMILES: CS(=O)(=O)c1ccc(C(=O)N2CCC(c3cc(N)on3)CC2)cc1F] | 83 |
| 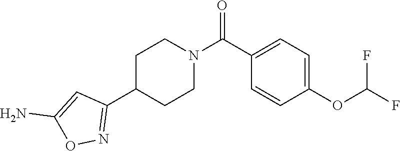 [SMILES: Nc1cc(C2CCN(C(=O)c3ccc(OC(F)F)cc3)CC2)no1] | 84 |
| 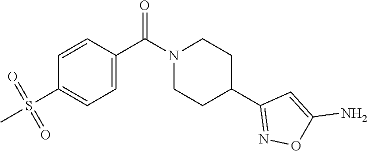 [SMILES: CS(=O)(=O)c1ccc(C(=O)N2CCC(c3cc(N)on3)CC2)cc1] | 85 |
| 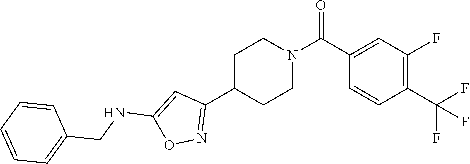 [SMILES: O=C(c1ccc(C(F)(F)F)c(F)c1)N1CCC(c2cc(NCc3ccccc3)on2)CC1] | 86 |
| 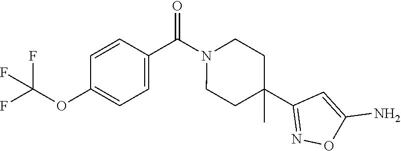 [SMILES: CC1(c2cc(N)on2)CCN(C(=O)c2ccc(OC(F)(F)F)cc2)CC1] | 87 |
| 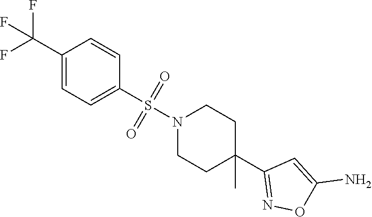 [SMILES: CC1(c2cc(N)on2)CCN(S(=O)(=O)c2ccc(C(F)(F)F)cc2)CC1] | 88 |
| 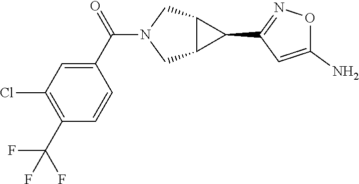 [SMILES: Nc1cc([C@H]2[C@@H]3CN(C(=O)c4ccc(C(F)(F)F)c(Cl)c4)C[C@@H]32)no1] | 89 |
| 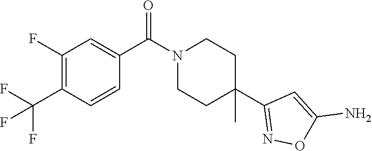 [SMILES: CC1(c2cc(N)on2)CCN(C(=O)c2ccc(C(F)(F)F)c(F)c2)CC1] | 90 |
| 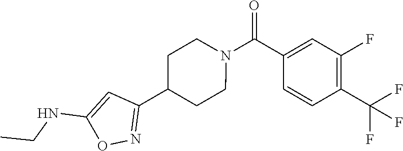 [SMILES: CCNc1cc(C2CCN(C(=O)c3ccc(C(F)(F)F)c(F)c3)CC2)no1] | 91 |
| 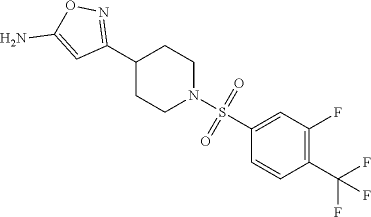 [SMILES: Nc1cc(C2CCN(S(=O)(=O)c3ccc(C(F)(F)F)c(F)c3)CC2)no1] | 92 |
| 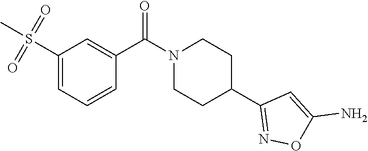 [SMILES: CS(=O)(=O)c1cccc(C(=O)N2CCC(c3cc(N)on3)CC2)c1] | 93 |
| 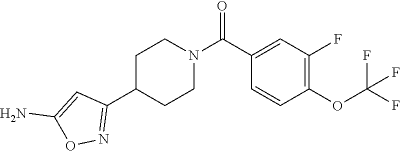 [SMILES: Nc1cc(C2CCN(C(=O)c3ccc(OC(F)(F)F)c(F)c3)CC2)no1] | 94 |
| 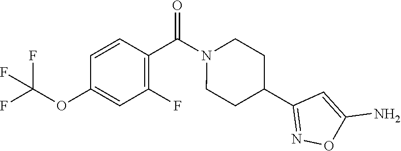 [SMILES: Nc1cc(C2CCN(C(=O)c3ccc(OC(F)(F)F)cc3F)CC2)no1] | 95 |
| 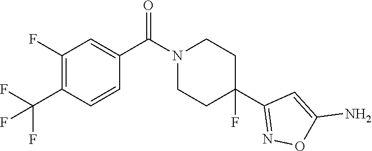 [SMILES: Nc1cc(C2(F)CCN(C(=O)c3ccc(C(F)(F)F)c(F)c3)CC2)no1] | 96 |
| 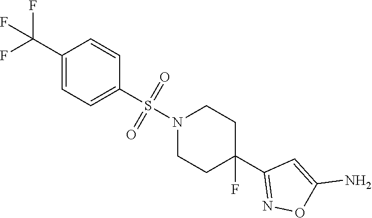 [SMILES: Nc1cc(C2(F)CCN(S(=O)(=O)c3ccc(C(F)(F)F)cc3)CC2)no1] | 97 |
| 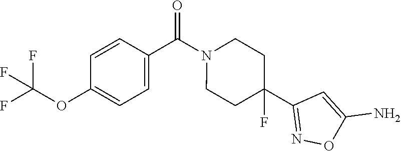 [SMILES: Nc1cc(C2(F)CCN(C(=O)c3ccc(OC(F)(F)F)cc3)CC2)no1] | 98 |
| 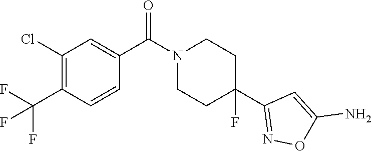 [SMILES: Nc1cc(C2(F)CCN(C(=O)c3ccc(C(F)(F)F)c(Cl)c3)CC2)no1] | 99 |
| 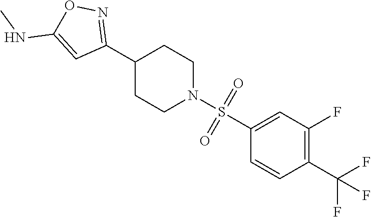 [SMILES: CNc1cc(C2CCN(S(=O)(=O)c3ccc(C(F)(F)F)c(F)c3)CC2)no1] | 100 |
| 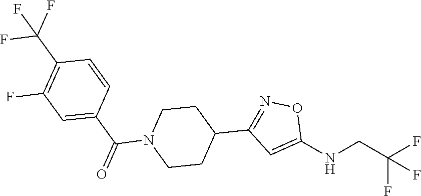 [SMILES: O=C(c1ccc(C(F)(F)F)c(F)c1)N1CCC(c2cc(NCC(F)(F)F)on2)CC1] | 101 |
| 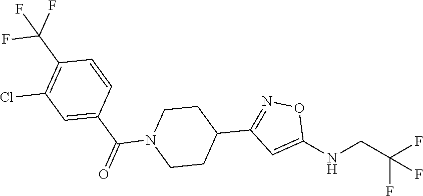 [SMILES: O=C(c1ccc(C(F)(F)F)c(Cl)c1)N1CCC(c2cc(NCC(F)(F)F)on2)CC1] | 102 |
| 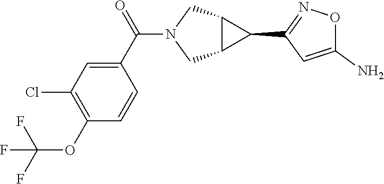 [SMILES: Nc1cc([C@H]2[C@@H]3CN(C(=O)c4ccc(OC(F)(F)F)c(Cl)c4)C[C@@H]32)no1] | 103 |
| 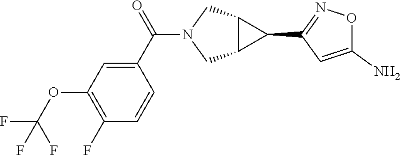 [SMILES: Nc1cc([C@H]2[C@@H]3CN(C(=O)c4ccc(F)c(OC(F)(F)F)c4)C[C@@H]32)no1] | 104 |
| 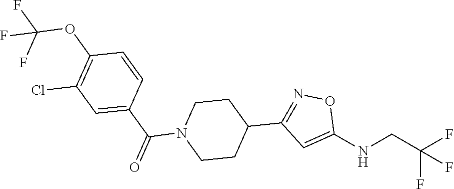 [SMILES: O=C(c1ccc(OC(F)(F)F)c(Cl)c1)N1CCC(c2cc(NCC(F)(F)F)on2)CC1] | 105 |
| 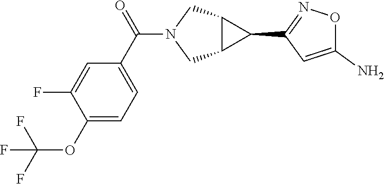 [SMILES: Nc1cc([C@H]2[C@@H]3CN(C(=O)c4ccc(OC(F)(F)F)c(F)c4)C[C@@H]32)no1] | 106 |
| 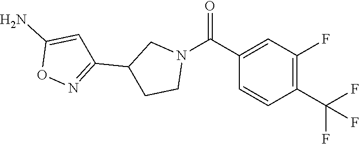 [SMILES: Nc1cc(C2CCN(C(=O)c3ccc(C(F)(F)F)c(F)c3)C2)no1] | 107 |
| 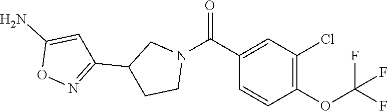 [SMILES: Nc1cc(C2CCN(C(=O)c3ccc(OC(F)(F)F)c(Cl)c3)C2)no1] | 108 |
| 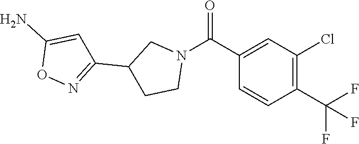 [SMILES: Nc1cc(C2CCN(C(=O)c3ccc(C(F)(F)F)c(Cl)c3)C2)no1] | 109 |
| 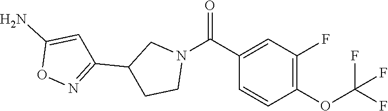 [SMILES: Nc1cc(C2CCN(C(=O)c3ccc(OC(F)(F)F)c(F)c3)C2)no1] | 110 |
| 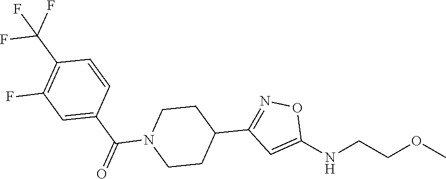 [SMILES: COCCNc1cc(C2CCN(C(=O)c3ccc(C(F)(F)F)c(F)c3)CC2)no1] | 111 |
| 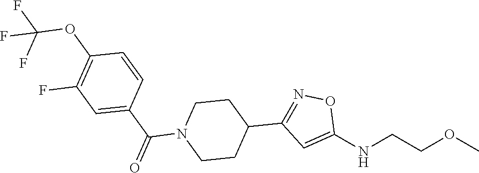 [SMILES: COCCNc1cc(C2CCN(C(=O)c3ccc(OC(F)(F)F)c(F)c3)CC2)no1] | 112 |
| 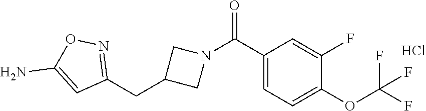 [SMILES: Nc1cc(CC2CN(C(=O)c3ccc(OC(F)(F)F)c(F)c3)C2)no1 ; Cl] | 113 |
| 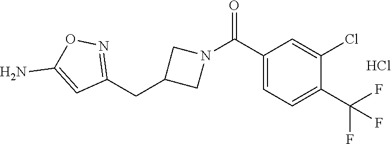 [SMILES: Nc1cc(CC2CN(C(=O)c3ccc(C(F)(F)F)c(Cl)c3)C2)no1 ; Cl] | 114 |
| 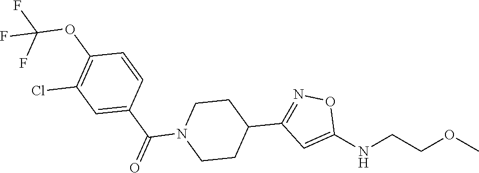 [SMILES: COCCNc1cc(C2CCN(C(=O)c3ccc(OC(F)(F)F)c(Cl)c3)CC2)no1] | 115 |
| 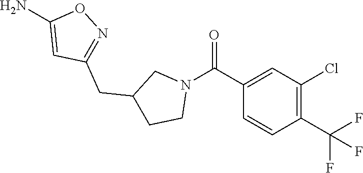 [SMILES: Nc1cc(CC2CCN(C(=O)c3ccc(C(F)(F)F)c(Cl)c3)C2)no1] | 116 |
| 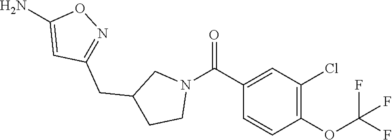 [SMILES: Nc1cc(CC2CCN(C(=O)c3ccc(OC(F)(F)F)c(Cl)c3)C2)no1] | 117 |
| 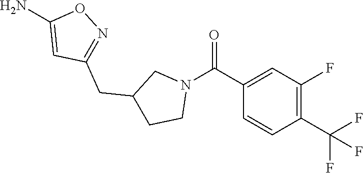 [SMILES: Nc1cc(CC2CCN(C(=O)c3ccc(C(F)(F)F)c(F)c3)C2)no1] | 118 |
| 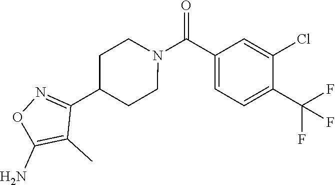 [SMILES: Cc1c(C2CCN(C(=O)c3ccc(C(F)(F)F)c(Cl)c3)CC2)noc1N] | 119 |
| 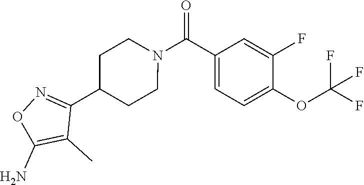 [SMILES: Cc1c(C2CCN(C(=O)c3ccc(OC(F)(F)F)c(F)c3)CC2)noc1N] | 120 |
| 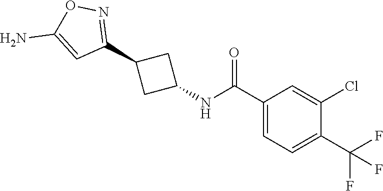 [SMILES: Nc1cc([C@H]2C[C@H](NC(=O)c3ccc(C(F)(F)F)c(Cl)c3)C2)no1] | 121 |
| 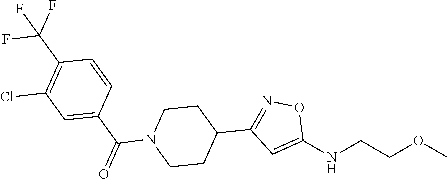 [SMILES: COCCNc1cc(C2CCN(C(=O)c3ccc(C(F)(F)F)c(Cl)c3)CC2)no1] | 122 |
| 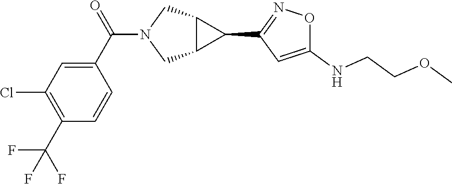 [SMILES: COCCNc1cc([C@H]2[C@@H]3CN(C(=O)c4ccc(C(F)(F)F)c(Cl)c4)C[C@@H]32)no1] | 123 |
| 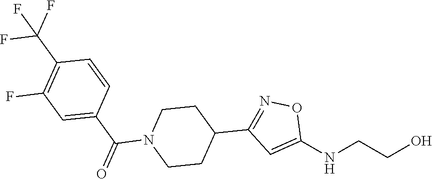 [SMILES: O=C(c1ccc(C(F)(F)F)c(F)c1)N1CCC(c2cc(NCCO)on2)CC1] | 124 |
| 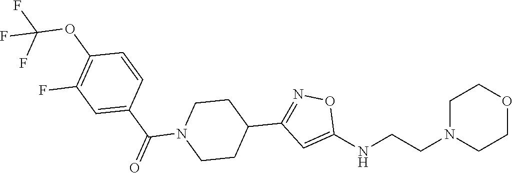 [SMILES: O=C(c1ccc(OC(F)(F)F)c(F)c1)N1CCC(c2cc(NCCN3CCOCC3)on2)CC1] | 125 |
| 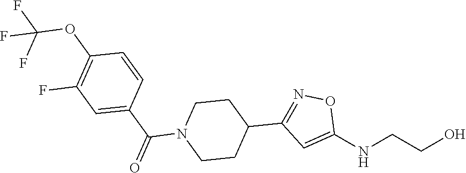 [SMILES: O=C(c1ccc(OC(F)(F)F)c(F)c1)N1CCC(c2cc(NCCO)on2)CC1] | 126 |
| 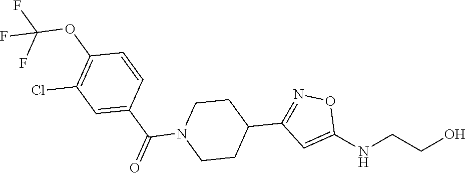 [SMILES: O=C(c1ccc(OC(F)(F)F)c(Cl)c1)N1CCC(c2cc(NCCO)on2)CC1] | 127 |
| 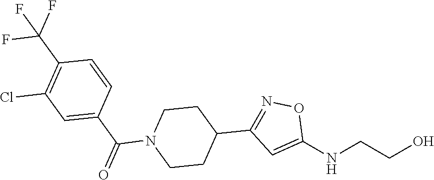 [SMILES: O=C(c1ccc(C(F)(F)F)c(Cl)c1)N1CCC(c2cc(NCCO)on2)CC1] | 128 |
| 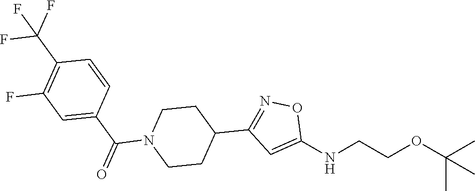 [SMILES: CC(C)(C)OCCNc1cc(C2CCN(C(=O)c3ccc(C(F)(F)F)c(F)c3)CC2)no1] | 129 |
| 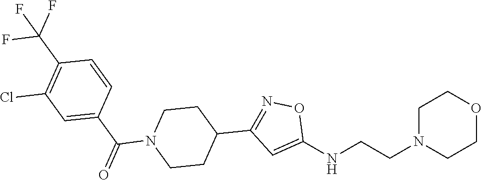 [SMILES: O=C(c1ccc(C(F)(F)F)c(Cl)c1)N1CCC(c2cc(NCCN3CCOCC3)on2)CC1] | 130 |
| 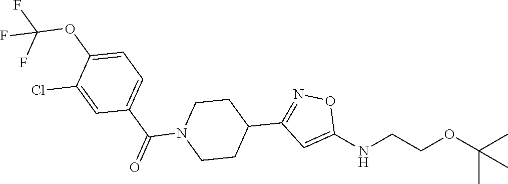 [SMILES: CC(C)(C)OCCNc1cc(C2CCN(C(=O)c3ccc(OC(F)(F)F)c(Cl)c3)CC2)no1] | 131 |
| 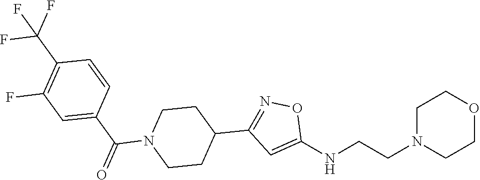 [SMILES: O=C(c1ccc(C(F)(F)F)c(F)c1)N1CCC(c2cc(NCCN3CCOCC3)on2)CC1] | 132 |
| 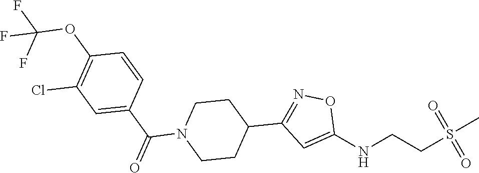 [SMILES: CS(=O)(=O)CCNc1cc(C2CCN(C(=O)c3ccc(OC(F)(F)F)c(Cl)c3)CC2)no1] | 133 |
| 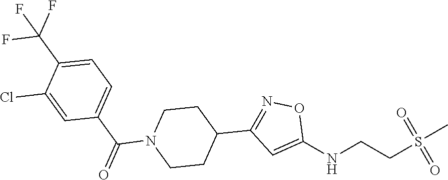 [SMILES: CS(=O)(=O)CCNc1cc(C2CCN(C(=O)c3ccc(C(F)(F)F)c(Cl)c3)CC2)no1] | 134 |
| 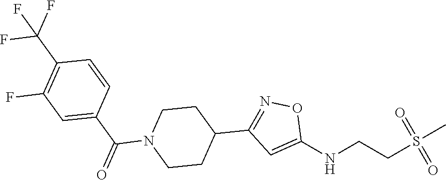 [SMILES: CS(=O)(=O)CCNc1cc(C2CCN(C(=O)c3ccc(C(F)(F)F)c(F)c3)CC2)no1] | 135 |
| 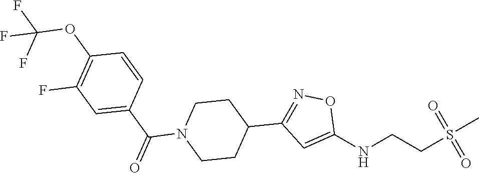 [SMILES: CS(=O)(=O)CCNc1cc(C2CCN(C(=O)c3ccc(OC(F)(F)F)c(F)c3)CC2)no1] | 136 |
| 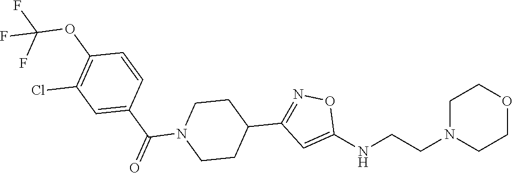 [SMILES: O=C(c1ccc(OC(F)(F)F)c(Cl)c1)N1CCC(c2cc(NCCN3CCOCC3)on2)CC1] | 137 |
| 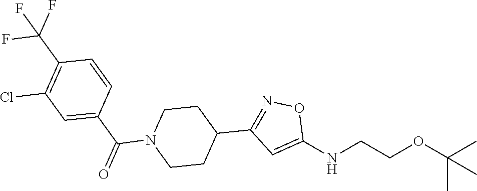 [SMILES: CC(C)(C)OCCNc1cc(C2CCN(C(=O)c3ccc(C(F)(F)F)c(Cl)c3)CC2)no1] | 138 |
| 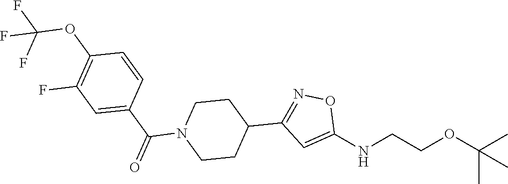 [SMILES: CC(C)(C)OCCNc1cc(C2CCN(C(=O)c3ccc(OC(F)(F)F)c(F)c3)CC2)no1] | 139 |
| 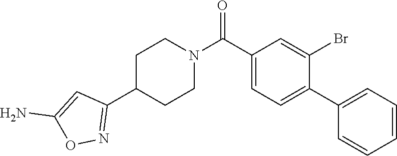 [SMILES: Nc1cc(C2CCN(C(=O)c3ccc(-c4ccccc4)c(Br)c3)CC2)no1] | 140 |
| 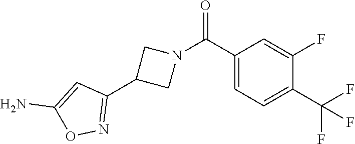 [SMILES: Nc1cc(C2CN(C(=O)c3ccc(C(F)(F)F)c(F)c3)C2)no1] | 141 |
| 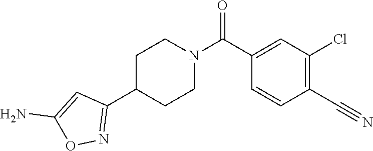 [SMILES: N#Cc1ccc(C(=O)N2CCC(c3cc(N)on3)CC2)cc1Cl] | 142 |
| 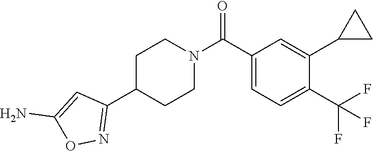 [SMILES: Nc1cc(C2CCN(C(=O)c3ccc(C(F)(F)F)c(C4CC4)c3)CC2)no1] | 143 |
| 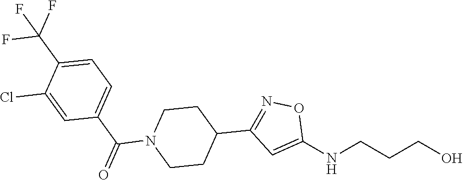 [SMILES: O=C(c1ccc(C(F)(F)F)c(Cl)c1)N1CCC(c2cc(NCCCO)on2)CC1] | 144 |
| 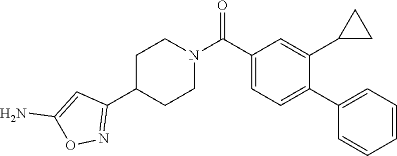 [SMILES: Nc1cc(C2CCN(C(=O)c3ccc(-c4ccccc4)c(C4CC4)c3)CC2)no1] | 145 |
| 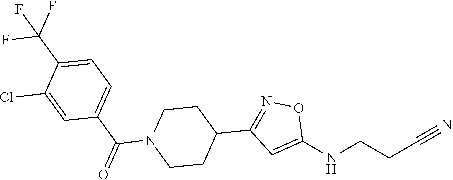 [SMILES: N#CCCNc1cc(C2CCN(C(=O)c3ccc(C(F)(F)F)c(Cl)c3)CC2)no1] | 146 |
| 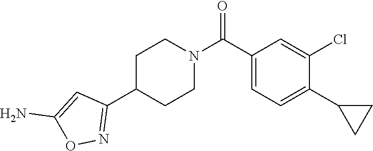 [SMILES: Nc1cc(C2CCN(C(=O)c3ccc(C4CC4)c(Cl)c3)CC2)no1] | 147 |
| 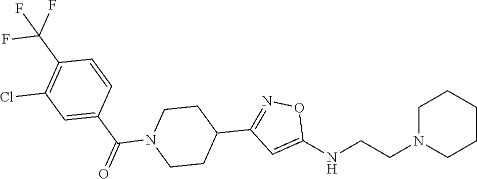 [SMILES: O=C(c1ccc(C(F)(F)F)c(Cl)c1)N1CCC(c2cc(NCCN3CCCCC3)on2)CC1] | 149 |
| 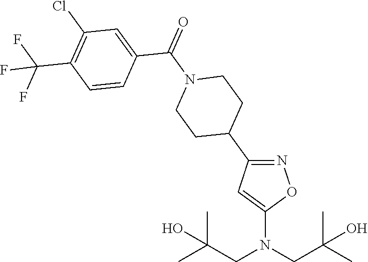 [SMILES: CC(C)(O)CN(CC(C)(C)O)c1cc(C2CCN(C(=O)c3ccc(C(F)(F)F)c(Cl)c3)CC2)no1] | 150 |
| 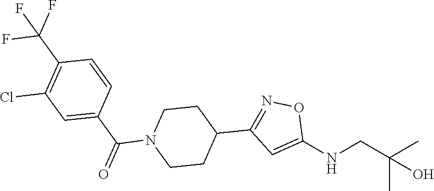 [SMILES: CC(C)(O)CNc1cc(C2CCN(C(=O)c3ccc(C(F)(F)F)c(Cl)c3)CC2)no1] | 151 |
| 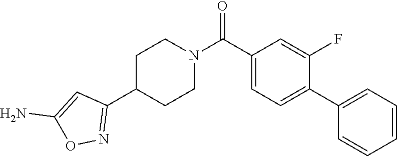 [SMILES: Nc1cc(C2CCN(C(=O)c3ccc(-c4ccccc4)c(F)c3)CC2)no1] | 152 |
| 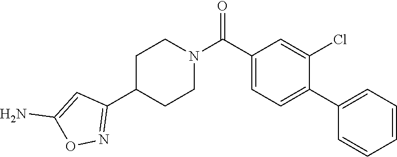 [SMILES: Nc1cc(C2CCN(C(=O)c3ccc(-c4ccccc4)c(Cl)c3)CC2)no1] | 153 |
| 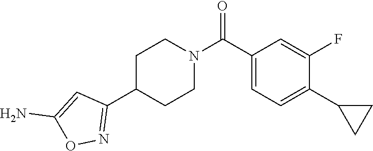 [SMILES: Nc1cc(C2CCN(C(=O)c3ccc(C4CC4)c(F)c3)CC2)no1] | 154 |
| 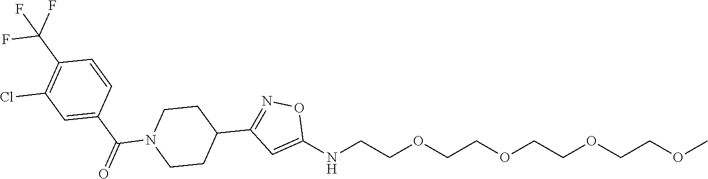 [SMILES: COCCOCCOCCOCCNc1cc(C2CCN(C(=O)c3ccc(C(F)(F)F)c(Cl)c3)CC2)no1] | 155 |
| 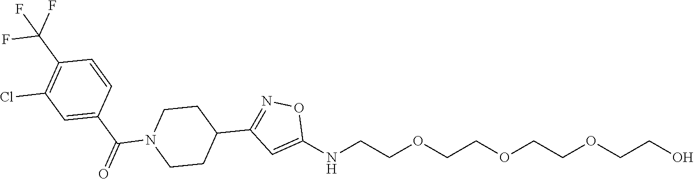 [SMILES: O=C(c1ccc(C(F)(F)F)c(Cl)c1)N1CCC(c2cc(NCCOCCOCCOCCO)on2)CC1] | 156 |
| 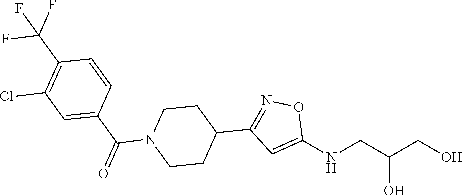 [SMILES: O=C(c1ccc(C(F)(F)F)c(Cl)c1)N1CCC(c2cc(NCC(O)CO)on2)CC1] | 157 |
| 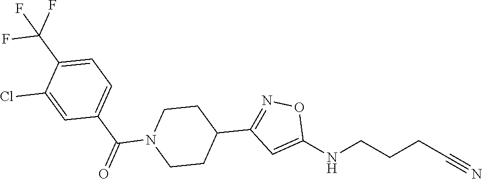 [SMILES: N#CCCCNc1cc(C2CCN(C(=O)c3ccc(C(F)(F)F)c(Cl)c3)CC2)no1] | 158 |
| 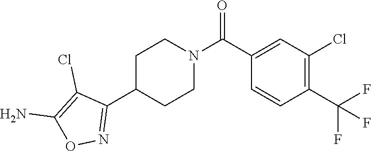 [SMILES: Nc1onc(C2CCN(C(=O)c3ccc(C(F)(F)F)c(Cl)c3)CC2)c1Cl] | 159 |
| 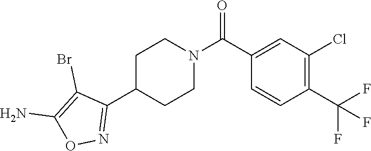 [SMILES: Nc1onc(C2CCN(C(=O)c3ccc(C(F)(F)F)c(Cl)c3)CC2)c1Br] | 160 |
| 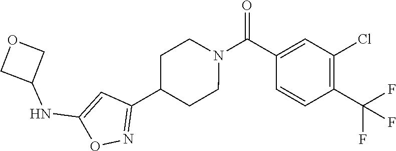 [SMILES: O=C(c1ccc(C(F)(F)F)c(Cl)c1)N1CCC(c2cc(NC3COC3)on2)CC1] | 161 |
| 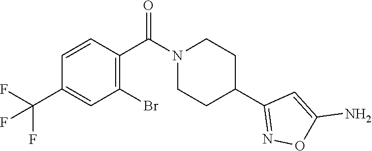 [SMILES: Nc1cc(C2CCN(C(=O)c3ccc(C(F)(F)F)cc3Br)CC2)no1] | 162 |
| 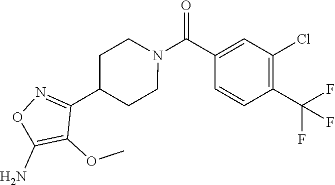 [SMILES: COc1c(C2CCN(C(=O)c3ccc(C(F)(F)F)c(Cl)c3)CC2)noc1N] | 163 |
| 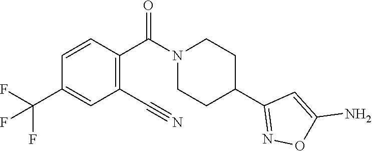 [SMILES: N#Cc1cc(C(F)(F)F)ccc1C(=O)N1CCC(c2cc(N)on2)CC1] | 164 |
| 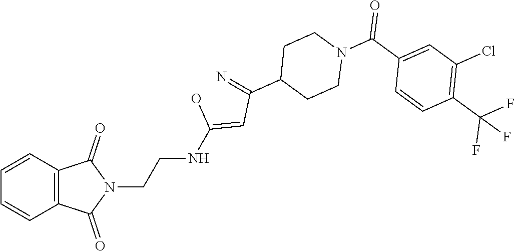 [SMILES: N=C(/C=C(\O)NCCN1C(=O)c2ccccc2C1=O)C1CCN(C(=O)c2ccc(C(F)(F)F)c(Cl)c2)CC1] | 165 |

41. A pharmaceutical composition comprising a compound or pharmaceutically acceptable salt thereof according to any one of claims 15 to 40 and a pharmaceutically acceptable carrier.
